(19) United States
(12) Patent Application Publication
(10) Pub. No.: US 2023/0010886 A1
(43) Pub. Date: Jan. 12, 2023

(54) SHP2 INHIBITORS AND USES THEREOF

(71) Applicant: SUZHOU PUHE BIOPHARMA CO., LTD., Beijing (CN)

(72) Inventors: Yinong XIE, Dover, DE (US); Lee E. BABISS, Dover, DE (US)

(21) Appl. No.: 17/762,590

(22) PCT Filed: Sep. 18, 2020
(86) PCT No.: PCT/US2020/051448

§ 371 Date: Mar. 22, 2022

Related U.S. Application Data
(60) Provisional application No. 62/904,377, filed on Sep. 23, 2019.
(60) Provisional application No. 62/904,493, filed on Sep. 23, 2019.

(51) Int. Cl. A61K 31/506; A61K 31/519; A61K 31/527; A61K 45/06; A61P 35/00

(52) U.S. Cl. CPC A61K 31/506; A61K 31/519; A61K 31/527; A61K 45/06; A61P 35/00

(57) ABSTRACT

Compounds of Formula 1 as inhibitors of protein tyrosine phosphatase SHP2 are disclosed. The pharmaceutical compositions comprising compounds of Formula 1, methods of synthesis of these compounds, methods of treatment for diseases associated with the aberrant activity of SHP2 such as cancer using these compounds or compositions containing these compounds are also disclosed.

### CROSS-REFERENCE TO RELATED APPLICATIONS

This application claims the benefit of U.S. provisional patent application numbers 62/904,377, filed Sep. 23, 2019; and 62/904,493, filed Sep. 23, 2019; all of which are incorporated by reference herein in their entirety.

### FIELD

The present disclosure relates to inhibitors of protein tyrosine phosphatase SHP2 (Src Homolgy-2 phosphatase) and their use in treating SHP2 mediated disorders. More specifically, this disclosure is directed to compounds that inhibit SHP2 and compositions comprising these compounds, methods of treating diseases associated with the aberrant activity of SHP2, and methods of synthesizing these compounds.

### BACKGROUND

Tyrosyl phosphorylation regulates human cellular processes from cell differentiation to growth and apoptosis etc. Tyrosyl phosphorylation is regulated by protein-tyrosine kinases (PTK) and protein-tyrosine phosphatases (PTP). The imbalance of regulation governed by PTK and PTP activity leads to various diseases.

SHP2 is a non-receptor protein tyrosine phosphatase (PTP) encoded by the Protein-tyrosine phosphatase non-receptor type 11 (PTPN11) gene. It contains two N-terminal Src homology 2 domains (N—SH2 and C—SH2), a catalytic domain, and a C-terminal tail. The protein exists in an inactive, auto-inhibited basal conformation that blocks the active site. This self-inhibition state is stabilized by a binding network involving residues from both the N—SH2 and catalytic domains. Stimulation by, for example, cytokines or growth factors results in enzymatic activation of SHP2 and makes the active site available for dephosphorylation of PTPN11 substrates.

SHP2 is widely expressed in most tissues and contributes to various cellular functions including proliferation, differentiation, cell cycle maintenance and migration. It is involved in signaling through the Ras-mitogen-activated protein kinase, the JAK-STAT, EGFR, or the phosphoinositol 3-kinase-AKT pathways.

Mutations in the PTPN11 gene and subsequently in SHP2 lead to hyperactivation of SHP2 catalytic activity, and have been identified in several human diseases, such as Noonan Syndrome, Leopard Syndrome, juvenile myelomonocytic leukemias, neuroblastoma, melanoma, acute myeloid leukemia and cancers of the breast, lung, melanoma, neuroblastoma, hepatocellular carcinoma, and colon. These mutations disrupt the auto-inhibition between the N—SH2 domains and the catalytic site allowing constitutive access of substrates to the catalytic site of the enzyme.

Additionally, there is growing evidence that PTPN11/SHP2 may be implicated in immune evasion during tumorigenesis, and hence a SHP2 inhibitor could stimulate the immune response in cancer patients.

Furthermore, SHP2 plays an important role in JAK/STAT3 pathway, with clear correlation between its phosphatase activity and systemic autoimmunity, thus a SHP2 inhibitor could be used to treat autoimmune diseases such as Lupus and Rheumatoid Arthritis.

Therefore, SHP2 represents a highly attractive target for the development of novel therapies for the treatment of various diseases associated with the aberrant activity of SHP2. The compounds of the present disclosure that are capable of inhibiting the activity of SHP2, possess great potential as novel small molecule therapies for the treatment of various diseases mentioned above.

### SUMMARY

This disclosure relates to compounds represented by Formula 1:

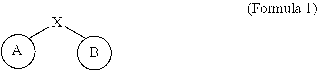

or a pharmaceutically acceptable salt thereof; wherein X is S, O, NR^{A}, CHR^{A}, SO, SO₂, CO, or a bond; Ring A is an optionally substituted aryl, heteroaryl, or bicyclic ring system; Ring B is an optionally substituted heterocyclic ring system, including non-aromatic ring system and heteroaryl, comprising a mono-cyclic ring, a bicyclic ring system, a tricyclic ring system, or a tetracyclic ring system, wherein the heterocyclic ring system contains at least 2 ring nitrogen atoms; and R^{A} is H or C₁₋₁₂ hydrocarbyl.

Some embodiments include a compound represented by Formula 1 or a pharmaceutically acceptable salt thereof; wherein X is S; Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated; Ring B is:

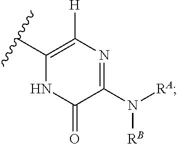

wherein R^{A} and R^{B} are independently H or C₁₋₁₂ hydrocarbyl, or —N(R^{A})(R^{B}) is an optionally substituted heterocyclic ring system, wherein the heterocyclic ring system is: a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system or the tricyclic ring system is a spiro, fused, or bridged ring system, wherein the heterocyclic ring system is saturated or partially saturated; and wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})-, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof.

This disclosure also relates to a method of treating a disease, a disorder, or a condition associated with the aberrant activity of SHP2, comprising administering a therapeutically effective amount of a compound represented by a formula shown below, or a pharmaceutically acceptable salt thereof, to a patient in need thereof,

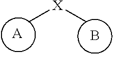

wherein X is S; Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated; Ring B is:

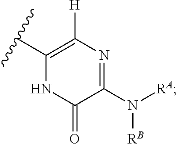

wherein R^{A} and R^{B} are independently H or C₁₋₁₂ hydrocarbyl, or —N(R^{A})(R^{B}) is an optionally substituted heterocyclic ring system, wherein the heterocyclic ring system is: a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system or the tricyclic ring system is a spiro, fused, or bridged ring system, wherein the heterocyclic ring system is saturated or partially saturated; and wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof; and wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

This disclosure also relates to a method of treating a disease, a disorder, or a condition associated with the aberrant activity of SHP2, comprising administering a therapeutically effective amount of a compound represented by a formula shown below, or a pharmaceutically acceptable salt thereof, to a patient in need thereof,

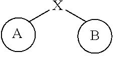

wherein X is S; Ring A is optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, or optionally substituted 3-(4-oxo-6,7,8,9-tetrahydro-4H-pyrido[1,2-a]pyrimidine-3-carboxamido)phenyl; Ring B is optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, optionally substituted 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 6-oxo-5-(2-azaspiro[3.4]octan-2-yl)-1,6-dihydropyrazin-2-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, optionally substituted 6-oxo-5-(spiro[indoline-2,4′-piperidin]-1′-yl)-1,6-dihydropyrazin-2-yl, optionally substituted 3-(1-amino-5-methoxy-1,3-dihydrospiro[indene-2,4′-piperidin]-1′-yl)-2(1H)-one-6-yl, optionally substituted 3-(4-amino-4,6-dihydrospiro[cyclopenta[d]thiazole-5,4′-piperidin]-1′-yl)-2(1H)-one-6-yl, optionally substituted 3-(1-amino-8-azaspiro[4.5]decan-8-yl)pyrazin-2(1H)-one-6-yl, optionally substituted 3-(4-amino-3-methyl-2-oxa-8-azaspiro[4.5]decan-8-yl-4-d)pyrazin-2(1H)-one-6-yl, optionally substituted 3-(1-amino-3-hydroxy-8-azaspiro[4.5]decan-8-yl)pyrazin-2(1H)-one-6-yl, or optionally substituted 5-((3S,4S)-4-amino-3-methyl-2-oxa-8-azaspiro[4.5]decan-8-yl-4-d)-6-oxo-1,6-dihydropyrazin-2-yl; wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof; and wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

Some embodiments include a method of treating diseases, disorders, or conditions associated with the aberrant activity of SHP2, such as but not limited to, cancer, and autoimmune disorders, comprising administering a therapeutically effective amount of a compound described herein, or any optionally substituted compound represented in Table 1 below, or a pharmaceutically acceptable salt thereof (referred to collectively herein as a “subject compound”), to a patient in need thereof.

Some embodiments include use of a compound described herein, such as a compound of Formula 1, a subject compound described herein in the manufacture of a medicament for the treatment of cancer, autoimmune diseases, inflammatory diseases, autoinflammatory conditions, and other SHP2 mediated disorders in a mammal.

Some embodiments include use of a compound represented by a formula,

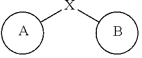

or a pharmaceutically acceptable salt thereof,

in the manufacture of a medicament for treating a disease, a disorder, or a condition associated with the aberrant activity of SHP2, wherein a therapeutically effective amount of the compound or the pharmaceutically acceptable salt is administered to a patient in need thereof, wherein X is S; Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated; Ring B is:

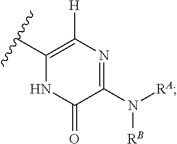

wherein R^{A} and R^{B} are independently H or C₁₋₁₂ hydrocarbyl, or —N(R^{A})(R^{B}) is an optionally substituted heterocyclic ring system, wherein the heterocyclic ring system is: a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system is a spiro, fused, or bridged ring system, wherein the heterocyclic ring system is saturated or partially saturated; wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A})(R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl, —C(O)N(R^{A})-heterocyclyl, or a combination thereof, and wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.

Some embodiments include use of a compound represented by a formula,

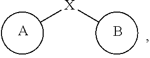

or a pharmaceutically acceptable salt thereof,
in the manufacture of a medicament for treating a disease, a disorder, or a condition associated with the aberrant activity of SHP2, wherein a therapeutically effective amount of the compound or the pharmaceutically acceptable salt is administered to a patient in need thereof, wherein X is S; wherein Ring A is optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, or optionally substituted 3-(4-oxo-6,7,8,9-tetrahydro-4H-pyrido[1,2-a]pyrimidine-3-carboxamido)phenyl; wherein Ring B is optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, optionally substituted 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 6-oxo-5-(2-azaspiro[3.4]octan-2-yl)-1,6-dihydropyrazin-2-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, optionally substituted 6-oxo-5-(spiro[indoline-2,4′-piperidin]-1′-yl)-1,6-dihydropyrazin-2-yl, optionally substituted 3-(1-amino-5-methoxy-1,3-dihydrospiro[indene-2,4′-piperidin]-1′-yl)-2(1H)-one-6-yl, optionally substituted 3-(4-amino-4,6-dihydrospiro[cyclopenta[d]thiazole-5,4′-piperidin]-1′-yl)-2(1H)-one-6-yl, optionally substituted 3-(1-amino-8-azaspiro[4.5]decan-8-yl)pyrazin-2(1H)-one-6-yl, optionally substituted 3-(4-amino-3-methyl-2-oxa-8-azaspiro[4.5]decan-8-yl-4-d)pyrazin-2(1H)-one-6-yl, optionally substituted 3-(1-amino-3-hydroxy-8-azaspiro[4.5]decan-8-yl)pyrazin-2(1H)-one-6-yl, or optionally substituted 5-((3S,4S)-4-amino-3-methyl-2-oxa-8-azaspiro[4.5]decan-8-yl-4-d)-6-oxo-1,6-dihydropyrazin-2-yl; and wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof; wherein each substituent has 0-20 carbon atoms and 0-10 heteroatoms, and wherein each heteroatom is independently N, O, S, F, C₁, or Br, provided that the substituent includes at least one C, N, O, S, F, C₁, or Br; and wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

Some embodiments include a pharmaceutical composition comprising a therapeutically effective amount of a subject compound described herein, or a pharmaceutically acceptable salt thereof, in combination with at least one pharmaceutically acceptable vehicle, diluent, or carrier.

Some embodiments include a process for making a pharmaceutical composition comprising combining a subject compound described herein and at least one pharmaceutically acceptable carrier.

Some embodiments include a medicament comprising a composition comprising a therapeutically effective amount of a subject compound.

Some embodiments include a kit comprising a medicament of above and a label indicating that the medicament is for treating a disease, disorders, or condition associated with the aberrant activity of SHP2.

Some embodiments include a subject compound described herein having superior anti-proliferation activities in various tumor types, for example lung cancer, esophageal cancer, pancreatic cancer, caecum cancer, head and neck cancer, colon cancer, melanoma, leukemia, or other metastatic solid tumors.

Some embodiments include a subject compound described herein that is very potent and selective with enzymatic IC₅₀ less than 10 nM; and has superior anti-tumor activities in various in vivo animal models. The administration of a subject compound described herein with a dose amount falls within the range of 1 mg/kg per day to 100 mg/kg per day could achieve the tumor regression or at least about 70% tumor growth inhibition in various in vivo animal models, such as but not limited to, KYSE-520 Xenograft Model, lung cancer H-358 Xenograft Model, pancreatic cancer Mia-Pa-Ca-2 Xenograft Model, and non-small cell lung cancer (NSCLC) with KRAS mutant Xenograft Model, etc.

Some embodiments include a method of treating diseases, disorders, or conditions associated with the aberrant activity of SHP2, such as but not limited to, cancer, and autoimmune disorders, such as but not limited to Lung cancer, non-small cell lung cancer, non-small cell lung cancer with KRAS mutant, esophageal cancer, pancreatic cancer, and caecum cancer, head and neck cancer, colon cancer, melanoma, leukemia, or other metastatic solid tumors comprising administering a therapeutically effective amount of a subject compound described herein, to a patient in need thereof. The patient can be a mammal such as an animal or a human being.

The SHP2 inhibitors described herein could provide advantageous therapeutic benefit, either alone or in combination with other therapeutic agents, for the treatment of various disease, disorders, or condition associated with the aberrant activity of SHP2, including cancer and autoimmune disorders, such as but not limited to Lung cancer, non-small cell lung cancer, non-small cell lung cancer with KRAS mutant, esophageal cancer, pancreatic cancer, and caecum cancer. In some embodiments, a combination of two SHP2 inhibitors described herein, such as a compound of Formula 1, provides higher efficacy in treating cancer and autoimmune disorders than either SHP2 inhibitor alone in mammals. In some embodiments, the combination of a SHP2 inhibitor described herein, such as a compound of Formula 1, with other agent such as a CDK4/6 inhibit or a MEK inhibitor, could provide higher efficacy in treating cancer and autoimmune disorders than the single agent alone in mammals.

### BRIEF DESCRIPTION OF THE DRAWINGS

FIG. 1 depicts the tumor volumes over the time after the start of treatment with compound 14 (SYB-020070), a reference compound of RMC-4550 (SYB-020078), and vehicle control in mice in an esophageal KYSE-520 Xenograft Model.

FIG. 2 depicts the tumor volumes over the time after the start of treatment with compound 14 (SYB-020070), compound 77 (SYB-020083), a reference compound of RMC-4550 (SYB-020078), and vehicle control in mice in lung cancer H-358 Xenograft studies.

FIG. 3 depicts the tumor volumes over the time after the start of treatment with compound 14 (SYB-020070), compound 77 (SYB-020083), alone or in combination with a MEK inhibitor SYB-020099 or a CDK4/6 inhibitor SYB-020097, and a reference compound of RMC-4550 (SYB-020078), and vehicle control in mice in pancreatic cancer Mia-Pa—Ca-2 Xenograft studies.

FIG. 4 depicts the tumor volumes over the time after the start of treatment with compound 14 (SYB-020070), alone or in combination a CDK4/6 inhibitor SYB-020097, and vehicle control in mice in non-small cell lung cancer (NSCLC) with KRAS mutant Xenograft studies.

### DETAILED DESCRIPTION

Unless otherwise indicated, any reference to a compound herein by structure, name, or any other means, includes pharmaceutically acceptable salts, such as sodium, potassium, and ammonium salts, or HCl, H₂SO₄, HCO₂H, and CF₃CO₂H salts; prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

If stereochemistry is not indicated, a name or structural depiction includes any stereoisomer or any mixture of stereoisomers.

Unless otherwise indicated, when a compound or chemical structural feature such as aryl is referred to as being “optionally substituted,” it includes a feature that has no substituents (i.e. unsubstituted), or a feature that is “substituted,” meaning that the feature has one or more substituents. The term “substituent” is broad, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of 15 g/mol to 50 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, a substituent may be an ordinary organic moiety known in the art, which may have a molecular weight of 15 g/mol to 200 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, C₁, Br, or I; provided that the substituent includes one C, N, O, S, P, Si, F, C₁, Br, or I atom. Examples of substituents include, but are not limited to, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, 0-carbamyl, N-carbamyl, 0-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, amino, phosphonic acid, etc.

For convenience, the term “molecular weight” is used with respect to a moiety or part of a molecule to indicate the sum of the atomic masses of the atoms in the moiety or part of a molecule, even though it may not be a complete molecule.

The term “treating” or “treatment” includes the diagnosis, cure, mitigation, treatment, or prevention of disease in man or other animals, or any activity that otherwise affects the structure or any function of the body of man or other animals.

A hydrogen atom in any position of a compound of Formula 1 may be replaced by a deuterium. In some embodiments, a compound of Formula 1 contains a deuterium atom or multiple deuterium atoms.

Some embodiments include a compound represented by Formula 1 or a pharmaceutically acceptable salt thereof; wherein X is S; Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated.

When Ring A is substituted, the substituted Ring A has one or more substituents. Each substituent of Ring A is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A})(R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof.

With respect to Formula 1, in some embodiments, Ring A is: optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, or optionally substituted 3-(4-oxo-6,7,8,9-tetrahydro-4H-pyrido[1,2-a]pyrimidine-3-carboxamido)phenyl. For example, Ring A may be 2,3-dichlorophenyl, 2,3-dichloropyridin-4-yl, or 2-amino-3-chloropyridin-4-yl.

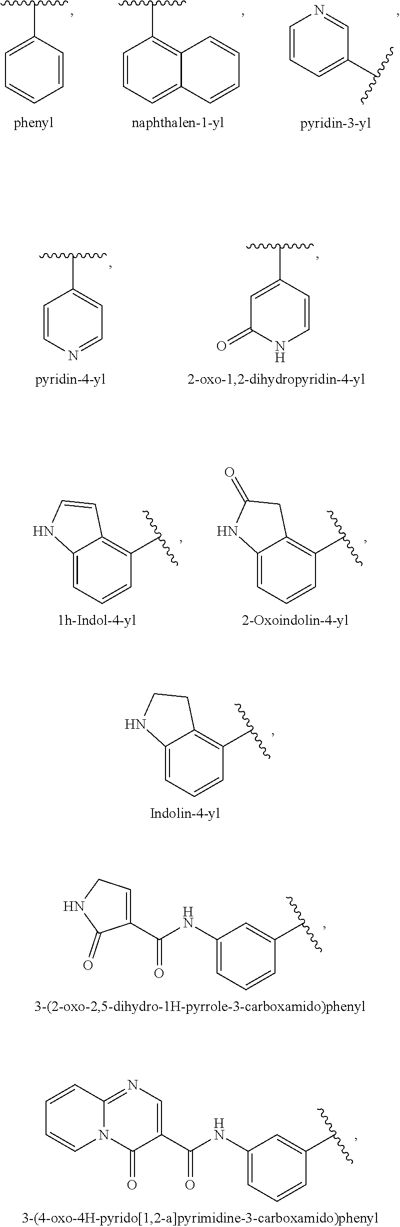

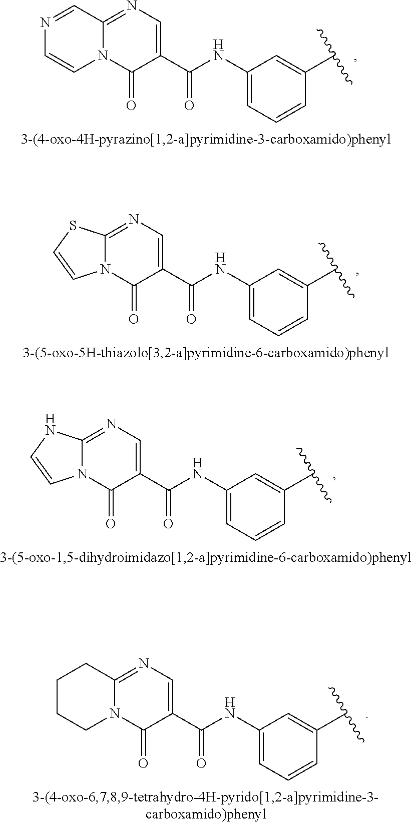 [SMILES: *c1cccc(NC(=O)c2cnc3cnccn3c2=O)c1 ; *c1cccc(NC(=O)c2cnc3sccn3c2=O)c1 ; *c1cccc(NC(=O)c2cnc3[nH]ccn3c2=O)c1 ; *c1cccc(NC(=O)c2cnc3n(c2=O)CCCC3)c1]

In some embodiments, Ring A is 6-membered aromatic ring.

In some embodiments, Ring A is 6-membered phenyl ring or heteroaryl ring.

In some embodiments, Ring A is 6-membered phenyl ring.

In some embodiments, Ring A is 6-membered heteroaryl ring.

In some embodiments, Ring A is 6-membered heteroaryl ring containing one ring N atom.

In some embodiments, Ring A is 6-membered phenyl ring or 6-membered heteroaryl ring containing one ring N atom.

In some embodiments, Ring A is a substituted pyridine.

In some embodiments, Ring A is a substituted phenyl.

In some embodiments, Ring A has 1 or 2 substituents.

In some embodiments, Ring A has 2 substituents.

In some embodiments, Ring A is unsubstituted.

In some embodiments, Ring A has a Cl substituent.

In some embodiments, Ring A has two Cl substituents.

In some embodiments, Ring A has two Cl substituents at 2- and 3-positions; for example, Ring A is 2,3-dichlorophenyl.

In some embodiments, Ring A has a CF₃ substituent.

In some embodiments, Ring A has a CF₃ substituent at 2-position.

In some embodiments, Ring A has an NH₂ substituent.

In some embodiments, Ring A has an NH₂ substituent and a Cl substituent.

In some embodiments, Ring A has an NH₂ substituent and a Cl substituent with Cl at 2-position and NH₂ at 5-position.

In some embodiments, Ring A has an NH₂ substituent and a Cl substituent with Cl at 2-position and NH₂ at 3-position.

In some embodiments, Ring A has an NH₂ substituent and a Cl substituent with Cl at 3-position and NH₂ at 2-position.

In some embodiments, Ring A has an —OCH₃ substituent.

In some embodiments, Ring A has an —OCH₃ substituent and a Cl substituent.

In some embodiments, Ring A has an —OCH₃ substituent and a Cl substituent with Cl at 2-position and —OCH₃ at 3-position.

In some embodiments, Ring A has an F substituent.

In some embodiments, Ring A has two F substituents.

In some embodiments, Ring A has two F substituents at same position.

In some embodiments, Ring A has two F substituents at 2- and 3-positions.

In some embodiments, Ring A has an F substituent and a Cl substituent.

In some embodiments, Ring A has an F substituent and a Cl substituent with Cl at 2-position and F at 3-position.

In some embodiments, Ring A has an acetyl substituent.

In some embodiments, Ring A has a CH₃ substituent.

In some embodiments, Ring A has two CH₃ substituents that are at same position.

In some embodiments, Ring A has a CH₃ substituent and a C₁ substituent.

In some embodiments, Ring A has a CH₃ substituent and a C₁ substituent with Cl at 2-position and CH₃ at 4-position.

In some embodiments, Ring A has a CH₃ substituent and a Cl substituent with Cl at 2-position and CH₃ at 3-position.

In some embodiments, Ring A has a CH₃ substituent and two F substituents.

In some embodiments, Ring A has a CH₃ substituent and two F substituents with two F at same position.

In some embodiments, Ring A has an OH substituent.

In some embodiments, Ring A has an OH substituent and a Cl substituent.

In some embodiments, Ring A has multiple substituents with any combination of the above substituents.

Some embodiments include a compound represented by Formula 1 or a pharmaceutically acceptable salt thereof, wherein Ring A is any one of the following:

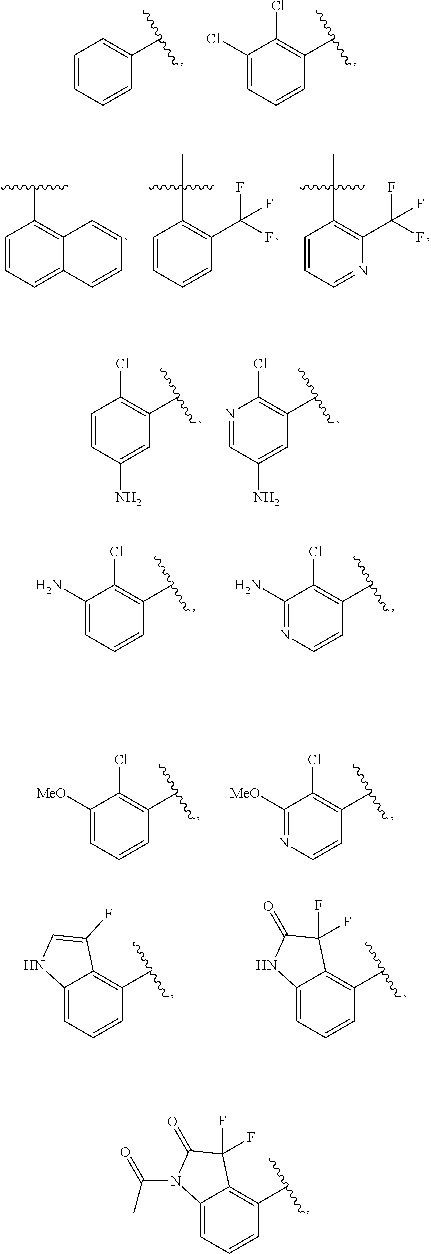

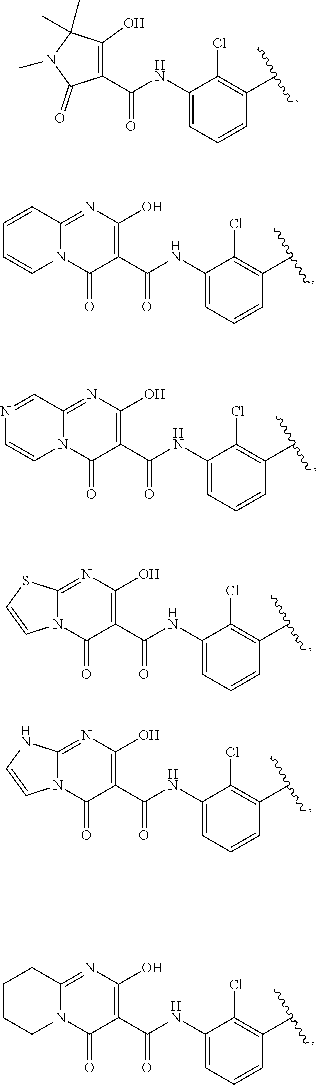 [SMILES: *c1cccc(NC(=O)C2=C(O)C(C)(C)N(C)C2=O)c1Cl ; *c1cccc(NC(=O)c2c(O)nc3ccccn3c2=O)c1Cl ; *c1cccc(NC(=O)c2c(O)nc3cnccn3c2=O)c1Cl ; *c1cccc(NC(=O)c2c(O)nc3sccn3c2=O)c1Cl ; *c1cccc(NC(=O)c2c(O)nc3[nH]ccn3c2=O)c1Cl ; *c1cccc(NC(=O)c2c(O)nc3n(c2=O)CCCC3)c1Cl]

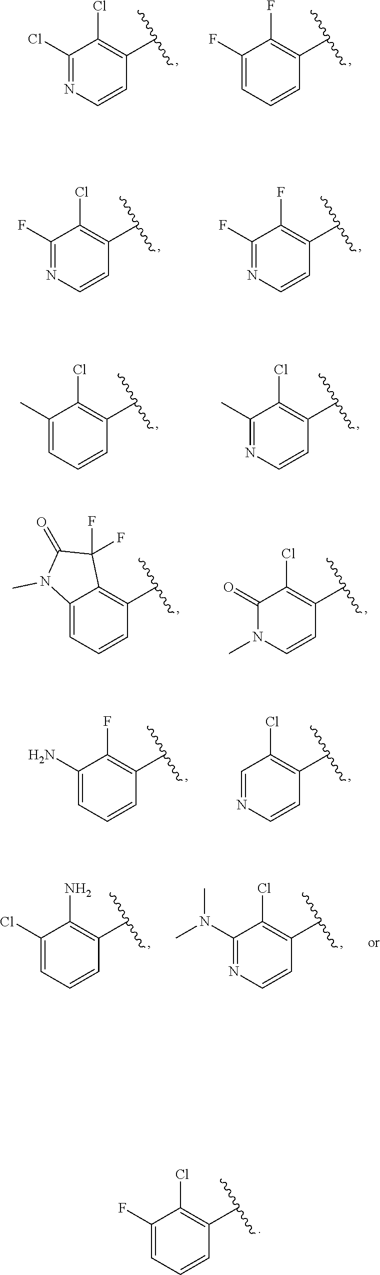

Some embodiments include a compound represented by Formula 1 or a pharmaceutically acceptable salt thereof; wherein X is S; and Ring B is:

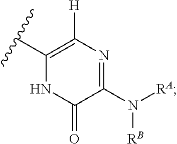

wherein R^{A} and R^{B} are independently H or C₁₋₁₂ hydrocarbyl, or —N(R^{A})(R^{B}) is an optionally substituted heterocyclic ring system, wherein the heterocyclic ring system is a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system is a spiro, fused, or bridged ring system, wherein the heterocyclic ring system is saturated or partially saturated.

When the Ring B is substituted, the substituted Ring B has one or more substituents. Each substituent of Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof.

With respect to Formula 1, in some embodiments, Ring B is: optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 4-oxo-6-(piperidin-1-yl)-4,5-dihydro-1H-pyrazolo[3,4-d]pyrimidin-3-yl, 4-oxo-6-(pyrrolidin-1-yl)-4,5-dihydro-1H-pyrazolo[3,4-d]pyrimidin-3-yl, optionally substituted 4-oxo-2-(piperidin-1-yl)-3,4-dihydroquinazolin-5-yl, optionally substituted 4-oxo-2-(piperidin-1-yl)-3,4-dihydropyrido[3,4-d]pyrimidin-5-yl, optionally substituted 7-oxo-2-(piperidin-1-yl)-7,8-dihydropyrido[2,3-d]pyrimidin-5-yl, optionally substituted 5-(piperidin-1-yl)-1H-pyrazolo[4,3-d]thiazol-3-yl, optionally substituted 7-oxo-6-(piperidin-4-yl)-6,7-dihydro-1H-pyrazolo[4,3-d]pyrimidin-3-yl, optionally substituted 6-oxo-8-(piperidin-1-yl)-6,7-dihydro-1H-purin-2-yl, optionally substituted 8-(piperidin-1-yl)-7H-purin-2-yl, optionally substituted 6-oxo-2-(pyrrolidin-1-yl)-1,6-dihydropyrimidin-5-yl, optionally substituted 6-oxo-2-(piperidin-1-yl)-1,6-dihydropyrimidin-5-yl, optionally substituted 6-oxo-2-(2-oxa-8-azaspiro[4.5]decan-8-yl)-1,6-dihydropyrimidin-5-yl, optionally substituted 2-(3,6-diazabicyclo[3.2.0]heptan-6-yl)-6-oxo-1,6-dihydropyrimidin-5-yl, optionally substituted 2-(hexahydropyrrolo[3,4-c]pyrrol-2(1H)-yl)-6-oxo-1,6-dihydropyrimidin-5-yl, optionally substituted 2-(3-azabicyclo[3.2.0]heptan-3-yl)-6-oxo-1,6-dihydropyrimidin-5-yl, optionally substituted 6-oxo-2-(2-azaspiro[3.4]octan-2-yl)-1,6-dihydropyrimidin-5-yl, optionally substituted 2-(3-azabicyclo[3.1.0]hexan-3-yl)-6-oxo-1,6-dihydropyrimidin-5-yl, optionally substituted 6-oxo-2-(2-azaspiro[3.4]octan-2-yl)-1,6-dihydropyrimidin-5-yl, optionally substituted 5-oxo-6-(2-oxa-8-azaspiro[4.5]decan-8-yl)-4,5-dihydro-1H-pyrazolo[3,4-b]pyrazin-3-yl, optionally substituted 5-oxo-6-(piperidin-1-yl)-4,5-dihydro-1H-pyrazolo[3,4-b]pyrazin-3-yl, optionally substituted 6-(piperidin-1-yl)-1H-pyrazolo[3,4-d]pyrimidin-3-yl, optionally substituted 6-oxo-5-(2-azaspiro[3.4]octan-2-yl)-1,6-dihydropyrazin-2-yl, optionally substituted 1-cyclohexyl-2-oxo-1,2-dihydropyridin-4-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 4-oxo-2-(2-oxa-8-azaspiro[4.5]decan-8-yl)-4,7-dihydro-3H-pyrrolo[2,3-d]pyrimidin-5-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, or optionally substituted 6-oxo-5-(spiro[indoline-2,4′-piperidin]-1′-yl)-1,6-dihydropyrazin-2-yl. The core structures for some suitable Ring B groups are listed in Table 1A below.

TABLE 1A

| Structure | Name |
|---|---|
| 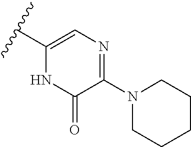 [SMILES: *c1cnc(N2CCCCC2)c(=O)[nH]1] | 6-oxo-5-(piperidin-1-yl)-1,6-dihydropyrazin-2-yl |
| 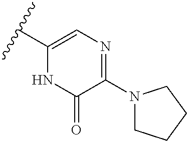 [SMILES: *c1cnc(N2CCCC2)c(=O)[nH]1] | 6-oxo-5-(pyrrolidin-1-yl)-1,6-dihydropyrazin-2-yl |
| 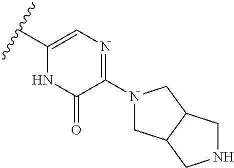 [SMILES: *c1cnc(N2CC3CNCC3C2)c(=O)[nH]1] | 5-(hexahydropyrrolo[3,4-c]pyrrol-2(1H)-yl)-6-oxo-1,6- dihydropyrazin-2-yl |
| 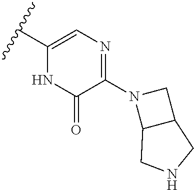 [SMILES: *c1cnc(N2CC3CNCC32)c(=O)[nH]1] | 5-(3,6-diazabicyclo[3.2.0]heptan-6-yl)-6-oxo-1,6- dihydropyrazin-2-yl |
| 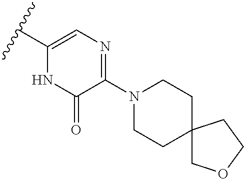 [SMILES: *c1cnc(N2CCC3(CCOC3)CC2)c(=O)[nH]1] | 6-oxo-5-(2-oxa-8-azaspiro[4.5]decan-8-yl)-1,6- dihydropyrazin-2-yl |
| 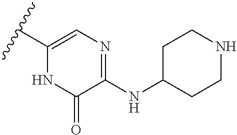 [SMILES: *c1cnc(NC2CCNCC2)c(=O)[nH]1] | 6-oxo-5-(piperidin-4-ylamino)-1,6-dihydropyrazin-2-yl |
| 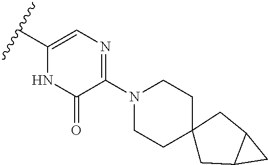 [SMILES: *c1cnc(N2CCC3(CC2)CC2CC2C3)c(=O)[nH]1] | 6-oxo-5-(spiro[bicyclo[3.1.0]hexane-3,4′-piperidin]-1′- yl)-1,6-dihydropyrazin-2-yl |
| 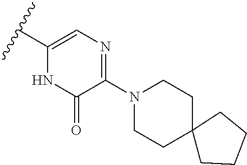 [SMILES: *c1cnc(N2CCC3(CCCC3)CC2)c(=O)[nH]1] | 6-oxo-5-(8-azaspiro[4.5]decan-8-yl)-1,6-dihydropyrazin- 2-yl |
| 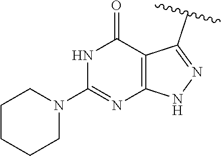 [SMILES: *c1n[nH]c2nc(N3CCCCC3)[nH]c(=O)c12] | 4-oxo-6-(piperidin-1-yl)-4,5-dihydro-1H-pyrazolo[3,4- d]pyrimidin-3-yl |
| 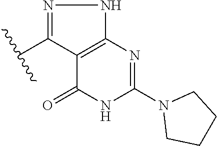 [SMILES: *c1n[nH]c2nc(N3CCCC3)[nH]c(=O)c12] | 4-oxo-6-(pyrrolidin-1-yl)-4,5-dihydro-1H-pyrazolo[3,4- d]pyrimidin-3-yl |
| 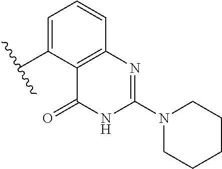 [SMILES: *c1cccc2nc(N3CCCCC3)[nH]c(=O)c12] | 4-oxo-2-(piperidin-1-yl)-3,4-dihydroquinazolin-5-yl |
| 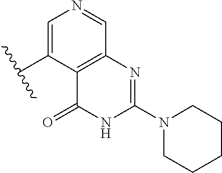 [SMILES: *c1cncc2nc(N3CCCCC3)[nH]c(=O)c12] | 4-oxo-2-(piperidin-1-yl)-3,4-dihydropyrido[3,4-d]- pyrimidin-5-yl |
| 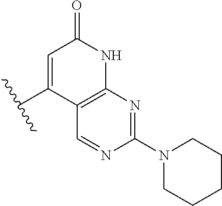 [SMILES: *c1cc(=O)[nH]c2nc(N3CCCCC3)ncc12] | 7-oxo-2-(piperidin-1-yl)-7,8-dihydropyrido[2,3-d]- pyrimidin-5-yl |
| 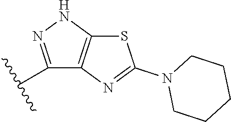 [SMILES: *c1n[nH]c2sc(N3CCCCC3)nc12] | 5-(piperidin-1-yl)-1H-pyrazolo[4,3-d]thiazol-3-yl |
| 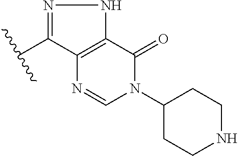 [SMILES: *c1n[nH]c2c(=O)n(C3CCNCC3)cnc12] | 7-oxo-6-(piperidin-4-yl)-6,7-dihydro-1H-pyrazolo[4,3- d]pyrimidin-3-yl |
| 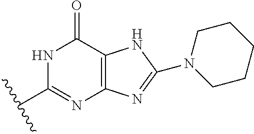 [SMILES: *c1nc2nc(N3CCCCC3)[nH]c2c(=O)[nH]1] | 6-oxo-8-(piperidin-1-yl)-6,7-dihydro-1H-purin-2-yl |
| 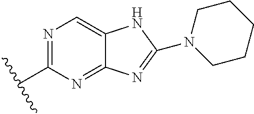 [SMILES: *c1ncc2[nH]c(N3CCCCC3)nc2n1] | 8-(piperidin-1-yl)-7H-purin-2-yl |
| 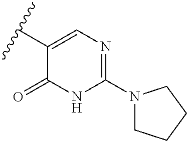 [SMILES: *c1cnc(N2CCCC2)[nH]c1=O] | 6-oxo-2-(pyrrolidin-1-yl)-1,6-dihydropyrimidin-5-yl |
| 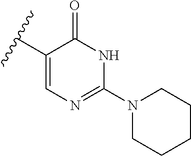 [SMILES: *c1cnc(N2CCCCC2)[nH]c1=O] | 6-oxo-2-(piperidin-1-yl)-1,6-dihydropyrimidin-5-yl |
| 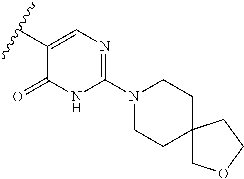 [SMILES: *c1cnc(N2CCC3(CCOC3)CC2)[nH]c1=O] | 6-oxo-2-(2-oxa-8-azaspiro[4.5]decan-8-yl)-1,6- dihydropyrimidin-5-yl |
| 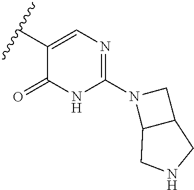 [SMILES: *c1cnc(N2CC3CNCC32)[nH]c1=O] | 2-(3,6-diazabicyclo[3.2.0]heptan-6-yl)-6-oxo-1,6- dihydropyrimidin-5-yl |
| 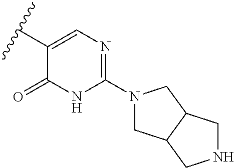 [SMILES: *c1cnc(N2CC3CNCC3C2)[nH]c1=O] | 2-(hexahydropyrrolo[3,4-c]pyrrol-2(1H)-yl)-6-oxo-1,6- dihydropyrimidin-5-yl |
| 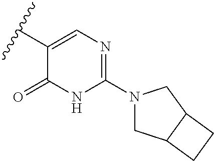 [SMILES: *c1cnc(N2CC3CCC3C2)[nH]c1=O] | 2-(3-azabicyclo[3.2.0]heptan-3-yl)-6-oxo-1,6- dihydropyrimidin-5-yl |
| 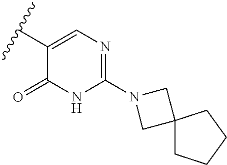 [SMILES: *c1cnc(N2CC3(CCCC3)C2)[nH]c1=O] | 6-oxo-2-(2-azaspiro[3.4]octan-2-yl)-1,6-dihydro- pyrimidin-5-yl |
| 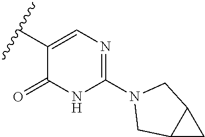 [SMILES: *c1cnc(N2CC3CC3C2)[nH]c1=O] | 2-(3-azabicyclo[3.1.0]hexan-3-yl)-6-oxo-1,6- dihydropyrimidin-5-yl |
| 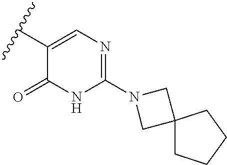 [SMILES: *c1cnc(N2CC3(CCCC3)C2)[nH]c1=O] | 6-oxo-2-(2-azaspiro[3.4]octan-2-yl)-1,6-dihydro- pyrimidin-5-yl |
| 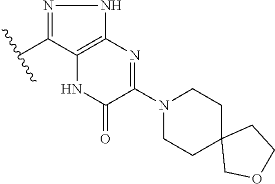 [SMILES: *c1n[nH]c2nc(N3CCC4(CCOC4)CC3)c(=O)[nH]c12] | 5-oxo-6-(2-oxa-8-azaspiro[4.5]decan-8-yl)-4,5-dihydro- 1H-pyrazolo[3,4-b]pyrazin-3-yl |
| 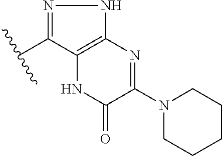 [SMILES: *c1n[nH]c2nc(N3CCCCC3)c(=O)[nH]c12] | 5-oxo-6-(piperidin-1-yl)-4,5-dihydro-1H-pyrazolo[3,4- b]pyrazin-3-yl |
| 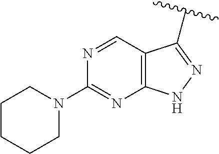 [SMILES: *c1n[nH]c2nc(N3CCCCC3)ncc12] | 6-(piperidin-1-yl)-1H-pyrazolo[3,4-d]pyrimidin-3-yl |
| 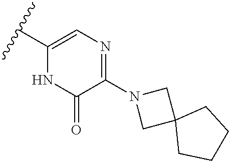 [SMILES: *c1cnc(N2CC3(CCCC3)C2)c(=O)[nH]1] | 6-oxo-5-(2-azaspiro[3.4]octan-2-yl)-1,6-dihydropyrazin- 2-yl |
| 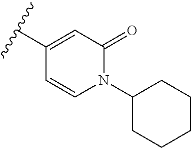 [SMILES: *c1ccn(C2CCCCC2)c(=O)c1] | 1-cyclohexyl-2-oxo-1,2-dihydropyridin-4-yl |
| 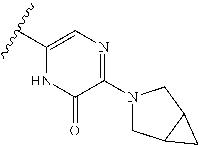 [SMILES: *c1cnc(N2CC3CC3C2)c(=O)[nH]1] | 5-(3-azabicyclo[3.1.0]hexan-3-yl)-6-oxo-1,6- dihydropyrazin-2-yl |
| 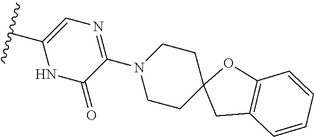 [SMILES: *c1cnc(N2CCC3(CC2)Cc2ccccc2O3)c(=O)[nH]1] | 6-oxo-5-(3H-spiro[benzofuran-2,4′-piperidin]-1′-yl)-1,6- dihydropyrazin-2-yl |
| 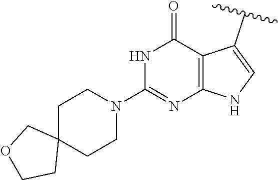 [SMILES: *c1c[nH]c2nc(N3CCC4(CCOC4)CC3)[nH]c(=O)c12] | 4-oxo-2-(2-oxa-8-azaspiro[4.5]decan-8-yl)-4,7-dihydro- 3H-pyrrolo[2,3-d]pyrimidin-5-yl |
| 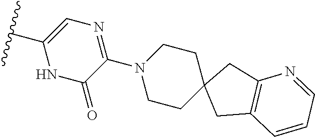 [SMILES: *c1cnc(N2CCC3(CC2)Cc2cccnc2C3)c(=O)[nH]1] | 5-(5,7-dihydrospiro[cyclopenta[b]pyridine-6,4′- piperidin]-1′-yl)-6-oxo-1,6-dihydropyrazin-2-yl |
| 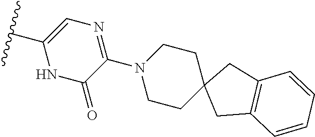 [SMILES: *c1cnc(N2CCC3(CC2)Cc2ccccc2C3)c(=O)[nH]1] | 5-(1,3-dihydrospiro[indene-2,4′-piperidin]-1′-yl)-6-oxo- 1,6-dihydropyrazin-2-yl |
| 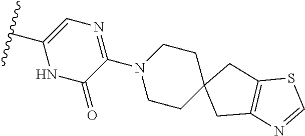 [SMILES: *c1cnc(N2CCC3(CC2)Cc2ncsc2C3)c(=O)[nH]1] | 5-(4,6-dihydrospiro[cyclopenta[d]thiazole-5,4′- piperidin]-1′-yl)-6-oxo-1,6-dihydropyrazin-2-yl |
| 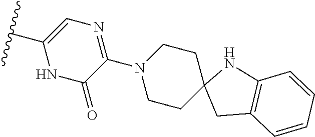 [SMILES: *c1cnc(N2CCC3(CC2)Cc2ccccc2N3)c(=O)[nH]1] | 6-oxo-5-(spiro[indoline-2,4′-piperidin]-1′-yl)-1,6- dihydropyrazin-2-yl |

In some embodiments, Ring B is a bicyclic or a tricyclic ring system.

In some embodiments, Ring B is a bicyclic ring system.

In some embodiments, Ring B is a deuterated bicyclic ring system.

In some embodiments, Ring B is a tricyclic ring system.

In some embodiments, Ring B is a bicyclic or a tricyclic ring system, having a —NH₂ substituent.

In some embodiments, Ring B is a spiro bicyclic ring system.

In some embodiments, Ring B is a spiro bicyclic ring system.

In some embodiments, Ring B is tricyclic ring system containing a spiro bicyclic ring system.

In some embodiments, Ring B is unsubstituted.

In some embodiments, Ring B has a —CH₃ substituent.

In some embodiments, Ring B has a —CH₂NH₂ substituent.

In some embodiments, Ring B has a —NH₂ substituent.

In some embodiments, Ring B has a —CH₂CH₂NH₂ substituent.

In some embodiments, Ring B has a 1-aminopropan-2-yl substituent.

In some embodiments, Ring B has a —CN substituent.

In some embodiments, Ring B has an —F substituent.

In some embodiments, Ring B has a —Cl substituent.

In some embodiments, Ring B has a —CH₂F substituent.

In some embodiments, Ring B has an —OH substituent.

In some embodiments, Ring B has an —OCH₃ substituent.

In some embodiments, Ring B has multiple substituents with any combination of the above substituents.

Some embodiments include a compound represented by Formula 1 or a pharmaceutically acceptable salt thereof, wherein Ring B is any one of the following:

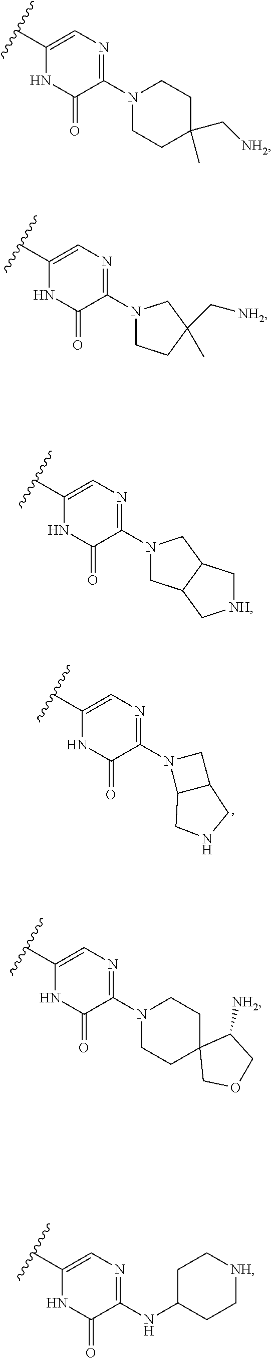 [SMILES: *c1cnc(N2CCC(C)(CN)CC2)c(=O)[nH]1 ; *c1cnc(N2CCC(C)(CN)C2)c(=O)[nH]1 ; *c1cnc(N2CC3CNCC3C2)c(=O)[nH]1 ; *c1cnc(N2CC3CNCC32)c(=O)[nH]1 ; *c1cnc(N2CCC3(CC2)COC[C@H]3N)c(=O)[nH]1 ; *c1cnc(NC2CCNCC2)c(=O)[nH]1]

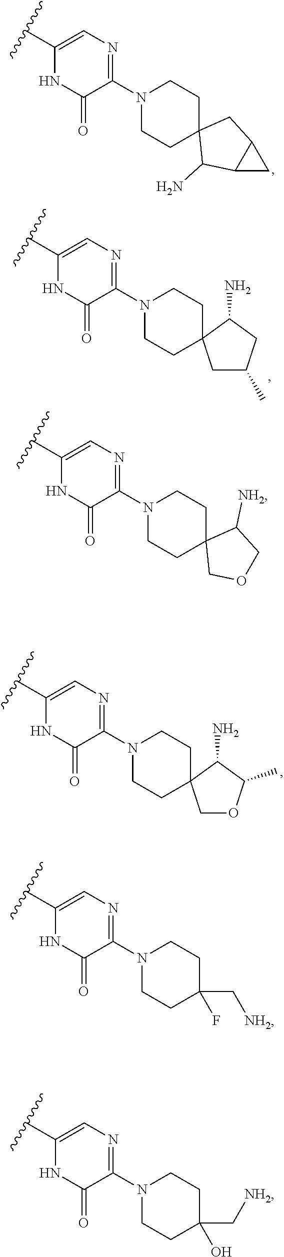 [SMILES: *c1cnc(N2CCC3(CC2)CC2CC2C3N)c(=O)[nH]1 ; *c1cnc(N2CCC3(CC2)C[C@@H](C)C[C@H]3N)c(=O)[nH]1 ; *c1cnc(N2CCC3(CC2)COCC3N)c(=O)[nH]1 ; *c1cnc(N2CCC3(CC2)CO[C@@H](C)[C@H]3N)c(=O)[nH]1 ; *c1cnc(N2CCC(F)(CN)CC2)c(=O)[nH]1 ; *c1cnc(N2CCC(O)(CN)CC2)c(=O)[nH]1]

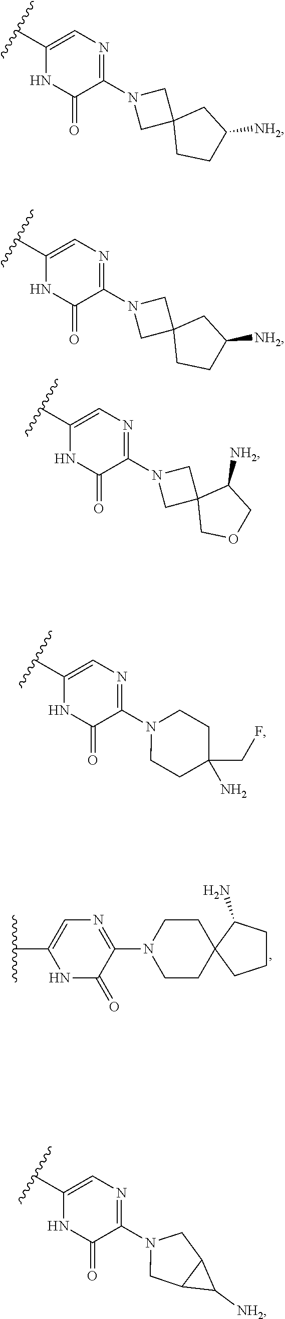 [SMILES: *c1cnc(N2CC3(CC[C@@H](N)C3)C2)c(=O)[nH]1 ; *c1cnc(N2CC3(CC[C@H](N)C3)C2)c(=O)[nH]1 ; *c1cnc(N2CC3(COC[C@@H]3N)C2)c(=O)[nH]1 ; *c1cnc(N2CCC(N)(CF)CC2)c(=O)[nH]1 ; *c1cnc(N2CCC3(CCC[C@H]3N)CC2)c(=O)[nH]1 ; *c1cnc(N2CC3C(N)C3C2)c(=O)[nH]1]

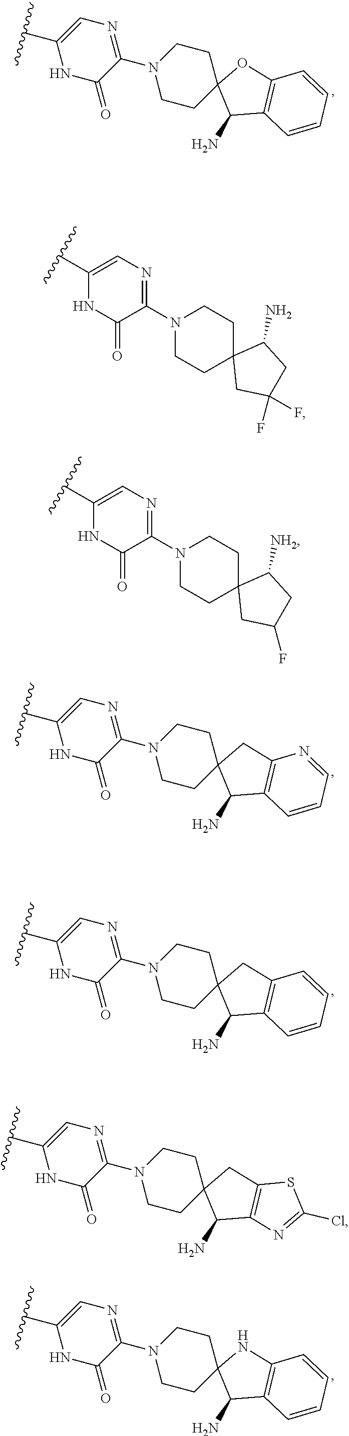

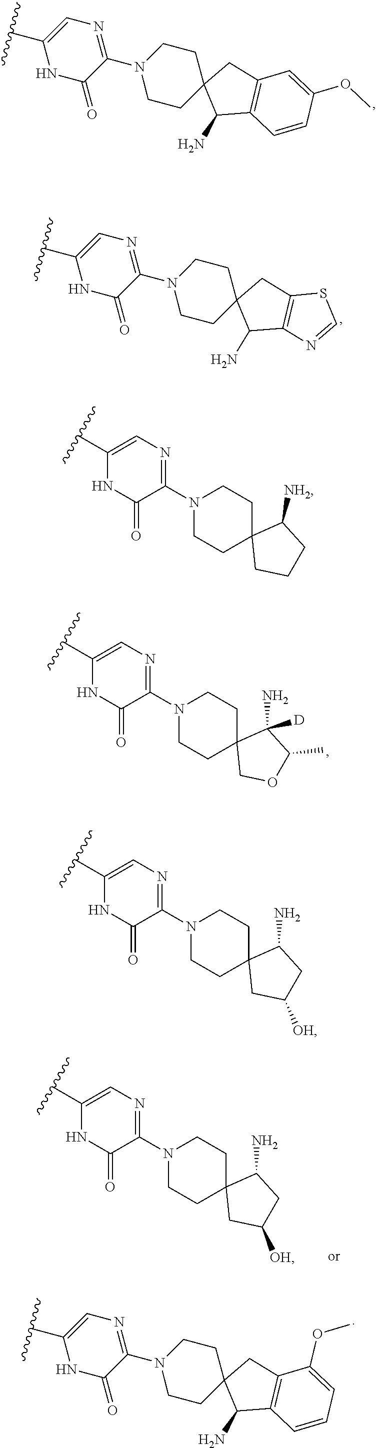

With respect to Formula 1, in some embodiments, X is S, O, NR^{A}, CHR^{A}, SO, SO₂, CO, or a bond. In some embodiments, X is S. In some embodiments, X is a bond. In some embodiments, X is O. In some embodiments, X is NH. In some embodiments, X is —CH(CH₃). In some embodiments, X is CH₂.

With respect to Formula 1, in some embodiments, R^{A} is H or C₁₋₆ hydrocarbyl. In some embodiments, R^{A} is H. In some embodiments, R^{A} is CH₃.

In Appendix A below, various possibilities for Ring A are depicted. Ring A could be any core structure in any of these depicted possibilities, wherein these core structures are optionally substituted.

### APPENDIX A

Phenyl,
2,3-dichlorophenyl,
naphthalen-1-yl,
2-(trifluoromethyl)phenyl,
2-(trifluoromethyl)pyridin-3-yl,
5-amino-2-chlorophenyl,
5-amino-2-chloropyridin-3-yl,
3-amino-2-chlorophenyl,
2-amino-3-chloropyridin-4-yl,
2-chloro-3-methoxyphenyl,
3-chloro-2-methoxypyridin-4-yl,
3-fluoro-1H-indol-4-yl,
3,3-difluoro-2-oxoindolin-4-yl,
1-acetyl-3,3-difluoroindolin-4-yl,
2-chloro-3-(4-hydroxy-1,5,5-trimethyl-2-oxo-2,5-dihydro-1H-pyrrole-3-carboxamido)phenyl,
2-chloro-3-(2-hydroxy-4-oxo-4H-pyrido[1,2-a]pyrimidine-3-carboxamido)phenyl,
2-chloro-3-(2-hydroxy-4-oxo-4H-pyrazino[1,2-a]pyrimidine-3-carboxamido)phenyl,
2-chloro-3-(7-hydroxy-5-oxo-5H-thiazolo[3,2-a]pyrimidine-6-carboxamido)phenyl,
2-chloro-3-(7-hydroxy-5-oxo-1,5-dihydroimidazo[1,2-a]pyrimidine-6-carboxamido)phenyl,
2-chloro-3-(2-hydroxy-4-oxo-6,7,8,9-tetrahydro-4H-pyrido[1,2-a]pyrimidine-3-carboxamido)phenyl,
2,3-dichloropyridin-4-yl,
2,3-difluorophenyl,
3-chloro-2-fluoropyridin-4-yl,
2,3-difluoropyridin-4-yl,
2-chloro-3-methylphenyl,
3-chloro-2-methylpyridin-4-yl,
3,3-difluoro-1-methyl-2-oxoindolin-4-yl,
3-chloro-1-methyl-2-oxo-1,2-dihydropyridin-4-yl,
2-chloro-3-fluorophenyl,
3-amino-2-fluorophenyl,
3-chloropyridin-4-yl,
3-chloro-2-(dimethylamino)pyridin-4-yl,

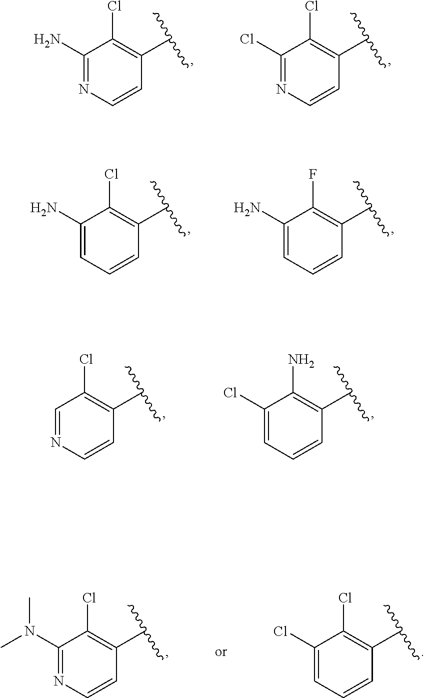

In Appendix B below, various possibilities for Ring B are depicted. Ring B could be any core structure in any of these depicted possibilities, wherein these core structures are optionally substituted.

### APPENDIX B

5-(4-(aminomethyl)-4-methylpiperidin-1-yl)-6-oxo-1,6-dihydropyrazin-2-yl,
5-(3-(aminomethyl)-3-methylpyrrolidin-1-yl)-6-oxo-1,6-dihydropyrazin-2-yl,
5-(hexahydropyrrolo[3,4-c]pyrrol-2(1H)-yl)-6-oxo-1,6-dihydropyrazin-2-yl,
5-(3,6-diazabicyclo[3.2.0]heptan-6-yl)-6-oxo-1,6-dihydropyrazin-2-yl,
(S)-5-(4-amino-2-oxa-8-azaspiro[4.5]decan-8-yl)-6-oxo-1,6-dihydropyrazin-2-yl,
6-oxo-5-(piperidin-4-ylamino)-1,6-dihydropyrazin-2-yl,
5-(2-aminospiro[bicyclo[3.1.0]hexane-3,4′-piperidin]-1′-yl)-6-oxo-1,6-dihydropyrazin-2-yl,
5-((1R,3R)-1-amino-3-methyl-8-azaspiro[4.5]decan-8-yl)-6-oxo-1,6-dihydropyrazin-2-yl,
(6-(4-(aminomethyl)-4-methylpiperidin-1-yl)-4-oxo-4,5-dihydro-1H-pyrazolo[3,4-d]pyrimidin-3-yl)amino,
6-(3-(aminomethyl)-3-methylpyrrolidin-1-yl)-4-oxo-4,5-dihydro-1H-pyrazolo[3,4-d]pyrimidin-3-yl,
7-amino-2-(4-(aminomethyl)-4-methylpiperidin-1-yl)-4-oxo-3,4-dihydroquinazolin-5-yl,
2-(4-(aminomethyl)-4-methylpiperidin-1-yl)-4-oxo-3,4-dihydropyrido[3,4-d]pyrimidin-5-yl,
2-(4-(aminomethyl)-4-methylpiperidin-1-yl)-7-oxo-7,8-dihydropyrido[2,3-d]pyrimidin-5-yl,
5-(4-(aminomethyl)-4-methylpiperidin-1-yl)-1H-pyrazolo[4,3-d]thiazol-3-yl,
6-(1-(1-aminopropan-2-yl)piperidin-4-yl)-7-oxo-6,7-dihydro-1H-pyrazolo[4,3-d]pyrimidin-3-yl,
8-(4-(aminomethyl)-4-methylpiperidin-1-yl)-6-oxo-6,7-dihydro-1H-purin-2-yl,
6-amino-8-(4-(aminomethyl)-4-methylpiperidin-1-yl)-7H-purin-2-yl,
4-amino-2-(3-(aminomethyl)-3-methylpyrrolidin-1-yl)-6-oxo-1,6-dihydropyrimidin-5-yl,
2-(4-(aminomethyl)-4-methylpiperidin-1-yl)-4-cyano-1-methyl-6-oxo-1,6-dihydropyrimidin-5-yl,
4-amino-2-(4-amino-2-oxa-8-azaspiro[4.5]decan-8-yl)-6-oxo-1,6-dihydropyrimidin-5-yl,
4-amino-2-(3,6-diazabicyclo[3.2.0]heptan-6-yI)-6-oxo-1,6-dihydropyrimidin-5-yl,
4-amino-2-(hexahydropyrrolo[3,4-c]pyrrol-2(1H)-yl)-6-oxo-1,6-dihydropyrimidin-5-yl,
4-amino-2-(4-(aminomethyl)-4-methylpiperidin-1-yl)-6-oxo-1,6-dihydropyrimidin-5-yl,
4-amino-2-(6-amino-3-azabicyclo[3.2.0]heptan-3-yl)-6-oxo-1,6-dihydropyrimidin-5-yl,
4-amino-2-(6-amino-2-azaspiro[3.4]octan-2-yl)-1-methyl-6-oxo-1,6-dihydropyrimidin-5-yl,
4-amino-2-(6-amino-3-azabicyclo[3.1.0]hexan-3-yl)-6-oxo-1,6-dihydropyrimidin-5-yl,
4-amino-2-(6-amino-2-azaspiro[3.4]octan-2-yl)-6-oxo-1,6-dihydropyrimidin-5-yl,
5-(4-(aminomethyl)-4-methylpiperidin-1-yl)-1-methyl-6-oxo-1,6-dihydropyrazin-2-yl,
5-(4-amino-2-oxa-8-azaspiro[4.5]decan-8-yl)-6-oxo-1,6-dihydropyrazin-2-yl,
(S)-5-(4-amino-2-oxa-8-azaspiro[4.5]decan-8-yl)-1-methyl-6-oxo-1,6-dihydropyrazin-2-yl,
5-(2-aminospiro[bicyclo[3.1.0]hexane-3,4′-piperidin]-1′-yl)-1-methyl-6-oxo-1,6-dihydropyrazin-2-yl,
5-((3S,4S)-4-amino-3-methyl-2-oxa-8-azaspiro[4.5]decan-8-yl)-6-oxo-1,6-dihydropyrazin-2-yl,
5-((3S,4S)-4-amino-3-methyl-2-oxa-8-azaspiro[4.5]decan-8-yl)-1-methyl-6-oxo-1,6-dihydropyrazin-2-yl,
5-((1R,3R)-1-amino-3-methyl-8-azaspiro[4.5]decan-8-yl)-1-methyl-6-oxo-1,6-dihydropyrazin-2-yl,
5-(4-(aminomethyl)-4-methylpiperidin-1-yl)-6-oxo-1,6-dihydropyrazin-2-yl,
6-(4-(aminomethyl)-4-methylpiperidin-1-yl)-4-oxo-4,5-dihydro-1H-pyrazolo[3,4-d]pyrimidin-3-yl,
6-(4-(aminomethyl)-4-methylpiperidin-1-yl)-4-methyl-5-oxo-4,5-dihydro-1H-pyrazolo[3,4-b]pyrazin-3-yl,
6-(4-(aminomethyl)-4-methylpiperidin-1-yl)-5-oxo-4,5-dihydro-1H-pyrazolo[3,4-b]pyrazin-3-yl,
6-(3-(aminomethyl)-3-methylpyrrolidin-1-yl)-5-methyl-4-oxo-4,5-dihydro-1H-pyrazolo[3,4-d]pyrimidin-3-yl,
6-(4-(aminomethyl)-4-methylpiperidin-1-yl)-5-methyl-4-oxo-4,5-dihydro-1H-pyrazolo[3,4-d]pyrimidin-3-yl,
6-(4-(aminomethyl)-4-methylpiperidin-1-yl)-1H-pyrazolo[3,4-d]pyrimidin-3-yl,
4-amino-2-(hexahydropyrrolo[3,4-c]pyrrol-2(1H)-yl)-1-methyl-6-oxo-1,6-dihydropyrimidin-5-yl,
4-amino-2-((3S,4S)-4-amino-3-methyl-2-oxa-8-azaspiro[4.5]decan-8-yl)-6-oxo-1,6-dihydropyrimidin-5-yl,
4-amino-2-((3S,4S)-4-amino-3-methyl-2-oxa-8-azaspiro[4.5]decan-8-yl)-1-methyl-6-oxo-1,6-dihydropyrimidin-5-yl,
4-amino-2-(6-amino-3-azabicyclo[3.1.0]hexan-3-yl)-1-methyl-6-oxo-1,6-dihydropyrimidin-5-yl,
4-amino-2-(6-amino-3-azabicyclo[3.2.0]heptan-3-yl)-1-methyl-6-oxo-1,6-dihydropyrimidin-5-yl,
5-(4-(aminomethyl)-4-fluoropiperidin-1-yl)-6-oxo-1,6-dihydropyrazin-2-yl,
5-(4-(aminomethyl)-4-hydroxypiperidin-1-yl)-6-oxo-1,6-dihydropyrazin-2-yl,
(R)-5-(6-amino-2-azaspiro[3.4]octan-2-yl)-6-oxo-1,6-dihydropyrazin-2-yl,
(S)-5-(6-amino-2-azaspiro[3.4]octan-2-yl)-6-oxo-1,6-dihydropyrazin-2-yl,
1-(3-aminocyclohexyl)-2-oxo-1,2-dihydropyridin-4-yl,
(R)-5-(4-amino-2-oxa-8-azaspiro[4.5]decan-8-yl)-6-oxo-1,6-dihydropyrazin-2-yl,
5-(4-amino-4-(fluoromethyl)piperidin-1-yl)-6-oxo-1,6-dihydropyrazin-2-yl,
5-((3S,4S)-4-amino-3-methyl-2-oxa-8-azaspiro[4.5]decan-8-yl)-6-oxo-1,6-dihydropyrazin-2-yl,
(R)-5-(1-amino-8-azaspiro[4.5]decan-8-yl)-6-oxo-1,6-dihydropyrazin-2-yl,
5-(6-amino-3-azabicyclo[3.1.0]hexan-3-yl)-6-oxo-1,6-dihydropyrazin-2-yl,
(R)-5-(3-amino-3H-spiro[benzofuran-2,4′-piperidin]-1′-yl)-6-oxo-1,6-dihydropyrazin-2-yl,
2-((3S,4S)-4-amino-3-methyl-2-oxa-8-azaspiro[4.5]decan-8-yl)-3-methyl-4-oxo-4,7-dihydro-3H-pyrrolo[2,3-d]pyrimidin-5-yl,
2-((3S,4S)-4-amino-3-methyl-2-oxa-8-azaspiro[4.5]decan-8-yl)-4-oxo-4,7-dihydro-3H-pyrrolo[2,3-d]pyrimidin-5-yl,
(R)-5-(1-amino-3,3-difluoro-8-azaspiro[4.5]decan-8-yl)-6-oxo-1,6-dihydropyrazin-2-yl,
5-((1R)-1-amino-3-fluoro-8-azaspiro[4.5]decan-8-yl)-6-oxo-1,6-dihydropyrazin-2-yl,
(S)-5-(5-amino-5,7-dihydrospiro[cyclopenta[b]pyridine-6,4′-piperidin]-1′-yl)-6-oxo-1,6-dihydropyrazin-2-yl,
(S)-5-(1-amino-1,3-dihydrospiro[indene-2,4′-piperidin]-1′-yl)-6-oxo-1,6-dihydropyrazin-2-yl,
(S)-5-(4-amino-2-chloro-4,6-dihydrospiro[cyclopenta[d]thiazole-5,4′-piperidin]-1′-yl)-6-oxo-1,6-dihydropyrazin-2-yl,
(R)-5-(3-aminospiro[indoline-2,4′-piperidin]-1′-yl)-6-oxo-1,6-dihydropyrazin-2-yl,
(S)-5-(1-amino-4-methoxy-1,3-dihydrospiro[indene-2,4′-piperidin]-1′-yl)-6-oxo-1,6-dihydropyrazin-2-yl,
(S)-(5-(1-amino-5-methoxy-1,3-dihydrospiro[indene-2,4′-piperidin]-1′-yl)-6-oxo-1,6-dihydropyrazin-2-yl)silver,
5-(4-amino-4,6-dihydrospiro[cyclopenta[d]thiazole-5,4′-piperidin]-1′-yl)-6-oxo-1,6-dihydropyrazin-2-yl,
(S)-5-(1-amino-8-azaspiro[4.5]decan-8-yl)-6-oxo-1,6-dihydropyrazin-2-yl,
5-((3S,4S)-4-amino-3-methyl-2-oxa-8-azaspiro[4.5]decan-8-yl-4-d)-6-oxo-1,6-dihydropyrazin-2-yl,
5-((1R,3R)-1-amino-3-hydroxy-8-azaspiro[4.5]decan-8-yl)-6-oxo-1,6-dihydropyrazin-2-yl,
5-((1R,3S)-1-amino-3-hydroxy-8-azaspiro[4.5]decan-8-yl)-6-oxo-1,6-dihydropyrazin-2-yl,

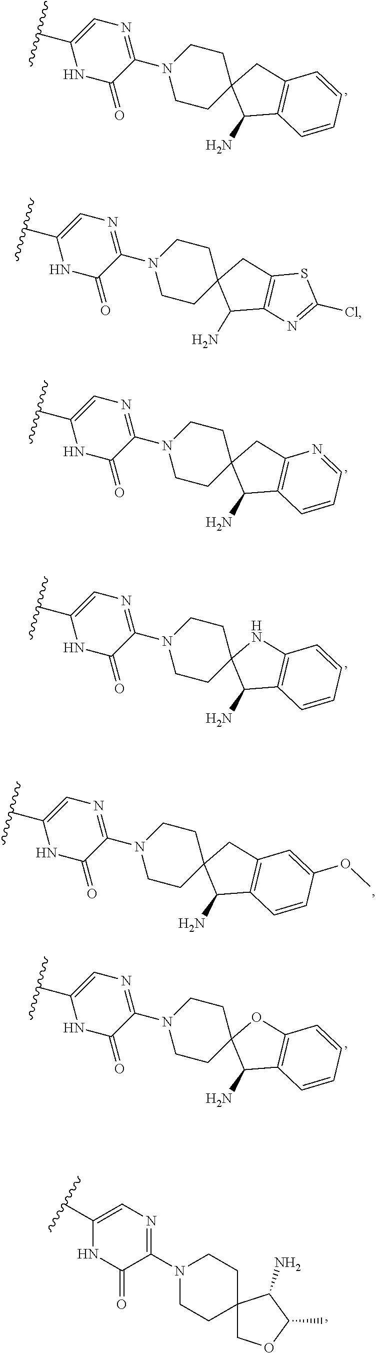

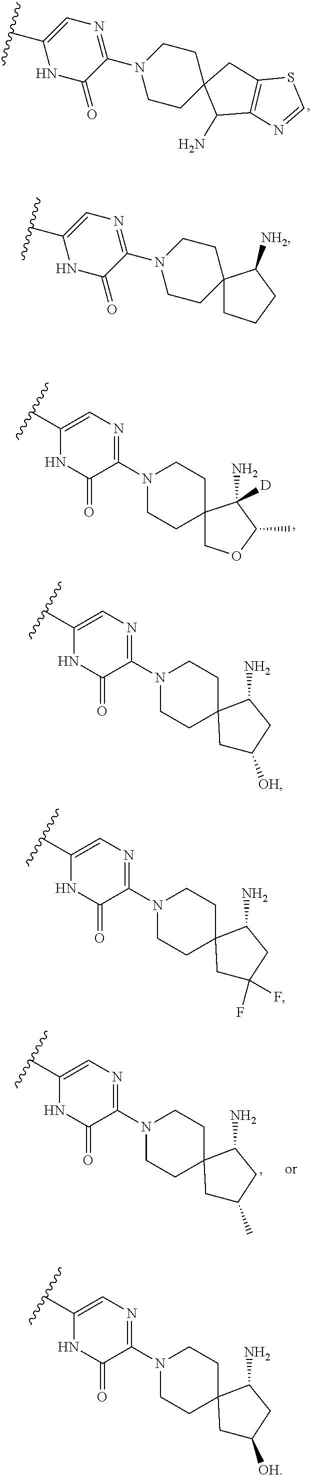

With respect to Formula 1, in some embodiments, X is S; Ring A is an optionally substituted 6-membered aryl or heteroaryl, such as an optionally substituted phenyl or an optionally substituted pyridine; Ring B is:

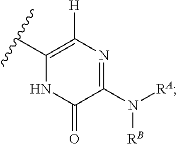

wherein —N(R^{A})(R^{B}) is an optionally substituted heterocyclic ring system. In some embodiments, the heterocyclic ring system is an optionally substituted bicyclic or tricyclic ring system. In some embodiments, the bicyclic ring system is an optionally substituted spiro bicyclic ring. In some embodiments, the tricyclic ring system contains an optionally substituted spiro bicyclic ring. In some embodiments, the bicyclic ring system or the tricyclic ring system has an amino substituent. In some embodiments, Ring A is:

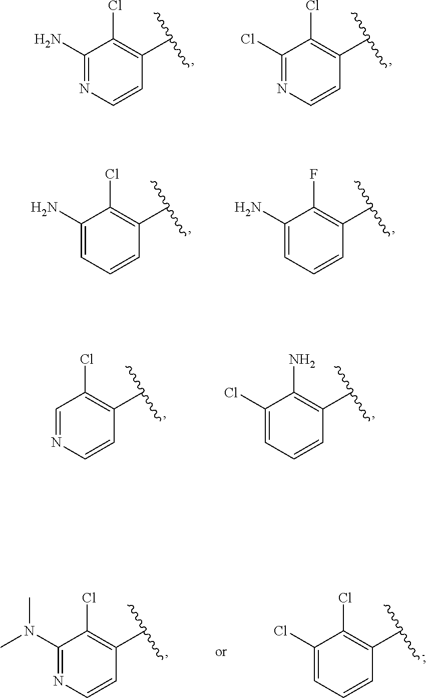

### and Ring B is:

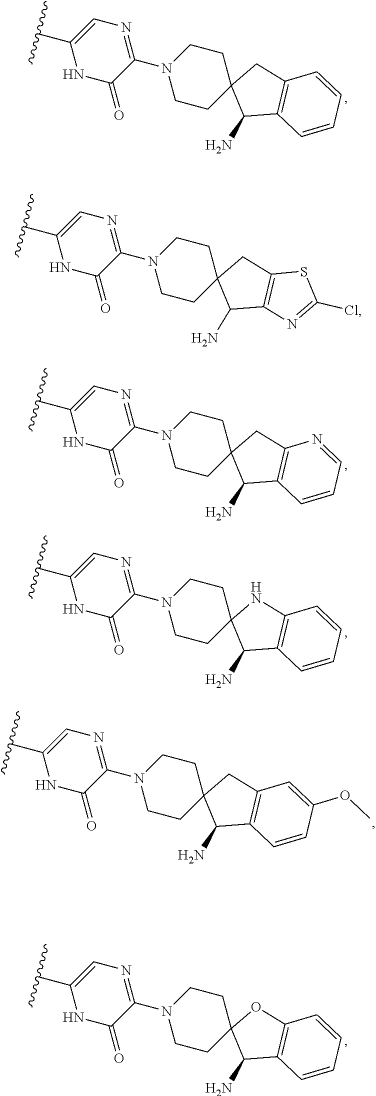 [SMILES: *c1cnc(N2CCC3(CC2)Cc2ccccc2[C@H]3N)c(=O)[nH]1 ; *c1cnc(N2CCC3(CC2)Cc2sc(Cl)nc2C3N)c(=O)[nH]1 ; *c1cnc(N2CCC3(CC2)Cc2ncccc2[C@H]3N)c(=O)[nH]1 ; *c1cnc(N2CCC3(CC2)Nc2ccccc2[C@H]3N)c(=O)[nH]1 ; *c1cnc(N2CCC3(CC2)Cc2cc(OC)ccc2[C@H]3N)c(=O)[nH]1 ; *c1cnc(N2CCC3(CC2)Oc2ccccc2[C@H]3N)c(=O)[nH]1]

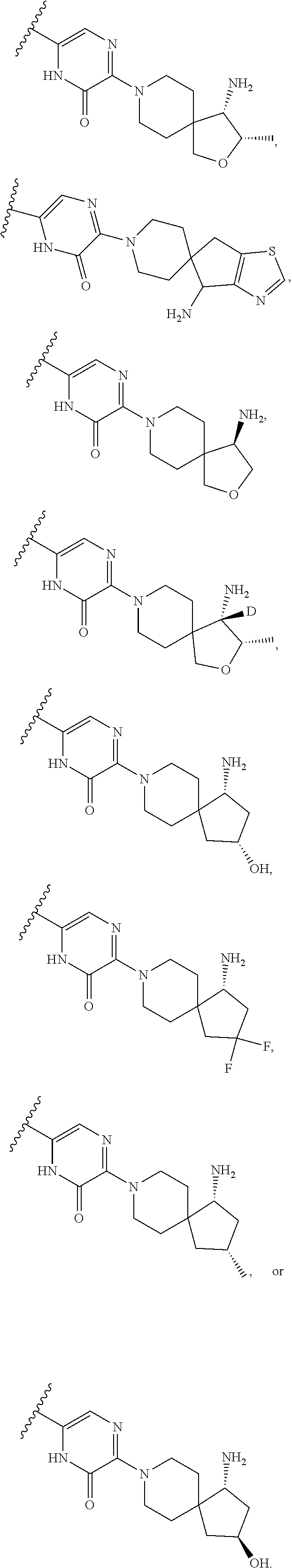

Some embodiments include one of the compounds in Table 1, wherein any of the compounds in Table 1 below may be optionally substituted.

TABLE 1

| Com- | | |
|---|---|---|
| pound | | |
| ID | Structure | Compound name |
| 1 | 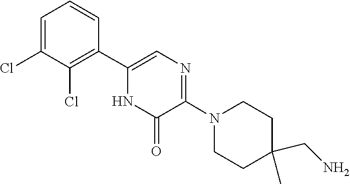 [SMILES: CC1(CN)CCN(c2ncc(-c3cccc(Cl)c3Cl)[nH]c2=O)CC1] | 3-(4-(aminomethyl)-4- methylpiperidin-1-yl)-6-(2,3- dichlorophenyl)pyrazin-2(1H)- one |
| 2 | 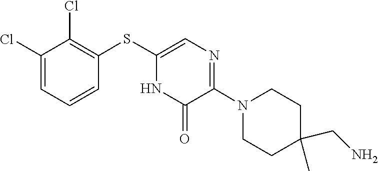 [SMILES: CC1(CN)CCN(c2ncc(Sc3cccc(Cl)c3Cl)[nH]c2=O)CC1] | 3-(4-(aminomethyl)-4- methylpiperidin-1-yl)-6-((2,3- dichlorophenyl)thio)pyrazin-2 (1H)-one |
| 3 | 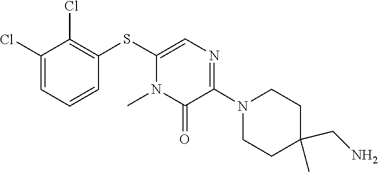 [SMILES: Cn1c(Sc2cccc(Cl)c2Cl)cnc(N2CCC(C)(CN)CC2)c1=O] | 3-(4-(aminomethyl)-4- methylpiperidin-1-yl)-6-((2,3- dichlorophenyl)thio)-1- methylpyrazin-2(1H)-one |
| 4 | 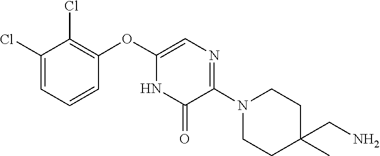 [SMILES: CC1(CN)CCN(c2ncc(Oc3cccc(Cl)c3Cl)[nH]c2=O)CC1] | 3-(4-(aminomethyl)-4- methylpiperidin-1-yl)-6-(2,3- dichlorophenoxy)pyrazin-2(1H)- one |
| 5 | 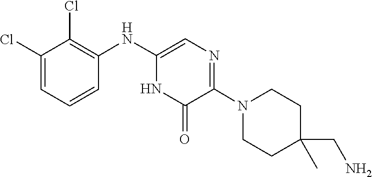 [SMILES: CC1(CN)CCN(c2ncc(Nc3cccc(Cl)c3Cl)[nH]c2=O)CC1] | 3-(4-(aminomethyl)-4- methylpiperidin-1-yl)-6-((2,3- dichlorophenyl)amino)pyrazin- 2(1H)-one |
| 6 | 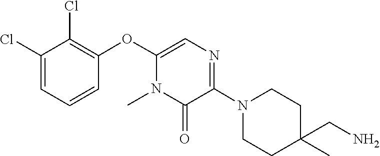 [SMILES: Cn1c(Oc2cccc(Cl)c2Cl)cnc(N2CCC(C)(CN)CC2)c1=O] | 3-(4-(aminomethyl)-4- methylpiperidin-1-yl)-6-(2,3- dichlorophenoxy)-1-methyl- pyrazin-2(1H)-one |
| 7 | 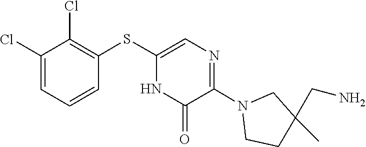 [SMILES: CC1(CN)CCN(c2ncc(Sc3cccc(Cl)c3Cl)[nH]c2=O)C1] | 3-(3-(aminomethyl)-3- methylpyrrolidin-1-yl)-6-((2,3- dichlorophenyl)thio)pyrazin-2 (1H)-one |
| 8 | 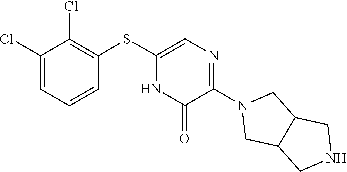 [SMILES: O=c1[nH]c(Sc2cccc(Cl)c2Cl)cnc1N1CC2CNCC2C1] | 6-((2,3-dichlorophenyl)thio)-3- (hexahydropyrrolo[3,4-c]pyrrol- 2(1H)-yl)pyrazin-2(1H)-one |
| 9 | 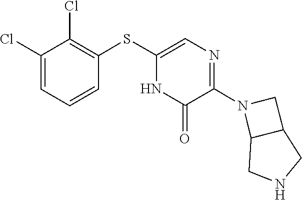 [SMILES: O=c1[nH]c(Sc2cccc(Cl)c2Cl)cnc1N1CC2CNCC21] | 3-(3,6-diazabicyclo[3.2.0]heptan- 6-yl)-6-((2,3- dichlorophenyl)thio)pyrazin-2 (1H)-one |
| 10 | 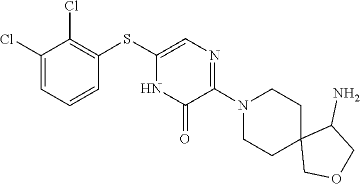 [SMILES: NC1COCC12CCN(c1ncc(Sc3cccc(Cl)c3Cl)[nH]c1=O)CC2] | 3-(4-amino-2-oxa-8- azaspiro[4.5]decan-8-yl)-6-((2,3- dichlorophenyl)thio)pyrazin-2 (1H)-one |
| 11 | 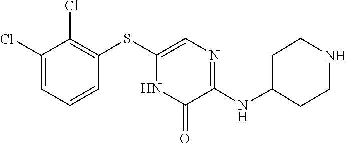 [SMILES: O=c1[nH]c(Sc2cccc(Cl)c2Cl)cnc1NC1CCNCC1] | 6-((2,3-dichlorophenyl)thio)-3- (piperidin-4-ylamino)pyrazin-2 (1H)-one |
| 12 | 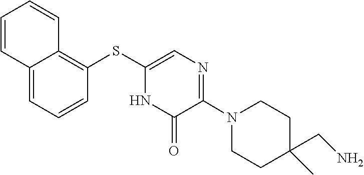 [SMILES: CC1(CN)CCN(c2ncc(Sc3cccc4ccccc34)[nH]c2=O)CC1] | 3-(4-(aminomethyl)-4- methylpiperidin-1-yl)-6- (naphthalen-1-ylthio)pyrazin-2 (1H)-one |
| 13 | 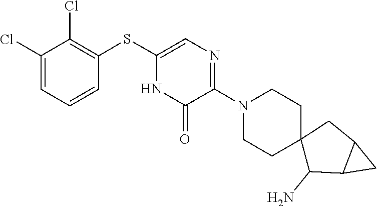 [SMILES: NC1C2CC2CC12CCN(c1ncc(Sc3cccc(Cl)c3Cl)[nH]c1=O)CC2] | 3-(2- aminospiro[bicyclo[3.1.0]hexane- 3,4′-piperidin]-1′-yl)-6-((2,3- dichlorophenyl)thio)pyrazin-2 (1H)-one |
| 14 | 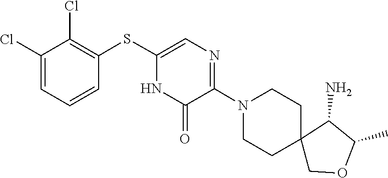 [SMILES: C[C@@H]1OCC2(CCN(c3ncc(Sc4cccc(Cl)c4Cl)[nH]c3=O)CC2)[C@@H]1N] | 3-((3S,4S)-4-amino-3-methyl-2- oxa-8-azaspiro[4.5]decan-8-yl)-6- ((2,3-dichlorophenyl)thio) pyrazin-2(1H)-one |
| 15 | 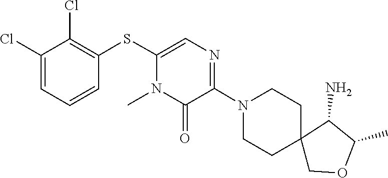 [SMILES: C[C@@H]1OCC2(CCN(c3ncc(Sc4cccc(Cl)c4Cl)n(C)c3=O)CC2)[C@@H]1N] | 3-((3S,4S)-4-amino-3-methyl-2- oxa-8-azaspiro[4.5]decan-8-yl)-6- ((2,3-dichlorophenyl)thio)-1- methylpyrazin-2(1H)-one |
| 16 | 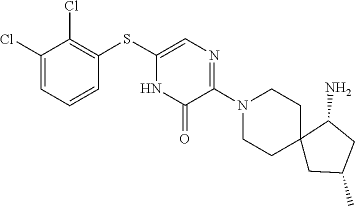 [SMILES: C[C@H]1C[C@@H](N)C2(CCN(c3ncc(Sc4cccc(Cl)c4Cl)[nH]c3=O)CC2)C1] | 3-((1R,3R)-1-amino-3-methyl-8- azaspiro[4.5]decan-8-yl)-6-((2,3- dichlorophenyl)thio)pyrazin-2 (1H)-one |
| 17 | 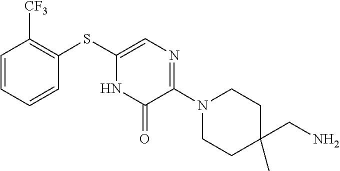 [SMILES: CC1(CN)CCN(c2ncc(Sc3ccccc3C(F)(F)F)[nH]c2=O)CC1] | 3-(4-(aminomethyl)-4- methylpiperidin-1-yl)-6-((2- (trifluoromethyl)phenyl)thio) pyrazin-2(1H)-one |
| 18 | 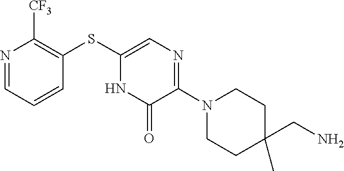 [SMILES: CC1(CN)CCN(c2ncc(Sc3cccnc3C(F)(F)F)[nH]c2=O)CC1] | 3-(4-(aminomethyl)-4- methylpiperidin-1-yl)-6-((2- (trifluoromethyl)pyridin-3- yl)thio)pyrazin-2(1H)-one |
| 19 | 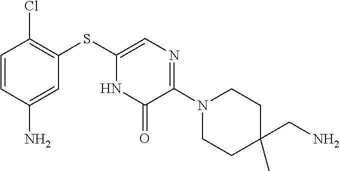 [SMILES: CC1(CN)CCN(c2ncc(Sc3cc(N)ccc3Cl)[nH]c2=O)CC1] | 6-((5-amino-2-chlorophenyl)thio)- (4-(aminomethyl)-4- methylpiperidin-1-yl)pyrazin-2 (1H)-one |
| 20 | 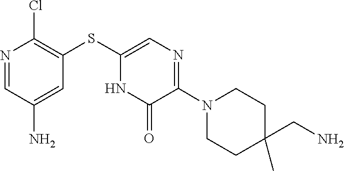 [SMILES: CC1(CN)CCN(c2ncc(Sc3cc(N)cnc3Cl)[nH]c2=O)CC1] | 6-((5-amino-2-chloropyridin-3- yl)thio)-3-(4-(aminomethyl)-4- methylpiperidin-1-yl)pyrazin-2 (1H)-one |
| 21 | 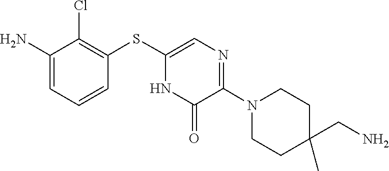 [SMILES: CC1(CN)CCN(c2ncc(Sc3cccc(N)c3Cl)[nH]c2=O)CC1] | 6-((3-amino-2-chlorophenyl)thio)- (4-(aminomethyl)-4- methylpiperidin-1-yl)pyrazin-2 (1H)-one |
| 22 | 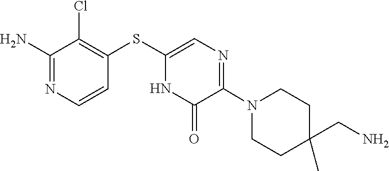 [SMILES: CC1(CN)CCN(c2ncc(Sc3ccnc(N)c3Cl)[nH]c2=O)CC1] | 6-((2-amino-3-chloropyridin-4- yl)thio)-3-(4-(aminomethyl)-4- methylpiperidin-1-yl)pyrazin-2 (1H)-one |
| 23 | 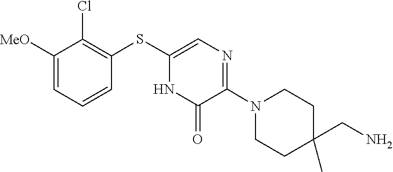 [SMILES: COc1cccc(Sc2cnc(N3CCC(C)(CN)CC3)c(=O)[nH]2)c1Cl] | 3-(4-(aminomethyl)-4- methylpiperidin-1-yl)-6- ((2-chloro-3- methoxyphenyl)thio)pyrazin-2 (1H)-one |
| 24 | 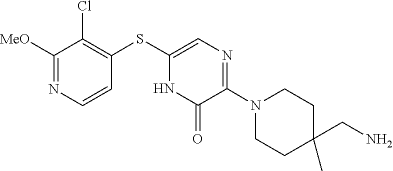 [SMILES: COc1nccc(Sc2cnc(N3CCC(C)(CN)CC3)c(=O)[nH]2)c1Cl] | 3-(4-(aminomethyl)-4- methylpiperidin-1-yl)-6- ((3-chloro-2-methoxypyridin-4- yl)thio)pyrazin-2(1H)-one |
| 25 | 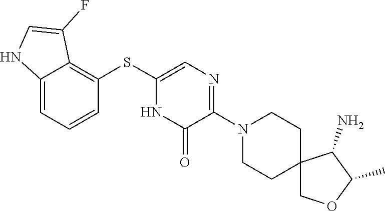 [SMILES: C[C@@H]1OCC2(CCN(c3ncc(Sc4cccc5[nH]cc(F)c45)[nH]c3=O)CC2)[C@@H]1N] | 3-((3S,4S)-4-amino-3-methyl-2- oxa-8-azaspiro[4.5]decan-8-yl)-6- ((3-fluoro-1H-indol-4-yl)thio) pyrazin-2(1H)-one |
| 26 | 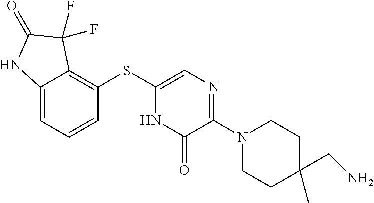 [SMILES: CC1(CN)CCN(c2ncc(Sc3cccc4c3C(F)(F)C(=O)N4)[nH]c2=O)CC1] | 4-((5-(4-(aminomethyl)-4- methylpiperidin-1-yl)-6-oxo-1,6- dihydropyrazin-2-yl)thio)-3,3- difluoroindolin-2-one |
| 27 | 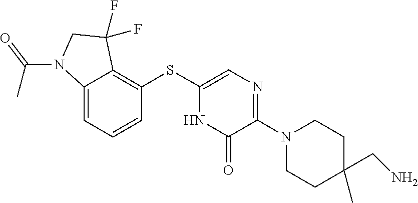 [SMILES: CC(=O)N1CC(F)(F)c2c(Sc3cnc(N4CCC(C)(CN)CC4)c(=O)[nH]3)cccc21] | 6-((1-acetyl-3,3-difluoroindolin- 4-yl)thio)-3-(4-(aminomethyl)-4- methylpiperidin-1-yl)pyrazin-2 (1H)-one |
| 28 | 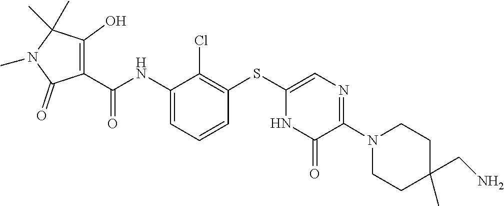 [SMILES: CN1C(=O)C(C(=O)Nc2cccc(Sc3cnc(N4CCC(C)(CN)CC4)c(=O)[nH]3)c2Cl)=C(O)C1(C)C] | N-(3-((5-(4-(aminomethyl)-4- methylpiperidin-1-yl)-6-oxo-1,6- dihydropyrazin-2-yl)thio)-2- chlorophenyl)-4-hydroxy-1,5,5- trimethyl-2-oxo-2,5-dihydro-1H- pyrrole-3-carboxamide |
| 29 | 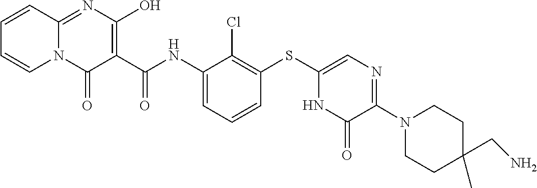 [SMILES: CC1(CN)CCN(c2ncc(Sc3cccc(NC(=O)c4c(O)nc5ccccn5c4=O)c3Cl)[nH]c2=O)CC1] | N-(3-((5-(4-(aminomethyl)-4- methylpiperidin-1-yl)-6-oxo-1,6- dihydropyrazin-2-yl)thio)-2- chlorophenyl)-2-hydroxy-4-oxo- 4H-pyrido[1,2-a]pyrimidine-3- carboxamide |
| 30 | 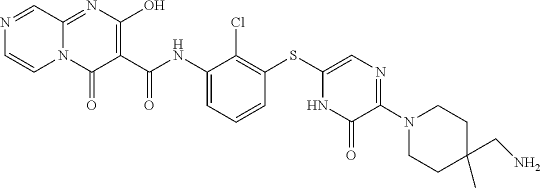 [SMILES: CC1(CN)CCN(c2ncc(Sc3cccc(NC(=O)c4c(O)nc5cnccn5c4=O)c3Cl)[nH]c2=O)CC1] | N-(3-((5-(4-(aminomethyl)-4- methylpiperidin-1-yl)-6-oxo-1,6- dihydropyrazin-2-yl)thio)-2- chlorophenyl)-2-hydroxy-4-oxo- 4H-pyrazino[1,2-a]pyrimidine-3- carboxamide |
| 31 | 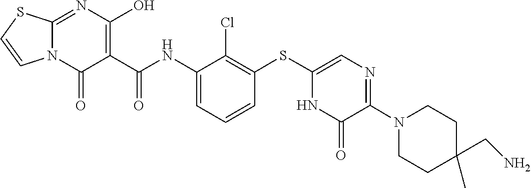 [SMILES: CC1(CN)CCN(c2ncc(Sc3cccc(NC(=O)c4c(O)nc5sccn5c4=O)c3Cl)[nH]c2=O)CC1] | N-(3-((5-(4-(aminomethyl)-4- methylpiperidin-1-yl)-6-oxo-1,6- dihydropyrazin-2-yl)thio)-2- chlorophenyl)-7-hydroxy-5-oxo- 5H-thiazolo[3,2-a]pyrimidine-6- carboxamide |
| 32 | 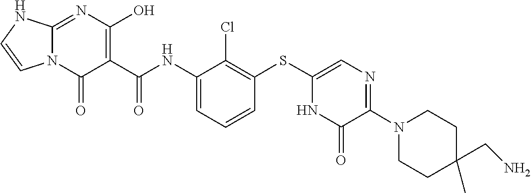 [SMILES: CC1(CN)CCN(c2ncc(Sc3cccc(NC(=O)c4c(O)nc5[nH]ccn5c4=O)c3Cl)[nH]c2=O)CC1] | N-(3-((5-(4-(aminomethyl)-4- methylpiperidin-1-yl)-6-oxo-1,6- dihydropyrazin-2-yl)thio)-2- chlorophenyl)-7-hydroxy-5-oxo- 1,5-dihydroimidazo[1,2- a]pyrimidine-6-carboxamide |
| 33 | 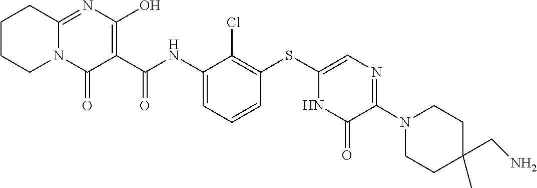 [SMILES: CC1(CN)CCN(c2ncc(Sc3cccc(NC(=O)c4c(O)nc5n(c4=O)CCCC5)c3Cl)[nH]c2=O)CC1] | N-(3-((5-(4-(aminomethyl)-4- methylpiperidin-1-yl)-6-oxo-1,6- dihydropyrazin-2-yl)thio)-2- chlorophenyl)-2-hydroxy-4-oxo- 6,7,8,9-tetrahydro-4H-pyrido [1,2-a]pyrimidine-3-carboxamide |
| 34 | 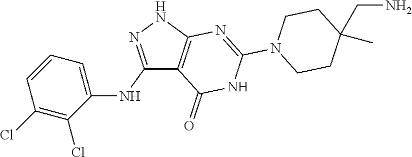 [SMILES: CC1(CN)CCN(c2nc3[nH]nc(Nc4cccc(Cl)c4Cl)c3c(=O)[nH]2)CC1] | 6-(4-(aminomethyl)-4- methylpiperidin-1-yl)-3-((2,3- dichlorophenyl)amino)-1,5- dihydro-4H-pyrazolo[3,4- d]pyrimidin-4-one |
| 35 | 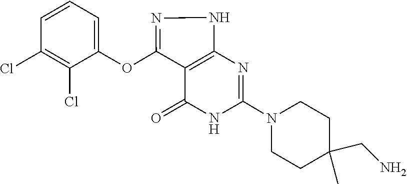 [SMILES: CC1(CN)CCN(c2nc3[nH]nc(Oc4cccc(Cl)c4Cl)c3c(=O)[nH]2)CC1] | 6-(4-(aminomethyl)-4- methylpiperidin-1-yl)-3-(2,3- dichlorophenoxy)-1,5-dihydro-4H- pyrazolo[3,4-d]pyrimidin-4-one |
| 36 | 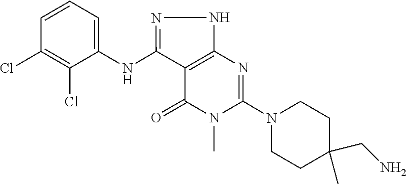 [SMILES: Cn1c(N2CCC(C)(CN)CC2)nc2[nH]nc(Nc3cccc(Cl)c3Cl)c2c1=O] | 6-(4-(aminomethyl)-4- methylpiperidin-1-yl)-3-((2,3- dichlorophenyl)amino)-5-methyl- 1,5-dihydro-4H-pyrazolo[3,4- d]pyrimidin-4-one |
| 37 | 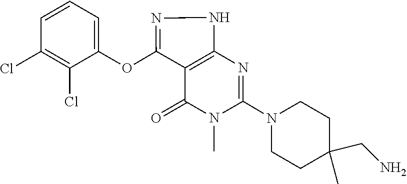 [SMILES: Cn1c(N2CCC(C)(CN)CC2)nc2[nH]nc(Oc3cccc(Cl)c3Cl)c2c1=O] | 6-(4-(aminomethyl)-4- methylpiperidin-1-yl)-3-(2,3- dichlorophenoxy)-5-methyl-1,5- dihydro-4H-pyrazolo[3,4- d]pyrimidin-4-one |
| 38 | 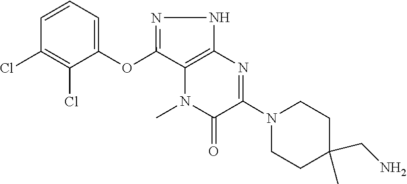 [SMILES: Cn1c(=O)c(N2CCC(C)(CN)CC2)nc2[nH]nc(Oc3cccc(Cl)c3Cl)c21] | 6-(4-(aminomethyl)-4- methylpiperidin-1-yl)-3-(2,3- dichlorophenoxy)-4-methyl-1,4- dihydro-5H-pyrazolo[3,4-b] pyrazin-5-one |
| 39 | 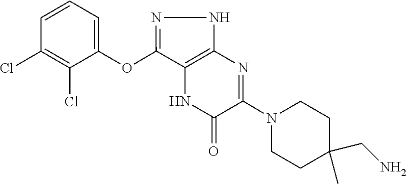 [SMILES: CC1(CN)CCN(c2nc3[nH]nc(Oc4cccc(Cl)c4Cl)c3[nH]c2=O)CC1] | 6-(4-(aminomethyl)-4- methylpiperidin-1-yl)-3-(2,3- dichlorophenoxy)-1,4-dihydro-5H- pyrazolo[3,4-b]pyrazin-5-one |
| 40 | 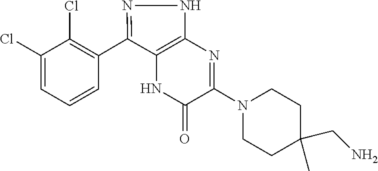 [SMILES: CC1(CN)CCN(c2nc3[nH]nc(-c4cccc(Cl)c4Cl)c3[nH]c2=O)CC1] | 6-(4-(aminomethyl)-4- methylpiperidin-1-yl)-3-(2,3- dichlorophenyl)-1,4-dihydro-5H- pyrazolo[3,4-b]pyrazin-5-one |
| 41 | 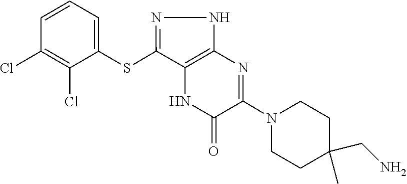 [SMILES: CC1(CN)CCN(c2nc3[nH]nc(Sc4cccc(Cl)c4Cl)c3[nH]c2=O)CC1] | 6-(4-(aminomethyl)-4- methylpiperidin-1-yl)-3-((2,3- dichlorophenyl)thio)-1,4-dihydro- 5H-pyrazolo[3,4-b]pyrazin-5-one |
| 42 | 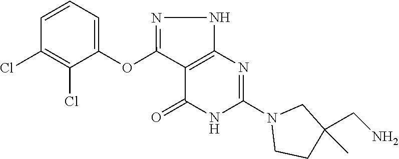 [SMILES: CC1(CN)CCN(c2nc3[nH]nc(Oc4cccc(Cl)c4Cl)c3c(=O)[nH]2)C1] | 6-(3-(aminomethyl)-3- methylpyrrolidin-1-yl)-3-(2,3- dichlorophenoxy)-1,5-dihydro-4H- pyrazolo[3,4-d]pyrimidin-4-one |
| 43 | 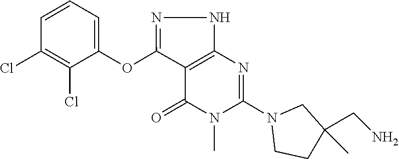 [SMILES: Cn1c(N2CCC(C)(CN)C2)nc2[nH]nc(Oc3cccc(Cl)c3Cl)c2c1=O] | 6-(3-(aminomethyl)-3- methylpyrrolidin-1-yl)-3-(2,3- dichlorophenoxy)-5-methyl-1,5- dihydro-4H-pyrazolo[3,4- d]pyrimidin-4-one |
| 44 | 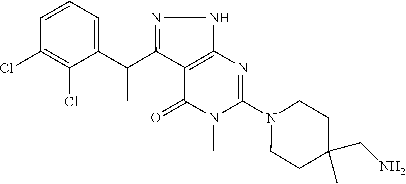 [SMILES: CC(c1cccc(Cl)c1Cl)c1n[nH]c2nc(N3CCC(C)(CN)CC3)n(C)c(=O)c12] | 6-(4-(aminomethyl)-4- methylpiperidin-1-yl)-3-(1-(2,3- dichlorophenyl)ethyl)-5-methyl- 1,5-dihydro-4H-pyrazolo[3,4- d]pyrimidin-4-one |
| 45 | 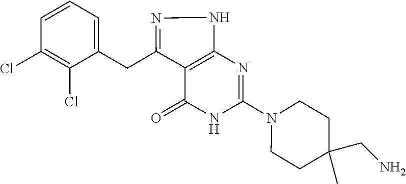 [SMILES: CC1(CN)CCN(c2nc3[nH]nc(Cc4cccc(Cl)c4Cl)c3c(=O)[nH]2)CC1] | 6-(4-(aminomethyl)-4- methylpiperidin-1-yl)-3-(2,3- dichlorobenzyl)-1,5-dihydro-4H- pyrazolo[3,4-d]pyrimidin-4-one |
| 46 | 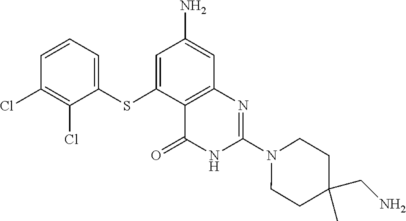 [SMILES: CC1(CN)CCN(c2nc3cc(N)cc(Sc4cccc(Cl)c4Cl)c3c(=O)[nH]2)CC1] | 7-amino-2-(4-(aminomethyl)-4- methylpiperidin-1-yl)-5-((2,3- dichlorophenyl)thio)quinazolin- 4(3H)-one |
| 47 | 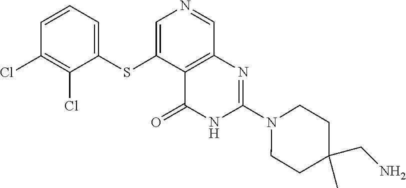 [SMILES: CC1(CN)CCN(c2nc3cncc(Sc4cccc(Cl)c4Cl)c3c(=O)[nH]2)CC1] | 2-(4-(aminomethyl)-4- methylpiperidin-1-yl)-5-((2,3- dichlorophenyl)thio)pyrido[3,4- d]pyrimidin-4(3H)-one |
| 48 | 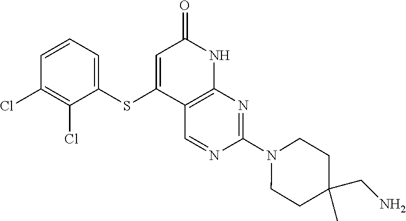 [SMILES: CC1(CN)CCN(c2ncc3c(Sc4cccc(Cl)c4Cl)cc(=O)[nH]c3n2)CC1] | 2-(4-(aminomethyl)-4- methylpiperidin-1-yl)-5-((2,3- dichlorophenyl)thio)pyrido[2,3- d]pyrimidin-7(8H)-one |
| 49 | 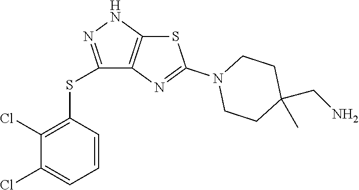 [SMILES: CC1(CN)CCN(c2nc3c(Sc4cccc(Cl)c4Cl)n[nH]c3s2)CC1] | (1-(3-((2,3-dichlorophenyl)thio)- 1H-pyrazolo[4,3-d]thiazol-5-yl)-4- methylpiperidin-4-yl) methanamine |
| 50 | 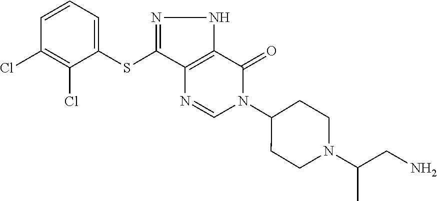 [SMILES: CC(CN)N1CCC(n2cnc3c(Sc4cccc(Cl)c4Cl)n[nH]c3c2=O)CC1] | 6-(1-(1-aminopropan-2-yl) piperidin-4-yl)-3-((2,3- dichlorophenyl)thio)-1,6-dihydro- 7H-pyrazolo[4,3-d]pyrimidin-7- one |
| 51 | 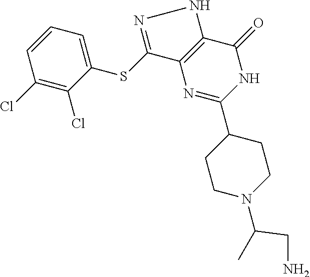 [SMILES: CC(CN)N1CCC(c2nc3c(Sc4cccc(Cl)c4Cl)n[nH]c3c(=O)[nH]2)CC1] | 5-(1-(1-aminopropan-2-yl) piperidin-4-yl)-3-((2,3- dichlorophenyl)thio)-1,6-dihydro- 7H-pyrazolo[4,3-d]pyrimidin-7- one |
| 52 | 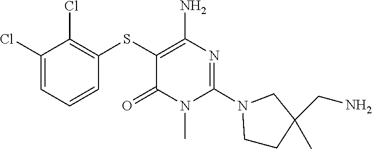 [SMILES: Cn1c(N2CCC(C)(CN)C2)nc(N)c(Sc2cccc(Cl)c2Cl)c1=O] | 6-amino-2-(3-(aminomethyl)-3- methylpyrrolidin-1-yl)-5-((2,3- dichlorophenyl)thio)-3- methylpyrimidin-4(3H)-one |
| 53 | 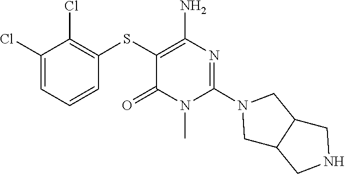 [SMILES: Cn1c(N2CC3CNCC3C2)nc(N)c(Sc2cccc(Cl)c2Cl)c1=O] | 6-amino-5-((2,3- dichlorophenyl)thio)-2- (hexahydropyrrolo[3,4-c]pyrrol- 2(1H)-yl)-3-methylpyrimidin-4 (3H)-one |
| 54 | 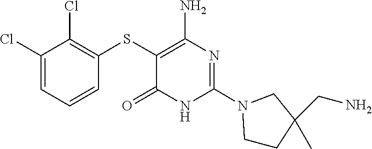 [SMILES: CC1(CN)CCN(c2nc(N)c(Sc3cccc(Cl)c3Cl)c(=O)[nH]2)C1] | 6-amino-2-(3-(aminomethyl)-3- methylpyrrolidin-1-yl)-5-((2,3- dichlorophenyl)thio)pyrimidin- 4(3H)-one |
| 55 | 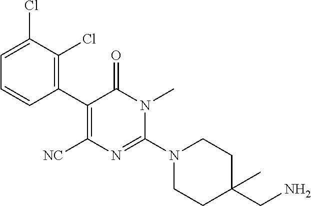 [SMILES: Cn1c(N2CCC(C)(CN)CC2)nc(C#N)c(-c2cccc(Cl)c2Cl)c1=O] | 2-(4-(aminomethyl)-4- methylpiperidin-1-yl)-5-(2,3- dichlorophenyl)-1-methyl-6-oxo- 1,6-dihydropyrimidine-4- carbonitrile |
| 56 | 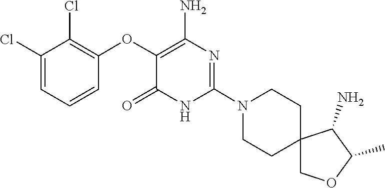 [SMILES: C[C@@H]1OCC2(CCN(c3nc(N)c(Oc4cccc(Cl)c4Cl)c(=O)[nH]3)CC2)[C@@H]1N] | 6-amino-2-((3S,4S)-4-amino-3- methyl-2-oxa-8-azaspiro[4.5] decan-8-yl)-5-(2,3- dichlorophenoxy)pyrimidin-4(3H)- one |
| 57 | 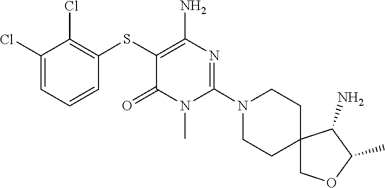 [SMILES: C[C@@H]1OCC2(CCN(c3nc(N)c(Sc4cccc(Cl)c4Cl)c(=O)n3C)CC2)[C@@H]1N] | 6-amino-2-((3S,4S)-4-amino-3- methyl-2-oxa-8-azaspiro[4.5] decan-8-yl)-5-(2,3- dichlorophenoxy)-3- methylpyrimidin-4(3H)-one |
| 58 | 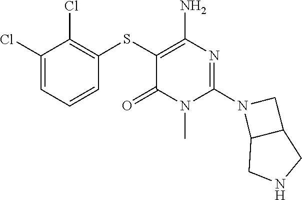 [SMILES: Cn1c(N2CC3CNCC32)nc(N)c(Sc2cccc(Cl)c2Cl)c1=O] | 6-amino-2-(3,6- diazabicyclo[3.2.0]heptan-6-yl)-5- ((2,3-dichlorophenyl)thio)-3- methylpyrimidin-4(3H)-one |
| 59 | 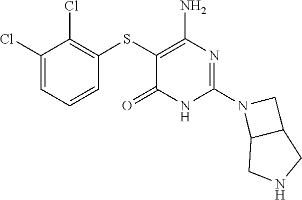 [SMILES: Nc1nc(N2CC3CNCC32)[nH]c(=O)c1Sc1cccc(Cl)c1Cl] | 6-amino-2-(3,6- diazabicyclo[3.2.0]heptan-6-yl)-5- ((2,3-dichlorophenyl)thio) pyrimidin-4(3H)-one |
| 60 | 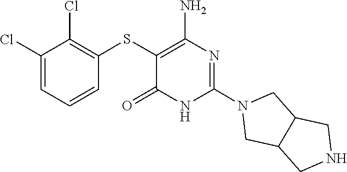 [SMILES: Nc1nc(N2CC3CNCC3C2)[nH]c(=O)c1Sc1cccc(Cl)c1Cl] | 6-amino-5-((2,3- dichlorophenyl)thio)-2- (hexahydropyrrolo[3,4-c]pyrrol- 2(1H)-yl)pyrimidin-4(3H)-one |
| 61 | 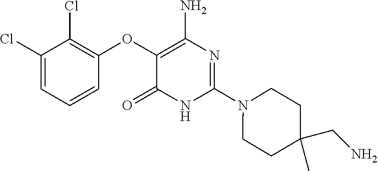 [SMILES: CC1(CN)CCN(c2nc(N)c(Oc3cccc(Cl)c3Cl)c(=O)[nH]2)CC1] | 6-amino-2-(4-(aminomethyl)- methylpiperidin-1-yl)-5-(2,3- dichlorophenoxy)pyrimidin-4(3H)- one |
| 62 | 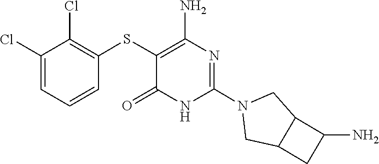 [SMILES: Nc1nc(N2CC3CC(N)C3C2)[nH]c(=O)c1Sc1cccc(Cl)c1Cl] | 6-amino-2-(6-amino-3- azabicyclo[3.2.0]heptan-3-yl)-5- ((2,3-dichlorophenyl)thio) pyrimidin-4(3H)-one |
| 63 | 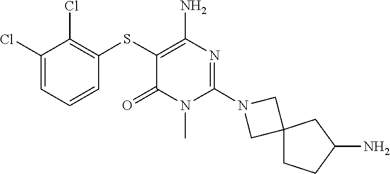 [SMILES: Cn1c(N2CC3(CCC(N)C3)C2)nc(N)c(Sc2cccc(Cl)c2Cl)c1=O] | 6-amino-2-(6-amino-2- azaspiro[3.4]octan-2-yl)-5-((2,3- dichlorophenyl)thio)-3- methylpyrimidin-4(3H)-one |
| 64 | 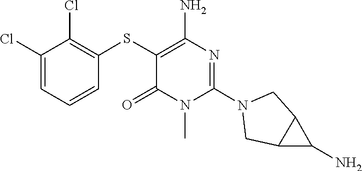 [SMILES: Cn1c(N2CC3C(N)C3C2)nc(N)c(Sc2cccc(Cl)c2Cl)c1=O] | 6-amino-2-(6-amino-3- azabicyclo[3.1.0]hexan-3-yl)-5- ((2,3-dichlorophenyl)thio)-3- methylpyrimidin-4(3H)-one |
| 65 | 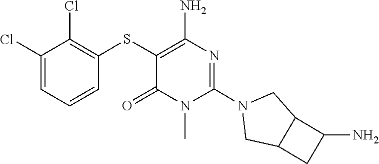 [SMILES: Cn1c(N2CC3CC(N)C3C2)nc(N)c(Sc2cccc(Cl)c2Cl)c1=O] | 6-amino-2-(6-amino-3- azabicyclo[3.2.0]heptan-3-yl)-5- ((2,3-dichlorophenyl)thio)-3- methylpyrimidin-4(3H)-one |
| 66 | 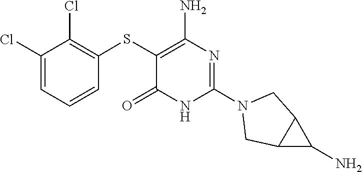 [SMILES: Nc1nc(N2CC3C(N)C3C2)[nH]c(=O)c1Sc1cccc(Cl)c1Cl] | 6-amino-2-(6-amino-3- azabicyclo[3.1.0]hexan-3-yl)-5- ((2,3-dichlorophenyl)thio) pyrimidin-4(3H)-one |
| 67 | 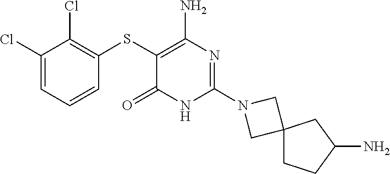 [SMILES: Nc1nc(N2CC3(CCC(N)C3)C2)[nH]c(=O)c1Sc1cccc(Cl)c1Cl] | 6-amino-2-(6-amino-2- azaspiro[3.4]octan-2-yl)-5-((2,3- dichlorophenyl)thio)pyrimidin- 4(3H)-one |
| 68 | 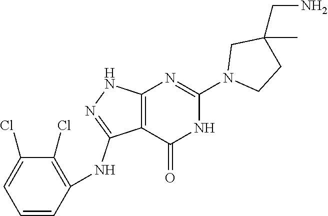 [SMILES: CC1(CN)CCN(c2nc3[nH]nc(Nc4cccc(Cl)c4Cl)c3c(=O)[nH]2)C1] | 6-(3-(aminomethyl)-3- methylpyrrolidin-1-yl)-3-((2,3- dichlorophenyl)amino)-1,5- dihydro-4H-pyrazolo[3,4- d]pyrimidin-4-one |
| 69 | 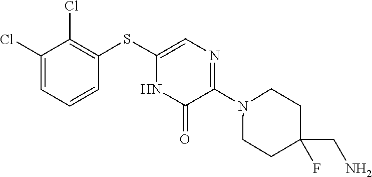 [SMILES: NCC1(F)CCN(c2ncc(Sc3cccc(Cl)c3Cl)[nH]c2=O)CC1] | 3-(4-(aminomethyl)-4- fluoropiperidin-1-yl)-6-((2,3- dichlorophenyl)thio)pyrazin-2 (1H)-one |
| 70 | 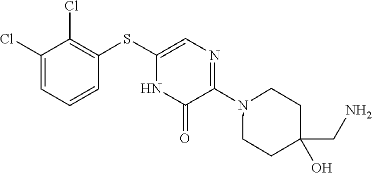 [SMILES: NCC1(O)CCN(c2ncc(Sc3cccc(Cl)c3Cl)[nH]c2=O)CC1] | 3-(4-(aminomethyl)-4- hydroxypiperidin-1-yl)-6-((2,3- dichlorophenyl)thio)pyrazin-2 (1H)-one |
| 71 | 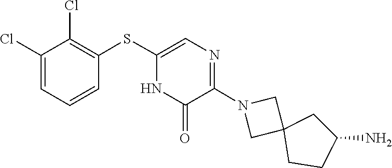 [SMILES: N[C@@H]1CCC2(C1)CN(c1ncc(Sc3cccc(Cl)c3Cl)[nH]c1=O)C2] | (R)-3-(6-amino-2- azaspiro[3.4]octan-2-yl)-6-((2,3- dichlorophenyl)thio)pyrazin-2 (1H)-one |
| 72 | 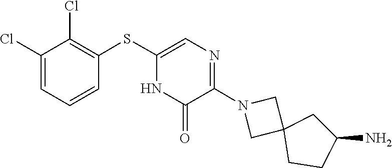 [SMILES: N[C@H]1CCC2(C1)CN(c1ncc(Sc3cccc(Cl)c3Cl)[nH]c1=O)C2] | (S)-3-(6-amino-2- azaspiro[3.4]octan-2-yl)-6-((2,3- dichlorophenyl)thio)pyrazin-2 (1H)-one |
| 73 | 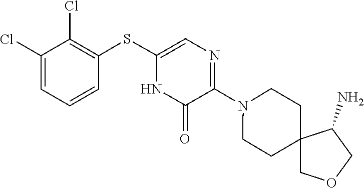 [SMILES: N[C@@H]1COCC12CCN(c1ncc(Sc3cccc(Cl)c3Cl)[nH]c1=O)CC2] | (S)-3-(4-amino-2-oxa-8- azaspiro[4.5]decan-8-yl)-6-((2,3- dichlorophenyl)thio)pyrazin-2 (1H)-one |
| 74 | 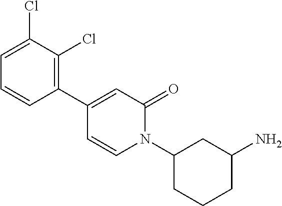 [SMILES: NC1CCCC(n2ccc(-c3cccc(Cl)c3Cl)cc2=O)C1] | 1-(3-aminocyclohexyl)-4-(2,3- dichlorophenyl)pyridin-2(1H)-one |
| 75 | 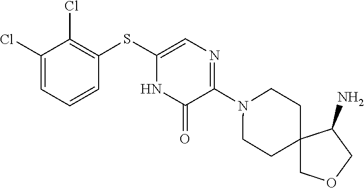 [SMILES: N[C@H]1COCC12CCN(c1ncc(Sc3cccc(Cl)c3Cl)[nH]c1=O)CC2] | (R)-3-(4-amino-2-oxa-8- azaspiro[4.5]decan-8-yl)-6-((2,3- dichlorophenyl)thio)pyrazin-2 (1H)-one |
| 76 | 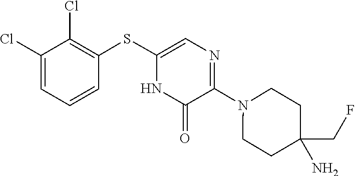 [SMILES: NC1(CF)CCN(c2ncc(Sc3cccc(Cl)c3Cl)[nH]c2=O)CC1] | 3-(4-amino-4- (fluoromethyl)piperidin-1-yl)-6- ((2,3-dichlorophenyl)thio)pyrazin- 2(1H)-one |
| 77 | 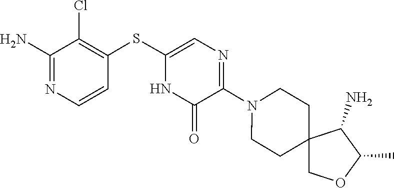 [SMILES: C[C@@H]1OCC2(CCN(c3ncc(Sc4ccnc(N)c4Cl)[nH]c3=O)CC2)[C@@H]1N] | 6-((2-amino-3-chloropyridin-4- yl)thio)-3-((3S,4S)-4-amino-3- methyl-2-oxa-8-azaspiro[4.5] decan-8-yl)pyrazin-2(1H)-one |
| 78 | 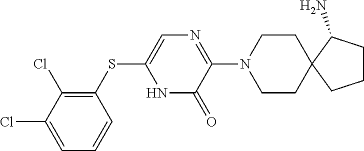 [SMILES: N[C@@H]1CCCC12CCN(c1ncc(Sc3cccc(Cl)c3Cl)[nH]c1=O)CC2] | (R)-3-(1-amino-8- azaspiro[4.5]decan-8-yl)-6-((2,3- dichlorophenyl)thio)pyrazin-2 (1H)-one |
| 79 | 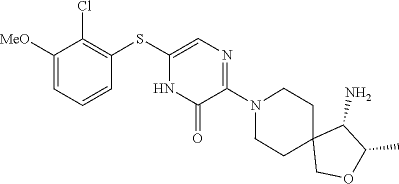 [SMILES: COc1cccc(Sc2cnc(N3CCC4(CC3)CO[C@@H](C)[C@H]4N)c(=O)[nH]2)c1Cl] | 3-((3S,4S)-4-amino-3-methyl-2- oxa-8-azaspiro[4.5]decan-8-yl)-6- ((2-chloro-3- methoxyphenyl)thio)pyrazin-2 (1H)-one |
| 80 | 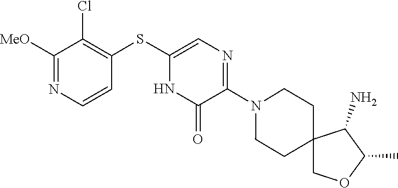 [SMILES: COc1nccc(Sc2cnc(N3CCC4(CC3)CO[C@@H](C)[C@H]4N)c(=O)[nH]2)c1Cl] | 3-((3S,4S)-4-amino-3-methyl-2- oxa-8-azaspiro[4.5]decan-8-yl)-6- ((3-chloro-2-methoxypyridin-4- yl)thio)pyrazin-2(1H)-one |
| 81 | 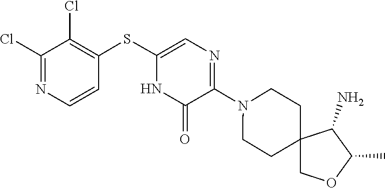 [SMILES: C[C@@H]1OCC2(CCN(c3ncc(Sc4ccnc(Cl)c4Cl)[nH]c3=O)CC2)[C@@H]1N] | 3-((3S,4S)-4-amino-3-methyl-2- oxa-8-azaspiro[4.5]decan-8-yl)-6- ((2,3-dichloropyridin-4- yl)thio)pyrazin-2(1H)-one |
| 82 | 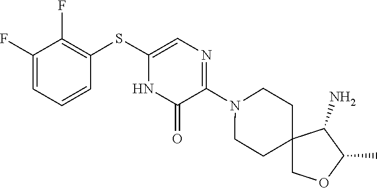 [SMILES: C[C@@H]1OCC2(CCN(c3ncc(Sc4cccc(F)c4F)[nH]c3=O)CC2)[C@@H]1N] | 3-((3S,4S)-4-amino-3-methyl-2- oxa-8-azaspiro[4.5]decan-8-yl)-6- ((2,3-difluorophenyl)thio)pyrazin- 2(1H)-one |
| 83 | 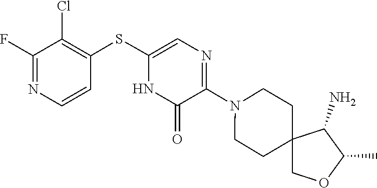 [SMILES: C[C@@H]1OCC2(CCN(c3ncc(Sc4ccnc(F)c4Cl)[nH]c3=O)CC2)[C@@H]1N] | 3-((3S,4S)-4-amino-3-methyl-2- oxa-8-azaspiro[4.5]decan-8-yl)-6- ((3-chloro-2-fluoropyridin-4- yl)thio)pyrazin-2(1H)-one |
| 84 | 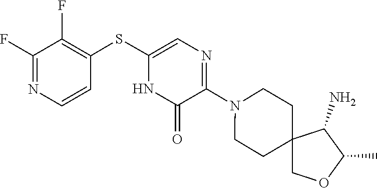 [SMILES: C[C@@H]1OCC2(CCN(c3ncc(Sc4ccnc(F)c4F)[nH]c3=O)CC2)[C@@H]1N] | 4-((5-(4-(aminomethyl)-4- methylpiperidin-1-yl)-6-oxo-1,6- dihydropyrazin-2-yl)thio)-3,3- difluoroindolin-2-one |
| 85 | 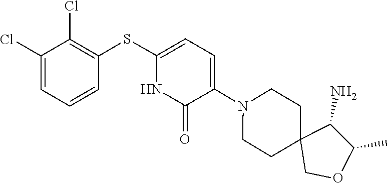 [SMILES: C[C@@H]1OCC2(CCN(c3ccc(Sc4cccc(Cl)c4Cl)[nH]c3=O)CC2)[C@@H]1N] | 3-((3S,4S)-4-amino-3-methyl-2- oxa-8-azaspiro[4.5]decan-8-yl)-6- ((2,3-dichlorophenyl)thio)pyridin- 2(1H)-one |
| 86 | 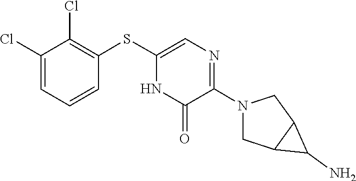 [SMILES: NC1C2CN(c3ncc(Sc4cccc(Cl)c4Cl)[nH]c3=O)CC12] | 3-(6-amino-3- azabicyclo[3.1.0]hexan-3-yl)-6- ((2,3-dichlorophenyl)thio)pyrazin- 2(1H)-one |
| 87 | 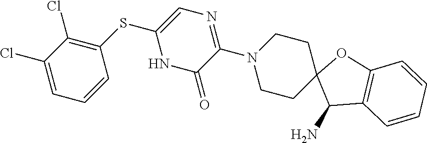 [SMILES: N[C@@H]1c2ccccc2OC12CCN(c1ncc(Sc3cccc(Cl)c3Cl)[nH]c1=O)CC2] | (R)-3-(3-amino-3H- spiro[benzofuran-2,4′-piperidin]- 1′-yl)-6-((2,3- dichlorophenyl)thio)pyrazin-2 (1H)-one |
| 88 | 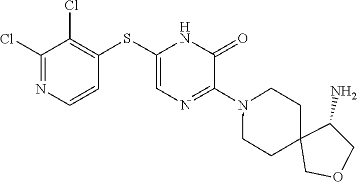 [SMILES: N[C@@H]1COCC12CCN(c1ncc(Sc3ccnc(Cl)c3Cl)[nH]c1=O)CC2] | (S)-3-(4-amino-2-oxa-8- azaspiro[4.5]decan-8-yl)-6-((2,3- dichloropyridin-4-yl)thio)pyrazin- 2(1H)-one |
| 89 | 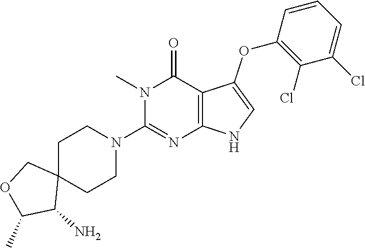 [SMILES: C[C@@H]1OCC2(CCN(c3nc4[nH]cc(Oc5cccc(Cl)c5Cl)c4c(=O)n3C)CC2)[C@@H]1N] | 2-((3S,4S)-4-amino-3-methyl-2- oxa-8-azaspiro[4.5]decan-8-yl)-5- (2,3-dichlorophenoxy)-3-methyl- 3,7-dihydro-4H-pyrrolo[2,3- d]pyrimidin-4-one |
| 90 | 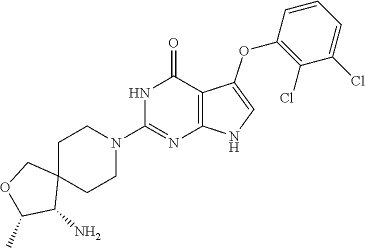 [SMILES: C[C@@H]1OCC2(CCN(c3nc4[nH]cc(Oc5cccc(Cl)c5Cl)c4c(=O)[nH]3)CC2)[C@@H]1N] | 2-((3S,4S)-4-amino-3-methyl-2- oxa-8-azaspiro[4.5]decan-8-yl)-5- (2,3-dichlorophenoxy)-3,7- dihydro-4H-pyrrolo[2,3-d] pyrimidin-4-one |
| 91 | 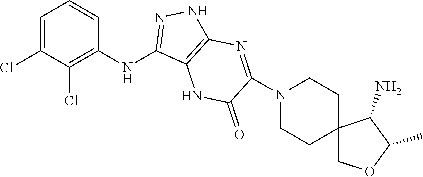 [SMILES: C[C@@H]1OCC2(CCN(c3nc4[nH]nc(Nc5cccc(Cl)c5Cl)c4[nH]c3=O)CC2)[C@@H]1N] | 6-((3S,4S)-4-amino-3-methyl-2- oxa-8-azaspiro[4.5]decan-8-yl)-3- ((2,3-dichlorophenyl)amino)-1,4- dihydro-5H-pyrazolo[3,4-b] pyrazin-5-one |
| 92 | 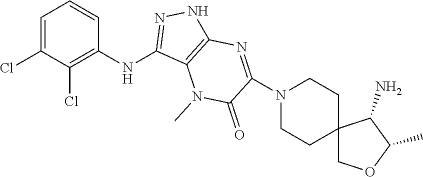 [SMILES: C[C@@H]1OCC2(CCN(c3nc4[nH]nc(Nc5cccc(Cl)c5Cl)c4n(C)c3=O)CC2)[C@@H]1N] | 6-((3S,4S)-4-amino-3-methyl-2- oxa-8-azaspiro[4.5]decan-8-yl)-3- ((2,3-dichlorophenyl)amino)-4- methyl-1,4-dihydro-5H- pyrazolo[3,4-b]pyrazin-5-one |
| 93 | 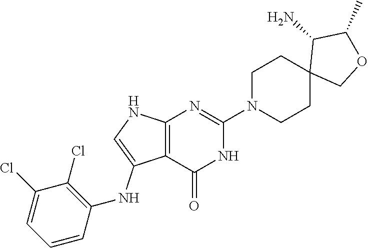 [SMILES: C[C@@H]1OCC2(CCN(c3nc4[nH]cc(Nc5cccc(Cl)c5Cl)c4c(=O)[nH]3)CC2)[C@@H]1N] | 2-((3S,4S)-4-amino-3-methyl-2- oxa-8-azaspiro[4.5]decan-8-yl)-5- ((2,3-dichlorophenyl)amino)-3,7- dihydro-4H-pyrrolo[2,3-d] pyrimidin-4-one |
| 94 | 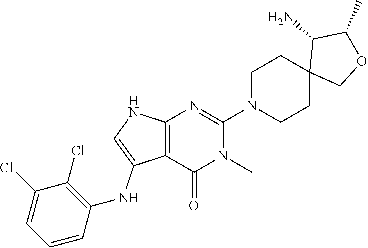 [SMILES: C[C@@H]1OCC2(CCN(c3nc4[nH]cc(Nc5cccc(Cl)c5Cl)c4c(=O)n3C)CC2)[C@@H]1N] | 2-((3S,4S)-4-amino-3-methyl-2- oxa-8-azaspiro[4.5]decan-8-yl)-5- ((2,3-dichlorophenyl)amino)-3- methyl-3,7-dihydro-4H-pyrrolo [2,3-d]pyrimidin-4-one |
| 95 | 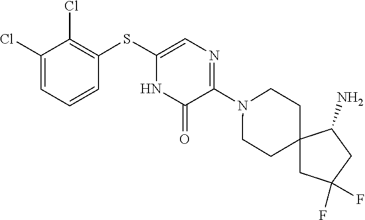 [SMILES: N[C@@H]1CC(F)(F)CC12CCN(c1ncc(Sc3cccc(Cl)c3Cl)[nH]c1=O)CC2] | (R)-3-(1-amino-3,3-difluoro-8- azaspiro[4.5]decan-8-yl)-6-((2,3- dichlorophenyl)thio)pyrazin-2 (1H)-one |
| 96 | 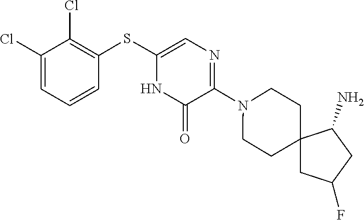 [SMILES: N[C@@H]1CC(F)CC12CCN(c1ncc(Sc3cccc(Cl)c3Cl)[nH]c1=O)CC2] | 3-((1R)-1-amino-3-fluoro-8- azaspiro[4.5]decan-8-yl)-6-((2,3- dichlorophenyl)thio)pyrazin-2 (1H)-one |
| 97 | 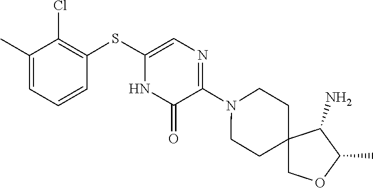 | 3-((3S,4S)-4-amino-3-methyl-2- oxa-8-azaspiro[4.5]decan-8-yl)-6- ((2-chloro-3- methylphenyl)thio)pyrazin-2(1H)- one |
| 98 | 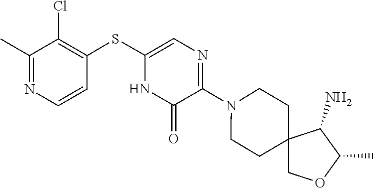 | 3-((3S,4S)-4-amino-3-methyl-2- oxa-8-azaspiro[4.5]decan-8-yl)-6- ((3-chloro-2-methylpyridin-4- yl)thio)pyrazin-2(1H)-one |
| 99 | 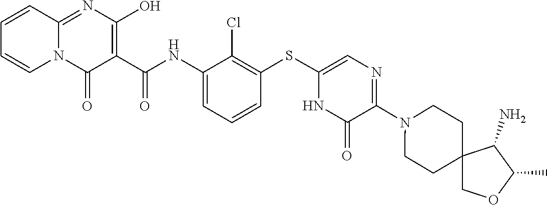 [SMILES: C[C@@H]1OCC2(CCN(c3ncc(Sc4cccc(NC(=O)c5c(O)nc6ccccn6c5=O)c4Cl)[nH]c3=O)CC2)[C@@H]1N] | N-(3-((5-((3S,4S)-4-amino-3- methyl-2-oxa-8-azaspiro[4.5] decan-8-yl)-6-oxo-1,6- dihydropyrazin-2-yl)thio)-2- chlorophenyl)-2-hydroxy-4-oxo- 4H-pyrido[1,2-a]pyrimidine-3- carboxamide |
| 100 | 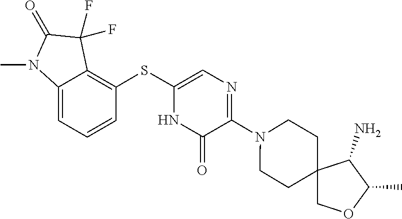 | 4-((5-((3S,4S)-4-amino-3-methyl- 2-oxa-8-azaspiro[4.5]decan-8-yl)- 6-oxo-1,6-dihydropyrazin-2-yl) thio)-3,3-difluoro-1- methylindolin-2-one |
| 101 | 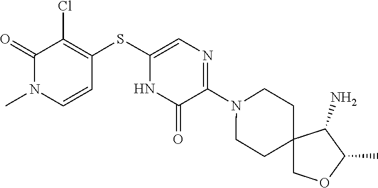 | 3-((3S,4S)-4-amino-3-methyl-2- oxa-8-azaspiro[4.5]decan-8-yl)-6- ((3-chloro-1-methyl-2-oxo-1,2- dihydropyridin-4-yl)thio)pyrazin- 2(1H)-one |
| 102 | 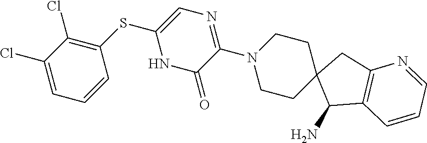 [SMILES: N[C@@H]1c2cccnc2CC12CCN(c1ncc(Sc3cccc(Cl)c3Cl)[nH]c1=O)CC2] | (S)-3-(5-amino-5,7- dihydrospiro[cyclopenta[b] pyridine-6,4′-piperidin]-1′-yl)-6- ((2,3-dichlorophenyl)thio)pyrazin- 2(1H)-one |
| 103 | 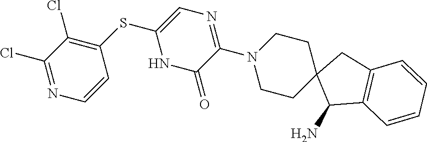 [SMILES: N[C@@H]1c2ccccc2CC12CCN(c1ncc(Sc3ccnc(Cl)c3Cl)[nH]c1=O)CC2] | (S)-3-(1-amino-1,3- dihydrospiro[indene-2,4′- piperidin]-1′-yl)-6-((2,3- dichloropyridin-4-yl)thio)pyrazin- 2(1H)-one |
| 104 | 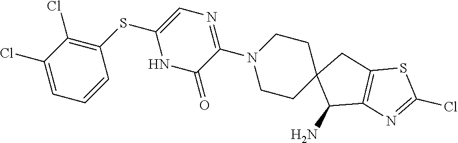 [SMILES: N[C@@H]1c2nc(Cl)sc2CC12CCN(c1ncc(Sc3cccc(Cl)c3Cl)[nH]c1=O)CC2] | (S)-3-(4-amino-2-chloro-4,6- dihydrospiro[cyclopenta[d] thiazole-5,4′-piperidin]-1′-yl)-6- ((2,3-dichlorophenyl)thio)pyrazin- 2(1H)-one |
| 105 | 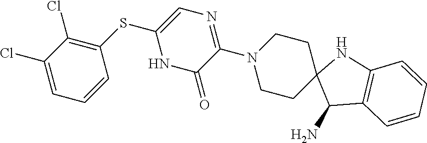 [SMILES: N[C@@H]1c2ccccc2NC12CCN(c1ncc(Sc3cccc(Cl)c3Cl)[nH]c1=O)CC2] | (R)-3-(3-aminospiro[indoline-2,4′- piperidin]-1′-yl)-6-((2,3- dichlorophenyl)thio)pyrazin- 2(1H)-one |
| 106 | 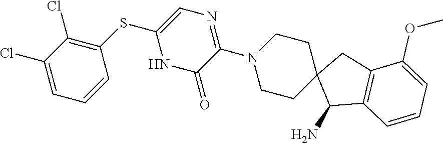 [SMILES: COc1cccc2c1CC1(CCN(c3ncc(Sc4cccc(Cl)c4Cl)[nH]c3=O)CC1)[C@@H]2N] | (S)-3-(1-amino-4-methoxy-1,3- dihydrospiro[indene-2,4′- piperidin]-1′-yl)-6-((2,3- dichlorophenyl)thio)pyrazin- 2(1H)-one |
| 107 | 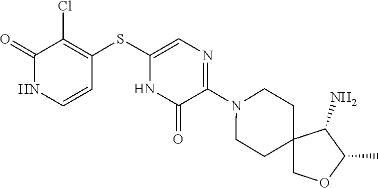 [SMILES: C[C@@H]1OCC2(CCN(c3ncc(Sc4cc[nH]c(=O)c4Cl)[nH]c3=O)CC2)[C@@H]1N] | 3-((3S,4S)-4-amino-3-methyl-2- oxa-8-azaspiro[4.5]decan-8-yl)-6- ((3-chloro-2-oxo-1,2-dihydro- pyridin-4-yl)thio)pyrazin-2(1H)- one |
| 108 | 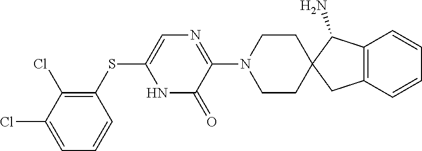 [SMILES: N[C@@H]1c2ccccc2CC12CCN(c1ncc(Sc3cccc(Cl)c3Cl)[nH]c1=O)CC2] | (S)-3-(1-amino-1,3) dihydrospiro[indene-2,4′- piperidin]-1′-yl)-6-((2,3- dichlorophenyl)thio)pyrazin-2 (1H)-one |
| 109 | 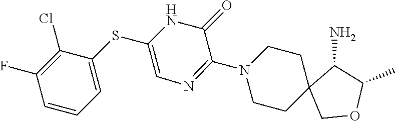 [SMILES: C[C@@H]1OCC2(CCN(c3ncc(Sc4cccc(F)c4Cl)[nH]c3=O)CC2)[C@@H]1N] | 3-((3S,4S)-4-amino-3-methyl-2- oxa-8-azaspiro[4.5]decan-8-yl)-6- ((2-chloro-3- fluorophenyl)thio)pyrazin-2(1H)- one |
| 110 | 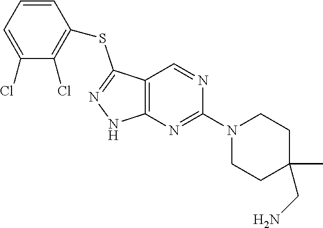 [SMILES: CC1(CN)CCN(c2ncc3c(Sc4cccc(Cl)c4Cl)n[nH]c3n2)CC1] | (1-(3-(2,3- dichlorophenyl)thio)-1H- pyrazolo[3,4-d]pyrimidin-6-yl)-4- methylpiperidin-4-yl) methanamine |
| 111 | 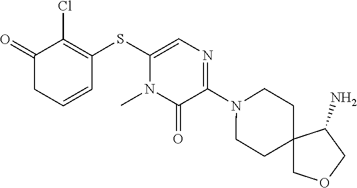 [SMILES: Cn1c(SC2=C(Cl)C(=O)CC=C2)cnc(N2CCC3(CC2)COC[C@H]3N)c1=O] | 3-(4-amino-2-oxa-8- azaspiro[4.5]decan-8-yl)-6-((2,3- dichlorophenyl)thio)-1- methylpyrazin-2(1H)-one |
| 112 | 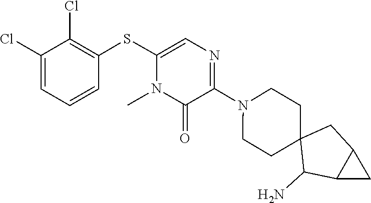 [SMILES: Cn1c(Sc2cccc(Cl)c2Cl)cnc(N2CCC3(CC2)CC2CC2C3N)c1=O] | 3-(2- aminospiro[bicyclo[3.1.0]hexane- 3,4′-piperidin]-1′-yl)-6-((2,3- dichlorophenyl)thio)-1- methylpyrazin-2(1H)-one |
| 113 | 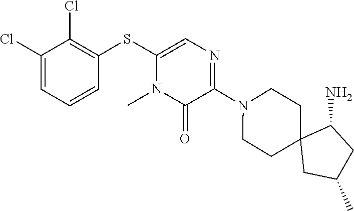 [SMILES: C[C@H]1C[C@@H](N)C2(CCN(c3ncc(Sc4cccc(Cl)c4Cl)n(C)c3=O)CC2)C1] | 3-((1R,3R)-1-amino-3-methyl-8- azaspiro[4.5]decan-8-yl)-6-((2,3- dichlorophenyl)thio)-1- methylpyrazin-2(1H)-one |
| 114 | 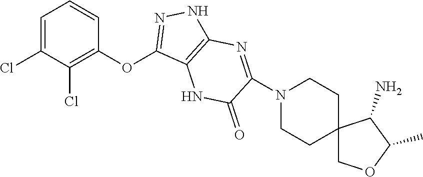 [SMILES: C[C@@H]1OCC2(CCN(c3nc4[nH]nc(Oc5cccc(Cl)c5Cl)c4[nH]c3=O)CC2)[C@@H]1N] | 6-((3S,4S)-4-amino-3-methyl-2- oxa-8-azaspiro[4.5]decan-8-yl)-3- (2,3-dichlorophenoxy)-1,4- dihydro-5H-pyrazolo[3,4-b] pyrazin-5-one |

Some embodiments include one of the compounds in Table 1B, wherein any of the compounds in Table 1B below may be optionally substituted.

TABLE 1B

| Com- | | |
|---|---|---|
| pound | | |
| ID | Structure | Compound name |
| 115 | 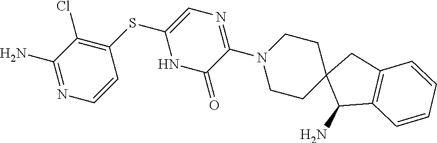 [SMILES: Nc1nccc(Sc2cnc(N3CCC4(CC3)Cc3ccccc3[C@H]4N)c(=O)[nH]2)c1Cl] | (S)-3-(1-amino-1,3- dihydrospiro[indene-2,4′- piperidin]-1′-yl)-6-((2-amino-3- chloropyridin-4-yl)thio)pyrazin- 2(1H)-one |
| 116 | 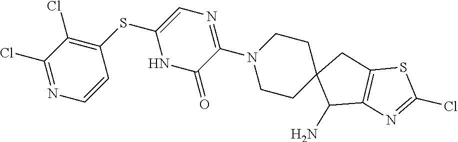 [SMILES: NC1c2nc(Cl)sc2CC12CCN(c1ncc(Sc3ccnc(Cl)c3Cl)[nH]c1=O)CC2] | 3-(4-amino-2-chloro-4,6- dihydrospiro[cyclopenta[d] thiazole-5,4′-piperidin]-1′-yl)-6- ((2,3-dichloropyridin-4- yl)thio)pyrazin-2(1H)-one |
| 117 | 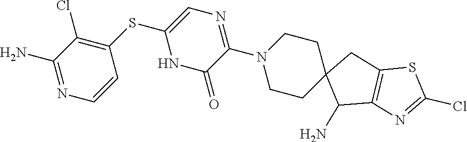 [SMILES: Nc1nccc(Sc2cnc(N3CCC4(CC3)Cc3sc(Cl)nc3C4N)c(=O)[nH]2)c1Cl] | 3-(4-amino-2-chloro-4,6- dihydrospiro[cyclopenta[d] thiazole-5,4′-piperidin]-1′-yl)-6- ((2-amino-3-chloropyridin-4- yl)thio)pyrazin-2(1H)-one |
| 118 | 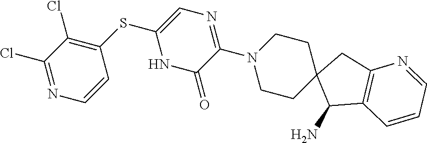 [SMILES: N[C@@H]1c2cccnc2CC12CCN(c1ncc(Sc3ccnc(Cl)c3Cl)[nH]c1=O)CC2] | (S)-3-(5-amino-5,7- dihydrospiro[cyclopenta[b] pyridine-6,4′-piperidin]-1′-yl)-6- ((2,3-dichloropyridin-4- yl)thio)pyrazin-2(1H)-one |
| 119 | 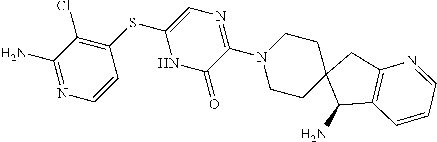 [SMILES: Nc1nccc(Sc2cnc(N3CCC4(CC3)Cc3ncccc3[C@H]4N)c(=O)[nH]2)c1Cl] | (S)-6-((2-amino-3-chloropyridin-4- yl)thio)-3-(5-amino-5,7- dihydrospiro[cyclopenta[b] pyridine-6,4′-piperidin]-1′- yl)pyrazin-2(1H)-one |
| 120 | 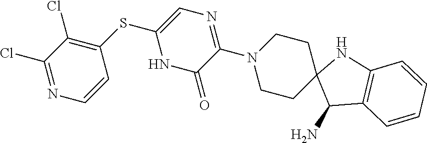 [SMILES: N[C@@H]1c2ccccc2NC12CCN(c1ncc(Sc3ccnc(Cl)c3Cl)[nH]c1=O)CC2] | (R)-3-(3-aminospiro[indoline-2,4′- piperidin]-1′-yl)-6-((2,3- dichloropyridin-4-yl)thio)pyrazin- 2(1H)-one |
| 121 | 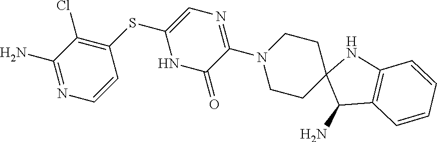 [SMILES: Nc1nccc(Sc2cnc(N3CCC4(CC3)Nc3ccccc3[C@H]4N)c(=O)[nH]2)c1Cl] | (R)-6-((2-amino-3-chloropyridin-4- yl)thio)-3-(3-aminospiro[indoline- 2,4′-piperidin]-1′-yl)pyrazin-2(1H)- one |
| 122 | 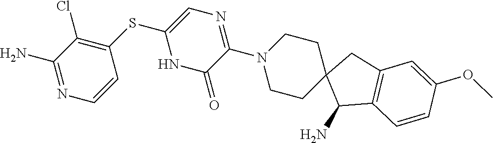 [SMILES: COc1ccc2c(c1)CC1(CCN(c3ncc(Sc4ccnc(N)c4Cl)[nH]c3=O)CC1)[C@@H]2N] | (S)-6-((2-amino-3-chloropyridin-4- yl)thio)-3-(1-amino-5-methoxy- 1,3-dihydrospiro[indene-2,4′- piperidin]-1′-yl)pyrazin-2(1H)-one |
| 123 | 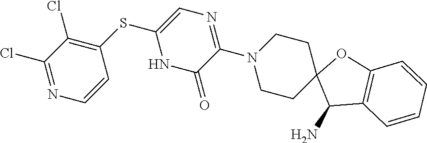 [SMILES: N[C@@H]1c2ccccc2OC12CCN(c1ncc(Sc3ccnc(Cl)c3Cl)[nH]c1=O)CC2] | (R)-3-(3-amino-3H- spiro[benzofuran-2,4′-piperidin]- 1′-yl)-6-((2,3-dichloropyridin-4- yl)thio)pyrazin-2(1H)-one |
| 124 | 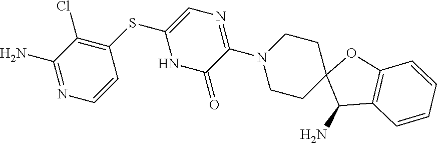 [SMILES: Nc1nccc(Sc2cnc(N3CCC4(CC3)Oc3ccccc3[C@H]4N)c(=O)[nH]2)c1Cl] | (R)-6-((2-amino-3-chloropyridin-4- yl)thio)-3-(3-amino-3H- spiro[benzofuran-2,4′-piperidin]- 1′-yl)pyrazin-2(1H)-one |
| 125 | 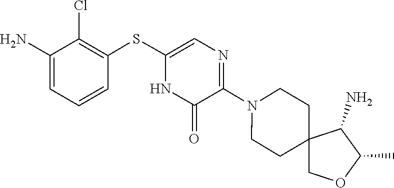 [SMILES: C[C@@H]1OCC2(CCN(c3ncc(Sc4cccc(N)c4Cl)[nH]c3=O)CC2)[C@@H]1N] | 6-((3-amino-2-chlorophenyl)thio)- ((3S,4S)-4-amino-3-methyl-2-oxa- 8-azaspiro[4.5]decan-8-yl)pyrazin- 2(1H)-one |
| 126 | 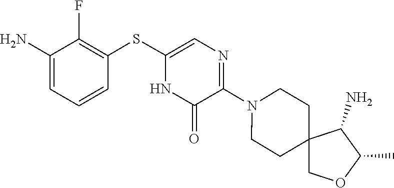 [SMILES: C[C@@H]1OCC2(CCN(c3ncc(Sc4cccc(N)c4F)[nH]c3=O)CC2)[C@@H]1N] | 6-((3-amino-2-fluorophenyl)thio)- 3-((3S,4S)-4-amino-3-methyl-2- oxa-8-azaspiro[4.5]decan-8- yl)pyrazin-2(1H)-one |
| 127 | 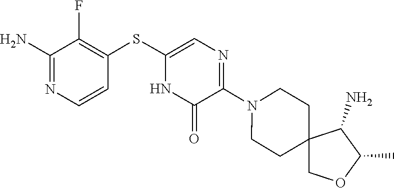 [SMILES: C[C@@H]1OCC2(CCN(c3ncc(Sc4ccnc(N)c4F)[nH]c3=O)CC2)[C@@H]1N] | 6-((2-amino-3-fluoropyridin-4- yl)thio)-3-((3S,4S)-4-amino-3- methyl-2-oxa-8-azaspiro[4.5] decan-8-yl)pyrazin-2(1H)-one |
| 128 | 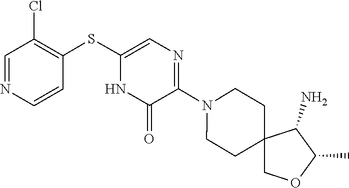 [SMILES: C[C@@H]1OCC2(CCN(c3ncc(Sc4ccncc4Cl)[nH]c3=O)CC2)[C@@H]1N] | 3-((3S,4S)-4-amino-3-methyl-2- oxa-8-azaspiro[4.5]decan-8-yl)-6- ((3-chloropyridin-4-yl)thio) pyrazin-2(1H)-one |
| 129 | 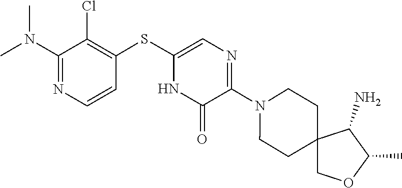 | 3-((3S,4S)-4-amino-3-methyl-2- oxa-8-azaspiro[4.5]decan-8-yl)-6- ((3-chloro-2- (dimethylamino)pyridin-4- yl)thio)pyrazin-2(1H)-one |
| 130 | 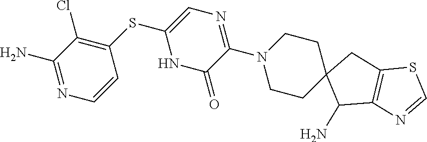 [SMILES: Nc1nccc(Sc2cnc(N3CCC4(CC3)Cc3scnc3C4N)c(=O)[nH]2)c1Cl] | 6-((2-amino-3-chloropyridin-4- yl)thio)-3-(4-amino-4,6- dihydrospiro[cyclopenta[d] thiazole-5,4′-piperidin]-1′- yl)pyrazin-2(1H)-one |
| 131 | 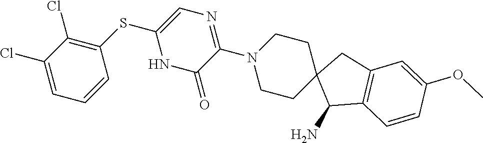 [SMILES: COc1ccc2c(c1)CC1(CCN(c3ncc(Sc4cccc(Cl)c4Cl)[nH]c3=O)CC1)[C@@H]2N] | (S)-3-(1-amino-5-methoxy-1,3- dihydrospiro[indene-2,4′- piperidin]-1′-yl)-6-((2,3- dichlorophenyl)thio)pyrazin-2 (1H)-one |
| 132 | 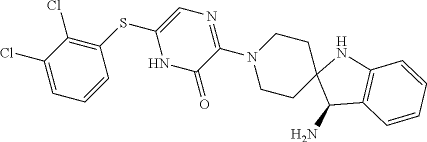 [SMILES: N[C@@H]1c2ccccc2NC12CCN(c1ncc(Sc3cccc(Cl)c3Cl)[nH]c1=O)CC2] | (R)-3-(3-aminospiro[indoline-2,4′- piperidin]-1′-yl)-6-((2,3- dichlorophenyl)thio)pyrazin-2 (1H)-one |
| 133 | 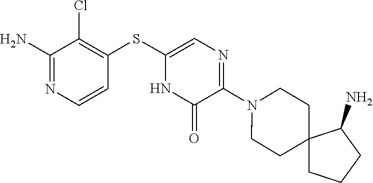 [SMILES: Nc1nccc(Sc2cnc(N3CCC4(CCC[C@@H]4N)CC3)c(=O)[nH]2)c1Cl] | (S)-6-((2-amino-3-chloropyridin-4- yl)thio)-3-(1-amino-8- azaspiro[4.5]decan-8-yl)pyrazin- 2(1H)-one |
| 134 | 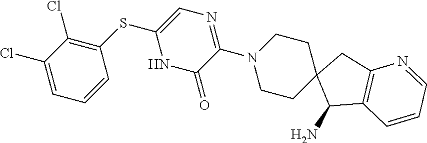 [SMILES: N[C@@H]1c2cccnc2CC12CCN(c1ncc(Sc3cccc(Cl)c3Cl)[nH]c1=O)CC2] | (S)-3-(5-amino-5,7- dihydrospiro[cyclopenta[b] pyridine-6,4′-piperidin]-1′-yl)-6- ((2,3-dichlorophenyl)thio)pyrazin- 2(1H)-one |
| 135 | 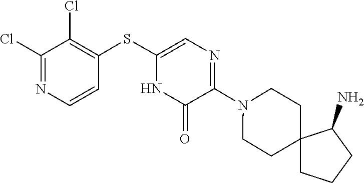 [SMILES: N[C@H]1CCCC12CCN(c1ncc(Sc3ccnc(Cl)c3Cl)[nH]c1=O)CC2] | (S)-3-(1-amino-8- azaspiro[4.5]decan-8-yl)-6-((2,3- dichloropyridin-4-yl)thio)pyrazin- 2(1H)-one |
| 136 | 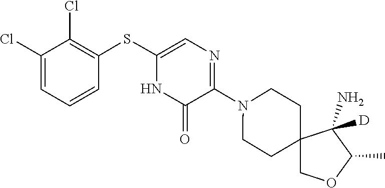 [SMILES: [2H][C@@]1(N)[C@H](C)OCC12CCN(c1ncc(Sc3cccc(Cl)c3Cl)[nH]c1=O)CC2] | 3-((3S,4S)-4-amino-3-methyl-2- oxa-8-azaspiro[4.5]decan-8-yl-4- d)-6-((2,3- dichlorophenyl)thio)pyrazin-2-ol |
| 137 | 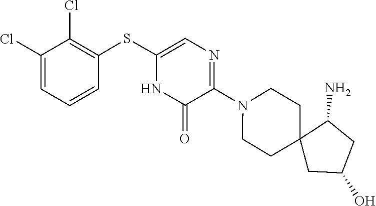 [SMILES: N[C@@H]1C[C@H](O)CC12CCN(c1ncc(Sc3cccc(Cl)c3Cl)[nH]c1=O)CC2] | 3-((1R,3R)-1-amino-3-hydroxy-8- azaspiro[4.5]decan-8-yl)-6-((2,3- dichlorophenyl)thio)pyrazin- 2(1H)-one |
| 138 | 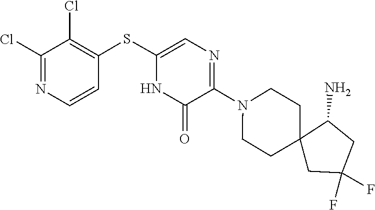 [SMILES: N[C@@H]1CC(F)(F)CC12CCN(c1ncc(Sc3ccnc(Cl)c3Cl)[nH]c1=O)CC2] | (R)-3-(1-amino-3,3-difluoro-8- azaspiro[4.5]decan-8-yl)-6-((2,3- dichloropyridin-4-yl)thio)pyrazin- 2(1H)-one |
| 139 | 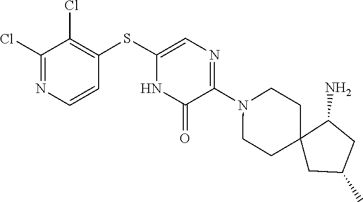 [SMILES: C[C@H]1C[C@@H](N)C2(CCN(c3ncc(Sc4ccnc(Cl)c4Cl)[nH]c3=O)CC2)C1] | 3-((1R,3R)-1-amino-3-methyl-8- azaspiro[4.5]decan-8-yl)-6-((2,3- dichloropyridin-4-yl)thio)pyrazin- 2(1H)-one |
| 140 | 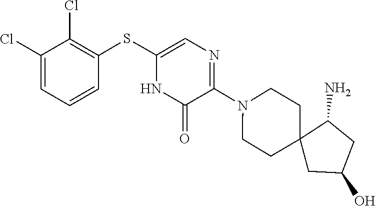 [SMILES: N[C@@H]1C[C@@H](O)CC12CCN(c1ncc(Sc3cccc(Cl)c3Cl)[nH]c1=O)CC2] | 3-((1R,3S)-1-amino-3-hydroxy-8- azaspiro[4.5]decan-8-yl)-6-((2,3- dichlorophenyl)thio)pyrazin- 2(1H)-one |
| 141 | 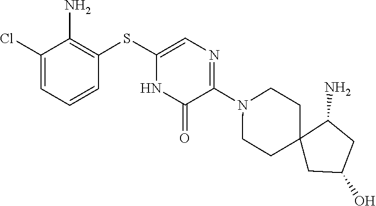 [SMILES: Nc1c(Cl)cccc1Sc1cnc(N2CCC3(CC2)C[C@@H](O)C[C@H]3N)c(=O)[nH]1] | 6-((2-amino-3-chlorophenyl)thio)- ((1R,3R)-1-amino-3-hydroxy-8- azaspiro[4.5]decan-8-yl)pyrazin- 2(1H)-one |

An example, not as an attempt to limit the scope of the disclosure, of a useful composition for a dosage form containing about 10-1000 mg of compound 83 is shown in Table 1C below:

TABLE 1C
Example of dosage form of compound 83

| Component | Amount (wt/wt) |
|---|---|
| Compound 83 | 30-70% |
| lubricant | 1-10% |
| diluent | 20-70% |
| disintegrant | 1-10% |

In some embodiments, a dosage form may comprise about 10-2000 mg of a subject compound described herein. In some embodiments, a dosage form may contain about 10-20 mg, about 20-30 mg, about 30-40 mg, about 40-50 mg, about 50-60 mg, about 60-70 mg, about 70-80 mg, about 80-90 mg, about 90-100 mg, about 100-150 mg, about 150-200 mg, about 200-250 mg, about 250-300 mg, about 300-350 mg, about 350-400 mg, about 400-450 mg, about 450-500 mg, about 500-600 mg, about 600-700 mg, about 700-800 mg, about 800-900 mg, about 900-1000 mg, about 1000-1500 mg, about 1500-2000 mg, about 10-50 mg, about 50-100 mg, about 100-200 mg, about 200-300 mg, about 300-400 mg, about 400-500 mg, about 10-2000 mg, about 10-1000 mg, about 10-500 mg, or any amount in a range bounded by any of the above values of a subject compound, such as a compound of Formula 1. The term “about 10-500 mg” described herein means about 10 mg to about 500 mg, and so on.

In some embodiments, a daily dose of a subject compound described herein may be in a range of about 1-100 mg/kg. In some embodiments, a daily dose may be about 1-5 mg/kg, about 5-10 mg/kg, about 10-15 mg/kg, about 15-20 mg/kg, about 20-25 mg/kg, about 25-30 mg/kg, about 30-35 mg/kg, about 35-40 mg/kg, about 40-45 mg/kg, about 45-50 mg/kg, about 50-55 mg/kg, about 55-60 mg/kg, about 60-65 mg/kg, about 65-70 mg/kg, about 70-75 mg/kg, about 75-80 mg/kg, about 80-85 mg/kg, about 85-90 mg/kg, about 60-95 mg/kg, about 95-100 mg/kg, about 1-60 mg/kg, about 1-50 mg/kg, about 1-40 mg/kg, about 1-30 mg/kg, about 1-10 mg/kg, about 10-20 mg/kg, about 20-30 mg/kg, about 30-40 mg/kg, about 40-50 mg/kg, about 50-60 mg/kg, about 60-70 mg/kg, about 70-80 mg/kg, about 80-90 mg/kg, about 90-100 mg/kg, about 1 mg/kg, about 5 mg/kg, about 10 mg/kg, about 20 mg/kg, about 30 mg/kg, about 40 mg/kg, about 50 mg/kg, about 60 mg/kg, or any amount in a range bounded by any of the above values of a subject compound, such as a compound of Formula 1. The term “about 1-60 mg/kg” described herein means about 1 mg/kg to about 60 mg/kg, and so on.

In some embodiments, the dosage form may comprise about 10-95% by weight of a subject compound described herein as compared to the total weight of the dosage form. In some embodiments, the dosage form may contain about 10-15%, about 15-20%, about 20-25%, about 25-30%, about 30-35%, about 35-40%, about 40-45%, about 45-50%, about 50-55%, about 55-60%, about 60-65%, about 65-70%, about 70-75%, about 75-80%, about 80-85%, about 85-90%, about 90-95%, about 10-20%, about 20-30%, about 30-40%, about 40-50%, about 50-60%, about 60-70%, about 70-80%, about 80-90%, about 10-30%, about 30-50%, about 50-70%, about 70-90%, or about 30-70%, about 30%, about 40%, about 50%, about 55%, about 60%, about 70% by weight of the total weight of the dosage form of a subject compound, such as a compound of Formula 1. The term “about 30-70%” described herein means about 30% to about 70%, and so on.

A pharmaceutical composition comprising a compound of Formula 1 may be adapted for oral, or parental, such as intravenous, intramuscular, topical, intraperitoneal, nasal, buccal, sublingual, or subcutaneous administration, or for administration via respiratory tract in the form of, for example, an aerosol or an air-suspended fine powder. The dosage of a compound of Formula 1 may vary depending on the route of administration, body weight, age, the type and condition of the disease being treated. A pharmaceutical composition provided herein may optionally comprise two or more compounds of the Formula 1 without an additional therapeutic agent, or may comprise an additional therapeutic agent (i.e., a therapeutic agent other than a compound provided herein). For example, the subject compounds can be administered simultaneously, sequentially, or separately in combination with at least one other therapeutic agent. The other therapeutic agent can be a small molecule, an antibody-drug conjugate, or a biologic. Therapeutic agents suitable for combination with a subject compound include, but are not limited to antibiotics, antiemetic agents, antidepressants, and antifungal agents, anti-inflammatory agents, antiviral agents, and anticancer agents that are known in the art. In some embodiments, the other therapeutic agents are chemotherapy agents, for example, mitotic inhibitors such as a taxane, a vinca alkaloid, paclitaxel; or tyrosine kinase inhibitors, for example Erlotinib; ALK inhibitors such as Crizotinib; BRAF inhibitors such as Vemurafanib; MEK inhibitors such as trametinib; or other anticancer agents, i.e. cisplatin, flutamide, gemcitabine, CTLA-4 inhibitors, PD-1 inhibitors and PD-L1 inhibitors. Such combination may offer significant advantages, including synergistic activity, in therapy. The pharmaceutical composition may be used for the treatment of cancer, autoimmune diseases, inflammatory diseases, autoinflammatory conditions, and other SHP2 mediated disorders in patients. The term “patient” herein means a mammal (e.g., a human or an animal). In some embodiments, the patient has cancer.

The pharmaceutical composition described herein can be prepared by combining a compound of Formula 1 with at least one pharmaceutical acceptable inert ingredient, such as a carrier, excipient, filler, lubricant, flavoring agent, buffer, etc., selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Some embodiments include a method of treating a SHP2 mediated disease or disorder comprising administering a therapeutically effective amount of a compound of Formula 1, or any compound described herein, or a pharmaceutically acceptable salt thereof (“subject compound”), or a pharmaceutical composition comprising a subject compound to a patient in need thereof. The term a “therapeutically effective amount” herein refers to an amount of a subject compound, or a pharmaceutical composition containing a subject compound, sufficient to be effective in inhibiting SHP2 and thus providing a benefit in the treatment of cancer, autoimmune diseases, inflammatory diseases, autoinflammatory conditions, and other SHP2 mediated disorders in patients, such as to delay or minimize symptoms associated with cancer, autoimmune, inflammatory diseases, and autoinflammatory conditions, or to ameliorate a disease or infection or cause thereof, or to prevent the further development of a disorder, or reducing the severity of symptoms that are otherwise expected to develop without treatment.

Many of the subject compounds described herein have superior anti-proliferation activities in various tumor types, for example lung cancer, esophageal cancer, pancreatic cancer, caecum cancer, head and neck cancer, colon cancer, melanoma, leukemia, or other metastatic solid tumors.

Many of the subject compounds described herein are very potent and selective, with enzymatic IC₅₀ less than 10 nM. The compounds described herein could display superior anti-tumor activities in various in vivo animal models. In some embodiments, administration of a subject compound described herein with a dose amount falls within the range of 1 mg/kg per day to 100 mg/kg per day could achieve the tumor regression or at least about 10%, at least about 20%, at least about 30%, at least about 40%, at least about 50%, at least about 60% at least about 70%, at least about 80%, at least about 90%, at least about 95%, about 10-20%, about 20-30%, about 30-40%, about 40-50%, about 50-60%, about 60-70%, about 70-80%, about 80-90%, about 90-100%, about 10-30%, about 30-50%, about 50-70%, about 70-90%, about 90-100%, about 50-55%, about 55-60%, about 60-65%, about 65-70%, about 70-75%, about 75-80%, about 80-85%, about 85-90%, about 90-95%, about 95-100%, about 60-80%, or about 65-75% tumor growth inhibition in various in vivo animal models. In some embodiments, administration of a subject compound described herein with a dose amount falls within the range of 1 mg/kg per day to 100 mg/kg per day could achieve the tumor regression or at least about 70% tumor growth inhibition in various in vivo animal models. Such in vivo animal models include, but not limit to, KYSE-520 Xenograft Model, Lung cancer H-358 Xenograft Model, Pancreatic cancer Mia-Pa—Ca-2 Xenograft Model, and non-small cell lung cancer (NSCLC) with KRAS mutant Xenograft Model, etc.

Therefore, the SHP2 inhibitors described herein, such as a compound of Formula 1 could be used in the treatment of cancer and autoimmune disorders, such as Lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors in mammals including animals and humans. The SHP2 inhibitors described herein could be effectively reduce tumor size or volume of various tumors or achieve tumor regression in mammals including animals and humans. In some embodiments, a combination of two SHP2 inhibitors described herein, such as a compound of Formula 1, provides higher efficacy in treating cancer and autoimmune disorders than either SHP2 inhibitor alone in mammals. In some embodiments, the combination of a SHP2 inhibitor described herein, such as a compound of Formula 1, with other agent such as a CDK4/6 inhibitor or a MEK inhibitor, could provide higher efficacy in treating cancer and autoimmune disorders than the single agent alone in mammals.

The following embodiments are contemplated:

### Embodiment 1. A Compound Represented by a Formula:

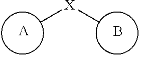

or a pharmaceutically acceptable salt thereof;

wherein X is S, O, NR^{A}, CHR^{A}, SO, SO₂, CO, or a bond;

Ring A is an optionally substituted aryl, heteroaryl or bicyclic ring system;

Ring B is an optionally substituted heterocyclic ring system, comprising a mono-cyclic ring, a bicyclic ring system, a tricyclic ring system, or a tetracyclic ring system, wherein the heterocyclic ring system contains heteroaryl and at least 2 ring nitrogen atoms; and

R^{A} is H or C₁₋₁₂ hydrocarbyl.

### Embodiment 2. The Compound of Embodiment 1, Wherein Ring a is Optionally Substituted Phenyl.

Embodiment 3. The Compound of Embodiment 1, Wherein Ring a is Optionally Substituted naphthalen-1-yl.
Embodiment 4. The Compound of Embodiment 1, Wherein Ring a is Optionally Substituted pyridin-3-yl.
Embodiment 5. The Compound of Embodiment 1, Wherein Ring a is Optionally Substituted pyridin-4-yl.
Embodiment 6. The Compound of Embodiment 1, Wherein Ring a is Optionally Substituted 2-oxo-1,2-dihydropyridin-4-yl.

### Embodiment 7. The Compound of Embodiment 1, Wherein Ring a is Optionally Substituted 1H-indol-4-yl.

Embodiment 8. The Compound of Embodiment 1, Wherein Ring a is Optionally Substituted 2-oxoindolin-4-yl.
Embodiment 9. The Compound of Embodiment 1, Wherein Ring a is Optionally Substituted indolin-4-yl.
Embodiment 10. The Compound of Embodiment 1, Wherein Ring a is Optionally Substituted 3-(2-oxo-2,5-dihydro-1H-pyrrole-3-carboxamido)phenyl.
Embodiment 11. The Compound of Embodiment 1, Wherein Ring a is Optionally Substituted 3-(4-oxo-4H-pyrido[1,2-a]pyrimidine-3-carboxamido)phenyl.
Embodiment 12. The Compound of Embodiment 1, Wherein Ring a is Optionally Substituted 3-(4-oxo-4H-pyrazino[1,2-a]pyrimidine-3-carboxamido)phenyl.
Embodiment 13. The Compound of Embodiment 1, Wherein Ring a is Optionally Substituted 3-(5-oxo-5H-thiazolo[3,2-a]pyrimidine-6-carboxamido)phenyl.
Embodiment 14. The Compound of Embodiment 1, Wherein Ring a is Optionally Substituted 3-(5-oxo-1,5-dihydroimidazo[1,2-a]pyrimidine-6-carboxamido)phenyl.
Embodiment 15. The Compound of Embodiment 1, Wherein Ring a is Optionally Substituted 3-(4-oxo-6,7,8,9-tetrahydro-4H-pyrido[1,2-a]pyrimidine-3-carboxamido)phenyl.
Embodiment 16. The Compound of Embodiment 1, Wherein Ring a is Optionally Substituted 2,3-dichlorophenyl.
Embodiment 17. The Compound of Embodiment 1, Wherein Ring a is Optionally Substituted 2,3-dichloro-pyridin-4-yl.
Embodiment 18. The compound of embodiment 1, wherein Ring A is optionally substituted 2-amino-3-chloropyridin-4-yl.
Embodiment 19 The compound of embodiment 1, wherein Ring A is optionally substituted 3-amino-2-fluorophenyl.
Embodiment 20. The compound of embodiment 1, wherein Ring A is optionally substituted, 3-chloropyridin-4-yl.
Embodiment 21. The compound of embodiment 1, wherein Ring A is optionally substituted 3-chloro-2-(dimethylamino)pyridin-4-yl.
Embodiment 22. The compound of embodiment 1, 2, 3, 4, 5, 6, 7, 8, 9, 10, 11, 12, 13, 14, 15, 16, 17, 18, 19, 20, or 21, wherein Ring A is unsubstituted.
Embodiment 23. The compound of embodiment 1, 2, 3, 4, 5, 6, 7, 8, 9, 10, 11, 12, 13, 14, 15, 16, 17, 18, 19, 20, or 21, wherein Ring A has a Cl substituent.
Embodiment 24. The compound of embodiment 1, 2, 3, 4, 5, 6, 7, 8, 9, 10, 11, 12, 13, 14, 15, 16, 17, 18, 19, 20, or 21, wherein Ring A has two Cl substituents.
Embodiment 25. The compound of embodiment 1, 2, 3, 4, 5, 6, 7, 8, 9, 10, 11, 12, 13, 14, 15, 16, 17, 18, 19, 20, or 21, wherein Ring A has a CF₃ substituent.
Embodiment 26. The compound of embodiment 1, 2, 3, 4, 5, 6, 7, 8, 9, 10, 11, 12, 13, 14, 15, 16, 17, 18, 19, 20, or 21, wherein Ring A has an NH₂ substituent.
Embodiment 27. The compound of embodiment 1, 2, 3, 4, 5, 6, 7, 8, 9, 10, 11, 12, 13, 14, 15, 16, 17, 18, 19, 20, or 21, wherein Ring A has a —OCH₃ substituent.
Embodiment 28. The compound of embodiment 1, 2, 3, 4, 5, 6, 7, 8, 9, 10, 11, 12, 13, 14, 15, 16, 17, 18, 19, 20, or 21, wherein Ring A has an F substituent.
Embodiment 29. The compound of embodiment 1, 2, 3, 4, 5, 6, 7, 8, 9, 10, 11, 12, 13, 14, 15, 16, 17, 18, 19, 20, or 21, wherein Ring A has an acetyl substituent.
Embodiment 30. The compound of embodiment 1, 2, 3, 4, 5, 6, 7, 8, 9, 10, 11, 12, 13, 14, 15, 16, 17, 18, 19, 20, or 21, wherein Ring A has a CH₃ substituent.
Embodiment 31. The compound of embodiment 1, 2, 3, 4, 5, 6, 7, 8, 9, 10, 11, 12, 13, 14, 15, 16, 17, 18, 19, 20, or 21, wherein Ring A has an OH substituent.
Embodiment 32. The compound of embodiment 1, 2, 3, 4, 5, 6, 7, 8, 9, 10, 11, 12, 13, 14, 15, 16, 17, 18, 19, 20, or 21, wherein Ring A has multiple substituents with any combination of the substituent of embodiment 23, 24, 25, 26, 27, or 28, 29, 30, or 31, at any positions that are chemically permissible.
Embodiment 33. The compound of embodiment 1, wherein Ring A is any one of the following: phenyl, 2,3-dichlorophenyl, naphthalen-1-yl, 2-(trifluoromethyl)phenyl, 2-(trifluoromethyl)pyridin-3-yl, 5-amino-2-chlorophenyl, 5-amino-2-chloropyridin-3-yl, 3-amino-2-chlorophenyl, 2-amino-3-chloropyridin-4-yl, 2-chloro-3-methoxyphenyl, 3-chloro-2-methoxypyridin-4-yl, 3-fluoro-1H-indol-4-yl, 3,3-difluoro-2-oxoindolin-4-yl, 1-acetyl-3,3-difluoroindolin-4-yl, 2-chloro-3-(4-hydroxy-1,5,5-trimethyl-2-oxo-2,5-dihydro-1H-pyrrole-3-carboxamido)phenyl, 2-chloro-3-(2-hydroxy-4-oxo-4H-pyrido[1,2-a]pyrimidine-3-carboxamido)phenyl, 2-chloro-3-(2-hydroxy-4-oxo-4H-pyrazino[1,2-a]pyrimidine-3-carboxamido)phenyl, 2-chloro-3-(7-hydroxy-5-oxo-5H-thiazolo[3,2-a]pyrimidine-6-carboxamido)phenyl, 2-chloro-3-(7-hydroxy-5-oxo-1,5-dihydroimidazo[1,2-a]pyrimidine-6-carboxamido)phenyl, 2-chloro-3-(2-hydroxy-4-oxo-6,7,8,9-tetrahydro-4H-pyrido[1,2-a]pyrimidine-3-carboxamido)phenyl, 2,3-dichloropyridin-4-yl, 2,3-difluorophenyl, 3-chloro-2-fluoropyridin-4-yl, 2,3-difluoropyridin-4-yl, 2-chloro-3-methylphenyl, 3-chloro-2-methylpyridin-4-yl, 3,3-difluoro-1-methyl-2-oxoindolin-4-yl, 3-chloro-1-methyl-2-oxo-1,2-dihydropyridin-4-yl, 2-chloro-3-fluorophenyl, 3-amino-2-fluorophenyl, 3-chloropyridin-4-yl, or 3-chloro-2-(dimethylamino)pyridin-4-yl.
Embodiment 34. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(piperidin-1-yl)-1,6-dihydropyrazin-2-yl.
Embodiment 35. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(pyrrolidin-1-yl)-1,6-dihydropyrazin-2-yl.
Embodiment 36. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(hexahydropyrrolo[3,4-c]pyrrol-2(1H)-yl)-6-oxo-1,6-dihydropyrazin-2-yl.
Embodiment 37. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(3,6-diazabicyclo[3.2.0]heptan-6-yl)-6-oxo-1,6-dihydropyrazin-2-yl.
Embodiment 38. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(2-oxa-8-azaspiro[4.5]decan-8-yl)-1,6-dihydropyrazin-2-yl.
Embodiment 39. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(piperidin-4-ylamino)-1,6-dihydropyrazin-2-yl.
Embodiment 40. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(spiro[bicyclo[3.1.0]hexane-3,4′-piperidin]-1′-yl)-1,6-dihydropyrazin-2-yl.
Embodiment 41. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(8-azaspiro[4.5]decan-8-yl)-1,6-dihydropyrazin-2-yl.
Embodiment 42. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 4-oxo-6-(piperidin-1-yl)-4,5-dihydro-1H-pyrazolo[3,4-d]pyrimidin-3-yl.
Embodiment 43. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 4-oxo-6-(pyrrolidin-1-yl)-4,5-dihydro-1H-pyrazolo[3,4-d]pyrimidin-3-yl.
Embodiment 44. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 4-oxo-2-(piperidin-1-yl)-3,4-dihydroquinazolin-5-yl.
Embodiment 45. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 4-oxo-2-(piperidin-1-yl)-3,4-dihydropyrido[3,4-d]pyrimidin-5-yl.
Embodiment 46. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 7-oxo-2-(piperidin-1-yl)-7,8-dihydropyrido[2,3-d]pyrimidin-5-yl.
Embodiment 47. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(piperidin-1-yl)-1H-pyrazolo[4,3-d]thiazol-3-yl.
Embodiment 48. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 7-oxo-6-(piperidin-4-yl)-6,7-dihydro-1H-pyrazolo[4,3-d]pyrimidin-3-yl.
Embodiment 49. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-8-(piperidin-1-yl)-6,7-dihydro-1H-purin-2-yl.
Embodiment 50. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 8-(piperidin-1-yl)-7H-purin-2-yl.
Embodiment 51. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-2-(pyrrolidin-1-yl)-1,6-dihydropyrimidin-5-yl.
Embodiment 52. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-2-(piperidin-1-yl)-1,6-dihydropyrimidin-5-yl.
Embodiment 53. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-2-(2-oxa-8-azaspiro[4.5]decan-8-yl)-1,6-dihydropyrimidin-5-yl.
Embodiment 54. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 2-(3,6-diazabicyclo[3.2.0]heptan-6-yl)-6-oxo-1,6-dihydropyrimidin-5-yl.
Embodiment 55. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 2-(hexahydropyrrolo[3,4-c]pyrrol-2(1H)-yl)-6-oxo-1,6-dihydropyrimidin-5-yl.
Embodiment 56. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 2-(3-azabicyclo[3.2.0]heptan-3-yl)-6-oxo-1,6-dihydropyrimidin-5-yl.
Embodiment 57. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-2-(2-azaspiro[3.4]octan-2-yl)-1,6-dihydropyrimidin-5-yl.
Embodiment 58. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-oxo-6-(2-oxa-8-azaspiro[4.5]decan-8-yl)-4,5-dihydro-1H-pyrazolo[3,4-b]pyrazin-3-yl.
Embodiment 59. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-oxo-6-(piperidin-1-yl)-4,5-dihydro-1H-pyrazolo[3,4-b]pyrazin-3-yl.
Embodiment 60. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-(piperidin-1-yl)-1H-pyrazolo[3,4-d]pyrimidin-3-yl.
Embodiment 61. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(2-azaspiro[3.4]octan-2-yl)-1,6-dihydropyrazin-2-yl.
Embodiment 62. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 1-cyclohexyl-2-oxo-1,2-dihydropyridin-4-yl.
Embodiment 63. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(3-azabicyclo[3.1.0]hexan-3-yl)-6-oxo-1,6-dihydropyrazin-2-yl.
Embodiment 64. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(3H-spiro[benzofuran-2,4′-piperidin]-1′-yl)-1,6-dihydropyrazin-2-yl.
Embodiment 65. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 4-oxo-2-(2-oxa-8-azaspiro[4.5]decan-8-yl)-4,7-dihydro-3H-pyrrolo[2,3-d]pyrimidin-5-yl.
Embodiment 66. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(5,7-dihydrospiro[cyclopenta[b]pyridine-6,4′-piperidin]-1′-yl)-6-oxo-1,6-dihydropyrazin-2-yl.
Embodiment 67. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(1,3-dihydrospiro[indene-2,4′-piperidin]-1′-yl)-6-oxo-1,6-dihydropyrazin-2-yl.
Embodiment 68. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(4,6-dihydrospiro[cyclopenta[d]thiazole-5,4′-piperidin]-1′-yl)-6-oxo-1,6-dihydropyrazin-2-yl.
Embodiment 69. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 6-oxo-5-(spiro[indoline-2,4′-piperidin]-1′-yl)-1,6-dihydropyrazin-2-yl.
Embodiment 70. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-((3S,4S)-4-amino-3-methyl-2-oxa-8-azaspiro[4.5]decan-8-yl)-6-oxo-1,6-dihydropyrazin-2-yl.
Embodiment 71. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted (S)-(5-(1-amino-5-methoxy-1,3-dihydrospiro[indene-2,4′-piperidin]-1′-yl)-6-oxo-1,6 dihydropyrazin-2-yl)silver.
Embodiment 72. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-(4-amino-4,6-dihydrospiro[cyclopenta[d]thiazole-5,4′-piperidin]-1′-yl)-6-oxo-1,6-dihydropyrazin-2-yl.
Embodiment 73. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted (S)-5-(1-amino-8-azaspiro[4.5]decan-8-yl)-6-oxo-1,6-dihydropyrazin-2-yl.
Embodiment 74. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-((3S,4S)-4-amino-3-methyl-2-oxa-8-azaspiro[4.5]decan-8-yl-4-d)-6-oxo-1,6-dihydropyrazin-2-yl.
Embodiment 75. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-((1R,3R)-1-amino-3-hydroxy-8-azaspiro[4.5]decan-8-yl)-6-oxo-1,6-dihydropyrazin-2-yl.
Embodiment 76. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is optionally substituted 5-((1R,3S)-1-amino-3-hydroxy-8-azaspiro[4.5]decan-8-yl)-6-oxo-1,6-dihydropyrazin-2-yl.
Embodiment 77. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B is unsubstituted.
Embodiment 78. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a —CH₃ substituent.
Embodiment 79. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has an —CH₂NH₂ substituent.
Embodiment 80. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has an —NH₂ substituent.
Embodiment 81. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a —CH₂CH₂NH₂ substituent.
Embodiment 82. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a 1-aminopropan-2-yl substituent.
Embodiment 83. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a —CN substituent.
Embodiment 84. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has an —F substituent.
Embodiment 85. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a —Cl substituent.
Embodiment 86. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has a —CH₂F substituent.
Embodiment 87. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has an —OH substituent.
Embodiment 88. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has an —OCH₃ substituent.
Embodiment 89. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, wherein Ring B has multiple substituents with any combination of the substituent of embodiment 78, 79, 80, 81, 82, 83, 84, 85, 86, 87, or 88, at any positions that are chemically permissible.
Embodiment 90. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33, wherein Ring B is any one of the following: 5-(4-(aminomethyl)-4-methylpiperidin-1-yl)-6-oxo-1,6-dihydropyrazin-2-yl, 5-(3-(aminomethyl)-3-methylpyrrolidin-1-yl)-6-oxo-1,6-dihydropyrazin-2-yl, 5-(hexahydropyrrolo[3,4-c]pyrrol-2(1H)-yl)-6-oxo-1,6-dihydropyrazin-2-yl, 5-(3,6-diazabicyclo[3.2.0]heptan-6-yl)-6-oxo-1,6-dihydropyrazin-2-yl, (S)-5-(4-amino-2-oxa-8-azaspiro[4.5]decan-8-yl)-6-oxo-1,6-dihydropyrazin-2-yl, 6-oxo-5-(piperidin-4-ylamino)-1,6-dihydropyrazin-2-yl, 5-(2-aminospiro[bicyclo[3.1.0]hexane-3,4′-piperidin]-1′-yl)-6-oxo-1,6-dihydropyrazin-2-yl, 5-((1R,3R)-1-amino-3-methyl-8-azaspiro[4.5]decan-8-yl)-6-oxo-1,6-dihydropyrazin-2-yl, (6-(4-(aminomethyl)-4-methylpiperidin-1-yl)-4-oxo-4,5-dihydro-1H-pyrazolo[3,4-d]pyrimidin-3-yl)amino, 6-(3-(aminomethyl)-3-methylpyrrolidin-1-yl)-4-oxo-4,5-dihydro-1H-pyrazolo[3,4-d]pyrimidin-3-yl, 7-amino-2-(4-(aminomethyl)-4-methylpiperidin-1-yl)-4-oxo-3,4-dihydroquinazolin-5-yl, 2-(4-(aminomethyl)-4-methylpiperidin-1-yl)-4-oxo-3,4-dihydropyrido[3,4-d]pyrimidin-5-yl, 2-(4-(aminomethyl)-4-methylpiperidin-1-yl)-7-oxo-7,8-dihydropyrido[2,3-d]pyrimidin-5-yl, 5-(4-(aminomethyl)-4-methylpiperidin-1-yl)-1H-pyrazolo[4,3-d]thiazol-3-yl, 6-(1-(1-aminopropan-2-yl)piperidin-4-yl)-7-oxo-6,7-dihydro-1H-pyrazolo[4,3-d]pyrimidin-3-yl, 8-(4-(aminomethyl)-4-methylpiperidin-1-yl)-6-oxo-6,7-dihydro-1H-purin-2-yl, 6-amino-8-(4-(aminomethyl)-4-methylpiperidin-1-yl)-7H-purin-2-yl, 4-amino-2-(3-(aminomethyl)-3-methylpyrrolidin-1-yl)-6-oxo-1,6-dihydropyrimidin-5-yl, 2-(4-(aminomethyl)-4-methylpiperidin-1-yl)-4-cyano-1-methyl-6-oxo-1,6-dihydropyrimidin-5-yl, 4-amino-2-(4-amino-2-oxa-8-azaspiro[4.5]decan-8-yl)-6-oxo-1,6-dihydropyrimidin-5-yl, 4-amino-2-(3,6-diazabicyclo[3.2.0]heptan-6-yl)-6-oxo-1,6-dihydropyrimidin-5-yl, 4-amino-2-(hexahydropyrrolo[3,4-c]pyrrol-2(1H)-yl)-6-oxo-1,6-dihydropyrimidin-5-yl, 4-amino-2-(4-(aminomethyl)-4-methylpiperidin-1-yl)-6-oxo-1,6-dihydropyrimidin-5-yl, 4-amino-2-(6-amino-3-azabicyclo[3.2.0]heptan-3-yl)-6-oxo-1,6-dihydropyrimidin-5-yl, 4-amino-2-(6-amino-2-azaspiro[3.4]octan-2-yl)-1-methyl-6-oxo-1,6-dihydropyrimidin-5-yl, 4-amino-2-(6-amino-3-azabicyclo[3.1.0]hexan-3-yl)-6-oxo-1,6-dihydropyrimidin-5-yl, 4-amino-2-(6-amino-2-azaspiro[3.4]octan-2-yl)-6-oxo-1,6-dihydropyrimidin-5-yl, 5-(4-(aminomethyl)-4-methylpiperidin-1-yl)-1-methyl-6-oxo-1,6-dihydropyrazin-2-yl, 5-(4-amino-2-oxa-8-azaspiro[4.5]decan-8-yl)-6-oxo-1,6-dihydropyrazin-2-yl, (S)-5-(4-amino-2-oxa-8-azaspiro[4.5]decan-8-yl)-1-methyl-6-oxo-1,6-dihydropyrazin-2-yl, 5-(2-aminospiro[bicyclo[3.1.0]hexane-3,4′-piperidin]-1′-yl)-1-methyl-6-oxo-1,6-dihydropyrazin-2-yl, 5-((3S,4S)-4-amino-3-methyl-2-oxa-8-azaspiro[4.5]decan-8-yl)-6-oxo-1,6-dihydropyrazin-2-yl, 5-((3S,4S)-4-amino-3-methyl-2-oxa-8-azaspiro[4.5]decan-8-yl)-1-methyl-6-oxo-1,6-dihydropyrazin-2-yl, 5-((1R,3R)-1-amino-3-methyl-8-azaspiro[4.5]decan-8-yl)-1-methyl-6-oxo-1,6-dihydropyrazin-2-yl, 5-(4-(aminomethyl)-4-methylpiperidin-1-yl)-6-oxo-1,6-dihydropyrazin-2-yl, 6-(4-(aminomethyl)-4-methylpiperidin-1-yl)-4-oxo-4,5-dihydro-1H-pyrazolo[3,4-d]pyrimidin-3-yl, 6-(4-(aminomethyl)-4-methylpiperidin-1-yl)-4-methyl-5-oxo-4,5-dihydro-1H-pyrazolo[3,4-b]pyrazin-3-yl, 6-(4-(aminomethyl)-4-methylpiperidin-1-yl)-5-oxo-4,5-dihydro-1H-pyrazolo[3,4-b]pyrazin-3-yl, 6-(3-(aminomethyl)-3-methylpyrrolidin-1-yl)-5-methyl-4-oxo-4,5-dihydro-1H-pyrazolo[3,4-d]pyrimidin-3-yl, 6-(4-(aminomethyl)-4-methylpiperidin-1-yl)-5-methyl-4-oxo-4,5-dihydro-1H-pyrazolo[3,4-d]pyrimidin-3-yl, 6-(4-(aminomethyl)-4-methylpiperidin-1-yl)-1H-pyrazolo[3,4-d]pyrimidin-3-yl, 4-amino-2-(hexahydropyrrolo[3,4-c]pyrrol-2(1H)-yl)-1-methyl-6-oxo-1,6-dihydropyrimidin-5-yl, 4-amino-2-((3S,4S)-4-amino-3-methyl-2-oxa-8-azaspiro[4.5]decan-8-yl)-6-oxo-1,6-dihydropyrimidin-5-yl, 4-amino-2-((3S,4S)-4-amino-3-methyl-2-oxa-8-azaspiro[4.5]decan-8-yl)-1-methyl-6-oxo-1,6-dihydropyrimidin-5-yl, 4-amino-2-(6-amino-3-azabicyclo[3.1.0]hexan-3-yl)-1-methyl-6-oxo-1,6-dihydropyrimidin-5-yl, 4-amino-2-(6-amino-3-azabicyclo[3.2.0]heptan-3-yl)-1-methyl-6-oxo-1,6-dihydropyrimidin-5-yl, 5-(4-(aminomethyl)-4-fluoropiperidin-1-yl)-6-oxo-1,6-dihydropyrazin-2-yl, 5-(4-(aminomethyl)-4-hydroxypiperidin-1-yl)-6-oxo-1,6-dihydropyrazin-2-yl, (R)-5-(6-amino-2-azaspiro[3.4]octan-2-yl)-6-oxo-1,6-dihydropyrazin-2-yl, (S)-5-(6-amino-2-azaspiro[3.4]octan-2-yl)-6-oxo-1,6-dihydropyrazin-2-yl, 1-(3-aminocyclohexyl)-2-oxo-1,2-dihydropyridin-4-yl, (R)-5-(4-amino-2-oxa-8-azaspiro[4.5]decan-8-yl)-6-oxo-1,6-dihydropyrazin-2-yl, 5-(4-amino-4-(fluoromethyl)piperidin-1-yl)-6-oxo-1,6-dihydropyrazin-2-yl, 5-((3S,4S)-4-amino-3-methyl-2-oxa-8-azaspiro[4.5]decan-8-yl)-6-oxo-1,6-dihydropyrazin-2-yl, (R)-5-(1-amino-8-azaspiro[4.5]decan-8-yl)-6-oxo-1,6-dihydropyrazin-2-yl, 5-(6-amino-3-azabicyclo[3.1.0]hexan-3-yl)-6-oxo-1,6-dihydropyrazin-2-yl, (R)-5-(3-amino-3H-spiro[benzofuran-2,4′-piperidin]-1′-yl)-6-oxo-1,6-dihydropyrazin-2-yl, 2-((3S,4S)-4-amino-3-methyl-2-oxa-8-azaspiro[4.5]decan-8-yl)-3-methyl-4-oxo-4,7-dihydro-3H-pyrrolo[2,3-d]pyrimidin-5-yl, 2-((3S,4S)-4-amino-3-methyl-2-oxa-8-azaspiro[4.5]decan-8-yl)-4-oxo-4,7-dihydro-3H-pyrrolo[2,3-d]pyrimidin-5-yl, (R)-5-(1-amino-3,3-difluoro-8-azaspiro[4.5]decan-8-yl)-6-oxo-1,6-dihydropyrazin-2-yl, 5-((1R)-1-amino-3-fluoro-8-azaspiro[4.5]decan-8-yl)-6-oxo-1,6-dihydropyrazin-2-yl, (S)-5-(5-amino-5,7-dihydrospiro[cyclopenta[b]pyridine-6,4′-piperidin]-1′-yl)-6-oxo-1,6-dihydropyrazin-2-yl, (S)-5-(1-amino-1,3-dihydrospiro[indene-2,4′-piperidin]-1′-yl)-6-oxo-1,6-dihydropyrazin-2-yl, (S)-5-(4-amino-2-chloro-4,6-dihydrospiro[cyclopenta[d]thiazole-5,4′-piperidin]-1′-yl)-6-oxo-1,6-dihydropyrazin-2-yl, (R)-5-(3-aminospiro[indoline-2,4′-piperidin]-1′-yl)-6-oxo-1,6-dihydropyrazin-2-yl, (S)-5-(1-amino-4-methoxy-1,3-dihydrospiro[indene-2,4′-piperidin]-1′-yl)-6-oxo-1,6-dihydropyrazin-2-yl, (S)-(5-(1-amino-5-methoxy-1,3-dihydrospiro[indene-2,4′-piperidin]-1′-yl)-6-oxo-1,6-dihydropyrazin-2-yl)silver, 5-(4-amino-4,6-dihydrospiro[cyclopenta[d]thiazole-5,4′-piperidin]-1′-yl)-6-oxo-1,6-dihydropyrazin-2-yl, (S)-5-(1-amino-8-azaspiro[4.5]decan-8-yl)-6-oxo-1,6-dihydropyrazin-2-yl, 5-((3S,4S)-4-amino-3-methyl-2-oxa-8-azaspiro[4.5]decan-8-yl-4-d)-6-oxo-1,6-dihydropyrazin-2-yl, 5-((1R,3R)-1-amino-3-hydroxy-8-azaspiro[4.5]decan-8-yl)-6-oxo-1,6-dihydropyrazin-2-yl, or 5-((1R,3S)-1-amino-3-hydroxy-8-azaspiro[4.5]decan-8-yl)-6-oxo-1,6-dihydropyrazin-2-yl.
Embodiment 91. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is S.
Embodiment 92. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is a bond.
Embodiment 93. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is O.
Embodiment 94. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is NH.
Embodiment 95. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is CH(CH₃).
Embodiment 96. The compound of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, wherein X is CH₂.
Embodiment 97. The compound of any preceding embodiment, wherein R^{A} is H.
Embodiment 98. The compound of any preceding embodiment, wherein R^{A} is CH₃.
Embodiment 99. A compound of any preceding embodiments, or a pharmaceutically acceptable salt thereof, wherein the compound is optionally substituted at any position that is chemically permissible.
Embodiment 100. The compound of any one of the preceding embodiments, wherein the compound is an R-enantiomer.
Embodiment 101. The compound of any one of the preceding embodiments, wherein the compound is an S-enantiomer.
Embodiment 102. The compound of any one of the preceding embodiments, wherein the compound is deuterated.
Embodiment 103. A compound, or a pharmaceutically acceptable salt thereof, wherein the compound is any one of the following compounds that is optionally substituted: 3-(4-(aminomethyl)-4-methylpiperidin-1-yl)-6-(2,3-dichlorophenyl)pyrazin-2(1H)-one, 3-(4-(aminomethyl)-4-methylpiperidin-1-yl)-6-((2,3-dichlorophenyl)thio)pyrazin-2(1H)-one, 3-(4-(aminomethyl)-4-methylpiperidin-1-yl)-6-((2,3-dichlorophenyl)thio)-1-methylpyrazin-2(1H)-one, 3-(4-(aminomethyl)-4-methylpiperidin-1-yl)-6-(2,3-dichlorophenoxy)pyrazin-2(1H)-one, 3-(4-(aminomethyl)-4-methylpiperidin-1-yl)-6-((2,3-dichlorophenyl)amino)pyrazin-2(1H)-one, 3-(4-(aminomethyl)-4-methylpiperidin-1-yl)-6-(2,3-dichlorophenoxy)-1-methylpyrazin-2(1H)-one, 3-(3-(aminomethyl)-3-methylpyrrolidin-1-yl)-6-((2,3-dichlorophenyl)thio)pyrazin-2(1H)-one, 6-((2,3-dichlorophenyl)thio)-3-(hexahydropyrrolo[3,4-c]pyrrol-2(1H)-yl)pyrazin-2(1H)-one, 3-(3,6-diazabicyclo[3.2.0]heptan-6-yl)-6-((2,3-dichlorophenyl)thio)pyrazin-2(1H)-one, 3-(4-amino-2-oxa-8-azaspiro[4.5]decan-8-yl)-6-((2,3-dichlorophenyl)thio)pyrazin-2(1H)-one, 6-((2,3-dichlorophenyl)thio)-3-(piperidin-4-ylamino)pyrazin-2(1H)-one, 3-(4-(aminomethyl)-4-methylpiperidin-1-yl)-6-(naphthalen-1-ylthio)pyrazin-2(1H)-one, 3-(2-aminospiro[bicyclo[3.1.0]hexane-3,4′-piperidin]-1′-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((2,3-dichlorophenyl)thio)-1-methylpyrazin-2(1H)-one, 3-((1R,3R)-1-amino-3-methyl-8-azaspiro[4.5]decan-8-yl)-6-((2,3-dichlorophenyl)thio)pyrazin-2(1H)-one, 3-(4-(aminomethyl)-4-methylpiperidin-1-yl)-6-((2-(trifluoromethyl)phenyl)thio)pyrazin-2(1H)-one, 3-(4-(aminomethyl)-4-methylpiperidin-1-yl)-6-((2-(trifluoromethyl)pyridin-3-yl)thio)pyrazin-2(1H)-one, 6-((5-amino-2-chlorophenyl)thio)-3-(4-(aminomethyl)-4-methylpiperidin-1-yl)pyrazin-2(1H)-one, 6-((5-amino-2-chloropyridin-3-yl)thio)-3-(4-(aminomethyl)-4-methylpiperidin-1-yl)pyrazin-2(1H)-one, 6-((3-amino-2-chlorophenyl)thio)-3-(4-(aminomethyl)-4-methylpiperidin-1-yl)pyrazin-2(1H)-one, 6-((2-amino-3-chloropyridin-4-yl)thio)-3-(4-(aminomethyl)-4-methylpiperidin-1-yl)pyrazin-2(1H)-one, 3-(4-(aminomethyl)-4-methylpiperidin-1-yl)-6-((2-chloro-3-methoxyphenyl)thio)pyrazin-2(1H)-one, 3-(4-(aminomethyl)-4-methylpiperidin-1-yl)-6-((3-chloro-2-methoxypyridin-4-yl)thio)pyrazin-2(1H)-one, 3-((3S,4S)-4-amino-3-methyl-2-oxa-8-azaspiro[4.5]decan-8-yl)-6-((3-fluoro-1H-indol-4-yl)thio)pyrazin-2(1H)-one, 4-((5-(4-(aminomethyl)-4-methylpiperidin-1-yl)-6-oxo-1,6-dihydropyrazin-2-yl)thio)-3,3-difluoroindolin-2-one, 6-((1-acetyl-3,3-difluoroindolin-4-yl)thio)-3-(4-(aminomethyl)-4-methylpiperidin-1-yl)pyrazin-2(1H)-one, N-(3-((5-(4-(aminomethyl)-4-methylpiperidin-1-yl)-6-oxo-1,6-dihydropyrazin-2-yl)thio)-2-chlorophenyl)-4-hydroxy-1,5,5-trimethyl-2-oxo-2,5-dihydro-1H-pyrrole-3-carboxamide, N-(3-((5-(4-(aminomethyl)-4-methylpiperidin-1-yl)-6-oxo-1,6-dihydropyrazin-2-yl)thio)-2-chlorophenyl)-2-hydroxy-4-oxo-4H-pyrido[1,2-a]pyrimidine-3-carboxamide, N-(3-((5-(4-(aminomethyl)-4-methylpiperidin-1-yl)-6-oxo-1,6-dihydropyrazin-2-yl)thio)-2-chlorophenyl)-2-hydroxy-4-oxo-4H-pyrazino[1,2-a]pyrimidine-3-carboxamide, N-(3-((5-(4-(aminomethyl)-4-methylpiperidin-1-yl)-6-oxo-1,6-dihydropyrazin-2-yl)thio)-2-chlorophenyl)-7-hydroxy-5-oxo-5H-thiazolo[3,2-a]pyrimidine-6-carboxamide, N-(3-((5-(4-(aminomethyl)-4-methylpiperidin-1-yl)-6-oxo-1,6-dihydropyrazin-2-yl)thio)-2-chlorophenyl)-7-hydroxy-5-oxo-1,5-dihydroimidazo[1,2-a]pyrimidine-6-carboxamide, N-(3-((5-(4-(aminomethyl)-4-methylpiperidin-1-yl)-6-oxo-1,6-dihydropyrazin-2-yl)thio)-2-chlorophenyl)-2-hydroxy-4-oxo-6,7,8,9-tetrahydro-4H-pyrido[1,2-a]pyrimidine-3-carboxamide, 6-(4-(aminomethyl)-4-methylpiperidin-1-yl)-3-((2,3-dichlorophenyl)amino)-1,5-dihydro-4H-pyrazolo[3,4-d]pyrimidin-4-one, 6-(4-(aminomethyl)-4-methylpiperidin-1-yl)-3-(2,3-dichlorophenoxy)-1,5-dihydro-4H-pyrazolo[3,4-d]pyrimidin-4-one, 6-(4-(aminomethyl)-4-methylpiperidin-1-yl)-3-((2,3-dichlorophenyl)amino)-5-methyl-1,5-dihydro-4H-pyrazolo[3,4-d]pyrimidin-4-one, 6-(4-(aminomethyl)-4-methylpiperidin-1-yl)-3-(2,3-dichlorophenoxy)-5-methyl-1,5-dihydro-4H-pyrazolo[3,4-d]pyrimidin-4-one, 6-(4-(aminomethyl)-4-methylpiperidin-1-yl)-3-(2,3-dichlorophenoxy)-4-methyl-1,4-dihydro-5H-pyrazolo[3,4-b]pyrazin-5-one, 6-(4-(aminomethyl)-4-methylpiperidin-1-yl)-3-(2,3-dichlorophenoxy)-1,4-dihydro-5H-pyrazolo[3,4-b]pyrazin-5-one, 6-(4-(aminomethyl)-4-methylpiperidin-1-yl)-3-(2,3-dichlorophenyl)-1,4-dihydro-5H-pyrazolo[3,4-b]pyrazin-5-one, 6-(4-(aminomethyl)-4-methylpiperidin-1-yl)-3-((2,3-dichlorophenyl)thio)-1,4-dihydro-5H-pyrazolo[3,4-b]pyrazin-5-one, 6-(3-(aminomethyl)-3-methylpyrrolidin-1-yl)-3-(2,3-dichlorophenoxy)-1,5-dihydro-4H-pyrazolo[3,4-d]pyrimidin-4-one, 6-(3-(aminomethyl)-3-methylpyrrolidin-1-yl)-3-(2,3-dichlorophenoxy)-5-methyl-1,5-dihydro-4H-pyrazolo[3,4-d]pyrimidin-4-one, 6-(4-(aminomethyl)-4-methylpiperidin-1-yl)-3-(1-(2,3-dichlorophenyl)ethyl)-5-methyl-1,5-dihydro-4H-pyrazolo[3,4-d]pyrimidin-4-one, 6-(4-(aminomethyl)-4-methylpiperidin-1-yl)-3-(2,3-dichlorobenzyl)-1,5-dihydro-4H-pyrazolo[3,4-d]pyrimidin-4-one, 7-amino-2-(4-(aminomethyl)-4-methylpiperidin-1-yl)-5-((2,3-dichlorophenyl)thio)quinazolin-4(3H)-one, 2-(4-(aminomethyl)-4-methylpiperidin-1-yl)-5-((2,3-dichlorophenyl)thio)pyrido[3,4-d]pyrimidin-4(3H)-one, 2-(4-(aminomethyl)-4-methylpiperidin-1-yl)-5-((2,3-dichlorophenyl)thio)pyrido[2,3-d]pyrimidin-7(8H)-one, (1-(3-((2,3-dichlorophenyl)thio)-1H-pyrazolo[4,3-d]thiazol-5-yl)-4-methylpiperidin-4-yl)methanamine, 6-(1-(1-aminopropan-2-yl)piperidin-4-yl)-3-((2,3-dichlorophenyl)thio)-1,6-dihydro-7H-pyrazolo[4,3-d]pyrimidin-7-one, 5-(1-(1-aminopropan-2-yl)piperidin-4-yl)-3-((2,3-dichlorophenyl)thio)-1,6-dihydro-7H-pyrazolo[4,3-d]pyrimidin-7-one, 6-amino-2-(3-(aminomethyl)-3-methylpyrrolidin-1-yl)-5-((2,3-dichlorophenyl)thio)-3-methylpyrimidin-4(3H)-one, 6-amino-5-((2,3-dichlorophenyl)thio)-2-(hexahydropyrrolo[3,4-c]pyrrol-2(1H)-yl)-3-methylpyrimidin-4(3H)-one, 6-amino-2-(3-(aminomethyl)-3-methylpyrrolidin-1-yl)-5-((2,3-dichlorophenyl)thio)pyrimidin-4(3H)-one, 2-(4-(aminomethyl)-4-methylpiperidin-1-yl)-5-(2,3-dichlorophenyl)-1-methyl-6-oxo-1,6-dihydropyrimidine-4-carbonitrile, 6-amino-2-((3S,4S)-4-amino-3-methyl-2-oxa-8-azaspiro[4.5]decan-8-yl)-5-(2,3-dichlorophenoxy)pyrimidin-4(3H)-one, 6-amino-2-((3S,4S)-4-amino-3-methyl-2-oxa-8-azaspiro[4.5]decan-8-yl)-5-(2,3-dichlorophenoxy)-3-methylpyrimidin-4(3H)-one, 6-amino-2-(3,6-diazabicyclo[3.2.0]heptan-6-yl)-5-((2,3-dichlorophenyl)thio)-3-methylpyrimidin-4(3H)-one, 6-amino-2-(3,6-diazabicyclo[3.2.0]heptan-6-yl)-5-((2,3-dichlorophenyl)thio)pyrimidin-4(3H)-one, 6-amino-5-((2,3-dichlorophenyl)thio)-2-(hexahydropyrrolo[3,4-c]pyrrol-2(1H)-yl)pyrimidin-4(3H)-one, 6-amino-2-(4-(aminomethyl)-4-methylpiperidin-1-yl)-5-(2,3-dichlorophenoxy)pyrimidin-4(3H)-one, 6-amino-2-(6-amino-3-azabicyclo[3.2.0]heptan-3-yl)-5-((2,3-dichlorophenyl)thio)pyrimidin-4(3H)-one, 6-amino-2-(6-amino-2-azaspiro[3.4]octan-2-yl)-5-((2,3-dichlorophenyl)thio)-3-methylpyrimidin-4(3H)-one, 6-amino-2-(6-amino-3-azabicyclo[3.1.0]hexan-3-yl)-5-((2,3-dichlorophenyl)thio)-3-methylpyrimidin-4(3H)-one, 6-amino-2-(6-amino-3-azabicyclo[3.2.0]heptan-3-yl)-5-((2,3-dichlorophenyl)thio)-3-methylpyrimidin-4(3H)-one, 6-amino-2-(6-amino-3-azabicyclo[3.1.0]hexan-3-yl)-5-((2,3-dichlorophenyl)thio)pyrimidin-4(3H)-one, 6-amino-2-(6-amino-2-azaspiro[3.4]octan-2-yl)-5-((2,3-dichlorophenyl)thio)pyrimidin-4(3H)-one, 6-(3-(aminomethyl)-3-methylpyrrolidin-1-yl)-3-((2,3-dichlorophenyl)amino)-1,5-dihydro-4H-pyrazolo[3,4-d]pyrimidin-4-one, 3-(4-(aminomethyl)-4-fluoropiperidin-1-yl)-6-((2,3-dichlorophenyl)thio)pyrazin-2(1H)-one, 3-(4-(aminomethyl)-4-hydroxypiperidin-1-yl)-6-((2,3-dichlorophenyl)thio)pyrazin-2(1H)-one, (R)-3-(6-amino-2-azaspiro[3.4]octan-2-yl)-6-((2,3-dichlorophenyl)thio)pyrazin-2(1H)-one, (S)-3-(6-amino-2-azaspiro[3.4]octan-2-yl)-6-((2,3-dichlorophenyl)thio)pyrazin-2(1H)-one, (S)-3-(4-amino-2-oxa-8-azaspiro[4.5]decan-8-yl)-6-((2,3-dichlorophenyl)thio)pyrazin-2(1H)-one, 1-(3-aminocyclohexyl)-4-(2,3-dichlorophenyl)pyridin-2(1H)-one, (R)-3-(4-amino-2-oxa-8-azaspiro[4.5]decan-8-yl)-6-((2,3-dichlorophenyl)thio)pyrazin-2(1H)-one, 3-(4-amino-4-(fluoromethyl)piperidin-1-yl)-6-((2,3-dichlorophenyl)thio)pyrazin-2(1H)-one, 6-((2-amino-3-chloropyridin-4-yl)thio)-3-((3S,4S)-4-amino-3-methyl-2-oxa-8-azaspiro[4.5]decan-8-yl)pyrazin-2(1H)-one, (R)-3-(1-amino-8-azaspiro[4.5]decan-8-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((2-chloro-3-methoxyphenyl)thio)pyrazin-2(1H)-one, 3-((3S,4S)-4-amino-3-methyl-2-oxa-8-azaspiro[4.5]decan-8-yl)-6-((3-chloro-2-methoxypyridin-4-yl)thio)pyrazin-2(1H)-one, 3-((3S,4S)-4-amino-3-methyl-2-oxa-8-azaspiro[4.5]decan-8-yl)-6-((2,3-dichloropyridin-4-yl)thio)pyrazin-2(1H)-one, 3-((3S,4S)-4-amino-3-methyl-2-oxa-8-azaspiro[4.5]decan-8-yl)-6-((2,3-difluorophenyl)thio)pyrazin-2(1H)-one, 3-((3S,4S)-4-amino-3-methyl-2-oxa-8-azaspiro[4.5]decan-8-yl)-6-((3-chloro-2-fluoropyridin-4-yl)thio)pyrazin-2(1H)-one, 4-((5-(4-(aminomethyl)-4-methylpiperidin-1-yl)-6-oxo-1,6-dihydropyrazin-2-yl)thio)-3,3-difluoroindolin-2-one, 3-((3S,4S)-4-amino-3-methyl-2-oxa-8-azaspiro[4.5]decan-8-yl)-6-((2,3-dichlorophenyl)thio)pyridin-2(1H)-one, 3-(6-amino-3-azabicyclo[3.1.0]hexan-3-yl)-6-((2,3-dichlorophenyl)thio)pyrazin-2(1H)-one, (R)-3-(3-amino-3H-spiro[benzofuran-2,4′-piperidin]-1′-yl)-6-((2,3-dichlorophenyl)thio)pyrazin-2(1H)-one, (S)-3-(4-amino-2-oxa-8-azaspiro[4.5]decan-8-yl)-6-((2,3-dichloropyridin-4-yl)thio)pyrazin-2(1H)-one, 2-((3S,4S)-4-amino-3-methyl-2-oxa-8-azaspiro[4.5]decan-8-yl)-5-(2,3-dichlorophenoxy)-3-methyl-3,7-dihydro-4H-pyrrolo[2,3-d]pyrimidin-4-one, 2-((3S,4S)-4-amino-3-methyl-2-oxa-8-azaspiro[4.5]decan-8-yl)-5-(2,3-dichlorophenoxy)-3,7-dihydro-4H-pyrrolo[2,3-d]pyrimidin-4-one, 6-((3S,4S)-4-amino-3-methyl-2-oxa-8-azaspiro[4.5]decan-8-yl)-3-((2,3-dichlorophenyl)amino)-1,4-dihydro-5H-pyrazolo[3,4-b]pyrazin-5-one, 6-((3S,4S)-4-amino-3-methyl-2-oxa-8-azaspiro[4.5]decan-8-yl)-3-((2,3-dichlorophenyl)amino)-4-methyl-1,4-dihydro-5H-pyrazolo[3,4-b]pyrazin-5-one, 2-((3S,4S)-4-amino-3-methyl-2-oxa-8-azaspiro[4.5]decan-8-yl)-5-((2,3-dichlorophenyl)amino)-3,7-dihydro-4H-pyrrolo[2,3-d]pyrimidin-4-one, 2-((3S,4S)-4-amino-3-methyl-2-oxa-8-azaspiro[4.5]decan-8-yl)-5-((2,3-dichlorophenyl)amino)-3-methyl-3,7-dihydro-4H-pyrrolo[2,3-d]pyrimidin-4-one, (R)-3-(1-amino-3,3-difluoro-8-azaspiro[4.5]decan-8-yl)-6-((2,3-dichlorophenyl)thio)pyrazin-2(1H)-one, 3-((1R)-1-amino-3-fluoro-8-azaspiro[4.5]decan-8-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((2-chloro-3-methylphenyl)thio)pyrazin-2(1H)-one, 3-((3S,4S)-4-amino-3-methyl-2-oxa-8-azaspiro[4.5]decan-8-yl)-6-((3-chloro-2-methylpyridin-4-yl)thio)pyrazin-2(1H)-one, N-(3-((5-((3S,4S)-4-amino-3-methyl-2-oxa-8-azaspiro[4.5]decan-8-yl)-6-oxo-1,6-dihydropyrazin-2-yl)thio)-2-chlorophenyl)-2-hydroxy-4-oxo-4H-pyrido[1,2-a]pyrimidine-3-carboxamide, 4-((5-((3S,4S)-4-amino-3-methyl-2-oxa-8-azaspiro[4.5]decan-8-yl)-6-oxo-1,6-dihydropyrazin-2-yl)thio)-3,3-difluoro-1-methylindolin-2-one, 3-((3S,4S)-4-amino-3-methyl-2-oxa-8-azaspiro[4.5]decan-8-yl)-6-((3-chloro-1-methyl-2-oxo-1,2-dihydropyridin-4-yl)thio)pyrazin-2(1H)-one, (S)-3-(5-amino-5,7-dihydrospiro[cyclopenta[b]pyridine-6,4′-piperidin]-1′-yl)-6-((2,3-dichlorophenyl)thio)pyrazin-2(1H)-one, (S)-3-(1-amino-1,3-dihydrospiro[indene-2,4′-piperidin]-1′-yl)-6-((2,3-dichloropyridin-4-yl)thio)pyrazin-2(1H)-one, (S)-3-(4-amino-2-chloro-4,6-dihydrospiro[cyclopenta[d]thiazole-5,4′-piperidin]-1′-yl)-6-((2,3-dichlorophenyl)thio)pyrazin-2(1H)-one, (R)-3-(3-aminospiro[indoline-2,4′-piperidin]-1′-yl)-6-((2,3-dichlorophenyl)thio)pyrazin-2(1H)-one, (S)-3-(1-amino-4-methoxy-1,3-dihydrospiro[indene-2,4′-piperidin]-1′-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((3-chloro-2-oxo-1,2-dihydropyridin-4-yl)thio)pyrazin-2(1H)-one, (S)-3-(1-amino-1,3-dihydrospiro[indene-2,4′-piperidin]-1′-yl)-6-((2,3-dichlorophenyl)thio)pyrazin-2(1H)-one, 3-((3S,4S)-4-amino-3-methyl-2-oxa-8-azaspiro[4.5]decan-8-yl)-6-((2-chloro-3-fluorophenyl)thio)pyrazin-2(1H)-one, (1-(3-((2,3-dichlorophenyl)thio)-1H-pyrazolo[3,4-d]pyrimidin-6-yl)-4-methylpiperidin-4-yl)methanamine, 3-(4-amino-2-oxa-8-azaspiro[4.5]decan-8-yl)-6-((2,3-dichlorophenyl)thio)-1-methylpyrazin-2(1H)-one, 3-(2-aminospiro[bicyclo[3.1.0]hexane-3,4′-piperidin]-1′-yl)-6-((2,3-dichlorophenyl)thio)-1-methylpyrazin-2(1H)-one, 3-((1R,3R)-1-amino-3-methyl-8-azaspiro[4.5]decan-8-yl)-6-((2,3-dichlorophenyl)thio)-1-methylpyrazin-2(1H)-one, 6-((3S,4S)-4-amino-3-methyl-2-oxa-8-azaspiro[4.5]decan-8-yl)-3-(2,3-dichlorophenoxy)-1,4-dihydro-5H-pyrazolo[3,4-b]pyrazin-5-one, (S)-3-(1-amino-1,3-dihydrospiro[indene-2,4′-piperidin]-1′-yl)-6-((2-amino-3-chloropyridin-4 yl)thio)pyrazin-2(1H)-one, 3-(4-amino-2-chloro-4,6-dihydrospiro[cyclopenta[d]thiazole-5,4′-piperidin]-1′-yl)-6-((2,3-dichloropyridin-4-yl)thio)pyrazin-2(1H)-one, 3-(4-amino-2-chloro-4,6-dihydrospiro[cyclopenta[d]thiazole-5,4′-piperidin]-1′-yl)-6-((2-amino-3-chloropyridin-4 yl)thio)pyrazin-2(1H)-one, (S)-3-(5-amino-5,7-dihydrospiro[cyclopenta[b]pyridine-6,4′-piperidin]-1′-yl)-6-((2,3-dichloropyridin-4-yl)thio)pyrazin-2(1H)-one, (S)-6-((2-amino-3-chloropyridin-4-yl)thio)-3-(5-amino-5,7-dihydrospiro[cyclopenta[b]pyridine-6,4′-piperidin]-1′-yl)pyrazin-2(1H)-one, (R)-3-(3-aminospiro[indoline-2,4′-piperidin]-1′-yl)-6-((2,3-dichloropyridin-4-yl)thio)pyrazin-2(1H)-one, (R)-6-((2-amino-3-chloropyridin-4-yl)thio)-3-(3-aminospiro[indoline-2,4′-piperidin]-1′-yl)pyrazin-2(1H)-one, (S)-6-((2-amino-3-chloropyridin-4-yl)thio)-3-(1-amino-5-methoxy-1,3-dihydrospiro[indene-2,4′-piperidin]-1′-yl)pyrazin-2(1H)-one, (R)-3-(3-amino-3H-spiro[benzofuran-2,4′-piperidin]-1′-yl)-6-((2,3-dichloropyridin-4-yl)thio)pyrazin-2(1H)-one, (R)-6-((2-amino-3-chloropyridin-4-yl)thio)-3-(3-amino-3H-spiro[benzofuran-2,4′-piperidin]-1′-yl)pyrazin-2(1H)-one, 6-((3-amino-2-chlorophenyl)thio)-3-((3S,4S)-4-amino-3-methyl-2-oxa-8-azaspiro[4.5]decan-8-yl)pyrazin-2(1H)-one, 6-((3-amino-2-fluorophenyl)thio)-3-((3S,4S)-4-amino-3-methyl-2-oxa-8-azaspiro[4.5]decan-8-yl)pyrazin-2(1H)-one, 6-((2-amino-3-fluoropyridin-4-yl)thio)-3-((3S,4S)-4-amino-3-methyl-2-oxa-8-azaspiro[4.5]decan-8-yl)pyrazin-2(1H)-one, 3-((3S,4S)-4-amino-3-methyl-2-oxa-8-azaspiro[4.5]decan-8-yl)-6-((3-chloropyridin-4-yl)thio)pyrazin-2(1H)-one, 3-((3S,4S)-4-amino-3-methyl-2-oxa-8-azaspiro[4.5]decan-8-yl)-6-((3-chloro-2-(dimethylamino)pyridin-4-yl)thio)pyrazin-2(1H)-one, 6-((2-amino-3-chloropyridin-4-yl)thio)-3-(4-amino-4,6-dihydrospiro[cyclopenta[d]thiazole-5,4′-piperidin]-1′-yl)pyrazin-2(1H)-one, (S)-3-(1-amino-5-methoxy-1,3-dihydrospiro[indene-2,4′-piperidin]-1′-yl)-6-((2,3-dichlorophenyl)thio)pyrazin-2(1H)-one, (R)-3-(3-aminospiro[indoline-2,4′-piperidin]-1′-yl)-6-((2,3-dichlorophenyl)thio)pyrazin-2(1H)-one, (S)-6-((2-amino-3-chloropyridin-4-yl)thio)-3-(1-amino-8-azaspiro[4.5]decan-8-yl)pyrazin-2(1H)-one, (S)-3-(5-amino-5,7-dihydrospiro[cyclopenta[b]pyridine-6,4′-piperidin]-1′-yl)-6-((2,3-dichlorophenyl)thio)pyrazin-2(1H)-one, (S)-3-(1-amino-8-azaspiro[4.5]decan-8-yl)-6-((2,3-dichloropyridin-4-yl)thio)pyrazin-2(1H)-one, 3-((3S,4S)-4-amino-3-methyl-2-oxa-8-azaspiro[4.5]decan-8-yl-4-d)-6-((2,3-dichlorophenyl)thio)pyrazin-2-ol, 3-((1R,3R)-1-amino-3-hydroxy-8-azaspiro[4.5]decan-8-yl)-6-((2,3-dichlorophenyl)thio)pyrazin-2(1H)-one, (R)-3-(1-amino-3,3-difluoro-8-azaspiro[4.5]decan-8-yl)-6-((2,3-dichloropyridin-4-yl)thio)pyrazin-2(1H)-one, 3-((1R,3R)-1-amino-3-methyl-8-azaspiro[4.5]decan-8-yl)-6-((2,3-dichloropyridin-4-yl)thio)pyrazin-2(1H)-one, or 3-((1R,3S)-1-amino-3-hydroxy-8-azaspiro[4.5]decan-8-yl)-6-((2,3-dichlorophenyl)thio)pyrazin-2(1H)-one.
Embodiment 104. Any substituent of a compound of embodiment 1 has a molecular weight of about 15 g/mol to about 200 g/mol.
Embodiment 105. A pharmaceutical composition comprising a compound of any preceding embodiments or a pharmaceutically acceptable salt thereof, and a pharmaceutically acceptable vehicle, diluent, or carrier.
Embodiment 106. A method of treating a mammal, including a human, having a disease, disorders, or condition associated with the aberrant activity of SHP2, including cancer and autoimmune disorders, comprising administering to a mammal in need thereof a therapeutically effective amount of the compound of any preceding embodiment.
Embodiment 107. A medicament comprising a composition comprising a therapeutically effective amount of the compound of any preceding embodiment.
Embodiment 108. A kit comprising a medicament of embodiment 100 and a label indicating that the medicament is for treating a disease, disorders, or condition associated with the aberrant activity of SHP2.
Embodiment 1a. A method of treating a disease, a disorder, or a condition associated with the aberrant activity of SHP2, comprising administering a therapeutically effective amount of a compound represented by a formula,

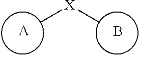

or a pharmaceutically acceptable salt thereof, to a patient in need thereof,

wherein X is S;

Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated;

Ring B is:

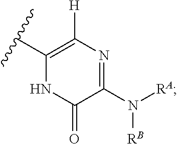

wherein R^{A} and R^{B} are independently H or C₁₋₁₂ hydrocarbyl, or —N(R^{A})(R^{B}) is an optionally substituted heterocyclic ring system, wherein the heterocyclic ring system is a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system or the tricyclic ring system is a spiro, fused, or bridged ring system, wherein the heterocyclic ring system is saturated or partially saturated;
wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl —C(O)N(R^{A})-heterocyclyl, or a combination thereof, and

wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.

Embodiment 2a. Use of a compound represented by a formula,

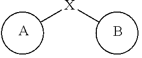

or a pharmaceutically acceptable salt thereof,
in the manufacture of a medicament for treating a disease, a disorder, or a condition associated with the aberrant activity of SHP2, wherein a therapeutically effective amount of the compound or the pharmaceutically acceptable salt is administered to a patient in need thereof,

wherein X is S;

Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated;

Ring B is:

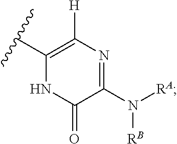

wherein R^{A} and R^{B} are independently H or C₁₋₁₂ hydrocarbyl, or —N(R^{A})(R^{B}) is an optionally substituted heterocyclic ring system, wherein the heterocyclic ring system is a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system or the tricyclic ring system is a spiro, fused, or bridged ring system, wherein the heterocyclic ring system is saturated or partially saturated;

wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl, —C(O)N(R^{A})-heterocyclyl, or a combination thereof, and wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.

Embodiment 3a. The method or the use of embodiment 1a or 2a, wherein Ring A is optionally substituted phenyl.
Embodiment 4a. The method or the use of embodiment 1a or 2a, wherein Ring A is optionally substituted pyridinyl.
Embodiment 5a. The method or the use of embodiment 1a or 2a, wherein Ring A is optionally substituted pyridin-4-yl.
Embodiment 6a. The method or the use of embodiment 1a or 2a, wherein Ring A is optionally substituted 2,3-dichlorophenyl.
Embodiment 7a. The method or the use of embodiment 1a or 2a, wherein Ring A is optionally substituted 2,3-dichloro-pyridin-4-yl.
Embodiment 8a. The method or the use of embodiment 1a or 2a, wherein Ring A is optionally substituted 2-amino-3-chloropyridin-4-yl.
Embodiment 9a. The method or the use of embodiment 1a or 2a, wherein Ring A is:

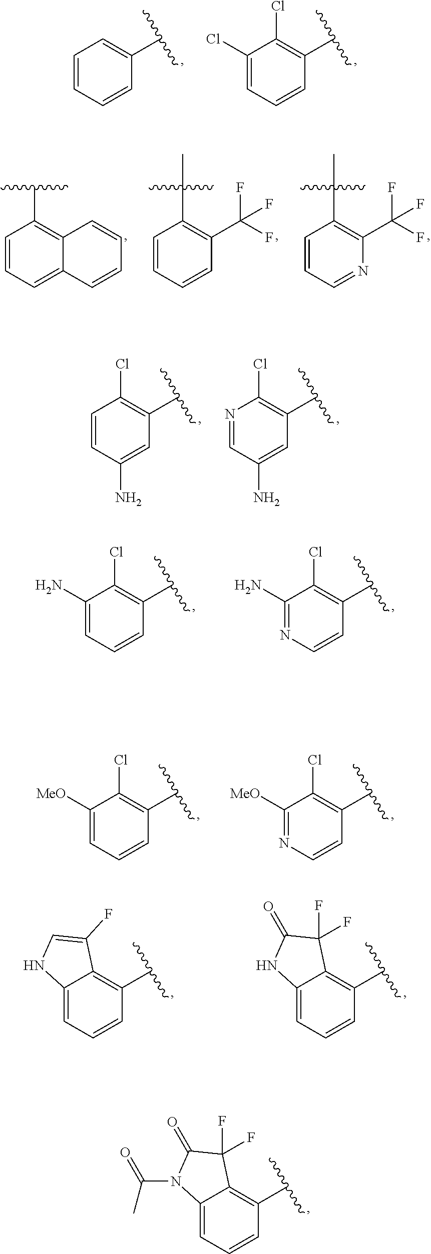

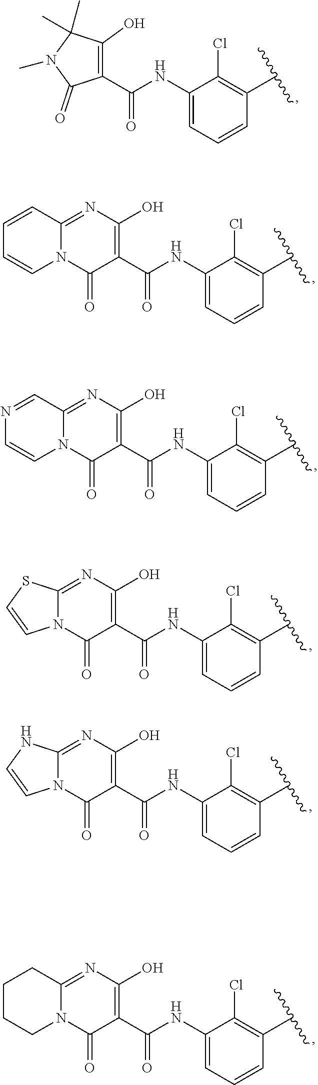 [SMILES: *c1cccc(NC(=O)C2=C(O)C(C)(C)N(C)C2=O)c1Cl ; *c1cccc(NC(=O)c2c(O)nc3ccccn3c2=O)c1Cl ; *c1cccc(NC(=O)c2c(O)nc3cnccn3c2=O)c1Cl ; *c1cccc(NC(=O)c2c(O)nc3sccn3c2=O)c1Cl ; *c1cccc(NC(=O)c2c(O)nc3[nH]ccn3c2=O)c1Cl ; *c1cccc(NC(=O)c2c(O)nc3n(c2=O)CCCC3)c1Cl]

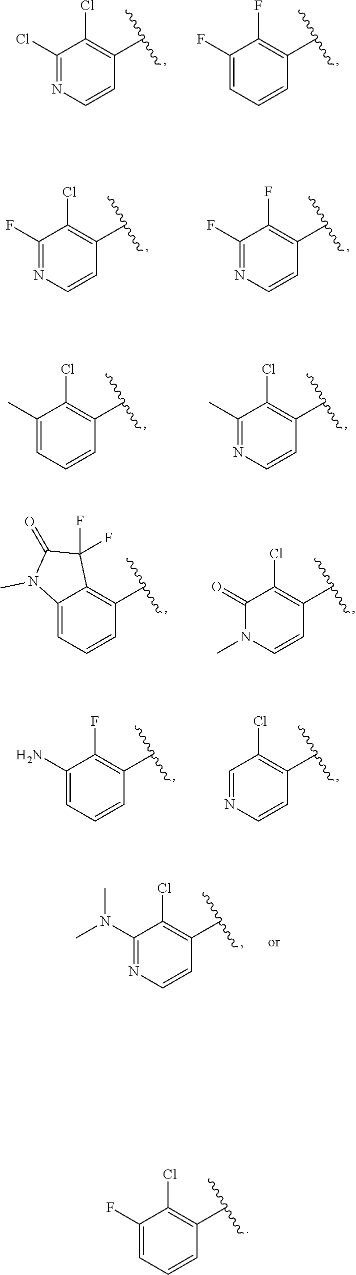

Embodiment 10a. The method or the use of embodiment 1a, 2a, 3a, 4a, 5a, 6a, 7a, 8a, or 9a, wherein Ring B is 5-((3S,4S)-4-amino-3-methyl-2-oxa-8-azaspiro[4.5]decan-8-yl)-6-oxo-1,6-dihydropyrazin-2-yl.
Embodiment 11a. The method or the use of embodiment 1a, 2a, 3a, 4a, 5a, 6a, 7a, 8a, or 9a, wherein Ring B is:

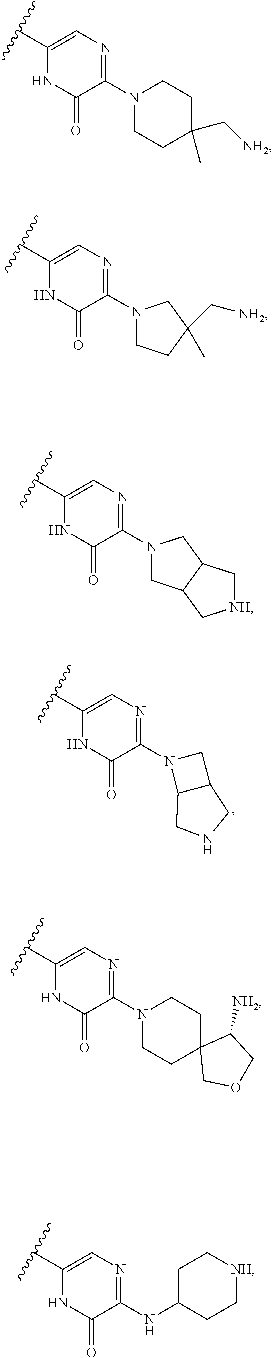 [SMILES: *c1cnc(N2CCC(C)(CN)CC2)c(=O)[nH]1 ; *c1cnc(N2CCC(C)(CN)C2)c(=O)[nH]1 ; *c1cnc(N2CC3CNCC3C2)c(=O)[nH]1 ; *c1cnc(N2CC3CNCC32)c(=O)[nH]1 ; *c1cnc(N2CCC3(CC2)COC[C@H]3N)c(=O)[nH]1 ; *c1cnc(NC2CCNCC2)c(=O)[nH]1]

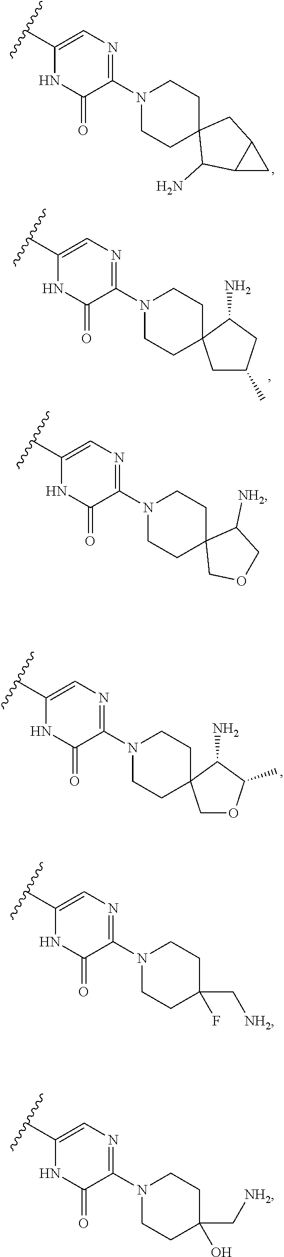 [SMILES: *c1cnc(N2CCC3(CC2)CC2CC2C3N)c(=O)[nH]1 ; *c1cnc(N2CCC3(CC2)C[C@@H](C)C[C@H]3N)c(=O)[nH]1 ; *c1cnc(N2CCC3(CC2)COCC3N)c(=O)[nH]1 ; *c1cnc(N2CCC3(CC2)CO[C@@H](C)[C@H]3N)c(=O)[nH]1 ; *c1cnc(N2CCC(F)(CN)CC2)c(=O)[nH]1 ; *c1cnc(N2CCC(O)(CN)CC2)c(=O)[nH]1]

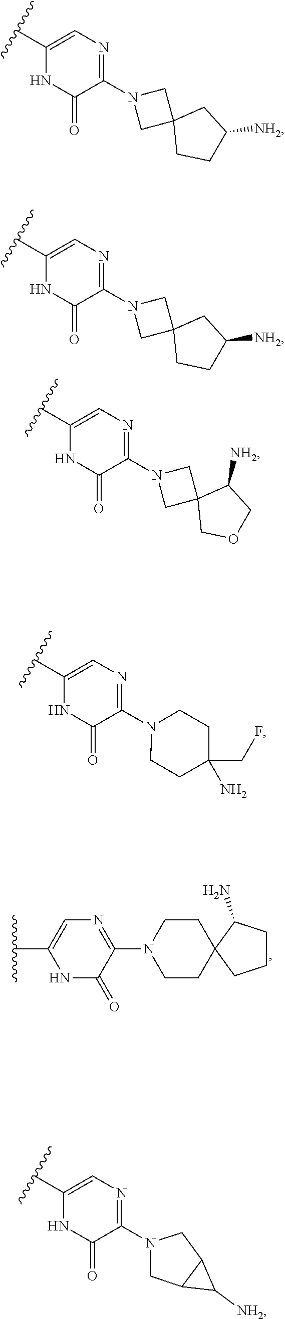 [SMILES: *c1cnc(N2CC3(CC[C@@H](N)C3)C2)c(=O)[nH]1 ; *c1cnc(N2CC3(CC[C@H](N)C3)C2)c(=O)[nH]1 ; *c1cnc(N2CC3(COC[C@@H]3N)C2)c(=O)[nH]1 ; *c1cnc(N2CCC(N)(CF)CC2)c(=O)[nH]1 ; *c1cnc(N2CCC3(CCC[C@H]3N)CC2)c(=O)[nH]1 ; *c1cnc(N2CC3C(N)C3C2)c(=O)[nH]1]

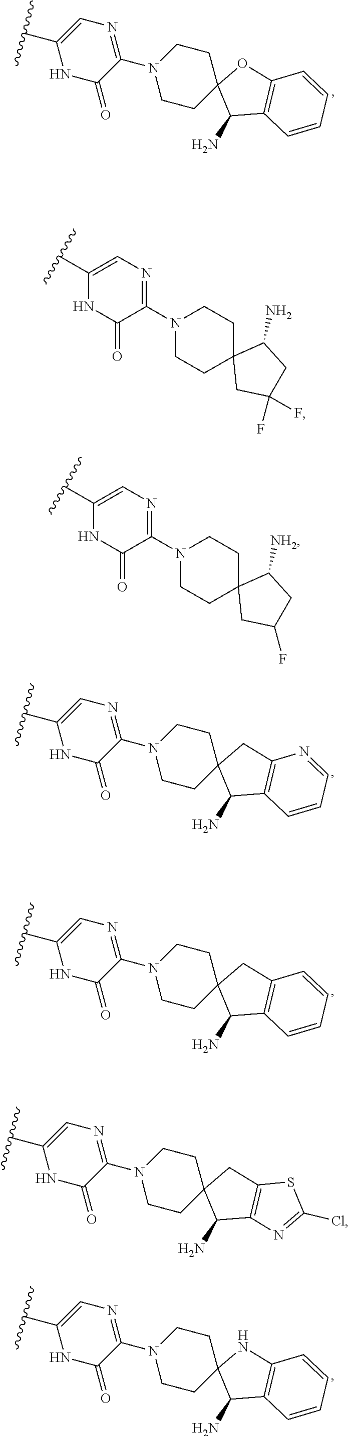

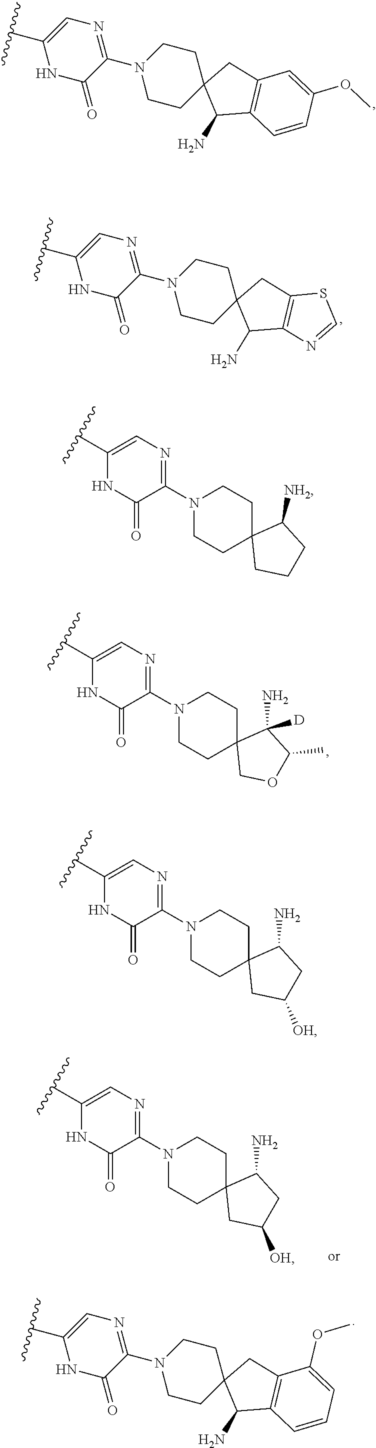

Embodiment 12a. The method or the use of embodiment 1a, 2a, 3a, 4a, 5a, 6a, 7a, 8a, 9a, 10a, or 11a, wherein the compound is an R-enantiomer.
Embodiment 13a. The method or the use of embodiment 1a, 2a, 3a, 4a, 5a, 6a, 7a, 8a, 9a, 10a, or 11a, wherein the compound is an S-enantiomer.
Embodiment 14a. The method or the use of embodiment 1a, 2a, 3a, 4a, 5a, 6a, 7a, 8a, 9a, 10a, 11a, 12a, or 13a, or a pharmaceutically acceptable salt thereof, wherein the compound is:

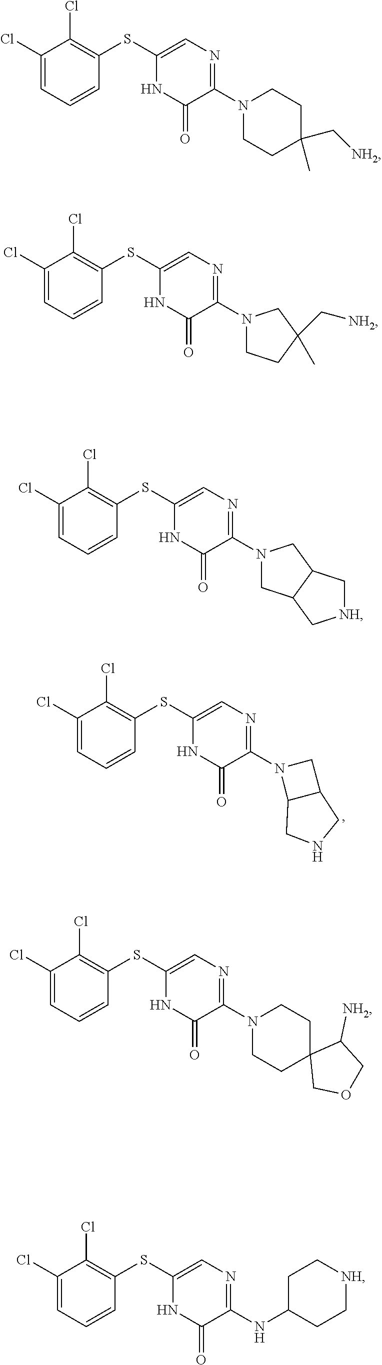 [SMILES: CC1(CN)CCN(c2ncc(Sc3cccc(Cl)c3Cl)[nH]c2=O)CC1 ; CC1(CN)CCN(c2ncc(Sc3cccc(Cl)c3Cl)[nH]c2=O)C1 ; O=c1[nH]c(Sc2cccc(Cl)c2Cl)cnc1N1CC2CNCC2C1 ; O=c1[nH]c(Sc2cccc(Cl)c2Cl)cnc1N1CC2CNCC21 ; NC1COCC12CCN(c1ncc(Sc3cccc(Cl)c3Cl)[nH]c1=O)CC2 ; O=c1[nH]c(Sc2cccc(Cl)c2Cl)cnc1NC1CCNCC1]

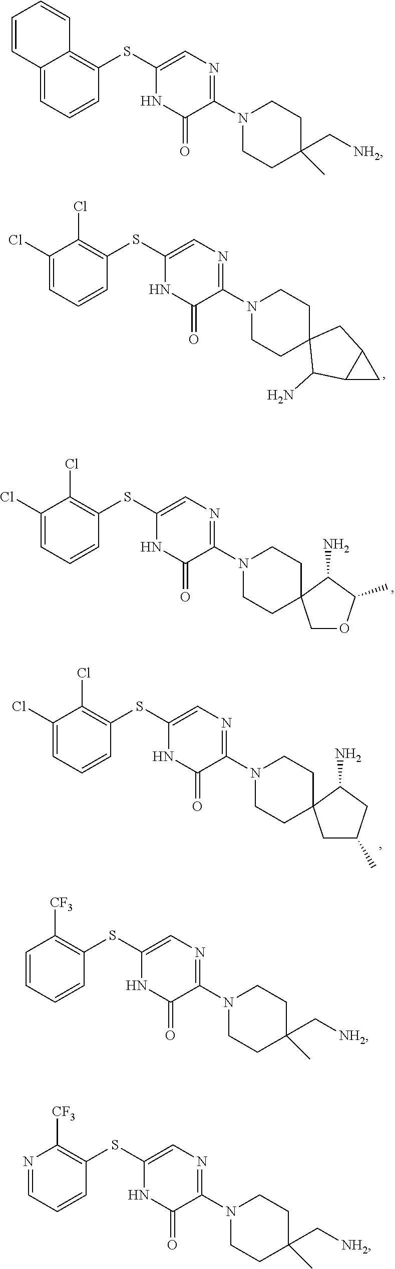 [SMILES: CC1(CN)CCN(c2ncc(Sc3cccc4ccccc34)[nH]c2=O)CC1 ; NC1C2CC2CC12CCN(c1ncc(Sc3cccc(Cl)c3Cl)[nH]c1=O)CC2 ; C[C@@H]1OCC2(CCN(c3ncc(Sc4cccc(Cl)c4Cl)[nH]c3=O)CC2)[C@@H]1N ; C[C@H]1C[C@@H](N)C2(CCN(c3ncc(Sc4cccc(Cl)c4Cl)[nH]c3=O)CC2)C1 ; CC1(CN)CCN(c2ncc(Sc3ccccc3C(F)(F)F)[nH]c2=O)CC1 ; CC1(CN)CCN(c2ncc(Sc3cccnc3C(F)(F)F)[nH]c2=O)CC1]

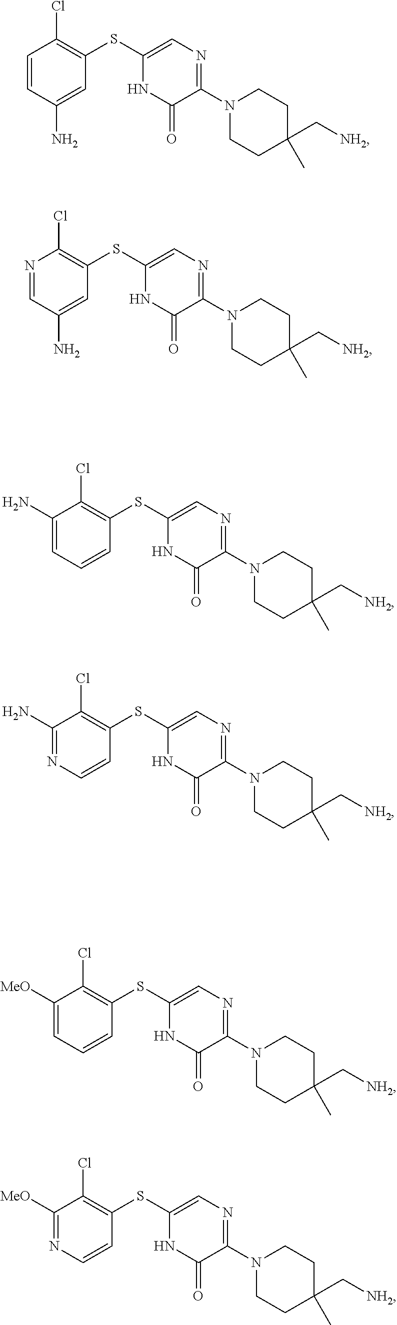 [SMILES: CC1(CN)CCN(c2ncc(Sc3cc(N)ccc3Cl)[nH]c2=O)CC1 ; CC1(CN)CCN(c2ncc(Sc3cc(N)cnc3Cl)[nH]c2=O)CC1 ; CC1(CN)CCN(c2ncc(Sc3cccc(N)c3Cl)[nH]c2=O)CC1 ; CC1(CN)CCN(c2ncc(Sc3ccnc(N)c3Cl)[nH]c2=O)CC1 ; COc1cccc(Sc2cnc(N3CCC(C)(CN)CC3)c(=O)[nH]2)c1Cl ; COc1nccc(Sc2cnc(N3CCC(C)(CN)CC3)c(=O)[nH]2)c1Cl]

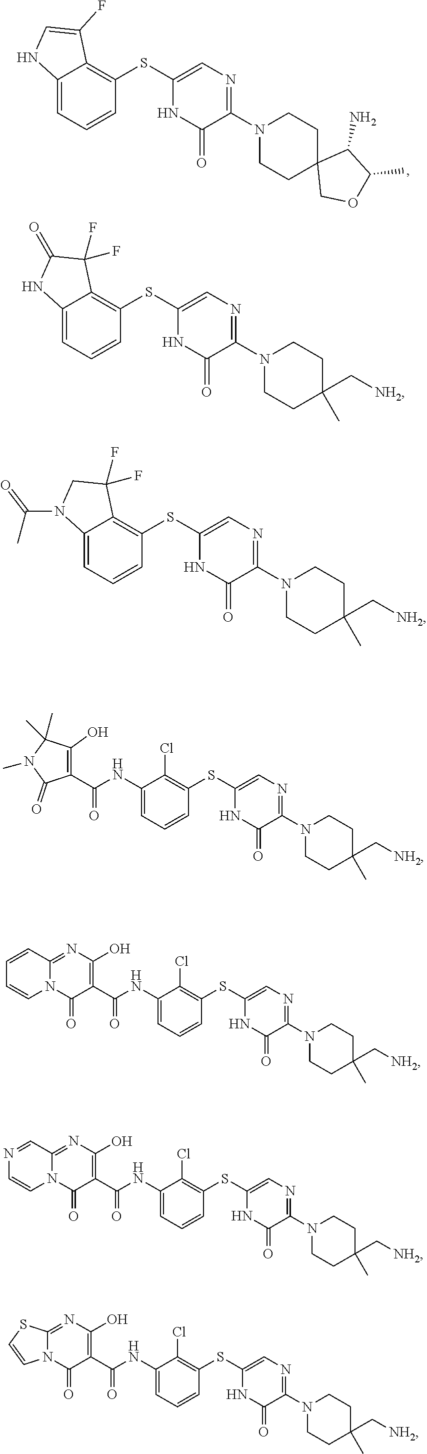

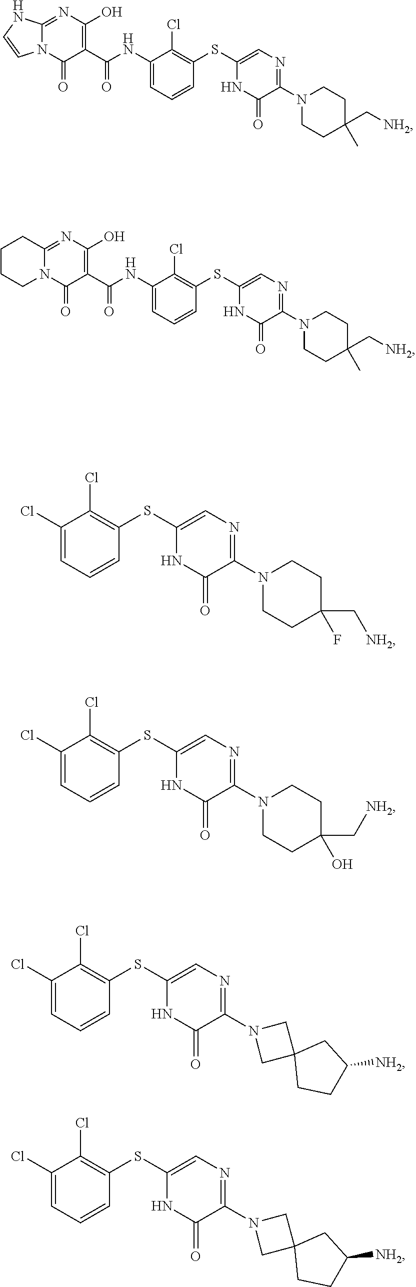 [SMILES: CC1(CN)CCN(c2ncc(Sc3cccc(NC(=O)c4c(O)nc5[nH]ccn5c4=O)c3Cl)[nH]c2=O)CC1 ; CC1(CN)CCN(c2ncc(Sc3cccc(NC(=O)c4c(O)nc5n(c4=O)CCCC5)c3Cl)[nH]c2=O)CC1 ; NCC1(F)CCN(c2ncc(Sc3cccc(Cl)c3Cl)[nH]c2=O)CC1 ; NCC1(O)CCN(c2ncc(Sc3cccc(Cl)c3Cl)[nH]c2=O)CC1 ; N[C@@H]1CCC2(C1)CN(c1ncc(Sc3cccc(Cl)c3Cl)[nH]c1=O)C2 ; N[C@H]1CCC2(C1)CN(c1ncc(Sc3cccc(Cl)c3Cl)[nH]c1=O)C2]

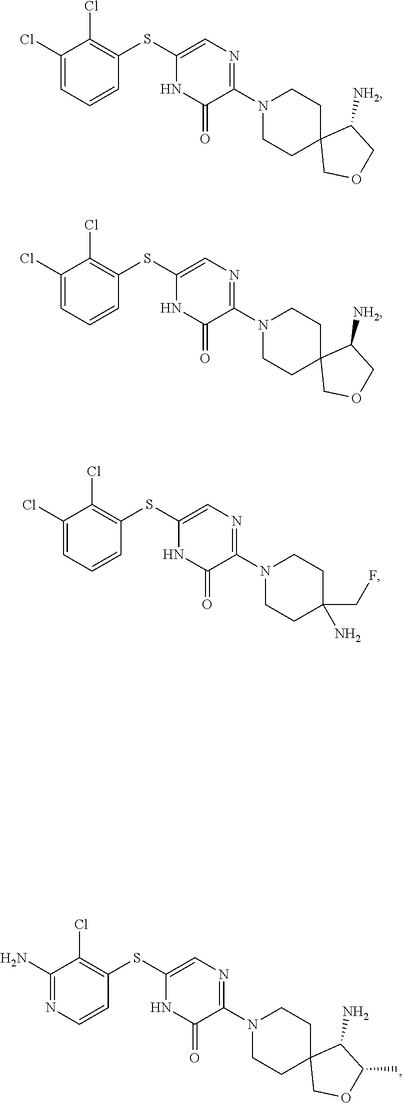 [SMILES: N[C@@H]1COCC12CCN(c1ncc(Sc3cccc(Cl)c3Cl)[nH]c1=O)CC2 ; N[C@H]1COCC12CCN(c1ncc(Sc3cccc(Cl)c3Cl)[nH]c1=O)CC2 ; NC1(CF)CCN(c2ncc(Sc3cccc(Cl)c3Cl)[nH]c2=O)CC1 ; C[C@@H]1OCC2(CCN(c3ncc(Sc4ccnc(N)c4Cl)[nH]c3=O)CC2)[C@@H]1N]

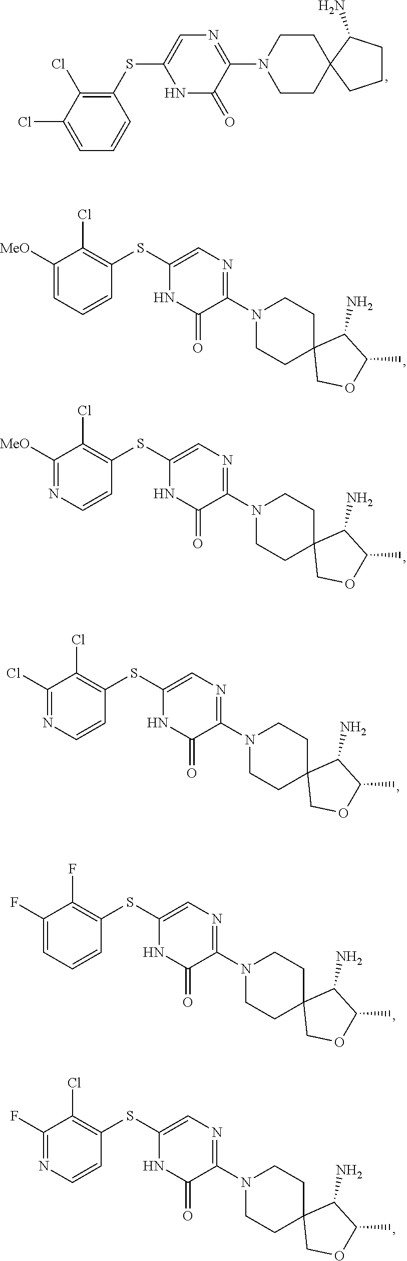 [SMILES: N[C@@H]1CCCC12CCN(c1ncc(Sc3cccc(Cl)c3Cl)[nH]c1=O)CC2 ; COc1cccc(Sc2cnc(N3CCC4(CC3)CO[C@@H](C)[C@H]4N)c(=O)[nH]2)c1Cl ; COc1nccc(Sc2cnc(N3CCC4(CC3)CO[C@@H](C)[C@H]4N)c(=O)[nH]2)c1Cl ; C[C@@H]1OCC2(CCN(c3ncc(Sc4ccnc(Cl)c4Cl)[nH]c3=O)CC2)[C@@H]1N ; C[C@@H]1OCC2(CCN(c3ncc(Sc4cccc(F)c4F)[nH]c3=O)CC2)[C@@H]1N ; C[C@@H]1OCC2(CCN(c3ncc(Sc4ccnc(F)c4Cl)[nH]c3=O)CC2)[C@@H]1N]

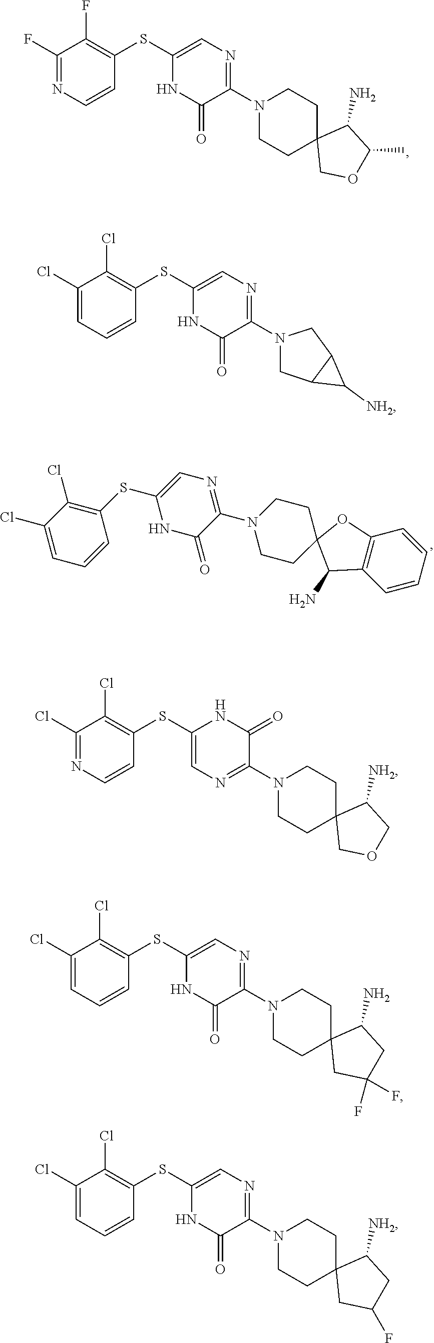 [SMILES: C[C@@H]1OCC2(CCN(c3ncc(Sc4ccnc(F)c4F)[nH]c3=O)CC2)[C@@H]1N ; NC1C2CN(c3ncc(Sc4cccc(Cl)c4Cl)[nH]c3=O)CC12 ; N[C@@H]1c2ccccc2OC12CCN(c1ncc(Sc3cccc(Cl)c3Cl)[nH]c1=O)CC2 ; N[C@@H]1COCC12CCN(c1ncc(Sc3ccnc(Cl)c3Cl)[nH]c1=O)CC2 ; N[C@@H]1CC(F)(F)CC12CCN(c1ncc(Sc3cccc(Cl)c3Cl)[nH]c1=O)CC2 ; N[C@@H]1CC(F)CC12CCN(c1ncc(Sc3cccc(Cl)c3Cl)[nH]c1=O)CC2]

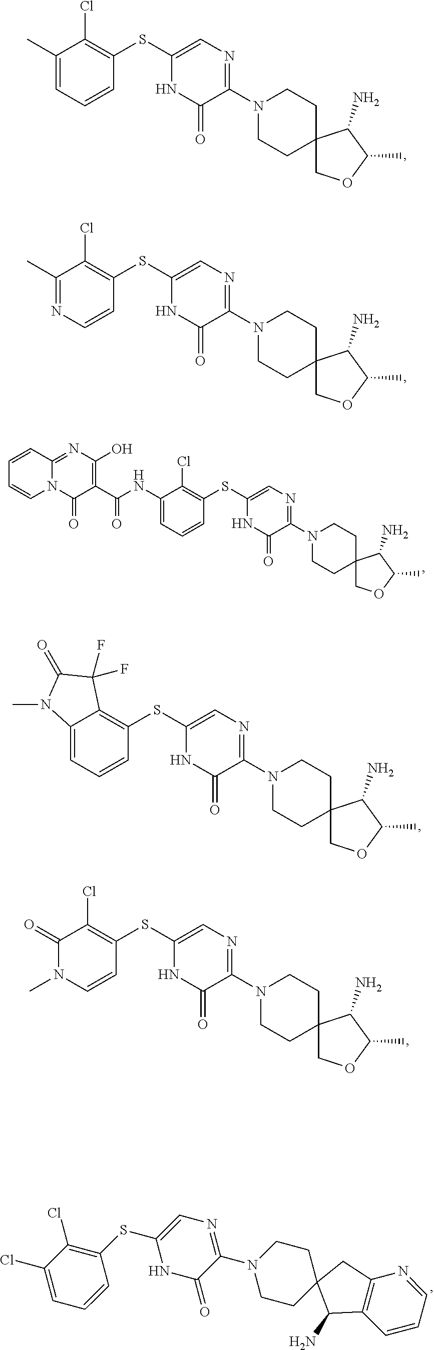

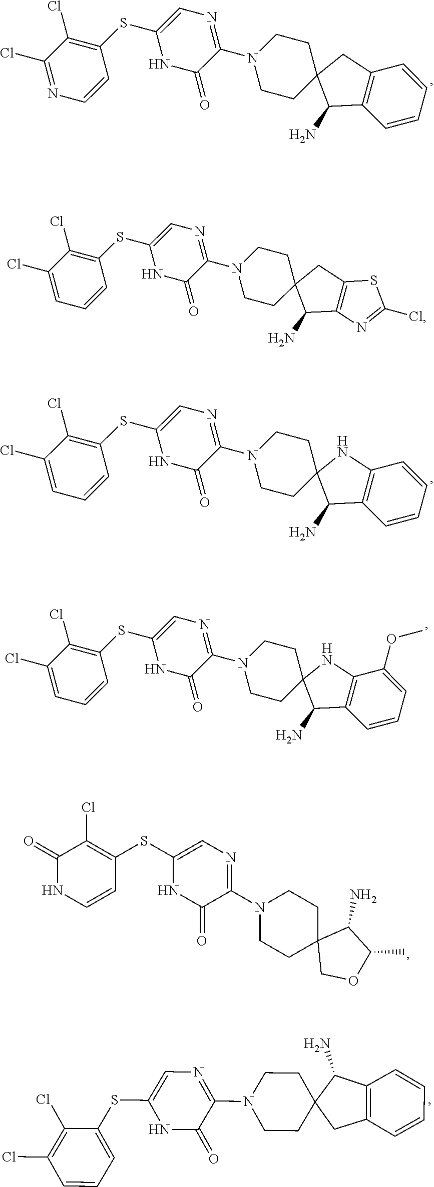 [SMILES: N[C@@H]1c2ccccc2CC12CCN(c1ncc(Sc3ccnc(Cl)c3Cl)[nH]c1=O)CC2 ; N[C@@H]1c2nc(Cl)sc2CC12CCN(c1ncc(Sc3cccc(Cl)c3Cl)[nH]c1=O)CC2 ; N[C@@H]1c2ccccc2NC12CCN(c1ncc(Sc3cccc(Cl)c3Cl)[nH]c1=O)CC2 ; COc1cccc2c1NC1(CCN(c3ncc(Sc4cccc(Cl)c4Cl)[nH]c3=O)CC1)[C@@H]2N ; C[C@@H]1OCC2(CCN(c3ncc(Sc4cc[nH]c(=O)c4Cl)[nH]c3=O)CC2)[C@@H]1N ; N[C@@H]1c2ccccc2CC12CCN(c1ncc(Sc3cccc(Cl)c3Cl)[nH]c1=O)CC2]

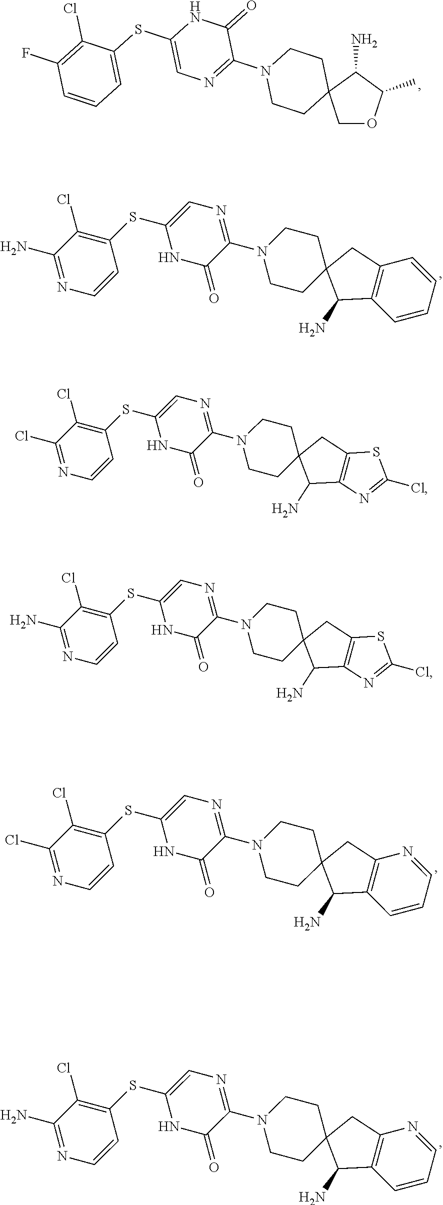 [SMILES: C[C@@H]1OCC2(CCN(c3ncc(Sc4cccc(F)c4Cl)[nH]c3=O)CC2)[C@@H]1N ; Nc1nccc(Sc2cnc(N3CCC4(CC3)Cc3ccccc3[C@H]4N)c(=O)[nH]2)c1Cl ; NC1c2nc(Cl)sc2CC12CCN(c1ncc(Sc3ccnc(Cl)c3Cl)[nH]c1=O)CC2 ; Nc1nccc(Sc2cnc(N3CCC4(CC3)Cc3sc(Cl)nc3C4N)c(=O)[nH]2)c1Cl ; N[C@@H]1c2cccnc2CC12CCN(c1ncc(Sc3ccnc(Cl)c3Cl)[nH]c1=O)CC2 ; Nc1nccc(Sc2cnc(N3CCC4(CC3)Cc3ncccc3[C@H]4N)c(=O)[nH]2)c1Cl]

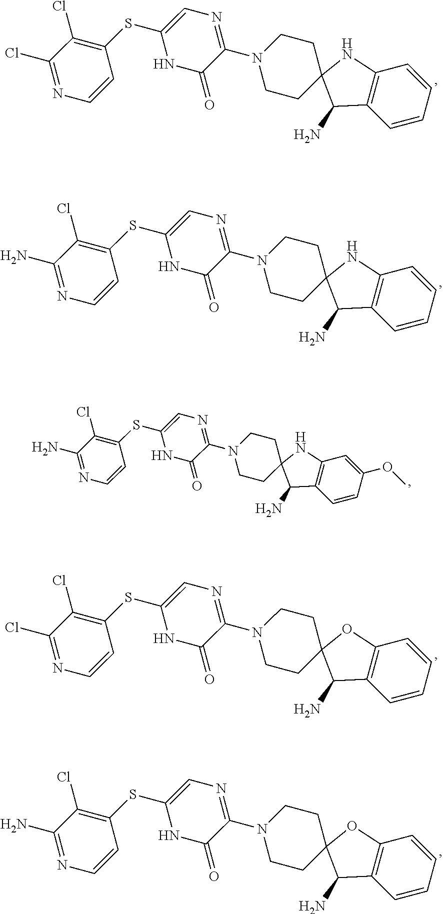 [SMILES: N[C@@H]1c2ccccc2NC12CCN(c1ncc(Sc3ccnc(Cl)c3Cl)[nH]c1=O)CC2 ; Nc1nccc(Sc2cnc(N3CCC4(CC3)Nc3ccccc3[C@H]4N)c(=O)[nH]2)c1Cl ; COc1ccc2c(c1)NC1(CCN(c3ncc(Sc4ccnc(N)c4Cl)[nH]c3=O)CC1)[C@@H]2N ; N[C@@H]1c2ccccc2OC12CCN(c1ncc(Sc3ccnc(Cl)c3Cl)[nH]c1=O)CC2 ; Nc1nccc(Sc2cnc(N3CCC4(CC3)Oc3ccccc3[C@H]4N)c(=O)[nH]2)c1Cl]

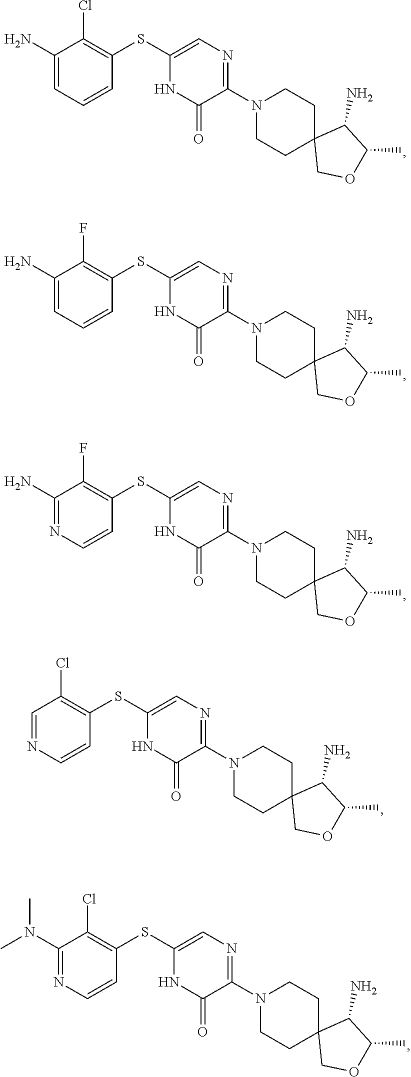

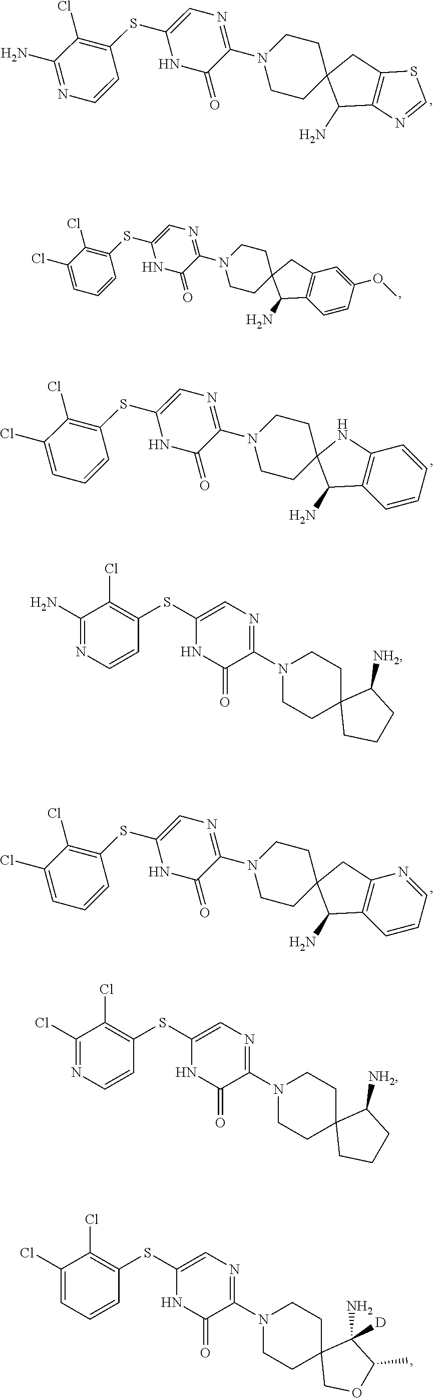

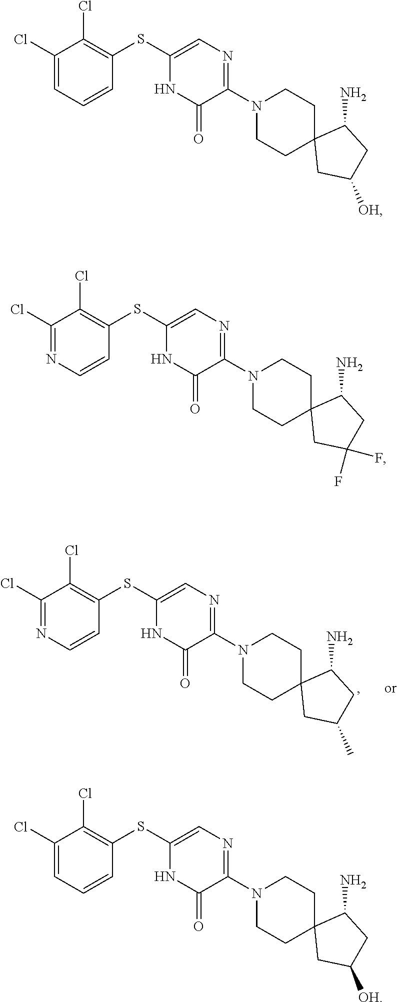 [SMILES: N[C@@H]1C[C@H](O)CC12CCN(c1ncc(Sc3cccc(Cl)c3Cl)[nH]c1=O)CC2 ; N[C@@H]1CC(F)(F)CC12CCN(c1ncc(Sc3ccnc(Cl)c3Cl)[nH]c1=O)CC2 ; C[C@H]1C[C@@H](N)C2(CCN(c3ncc(Sc4ccnc(Cl)c4Cl)[nH]c3=O)CC2)C1 ; N[C@@H]1C[C@@H](O)CC12CCN(c1ncc(Sc3cccc(Cl)c3Cl)[nH]c1=O)CC2]

Embodiment 15a. The method or the use of embodiment 1a, 2a, 3a, 4a, 5a, 6a, 7a, 8a, 9a, 10a, 11a, 12a, 13a, or 14a, wherein any substituent of the compound has a molecular weight of about 15 g/mol to about 500 g/mol.
Embodiment 16a. The method or the use of embodiment 1a, 2a, 3a, 4a, 5a, 6a, 7a, 8a, 9a, 10a, 11a, 12a, 13a, 14a, or 15a, wherein the compound is deuterated.
Embodiment 17a. The method or the use of embodiment 1a, 2a, 3a, 4a, 5a, 6a, 7a, 8a, 9a, 10a, 11a, 12a, 13a, 14a, 15a, or 16a, wherein the compound or a pharmaceutically acceptable salt thereof is in a dosage form comprising a pharmaceutically acceptable vehicle, diluent, or carrier.
Embodiment 18a. A method of treating a disease, a disorder, or a condition associated with the aberrant activity of SHP2, comprising administering a therapeutically effective amount of a compound represented by a formula,

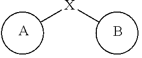

or a pharmaceutically acceptable salt thereof, to a patient in need thereof,

wherein X is S;

wherein Ring A is optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, or optionally substituted 3-(4-oxo-6,7,8,9-tetrahydro-4H-pyrido[1,2-a]pyrimidine-3-carboxamido)phenyl;
wherein Ring B is optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, optionally substituted 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 6-oxo-5-(2-azaspiro[3.4]octan-2-yl)-1,6-dihydropyrazin-2-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, optionally substituted 6-oxo-5-(spiro[indoline-2,4′-piperidin]-1′-yl)-1,6-dihydropyrazin-2-yl, optionally substituted 3-(1-amino-5-methoxy-1,3-dihydrospiro[indene-2,4′-piperidin]-1′-yl)-2(1H)-one-6-yl, optionally substituted 3-(4-amino-4,6-dihydrospiro[cyclopenta[d]thiazole-5,4′-piperidin]-1′-yl)-2(1H)-one-6-yl, optionally substituted 3-(1-amino-8-azaspiro[4.5]decan-8-yl)pyrazin-2(1H)-one-6-yl, optionally substituted 3-(4-amino-3-methyl-2-oxa-8-azaspiro[4.5]decan-8-yl-4-d)pyrazin-2(1H)-one-6-yl, optionally substituted 3-(1-amino-3-hydroxy-8-azaspiro[4.5]decan-8-yl)pyrazin-2(1H)-one-6-yl, or optionally substituted 5-((3S,4S)-4-amino-3-methyl-2-oxa-8-azaspiro[4.5]decan-8-yl-4-d)-6-oxo-1,6-dihydropyrazin-2-yl; and

wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl, —C(O)N(R^{A})-heterocyclyl, or a combination thereof; wherein each substituent has 0-20 carbon atoms and 0-10 heteroatoms, and wherein each heteroatom is independently N, O, S, F, C₁, or Br, provided that the substituent includes at least one C, N, O, S, F, C₁, or Br; and

wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

Embodiment 19a. Use of a compound represented by a formula,

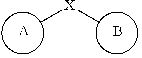

or a pharmaceutically acceptable salt thereof,
in the manufacture of a medicament for treating a disease, a disorder, or a condition associated with the aberrant activity of SHP2, wherein a therapeutically effective amount of the compound or the pharmaceutically acceptable salt is administered to a patient in need thereof,

wherein X is S;

wherein Ring A is optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, or optionally substituted 3-(4-oxo-6,7,8,9-tetrahydro-4H-pyrido[1,2-a]pyrimidine-3-carboxamido)phenyl;

wherein Ring B is optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, optionally substituted 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 6-oxo-5-(2-azaspiro[3.4]octan-2-yl)-1,6-dihydropyrazin-2-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, optionally substituted 6-oxo-5-(spiro[indoline-2,4′-piperidin]-1′-yl)-1,6-dihydropyrazin-2-yl, optionally substituted 3-(1-amino-5-methoxy-1,3-dihydrospiro[indene-2,4′-piperidin]-1′-yl)-2(1H)-one-6-yl, optionally substituted 3-(4-amino-4,6-dihydrospiro[cyclopenta[d]thiazole-5,4′-piperidin]-1′-yl)-2(1H)-one-6-yl, optionally substituted 3-(1-amino-8-azaspiro[4.5]decan-8-yl)pyrazin-2(1H)-one-6-yl, optionally substituted 3-(4-amino-3-methyl-2-oxa-8-azaspiro[4.5]decan-8-yl-4-d)pyrazin-2(1H)-one-6-yl, optionally substituted 3-(1-amino-3-hydroxy-8-azaspiro[4.5]decan-8-yl)pyrazin-2(1H)-one-6-yl, or optionally substituted 5-((3S,4S)-4-amino-3-methyl-2-oxa-8-azaspiro[4.5]decan-8-yl-4-d)-6-oxo-1,6-dihydropyrazin-2-yl; and

wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl, —C(O)N(R^{A})-heterocyclyl, or a combination thereof; wherein each substituent has 0-20 carbon atoms and 0-10 heteroatoms, and wherein each heteroatom is independently N, O, S, F, C₁, or Br, provided that the substituent includes at least one C, N, O, S, F, C₁, or Br; and

wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

Embodiment 20a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises lung cancer.
Embodiment 21a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises non-small cell lung cancer.
Embodiment 22a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises non-small cell lung cancer with KRAS mutant.
Embodiment 23a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises esophageal cancer.
Embodiment 24a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises pancreatic cancer.
Embodiment 25a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition comprises caecum cancer.
Embodiment 26a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition is head and neck cancer.
Embodiment 27a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition is colon cancer.
Embodiment 28a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition is melanoma.
Embodiment 29a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition is leukemia.
Embodiment 30a. The method or the use of embodiment 1a, 2a, 3a, 4a, 5a, 6a, 7a, 8a, 9a, 10a, 11a, 12a, 13a, 14a, 15a, 16a, 17a, 18a, or 19a, wherein the disease, the disorder, or the condition is a metastatic solid tumor.
Embodiment 31a. The method or the use of embodiment 1a, 2a, 3a, 4a, 5a, 6a, 7a, 8a, 9a, 10a, 11a, 12a, 13a, 14a, 15a, 16a, 17a, 18a, or 19a, 20a, 21a, 22a, 23a, 24a, 25a, 26a, 27a, 28a, 29a, or 30a, wherein administration of a combination of two compounds of embodiment ha, 2a, 3a, 4a, 5a, 6a, 7a, 8a, 9a, 10a, 11a, 12a, 13a, 14a, 15a, 16a, 17a, 18a, or 19a, 20a, 21a, 22a, 23a, 24a, 25a, 26a, 27a, 28a, 29a, or 30a, provides higher efficacy in treating the disease, the disorder, or the condition than each single compound alone in the patient, and wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.
Embodiment 32a. The method or the use of embodiment 1a, 2a, 3a, 4a, 5a, 6a, 7a, 8a, 9a, 10a, 11a, 12a, 13a, 14a, 15a, 16a, 17a, 18a, or 19a, 20a, 21a, 22a, 23a, 24a, 25a, 26a, 27a, 28a, 29a, or 30a, wherein administration of a combination of the compound of embodiment ha, 2a, 3a, 4a, 5a, 6a, 7a, 8a, 9a, 10a, 11a, 12a, 13a, 14a, 15a, 16a, 17a, 18a, or 19a, 20a, 21a, 22a, 23a, 24a, 25a, 26a, 27a, 28a, 29a, or 30a, with other agent, provides higher efficacy in treating the disease, the disorder, or the condition than the compound or the agent alone in the patient, and wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.

### EXPERIMENTAL

### General Synthetic Methods

The compounds of the present invention, or their pharmaceutically acceptable salts, can be synthesized using the methods described below in schemes 1-6. It will be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that optimum reaction conditions may vary with the particular reactants or solvents used, but such conditions can be determined by the person skilled in the art, using routine optimization procedures. Additionally, one skilled in the art will recognize that in many cases, these compounds will be mixtures of stereoisomers that may be separated at various stages of the synthetic schemes using conventional techniques, such as, but limited to, crystallization, normal-phase chromatography, reversed phase chromatography and chiral chromatography, to afford single enantiomers. For all the protection and deprotection methods, see Philip J. Kocienski, in “Protecting Groups”, Georg Thieme Verlag Stuttgart, N.Y., 1994 and, Theodora W. Greene and Peter G. M. Wuts in “Protective Groups in Organic Synthesis”, Wiley Interscience, 3rd Edition 1999. The schemes 1-6 are representative of methods useful in synthesizing the compounds of the present invention. They are not to constrain the scope of the invention in any way.

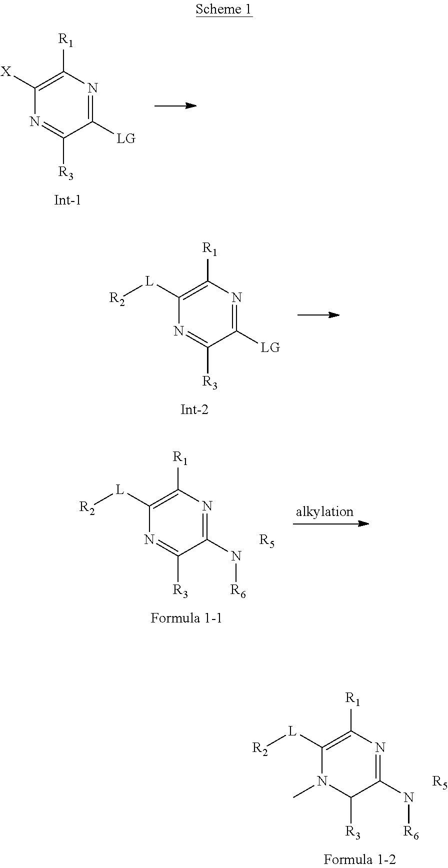

Scheme 1 illustrates a method for preparing compounds of Formula 1. In which L is S, O, N or a bond; R₁ is H, C₁-C₆ alkyl, NH₂, or CN; R₂ is aryl, heterocycloalkyl or heteroaryl; R₃ is C₁-C₆ alkyl, OR₁; R₅ is alkyl, H; R₆ is alkyl, H; R₅ and R₆ together with the atom or atoms to which they are attached, can combine to form a monocyclic or polycyclic C₃-C₁₂ cycloalkyl or heterocycle.

Compound int-1 is treated with aryl or alkyl boronic acids or esters or salts (where L is a bond) under suitable metal catalysts (such Pd₂(dba)₃, or the like), suitable ligands (such as dppf, or the like), suitable bases (such as Cs₂CO₃, or the like), suitable solvents (such as DMF, or the like) to provide a product of int-2. In other cases, where the L of Formula 1 is O, N or S, compound int-1 was reacted with corresponding phenols, thiophenols, thioalcohols or amines under suitable metal catalysts (such as Cul, Pd₂(dba)₃), suitable ligands (such as TMEDA, XPHOS, Xantphos, or the like), suitable salts or bases (such as Cs₂CO₃, K₃PO₄ or the like), suitable solvents (such as DMF, dioxane or the like) to provide a product of int-2. The reaction temperature is ranged from 80° C. to 140° C., and the reaction takes 1-24 hours to complete. Compound int-2 reacted with amines with or without bases (such as DIPEA), in suitable solvents (such as DMF, NMP or the like) under the temperature range from 80° C. to 140° C. to offer compound of Formula 1. If desired, further transformations may be performed to provide a product of Formula 1-2. For example, the compound of Formula 1-1 wherein R₃═OH may be subjected to an alkylation reaction to provide a compound of Formula 1-2.

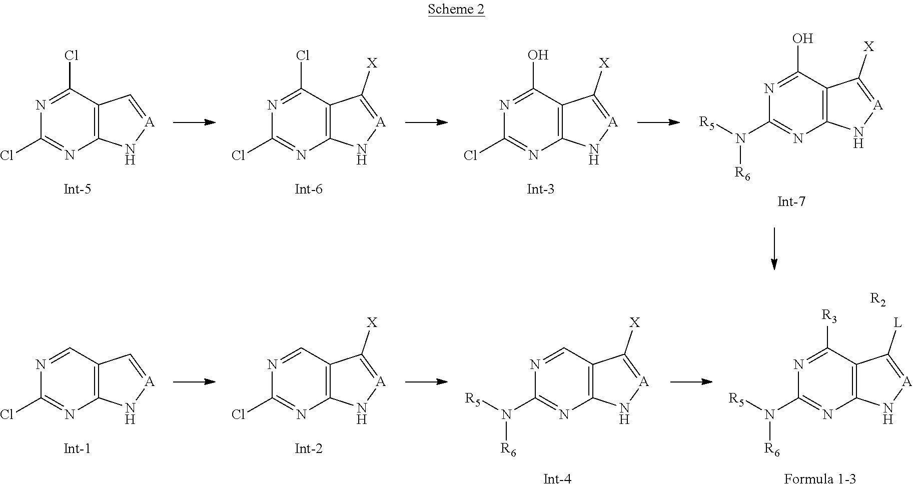

Scheme 2 illustrates a method for preparing compounds of Formula 1-3. Compound Int-1 was first halogenated to provide intermediate Int-2. The activated chlorine in Int-2 was displaced by an amine to afford Int-4. Int-4 reacted with an aryl amine or phenol to provide target compounds of Formula 1-3 (R₃═H).

For the compounds of Formula 1-3 where R₃ was OH, Int-6 was regioselectively hydrolyzed to provide Int-3, and then followed by substitutions and couplings to offer target compounds.

Deprotection steps can be incorporated either before or after the coupling reactions.

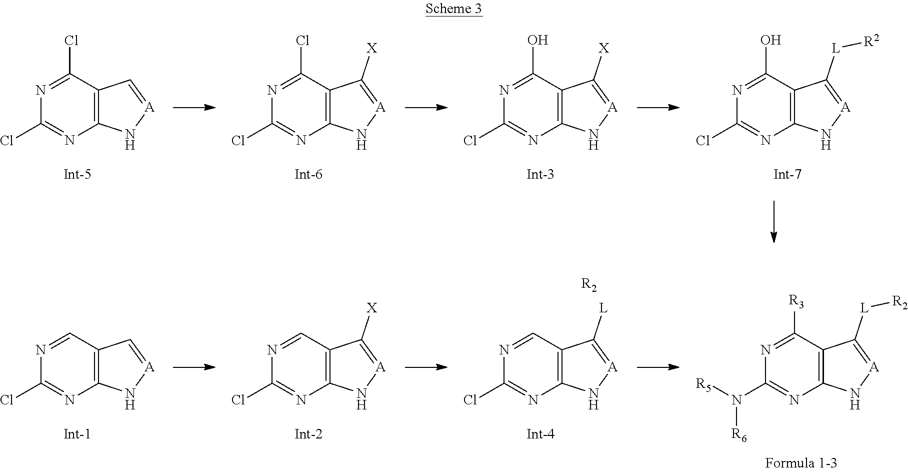

Additionally, the compounds of Formula 1-3 can be synthesized using an alternative way, as illustrated in Scheme 3. The order of reaction steps may alter. Coupling reactions may occur before the amine displacement.

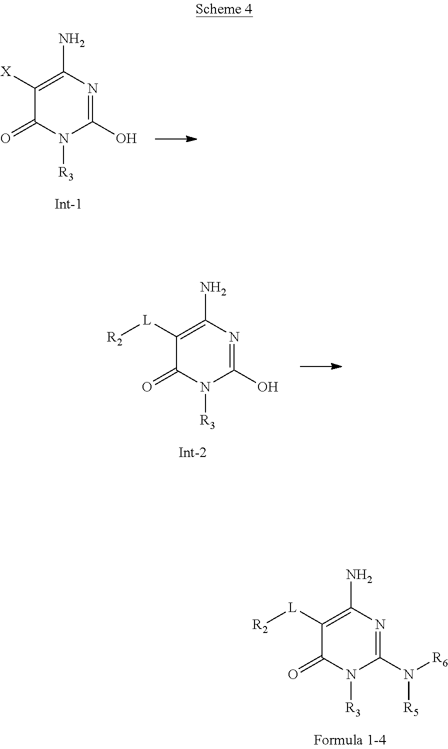

Scheme 4 illustrates a method for preparing compounds of Formula 1-4. Int-1 was reacted with an aryl amine or phenol in the presence of a suitable metal catalyst (such as Cul, or the like), a suitable ligand (such as TMEDA, TMHD, or the like), a suitable salt (such as K₃PO₄, or the like) and a suitable solvent (such as dioxane or the like). The reaction proceeds at a temperature ranged 80° C. to 140° C., with the reaction time from 1-24 hours. Int-2 reacted with an amine in the presence of a suitable coupling reagent (such as BOP—Cl, or the like), a suitable base (such DIEPA, DBU, or the like), and a suitable solvent (such as DMF, THF or the like). The reaction proceeded at temperature 80° C. to 130° C. with 1-24 hours to finish.

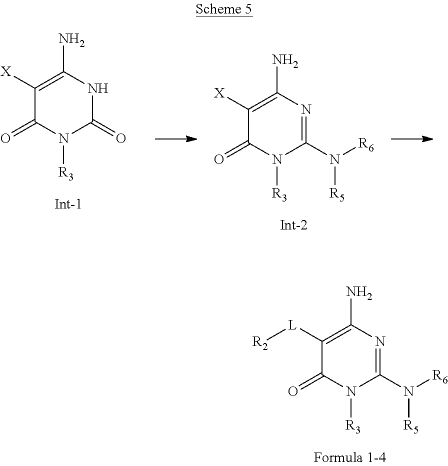

Alternatively, as illustrated in Scheme 5, the order of reactions can be modified to change the overall synthesis to allow for variations at different positions of the molecule at different stages of the preparation. For example, in Scheme 5, compound of Formula Int-1 is activated and reacted with an amine to provide Int-2 first, and then followed with the coupling reaction, to provide compound of Formula 1-4.

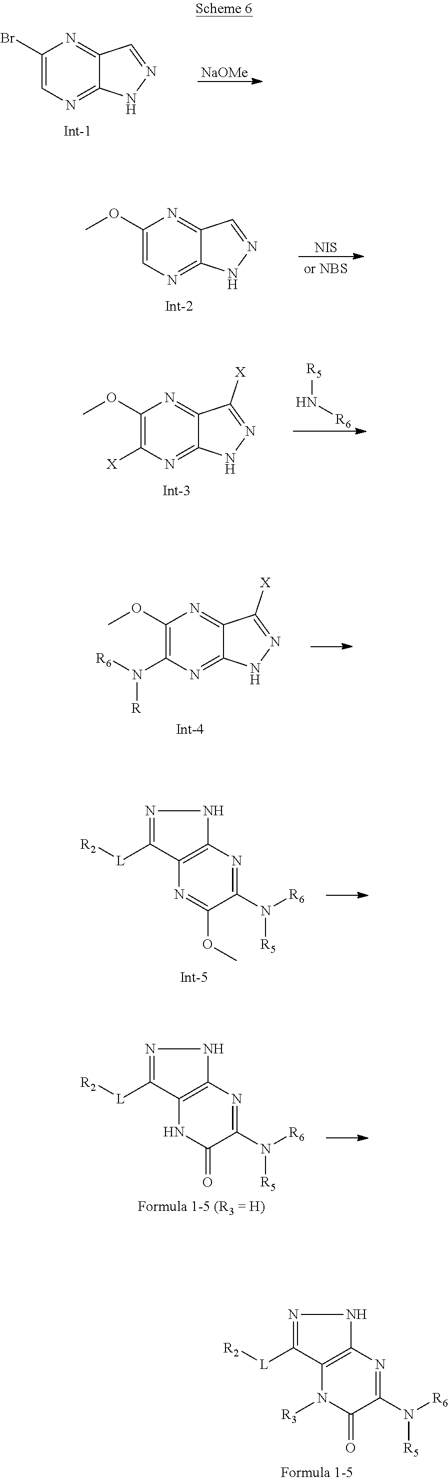

Scheme 6 illustrates a method for preparing compounds of Formula 1-5.

Bromine of Int-1 was displaced with methoxy group to afford Int-2, and then Int-2 was halogenated with NBS or NIS to offer Int-3. From Int-3, after the substitution, coupling and deprotection compounds of formula 1-5 (R₃═H) were synthesized. The compound of Formula 1-5 wherein R 3═H may be subjected to an alkylation reaction to provide another series compound of Formula 1-5.

### EXPERIMENTAL PROCEDURES AND EXAMPLES

Experiments were generally carried out under inert atmosphere (nitrogen or argon), particularly in cases where oxygen-or moisture-sensitive reagents or intermediates were employed. Commercial solvents and reagents were generally used without further purification, including anhydrous solvents where appropriate. Products were generally dried under vacuum before being carried on to further reactions or submitted for biological testing. Mass spectrometry data is reported from liquid chromatography-mass spectrometry (LCMS) instrumentation. Mass spectra, MS (m/z), were recorded using either electrospray ionization (ESI) or atmospheric pressure chemical ionization (APCI). Where relevant and unless otherwise stated the m/z data provided are for isotopes 19F, 35Cl, 79Br and 1271. Chemical shifts for nuclear magnetic resonance (NMR) data are expressed in parts per million (ppm, δ) referenced to residual peaks from the deuterated solvents employed, using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d6-DMSO, derterodimethylsulphoxide; and CD₃OD, deuteromethanol.

In general, reactions were followed by thin layer chromatography (TLC) and/or liquid chromatography-mass spectrometry (LCMS) and subjected to work-up when appropriate. Purification was carried out by chromatographic and/or HPLC.

Unless noted otherwise, all reactants were obtained commercially.

### Example 1

### Preparation of 2-(4-(Aminomethyl)-4-Methylpiperidin-1-Yl)-5-(2,3-Dichlorophenyl)-1-Methyl-6-Oxo-1,6-Dihydropyrimidine-4-Carbonitrile

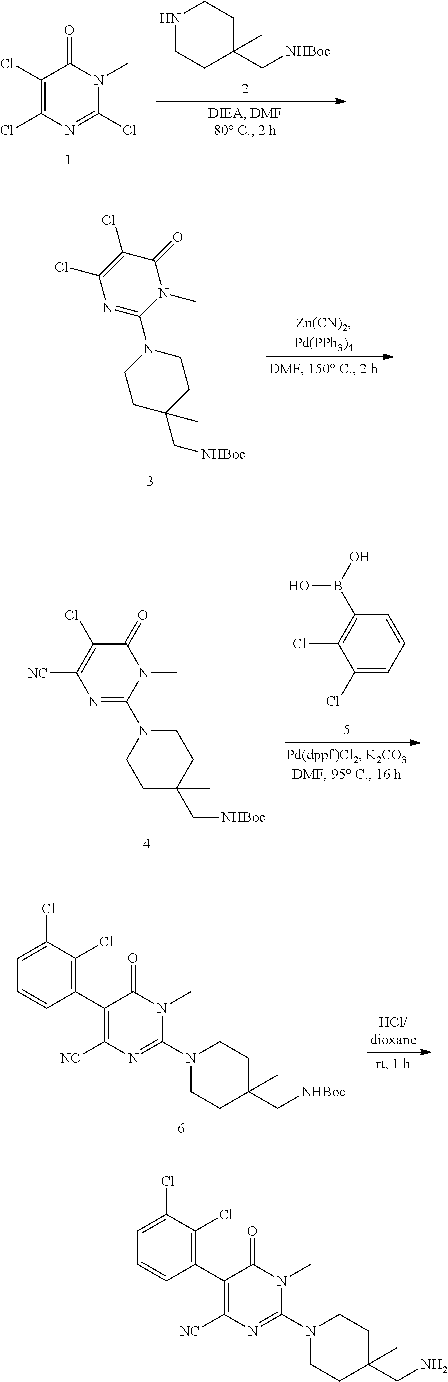

Step 1: preparation of tert-butyl ((1-(4,5-dichloro-1-methyl-6-oxo-1,6-dihydropyrimidin-2-yl)-4-methylpiperidin-4-yl)methyl)carbamate (3)

To a mixture of compound 1 (1.4 g, 1.0 eq) and compound 2 (1.5 g, 1.0 eq) in DMF (25 mL) was added DIEA (3.2 mL, 2.0 eq). The mixture was degassed and protected with nitrogen. The reaction was stirred at 80° C. for 2 h. EtOAc (150 mL) was added to the mixture under r.t. After standard work up procedure, the residue was purified by column chromatography to give compound 3 as a white solid (1.9 g, 71%).

Step 2: preparation of tert-butyl ((1-(5-chloro-4-cyano-1-methyl-6-oxo-1,6-dihydropyrimidin-2-yl)-4-methylpiperidin-4-yl)methyl)carbamate (4)

A mixture of compound 3 (200 mg, 1.0 eq), Zn(CN)₂ (57.9 g, 1.0 eq) and Pd(PPh₃)₄ (56 mg, 0.1 eq) in DMF was stirred at 150° C. for 2 h, and the mixture was cooled to rt. EtOAc was added, after standard work up procedure the residue was purified by prep-TLC to give compound 4 as a white solid (60 mg, 31%).

Step 3: preparation of tert-butyl ((1-(4-cyano-5-(2,3-dichlorophenyl)-1-methyl-6-oxo-1,6-dihydropyrimidin-2-yl)-4-methylpiperidin-4-yl)methyl)carbamate (6)

To a mixture of compound 4 (60 mg, 1.0 eq), K₂CO₃ (42 mg, 2 eq) and compound 5 (86 mg, 3.0 eq) in DMF (10 mL) was added Pd(dppf)Cl₂(11 mg, 0.1 eq). The resulting mixture was stirred at 95° C. overnight. The mixture was diluted with EtOAc. After standard work up procedure, the residue was purified by prep-TLC to give compound 6 as a white solid (20 mg, 26%).

Step 4: preparation of 2-(4-(aminomethyl)-4-methylpiperidin-1-yl)-5-(2,3-dichlorophenyl)-1-methyl-6-oxo-1,6-dihydropyrimidine-4-carbonitrile

HCl/dioxane (0.5 mL, 10.0 eq) was added to a solution of compound 6 (19 mg, 1.0 eq) in DCM (5.0 mL), and then the reaction was stirred at rt for 1 h. The mixture was concentrated and neutralized with ammonium hydroxide, purified by prep-HPLC to give the desired compound as a white solid (2.5 mg, 17%). LC-MS: [M+H]+: 406.1. ¹H NMR (400 MHz, CD₃OD) δ 7.65 (dd, J=8.0, 1.6 Hz, 1H), 7.40 (t, J=8.0 Hz, 1H), 7.33 (dd, J=8.0, 1.2 Hz, 1H), 3.55-3.48 (m, 4H), 3.34-3.31 (m, 2H), 2.66 (s, 1H), 1.71-1.66 (m, 2H), 1.55-1.52 (m, 2H), 1.10 (s, 3H).

### Example 2

### Preparation of 3-(4-(Aminomethyl)-4-Methylpiperidin-1-Yl)-6-(2,3-Dichlorophenyl) Pyrazin-2(1H)-One

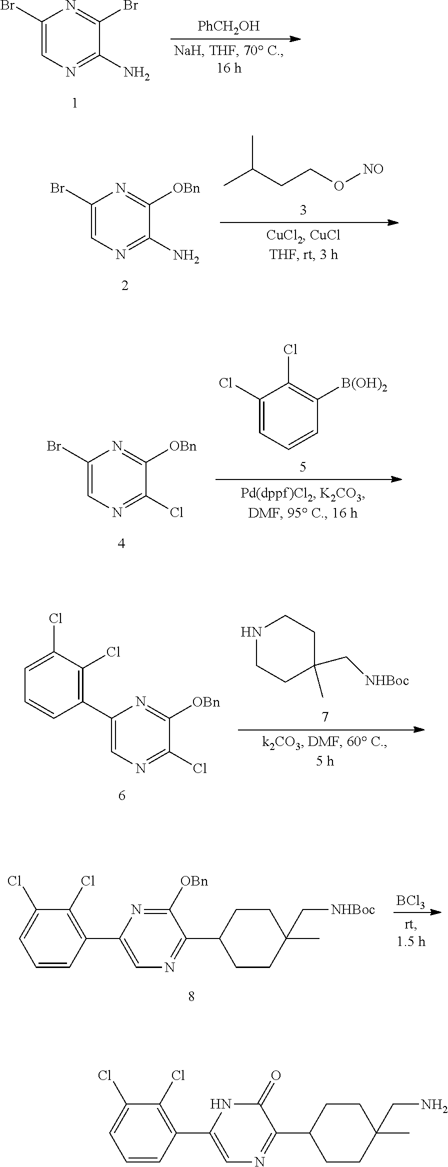

Step 1: Preparation of 3-(benzyloxy)-5-bromopyrazin-2-amine (2)

To a solution of benzyl alcohol (13.5 g, 1.05 eq) in THF (300 mL) was added NaH (60%, 5.7 g, 1.2 eq) at rt. The mixture was stirred at rt for 30 min. Compound 1 (30.0 g, 118 mmol, 1.0 eq) was added to the mixture. The mixture was heated to 70° C. and stirred overnight. The reaction was quenched by water, worked up under standard operation to give compound 2 as a yellow solid (25.0 g, 75%).

Step 2: preparation of 3-(benzyloxy)-5-bromo-2-chloropyrazine (4)

To a solution of compound 2 (9.0 g, 1.0 eq) in THF (30 mL) was added CuCl₂ (12.96 g, 3.0 eq) and CuCl (5.75 g, 2.0 eq) at rt. Compound 3 (12.9 mL, 3.0 eq) was added dropwise to the mixture 10 min later. The reaction was worked up under standard operation to give compound 4 as a yellow oil (4.0 g, 41%).

Step 3: preparation of 3-(benzyloxy)-2-chloro-5-(2,3-dichlorophenyl)pyrazine (6)

To a solution of compound 4 (3.0 g, 1.0 eq) in DMF (30 mL) was added K₂CO₃ (4.14 g, 3.0 eq), compound 5 (1.9 g, 1.0 eq) and Pd(dppf)Cl₂ (0.73 g, 0.1 eq). The resulting mixture was stirred at 95° C. under N₂ atmosphere overnight. The reaction was quenched by water and worked up under standard operation to give compound 6 as a yellow solid (1.5 g, 40%).

Step 4: preparation of tert-butyl ((1-(3-(benzyloxy)-5-(2,3-dichlorophenyl)pyrazin-2-yl)-4-methylpiperidin-4-yl)methyl)carbamate (8)

To a solution of compound 6 (500 mg, 1.0 eq) in DMF (5 mL) were added compound 7 (342 mg, 1.1 eq) and K₂CO₃ (660 mg, 3.5 eq). The mixture was stirred at 60° C. under N₂ atmosphere for 5 h. The reaction was quenched by water and worked up under standard operation to give compound 8 as a white solid (400 mg, 53%).

Step 5: preparation of 3-(4-(aminomethyl)-4-methylpiperidin-1-yl)-6-(2,3-dichlorophenyl)-pyrazin-2(1H)-one

To a solution of compound 8 (100 mg, 1.0 eq) in DCM (5 mL) was added BCl₃ (1M in DCM, 5.0 eq). The mixture was stirred at rt for 1.5 h. The reaction was quenched by MeOH (5 mL) and the mixture was concentrated and purified by prep-HPLC to give target compound as a white solid (7 mg, 10%). LC-MS: [M+H]+=367. ¹H NMR (400 MHz, DMSO-d₆) δ 7.67 (d, J=7.4 Hz, 1H), 7.52-7.34 (m, 2H), 6.91 (s, 1H), 4.10-4.03 (m, 2H), 3.45-3.32 (m, 2H), 2.88 (d, J=6.0 Hz, 1H), 2.39 (s, 1H), 1.50-1.41 (m, 2H), 1.26-1.24 (m, 2H), 0.90 (d, J=9.2 Hz, 3H).

### Example 3

### Preparation of 3-(4-(Aminomethyl)-4-Methylpiperidin-1-Yl)-6-((2,3-Dichlorophenyl)Thio)-1-Methylpyrazin-2(1H)-One

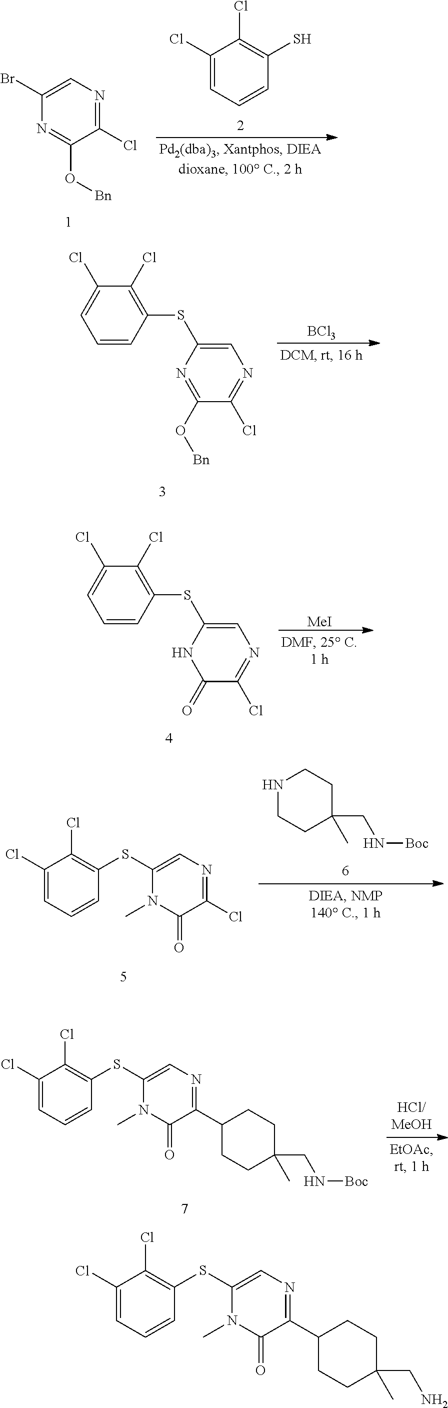

Step 1: preparation of 3-(benzyloxy)-2-chloro-5-((2,3-dichlorophenyl)thio)pyrazine (3)

To a mixture of compound 1 (3.0 g, 1.0 eq), compound 2 (1.79 g, 1.0 eq), Pd2(dba)3 (300 mg) and Xantphos (300 mg) in dioxane (50 mL) was added DIEA (3.0 mL, 1.8 eq), and then the mixture was degassed and protected with nitrogen. EtOAc (150 mL) was added to the mixture 2 hours later. After standard work up procedure the residue was purified by column chromatography to give compound 3 as a colorless oil (2.9 g, 73%).

Step 2: preparation of 3-chloro-6-((2,3-dichlorophenyl)thio)pyrazin-2(1H)-one (4)

BCl₃ (10.0 mL, 1M in DCM, 1.99 eq) was added to a solution of compound 3 (2.0 g, 1.0 eq) in DCM at rt and then the reaction was stirred at rt overnight. The reaction was quenched by sat. NaHCO₃ and stirred for 1 h. The suspension was then filtered and the solid was washed with DCM and water. The solid was dried to give the crude product 4 as a yellow solid. (3.0 g, 78%).

Step 3: preparation of 3-chloro-6-((2,3-dichlorophenyl)thio)-1-methylpyrazin-2(1H)-one (5)

Mel (1 mL) was added to a mixture of crude 4 (2.0 g, 1.0 eq) and K₂CO₃ (2.0 g, 5.6 eq) in DMF (10 mL) and the resulting mixture was stirred at 25° C. for 1 h. The mixture was diluted with EtOAc and then was washed with brine. The organic solution was dried over Na₂SO₄ and concentrated. The residue was purified by column chromatography to give compound 5 as a white solid (505 mg, 60%).

Step 4: preparation of tert-butyl ((1-(5-((2,3-dichlorophenyl)thio)-4-methyl-3-oxo-3,4-dihydropyrazin-2-yl)-4-methylpiperidin-4-yl)methyl)carbamate (7)

A mixture of compound 5 (96 mg, 1.0 eq), compound 6 (73 mg, 1.07 eq) and DIEA (0.7 mL, 1.8 eq) in NMP (2 mL) was stirred at 140° C. for 1 h. The mixture was diluted with EtOAc and then washed with brine. The organic solution was dried over Na₂SO₄ and concentrated. The residue was purified by column chromatography to give compound 7 as an off-shite solid (140 mg, 91%)

Step 3: preparation of 3-(4-(aminomethyl)-4-methylpiperidin-1-yl)-6-((2,3-dichlorophenyl)thio)-1-methylpyrazin-2(1H)-one

HCl/MeOH (1.0 mL, 10.0 eq) was added to the solution of compound 7 (51 mg, 1.0 eq) in EtOAc (2.0 mL) and then the reaction was stirred at rt for 1 h. The reaction solution was kept for 2 days and the obtained suspension was filtered. The solid was washed with EtOAc and the solid was dried to give the desired compound as HCl salt, white solid (24 mg, 53%). LC-MS: [M+H]⁺: 413.1. ¹H NMR (400 MHz, CD₃OD) δ 7.45 (d, J=8.0 Hz, 1H), 7.30-7.25 (m, 2H), 7.09 (d, J=8.0 Hz, 1H), 4.48-4.43 (m, 2H), 3.88-3.81 (m, 2H), 3.46 (s, 3H), 2.96 (s, 2H), 1.81-1.67 (m, 4H), 1.23 (s, 3H).

The compounds in Table A below were synthesized in the similar manner using appropriate reagents and conditions. The compounds listed in Table A are merely non-limiting examples. Other subject compounds could also be made using similar methods.

TABLE A

| Compound | |
|---|---|
| ID | ¹H-NMR & MS [M + 1]⁺ |
| 7 | LC-MS: [M + H]⁺: 385.1 |
| | ¹H NMR (400 MHz, CD₃OD) δ 7.34 (d, J = 8.4 Hz, 1H), 7.20 (t, J = 8.0 Hz, 1H), 7.13 (s, 1H), 6.93 |
| | (d, J = 7.6 Hz, 1H), 4.00-3.90 (m, 2H), 3.80-3.66 (m, 2H), 2.72 (s, 2H), 1.96-1.85 (m, 1H), |
| | 1.77-1.72 (m, 1H), 1.15 (s, 3H). |
| 2 | LC-MS: [M + H]⁺: 399.1 |
| | ¹H NMR (400 MHz, DMSO-d₆) δ 12.11 (br s, 1H), 7.97 (br s, 3H), 7.49 (d, J = 8.0 Hz, 1H), 7.35-7. [symbol] |
| | (m, 1H), 7.22 (s, 1H), 6.95 (d, J = 8.0 Hz, 1H), 4.27-4.24 (m, 2H), 3.58-3.53 (m, 2H), 2.76 (s, 2H), |
| | 1.58-1.41 (m, 4H), 1.08 (s, 3H). |
| 4 | LC-MS: [M + H]⁺: 383 |
| | ¹H NMR (400 MHz, CD₃OD) δ 8.57 (s, 1H), 7.33 (d, J = 8.0 Hz, 1H), 7.26 (t, J = 8.0 Hz, 1H), 7.06 ( [symbol] |
| | J = 7.6 Hz, 1H), 6.96 (s, 1H), 3.67 (br s, 2H), 3.15 (br s, 2H), 2.86 (br s, 2H), 1.66 (s, 2H), 1.53-1.5 |
| | (m, 2H), 1.12 (s, 3H). |
| 6 | LC-MS: [M + H]⁺: 397 |
| | ¹H NMR (400 MHz, CD₃OD) δ 7.47 (d, J = 8.4 Hz, 1H), 7.37 (t, J = 8.4 Hz, 1H), 7.21 (d, J = 8.4 Hz |
| | 1H), 6.42 (s, 1H), 3.94-3.91 (m, 2H), 3.53 (s, 3H), 3.28-3.25 (m, 2H), 2.86 (br s, 2H), 1.68-1.61 ( [symbol] |
| | 2H), 1.54-1.51 (m, 2H), 1.13 (s, 3H). |
| 5 | LC-MS: [M + H]⁺: 382.2 |
| | ¹H NMR (400 MHz, CD₃OD) δ 7.14 (t, J = 8.0 Hz, 1H), 7.03 (d, J = 8.0 Hz, 1H), 6.95-6.90 (m, 2H |
| | 4.03-3.96 (m, 2H), 3.40-3.31 (m, 2H), 2.90 (s, 2H), 1.70-1.64 (m, 2H), 1.69-1.63 (m, 2H), 1. [symbol] |
| | (s, 3H). |
| 12 | LC-MS: [M + H]⁺: 381.2 |
| | ¹H NMR (400 MHz, CD₃OD) δ 8.53 (s, 1H), 8.34 (d, J = 8.4 Hz, 1H), 7.91 (d, J = 7.6 Hz, 1H), 7.82 |
| | J = 8.0 Hz, 1H), 7.64-7.51 (m, 2H), 7.51-7.37 (m, 2H), 7.17 (s, 1H), 4.29-4.24 (m, 2H), 3.48- |
| | 3.42 (m, 2H), 2.85 (s, 2H), 1.67-1.55 (m, 2H), 1.52-1.48 (m, 2H), 1.15 (s, 3H). |
| 8 | LC-MS: [M + H]⁺: 383 |
| | ¹H NMR (400 MHz, DMSO) δ 7.46 (d, J = 8.0 Hz, 1H), 7.32 (d, J = 8.0 Hz, 1H), 7.13 (s, 1H), 6.94 ( [symbol] |
| | J = 8.4 Hz, 1H), 3.99-3.97 (m, 2H), 3.68-3.65 (m, 2H), 2.95-2.91 (m, 2H), 2.76-2.74 (m, 2H), |
| | 2.66-2.63 (m, 2H). |
| 9 | LC-MS: [M + H]⁺: 369.1 |
| | ¹H NMR (400 MHz, CD₃OD) δ 7.35 (dd, J = 8.0, 1.2 Hz, 1H), 7.20 (t, J = 8.0 Hz, 1H), 7.11 (s, 1H), |
| | 6.93 (dd, J = 8.0, 1.2 Hz, 1H), 5.27-5.22 (m, 1H), 4.42-4.38 (m, 1H), 3.95-3.91 (m, 1H), 3.63- |
| | 3.60 (m, 1H), 3.29-3.25 (m, 1H), 3.24-3.20 (m, 1H), 2.88-2.85 (m, 1H), 2.79-2.77 (m, 1H). |
| 11 | LC-MS: [M + H]⁺: 371 |
| | ¹H NMR (400 MHz, DMSO-d₆) δ 8.32 (s, 1H), 7.53 (d, J = 7.6 Hz, 1H), 7.48-7.46 (m, 1H), 7.31 ( [symbol] |
| | J = 8.0 Hz, 1H), 7.14 (s, 1H), 6.96-6.93 (m, 1H), 3.96-3.94 (m, 1H), 3.11-3.08 (m, 2H), 2.74-2. [symbol] |
| | (m, 2H), 1.88-1.85 (m, 2H), 1.65-1.56 (m, 2H). |
| 17 | LC-MS: [M + H]+: 399.2 |
| | ¹H NMR (400 MHz, CD₃OD) δ 8.53 (s, 1H), 7.71 (d, J = 7.6 Hz, 1H), 7.51 (t, J = 7.6 Hz, 1H), 7.36 ( |
| | J = 7.6 Hz, 1H), 7.24 (d, J = 8.0 Hz, 1H), 7.21 (s, 1H), 4.39-4.34 (m, 2H), 3.57-3.50 (m, 2H), 2.8 |
| | (s, 2H), 1.65-1.59 (m, 2H), 1.55-1.51 (m, 2H), 1.17 (s, 3H). |
| 19 | LC-MS: [M + H]⁺: 380.2 |
| | ¹H NMR (400 MHz, CD₃OD) δ 7.20 (s, 1H), 7.07 (d, J = 8.6 Hz, 1H), 6.53 (dd, J = 8.4, 2.6 Hz, 1H), |
| | 6.38 (d, J = 2.6 Hz, 1H), 4.35-4.32 (m, 2H), 3.54-3.48 (m, 2H), 2.89 (s, 4H), 1.70-1.45 (m, 4H), 1. [symbol] |
| | (s, 3H). |
| 21 | LC-MS: [M + H]⁺: 380 |
| | ¹H NMR (400 MHz, MeOD) δ 8.53 (s, 1H), 7.19 (s, 1H), 6.94 (t, J = 8.0 Hz, 1H), 6.66 (dd, J = 8.1, |
| | 1.2 Hz, 1H), 6.26 (dd, J = 7.8, 1.2 Hz, 1H), 4.42-4.24 (m, 2H), 3.51 (m, 2H), 2.86 (s, 2H), 1.61 ( [symbol] |
| | 2H), 1.52 (m, 2H), 1.16 (s, 3H). |
| 10 | LC-MS: [M + H]⁺: 427 |
| | ¹H NMR (400 MHz, DMSO) δ 8.26 (s, 1H), 7.48 (dd, J = 6.8 Hz, 1.2 Hz, 1H), 7.33 (t, J = 8.0 Hz, 1 [symbol] |
| | 7.22 (s, 1H), 6.95 (dd, J = 6.8 Hz, 1.2 Hz, 1H), 4.47-4.43 (m, 2H), 3.95(t, J = 8.4 Hz, 1H), 3.71 (d, [symbol] |
| | 8.4 Hz, 1H), 3.61 (d, J = 8.8 Hz, 1H), 3.30-3.28 (m, 3 H), 3.09-3.07 (m, 1H), 1.70-1.61 (m, 2H), |
| | 1.44-1.43 (m, 2H). |
| 18 | LC-MS: [M + H]⁺: 400 |
| | ¹H NMR (400 MHz, DMSO) δ 8.52 (d, J = 3.2 Hz, 1H), 8.31 (s, 1H), 7.39-7.62 (m, 2H), 7.22 (s, 1H |
| | 4.24-4.21 (m, 2H), 3.57-3.48 (m, 2H), 2.58 (s, 2H), 1.53-1.47 (m, 2H), 1.36-1.33 (m, 2H), 0.99 (s |
| | 3H). |
| 20 | LC-MS: [M + H]⁺: 381.2 |
| | ¹H NMR (400 MHz, CD₃OD) δ 7.52 (d, J = 2.8 Hz, 1H), 7.23 (s, 1H), 6.71 (d, J = 2.8 Hz, 1H), 4.28- |
| | 4.23 (m, 2H), 3.53-3.47 (m, 2H), 2.61 (s, 2H), 1.62-1.55 (m, 2H), 1.47-1.43 (m, 2H), 1.07 (s, 3H) |
| 24 | LC-MS: [M + H]⁺: 396 |
| | ¹H NMR (400 MHz, MeOD) δ 8.51 (s, 1H), 7.88 (d, J = 5.5 Hz, 1H), 7.25 (s, 1H), 6.50 (d, J = 5.5 H |
| | 1H), 4.41 (d, J = 13.9 Hz, 2H), 3.97 (s, 3H), 3.56 (m, 2H), 2.88 (s, 2H), 1.61 (m, 4H), 1.18 (s, 3H). |
| 69 | LC-MS: [M + H]⁺: 403.2 |
| | ¹H NMR (400 MHz, CD₃OD) δ 8.50 (s, 1H), 7.37 (dd, J = 8.0, 1.4 Hz, 1H), 7.26 (s, 1H), 7.21 (t, J = |
| | 8.0 Hz, 1H), 6.94 (dd, J = 8.0, 1.4 Hz, 1H), 4.82-4.79 (m, 2H), 3.37-3.34 (m, 2H), 3.14 (d, J = 19.8 |
| | Hz, 2H), 2.06-1.75 (m, 4H). |
| 70 | LC-MS: [M + H]⁺: 437 |
| | ¹H NMR (400 MHz, MeOD) δ 8.50 (s, 1H), 7.63 (dd, J = 8.0, 1.4 Hz, 1H), 7.42 (t, J = 7.8 Hz, 1H), |
| | 7.34 (dd, J = 7.6, 1.4 Hz, 1H), 4.68 (s, 2H), 4.36-4.24 (m, 1H), 4.05-3.92 (m, 1H), 3.85 (d, J = 9.1 |
| | Hz, 1H), 3.78-3.62 (m, 2H), 3.44 (t, J = 12.0 Hz, 1H), 3.08 (dt, J = 24.9, 6.9 Hz, 2H), 2.05-1.82 (m |
| | 3H), 1.75 (d, J = 12.9 Hz, 1H), 1.32 (d, J = 6.5 Hz, 3H).1 |
| 71 | LC-MS: [M + H]⁺: 397 |
| | ¹H NMR (400 MHz, MeOD) δ 8.54 (s, 1H), 7.35 (dd, J = 8.0, 1.4 Hz, 1H), 7.25-7.18 (m, 1H), 7.14 |
| | (s, 1H), 6.93 (dd, J = 8.0, 1.3 Hz, 1H), 3.30 (d, J = 1.6 Hz, 3H), 3.26 (s, 2H), 1.91 (s, 2H), 1.75 (t, J |
| | 10.0 Hz, 2H), 1.62 (d, J = 9.2 Hz, 2H). |
| 72 | LC-MS: [M + H]⁺: 397 |
| | ¹H NMR (400 MHz, DMSO) δ 8.36 (s, 1H), 7.47 (dd, J = 8.0, 1.3 Hz, 1H), 7.32 (t, J = 8.0 Hz, 1H), |
| | 7.12 (d, J = 5.2 Hz, 1H), 6.95 (d, J = 8.0 Hz, 1H), 5.67 (s, 1H), 3.53 (dd, J = 167.4, 77.9 Hz, 7H), 1. [symbol] |
| | (dd, J = 37.8, 25.8 Hz, 4H), 1.44 (s, 2H). |
| 14 | LC-MS: [M + H]⁺: 441.2 |
| | ¹H NMR (400 MHz, DMSO) δ 8.53 (s, 1H), 7.36 (d, J = 8.0 Hz, 1H), 7.21 (dd, J = 15.9, 7.8 Hz, 2H) |
| | 6.92 (d, J = 8.0 Hz, 1H), 4.65-4.39 (m, 2H), 4.35-4.14 (m, 1H), 3.92 (d, J = 8.9 Hz, 1H), 3.79 (d, J [symbol] |
| | 8.9 Hz, 1H), 3.31-3.21 (m, 3H), 2.01-1.57 (m, 4H), 1.27 (d, J = 6.4 Hz, 3H). |
| 15 | LC-MS: [M + H]⁺: 441.2 |
| | ¹H NMR (400 MHz, DMSO) δ 8.53 (s, 1H), 7.36 (d, J = 8.0 Hz, 1H), 7.21 (dd, J = 15.9, 7.8 Hz, 2H) |
| | 6.92 (d, J = 8.0 Hz, 1H), 4.65-4.39 (m, 2H), 4.35-4.14 (m, 4H), 3.92 (d, J = 8.9 Hz, 1H), 3.79 (d, J [symbol] |
| | 8.9 Hz, 1H), 3.31-3.21 (m, 3H), 2.01-1.57 (m, 4H), 1.27 (d, J = 6.4 Hz, 3H). |
| 22 | LC-MS: [M + H]⁺: 381.2 |
| | ¹H NMR (400 MHz, CD₃OD) δ 8.50 (s, 1H), 7.69 (d, J = 5.6 Hz, 1H), 7.23 (s, 1H), 6.17 (d, J = 5.6 H |
| | 1H), 4.58 (s, 1H), 4.41 (d, J = 14.0 Hz, 2H), 3.64-3.48 (m, 2H), 2.89 (s, 2H), 1.63-1.58 (m, 4H), 1. [symbol] |
| | (s, 3H). |
| 73 | LC-MS: [M + H]⁺: 427 |
| | ¹H NMR (400 MHz, MeOD) δ 8.49 (s, 1H), 7.37 (d, J = 7.8 Hz, 1H), 7.22 (dd, J = 16.3, 8.3 Hz, 2H) |
| | 6.92 (d, J = 8.1 Hz, 1H), 4.69-4.52 (m, 2H), 4.15 (dd, J = 10.1, 5.8 Hz, 1H), 3.90 (dd, J = 18.7, 9.1 |
| | Hz, 2H), 3.73 (d, J = 10.3 Hz, 1H), 3.46 (s, 3H), 1.83 (d, J = 9.8 Hz, 2H), 1.71 (d, J = 13.8 Hz, 2H). |
| 75 | LC-MS: [M + H]⁺: 427 |
| | ¹H NMR (400 MHz, MeOD) δ 8.53 (s, 1H), 7.36 (dd, J = 8.0, 1.3 Hz, 1H), 7.22 (dd, J = 15.0, 7.0 H |
| | 2H), 6.92 (dd, J = 8.1, 1.3 Hz, 1H), 4.71-4.50 (m, 2H), 4.13 (dd, J = 9.7, 6.1 Hz, 1H), 3.86 (s, 2H), |
| | 3.63 (dd, J = 9.7, 4.1 Hz, 1H), 3.35 (d, J = 11.1 Hz, 2H), 3.24-3.15 (m, 1H), 1.81 (td, J = 15.0, 4.0 |
| | Hz, 2H), 1.65 (d, J = 14.4 Hz, 2H). |
| 76 | LC-MS: [M + H]⁺: 403 |
| | ¹H NMR (400 MHz, MeOD) δ 8.49 (s, 1H), 7.37 (dd, J = 8.0, 1.3 Hz, 1H), 7.30-7.17 (m, 2H), |
| | 6.94 (dd, J = 8.1, 1.3 Hz, 1H), 3.36-3.31 (m, 2H), 3.30-3.24 (m, 2H), 3.16 (d, J = 19.9 Hz, 2H), |
| | 2.04-1.72 (m, 4H). |
| 77 | LC-MS: [M + H]⁺: 423.2 |
| | ¹H NMR (400 MHz, MeOD) δ 7.69 (d, J = 5.4 Hz, 1H), 7.24 (s, 1H), 6.17 (d, J = 5.5 Hz, 1H), 4.65- |
| | 4.55 (m, 4H), 4.35-4.22 (m, 1H), 3.94 (d, J = 9.0 Hz, 1H), 3.83 (d, J = 9.0 Hz, 1H), 3.31-3.21 (m, |
| | 1H), 1.93-1.49 (m, 4H), 1.28 (d, J = 6.5 Hz, 3H). |
| 78 | LC-MS: [M + H]⁺: 425 |
| | ¹H NMR (400 MHz, MeOD ) δ 7.36-7.34 (m, 1H), 7.22-7.18 (m, 2H), 6.92-6.90 (m, 1H), 4.38- |
| | 4.34 (m, 1H).3.27-.325 (m, 2H), 3.10-3.03 (m, 2H), 2.16-2.13 (m, 1H), 1.97-1.87 (m, 1H), 1.87- |
| | 1.66 (m, 8H), |
| 79 | LC-MS: [M + H]⁺: 437.2 |
| | ¹H NMR (400 MHz, DMSO) δ 7.22 (s, 1H), 7.18 (t, J = 8.2 Hz, 1H), 6.91 (d, J = 8.2 Hz, 1H), 6.55 |
| | (d, J = 8.0 Hz, 1H), 4.70-4.60 (m, 4H), 4.30-4.27 (m, 1H), 3.97 (d, J = 9.2 Hz, 1H), 3.86 (d, J = |
| | 11.6 Hz, 1H), 3.41 (d, J = 4.1 Hz, 1H), 3.26-3.08 (m, 2H), 1.94-1.61 (m, 4H), 1.31 |
| | (d, J = 6.5 Hz, 3H). |
| 80 | LC-MS: [M + H]⁺: 438 |
| | ¹H NMR (400 MHz, MeOD) δ 8.49 (s, 1H), 7.88 (d, J = 5.5 Hz, 1H), 7.25 (s, 1H), 6.50 (d, J = 5.5 |
| | Hz, 1H), 4.75-4.58 (m, 2H), 4.27 (dd, J = 6.5, 4.4 Hz, 1H), 4.06-3.90 (m, 4H), 3.84 (d, J = 9.1 Hz, |
| | 1H), 3.36 (s, 1H), 3.28-3.11 (m, 2H), 1.86 (dd, J = 17.1, 7.3 Hz, 3H), 1.67 (d, J = 13.5 Hz, 1H), |
| | 1.29 (d, J = 6.5 Hz, 3H). |
| 81 | LC-MS: [M + H]⁺: 442 |
| | ¹H NMR (400 MHz, MeOD) δ 8.56-8.32 (m, 1H), 8.10 (d, J = 5.3 Hz, 1H), 7.40-7.19 (m, 1H), |
| | 6.95 (d, J = 5.3 Hz, 1H), 4.77-4.62 (m, 2H), 4.33-4.20 (m, 1H), 3.97 (d, J = 9.1 Hz, 1H), 3.86 (d, |
| | J = 9.1 Hz, 1H), 3.43 (t, J = 16.6 Hz, 1H), 3.23 (m, 2H), 1.96-1.79 (m, 3H), 1.69 (d, J = 13.2 Hz, |
| | 1H), 1.31 (d, J = 6.5 Hz, 3H). |
| 84 | LC-MS: [M + H]⁺: 410.3 |
| | ¹H NMR (400 MHz, DMSO) δ 8.32 (s, 2H), 7.91 (d, J = 5.3 Hz, 1H), 7.25 (s, 1H), 7.05 (t, J = 5.1 |
| | Hz, 1H), 4.32-4.28 (m, 2H), 4.13-4.01 (m, 2H), 3.69 (d, J = 8.4 Hz, 1H), 3.51 (d, J = 8.5 Hz, 1H), |
| | 2.97 (d, J = 5.1 Hz, 1H), 1.79-1.65 (m, 2H), 1.59-1.42 (m, 2H), 1.10 (d, J = 6.4 Hz, 3H). |
| 107 | LC-MS: [M + H]⁺:424 |
| | ¹H NMR (400 MHz, MeOD) δ 8.52 (s, 1H), 7.35-7.18 (m, 2H), 5.98 (d, J = 7.1 Hz, 1H), 4.60 (m, |
| | 2H), 4.34-4.19 (m, 1H), 3.92 (d, J = 9.0 Hz, 1H), 3.81 (d, J = 9.0 Hz, 1H), 3.37 (d, J = 10.2 Hz, 1H) [symbol] |
| | 3.28-3.18 (m, 2H), 1.84 (m, 3H), 1.66 (d, J = 13.3 Hz, 1H), 1.27 (d, J = 6.5 Hz, 3H). |
| 109 | LC-MS: [M + H]⁺: 425 |
| | ¹H NMR (400 MHz, MeOD) δ 7.32 (m, 1H), 7.21m7.08 (m, 3H), 4.83m4.76 (m, 1H), 4.69 (d, J = |
| | 13.4 Hz, 1H), 4.32 (m, 1H), 4.01 (d, J = 9.2 Hz, 1H), 3.88 (d, J = 9.2 Hz, 1H), 3.70-3.46 (m, 3H), |
| | 2.09 (t, J = 10.7 Hz, 2H), 2.03-1.94 (m, 1H), 1.83 (d, J = 13.2 Hz, 1H), 1.33 (d, J = 6.5 Hz, 3H). |
| 82 | LC-MS: [M + H]⁺: 409 |
| | ¹H NMR (400 MHz, MeOD) δ 8.49 (s, 1H), 7.25 (s, 1H), 7.14 (m, 2H), 6.99 (m, 1H), 4.64-4.46 |
| | (m, 2H), 4.34-4.20 (m, 1H), 3.93 (d, J = 9.1 Hz, 1H), 3.82 (d, J = 9.1 Hz, 1H), 3.29-3.06 (m, 3H), |
| | 1.91-1.73 (m, 3H), 1.65 (d, J = 13.2 Hz, 1H), 1.28 (d, J = 6.5 Hz, 3H). |
| 83 | LC-MS: [M + H]⁺: 426.3 |
| | ¹H NMR (400 MHz, DMSO) δ 8.26 (s, 1H), 8.03 (s, 1H), 7.27 (s, 1H), 6.98 (s, 1H), 4.45-4.31 (m, |
| | 2H), 4.15-4.09 (m, 1H), 3.75 (d, J = 8.6 Hz, 1H), 3.58 (d, J = 8.6 Hz, 1H), 3.20-3.10 (m, 3H), 1.86- |
| | 1.41 (m, 4H), 1.13 (d, J = 6.5 Hz, 3H). |
| 26 | LC-MS: [M + H]⁺: 422 |
| | ¹H NMR (400 MHz, MeOD) δ 7.36 (t, J = 8.0 Hz, 1H), 7.23 (s, 1H), 6.77 (t, J = 7.8 Hz, 2H), |
| | 4.43-4.29 (m, 2H), 3.54 (m, 2H), 2.89 (s, 2H), 1.70-1.59 (m, 2H), 1.54 (d, J = 13.7 Hz, 2H), |
| | 1.18 (s, 3H). |
| 85 | LC-MS: [M + H]⁺: 439 |
| | ¹H NMR (400 MHz, DMSO) δ 7.53-7.48 (m, 1H), 7.33 (t, J = 8.0 Hz, 1H), 6.85 (d, J = 7.9 Hz, 1H), |
| | 6.74 (d, J = 7.4 Hz, 1H), 6.60 (d, J = 7.5 Hz, 1H), 4.31-4.06 (m, 1H), 3.79 (d, J = 8.8 Hz, 1H), 3.70- |
| | 3.47 (m, 3H), 3.28 (s, 1H), 2.73-2.58 (m, 2H), 1.79 (m, 2H), 1.62 (dm, 2H), 1.21 (t, J = 10.4 Hz, 3H). |
| 13 | LC-MS: [M + H]⁺: 437.1 |
| | ¹H NMR (400 MHz, DMSO) δ 7.48 (d, J = 7.4 Hz, 1H), 7.32 (t, J = 8.0 Hz, 1H), 7.21 (d, J = 1.2 Hz, |
| | 1H), 6.94 (m, 1H), 4.79-4.45 (m, 2H), 3.38 (d, J = 5.4 Hz, 1H), 3.21-2.81 (m, 3H), 2.09-1.71 (m, |
| | 2H), 1.70-1.32 (m, 6H), 0.75-0.33 (m, 2H) |
| 86 | LC-MS: [M + H]⁺: 369.0 |
| | ¹H NMR (400 MHz, MeOD) δ 7.42 (d, J = 7.8 Hz, 1H), 7.33-7.12 (m, 2H), 7.05 (s, 1H), 4.72-4.16 |
| | (m, 2H), 4.21-3.92 (m, 2H), 2.60 (s, 1H), 2.33 (s, 2H). |
| 108 | LC-MS: [M + H]⁺: 473.1 |
| | ¹H NMR (400 MHz, DMSO) δ 12.16 (s, 1H), 8.47 (s, 3H), 7.58 (d, J = 7.3 Hz, 1H), 7.50 (dd, J = |
| | 8.0, 1.3 Hz, 1H), 7.39-7.17 (m, 5H), 6.98 (m, 1H), 4.76 (s, 2H), 4.35 (d, J = 5.1 Hz, 1H), 3.33-3.13 |
| | (m, 3H), 3.00 (d, J = 16.2 Hz, 1H), 1.88-1.74 (m, 2H), 1.52 (t, J = 15.3 Hz, 2H). |
| 87 | LC-MS: [M + H]⁺: 475.1 |
| | ¹H NMR (400 MHz, MeOD) δ 7.54 (d, J = 7.4 Hz, 1H), 7.41 (m, 2H), 7.24 (d, J = 16.0 Hz, 2H), |
| | 7.12-7.04 (m, 2H), 7.00 (d, J = 8.2 Hz, 1H), 5.11 (d, J = 12.3 Hz, 1H), 4.83-4.76 (m, 1H), 4.68 (s, |
| | 1H), 3.70-3.41 (m, 2H), 2.28 (td, J = 12.9, 4.6 Hz, 1H), 2.10 (m, 1H), 2.03-1.87 (m, 2H). |
| 88 | LC-MS: [M + H]⁺: 428.1 |
| | ¹H NMR (400 MHz, CD₃OD) δ 8.17 (d, J = 4.4 Hz, 1H ), 7.27-7.20 (m, 2H), 4.68-4.63 (m, 1H), |
| | 4.22-4.18 (m, 1H), 4.00-3.83 (m, 3H), 3.73-3.57 (m, 4H), 2.09-2.01 (m, 2H), 1.92-1.84 (m, 2H) |
| 27 | LC-MS: [M + H]⁺: 492.2 |
| | ¹H NMR (400 MHz, MeOD) δ 8.18 (d, J = 8.3 Hz, 1H), 7.47 (t, J = 8.2 Hz, 1H), 7.10 (s, 1H), 7.03 |
| | (d, J = 7.9 Hz, 1H), 4.78 (s, 1H), 4.68-4.45 (m, 3H), 4.30 (m, 1H), 3.99 (d, J = 9.3 Hz, 1H), 3.88 |
| | (d, J = 9.2 Hz, 1H), 3.57-3.42 (m, 3H), 2.28 (s, 3H), 2.09-1.92 (m, 3H), 1.79 (d, J = 13.8 Hz, 1H), |
| | 1.32 (d, J = 6.5 Hz, 3H). |

[symbol]

indicates data missing or illegible when filed

### Example 4

### Preparation of 6-(4-(Aminomethyl)-4-Methylpiperidin-1-Yl)-3-((2, 3-Dichlorophenyl) Amino)-1, 5-Dihydro-4H-Pyrazolo [3, 4-d] Pyrimidin-4-One

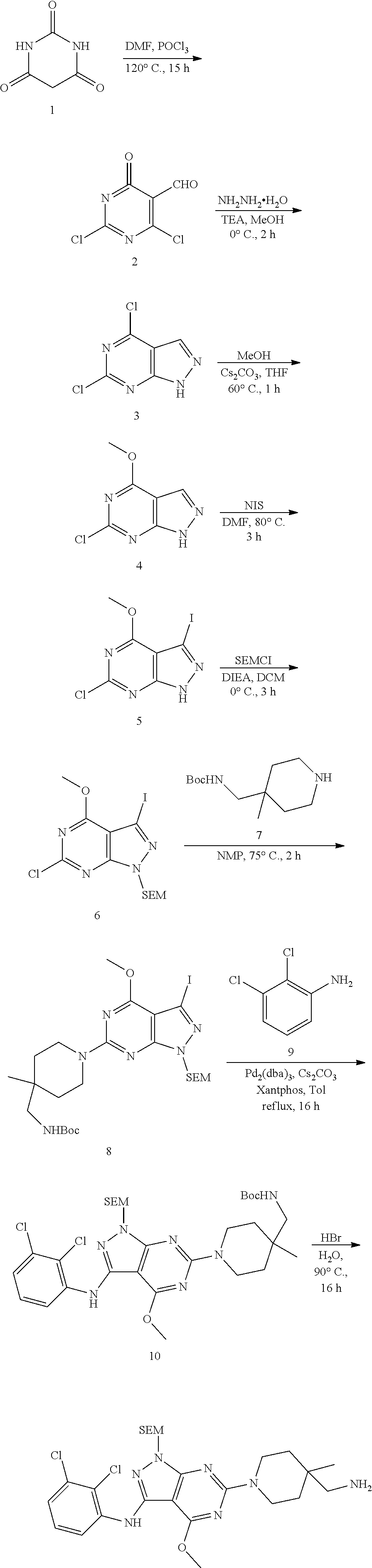

Step 1: preparation of 2, 4, 6-trichloropyrimidine-5-carbaldehyde (2)

A mixture of compound 1 (25.6 g, 1.0 eq) with POCl₃ (100 mL) and DMF (30 mL) was heated at 120° C. for 15 h, and then DMF was evaporated. Ice water was added to the residue and the solid formed was collected and dried to give compound 2 (9.2 g, 21%).

Step 2: preparation of 4, 6-dichloro-1H-pyrazolo [3, 4-d] pyrimidine (3)

To a solution of compound 2 (16.0 g, 1.0 eq) in methanol (80 mL) was added dropwise a solution of hydrazine monohydrate (4.55 g, 1.2 eq) in methanol at 0° C., and then a solution of triethylamine (15.28 g, 2.0 eq) in methanol was added dropwise at 0° C. The mixture was stirred at 0° C. for 2 h, then evaporated in vacuo. The residue was suspended in hot isopropyl alcohol and the insoluble materials were removed by filtration. The combined filtrate was concentrated in vacuo to give the title compound 3 as a yellow solid (9.0 g, 62%).

Step 3: preparation of 6-chloro-4-methoxy-1H-pyrazolo [3, 4-d] pyrimidine (4)

To a solution of compound 3 (480 mg, 2.54 mmol, 1.0 eq) in THF was added cesium carbonate (1.65 g, 2.0 eq) and methanol (3.0 mL), and the mixture was heated to 60° C. The reaction was quenched with water (10 mL) and worked up under standard procedure to afford compound 4 as a brown solid (468 mg, 99%).

Step 4: preparation of 6-chloro-3-iodo-4-methoxy-1H-pyrazolo [3, 4-d] pyrimidine (5)

To a solution of compound 4 (468 mg, 1.0 eq) in dry DMF (12 mL) was added N-iodosuccinimide (857 mg, 1.5 eq) and the reaction mixture was heated to 80° C. under stirring for 3 h. The reaction was quenched with water (10 mL) and worked up under standard process to afford compound 5 as a brown solid (522 mg, 66%).

Step 5: preparation of 6-chloro-3-iodo-4-methoxy-1-((2-(trimethylsilyl) ethoxy) methyl)-1H-pyrazolo [3, 4-d] pyrimidine (6)

To a solution of compound 5 (2.6 g, 1.0 eq) in DCM (20 mL) was added DIEA (2.37 g, 2.2 eq) at 0° C., and then SEMCI (1.67 g, 1.2 eq) was added dropwise. The reaction was quenched with water (10 mL) and worked up under standard process to afford compound 6 as a brown solid (1.55 g, 42%).

Step 6: preparation of tert-butyl ((1-(3-iodo-4-methoxy-1-((2-(trimethylsilyl) ethoxy) methyl)-1H-pyrazolo [3, 4-d] pyrimidin-6-yl)-4-methylpiperidin-4-yl) methyl) carbamate (8)

To a solution of compound 6 (520 mg, 1.0 eq) in NMP (10 mL) was added compound 7 (296 mg, 1.1 eq) at RT, and then the mixture was stirred at 75° C. for 2 h. The reaction was quenched with water (10 mL) and worked up under standard process to afford compound 8 as a white solid (450 mg, 60%).

Step 7: preparation of tert-butyl ((1-(3-((2,3-dichlorophenyl)amino)-4-methoxy-1-((2-(trimethylsilyl)ethoxy)methyl)-1H-pyrazolo[3,4-d]pyrimidin-6-yl)-4-methylpiperidin-4-yl)methyl)carbamate (10)

To a solution of compound 8 (200 mg, 1.0 eq) in toluene was added compound 9 (61.5 mg, 1.2 eq), cesium carbonate (123.7 mg, 1.2 eq) and xantphos (183.1 mg, 1.0 eq). After that, Pd₂(dba)₃ (5.0 mg) was added under the atmosphere of Ar. The mixture was stirred at 120° C. for 16 h. The reaction was quenched with water and worked up under standard process to give compound 10 as a white solid (42 mg, 20%).

Step 8: preparation of 6-(4-(aminomethyl)-4-methylpiperidin-1-yl)-3-((2, 3-dichlorophenyl) amino)-1, 5-dihydro-4H-pyrazolo [3, 4-d] pyrimidin-4-one

To a mixture of compound 10 (97.0 mg, 1.0 eq) in H₂O (4 mL) was added HBr (40%, 2 mL), and then the mixture was stirred at 90° C. for 16 h. The reaction was quenched by water followed by standard work up process. The crude product was purified by prep-HPLC to give desired compound, as HCOOH salt (white solid, 9.79 mg, 16%). LC-MS: [M+H]⁺=422.2. ¹H NMR (400 MHz, CD₃OD): δ 8.53 (brs, 1H), 8.37 (d, J=8.8 Hz, 1H), 7.24 (t, J=8.4 Hz, 1H), 7.05 (d, J=8.0 Hz, 1H), 4.03-3.98 (m, 2H), 3.50-3.43 (m, 2H), 2.87 (s, 2H), 1.64-1.56 (m, 4H), 1.17 (s, 3H).

The compounds in Table B below were synthesized in the similar manner using appropriate reagents and conditions. The compounds listed in Table B are merely non-limiting examples. Other subject compounds could also be made using similar methods.

TABLE B

| Compound | |
|---|---|
| ID | ¹H-NMR & MS [M + 1]⁺ |
| 35 | LC-MS: [M + H]⁺: 423.1 |
| | ¹H NMR (400 MHz, CD₃OD) δ 8.52 (br s, 3H), 7.38 (d, J = 8.0 Hz, 1H), 7.27 (t, J = 8.2 Hz, |
| | 1H), 7.14 (d, J = 8.8 Hz, 1H), 4.08-3.99 (m, 2H), 3.60-3.43 (m, 2H), 2.88 (s, 2H), 1.62-1.56 |
| | (m, 4H), 1.18 (s, 3H). |
| 68 | LC-MS: [M + H]⁺: 408 |
| | ¹H NMR (400 MHz, DMSO) δ 12.21 (s, 1H), 8.67-8.21 (m, 3H), 7.96 (s, 1H), 7.34 (t, J = 8.2 |
| | Hz, 1H), 7.13 (d, J = 7.8 Hz, 1H), 3.55 (s, 2H), 3.44 (d, J = 10.9 Hz, 1H), 3.20 (d, J = 10.8 Hz, |
| | 1H), 2.58 (s, 2H), 1.89 (s, 1H), 1.66 (d, J = 5.5 Hz, 1H), 1.05 (s, 3H). |

### Example 5

### Preparation of (1-(3-((2,3-Dichlorophenyl)Thio)-1H-Pyrazolo[3,4-d]Pyrimidin-6-Yl)-4-Methylpiperidin-4-Yl)Methanamine

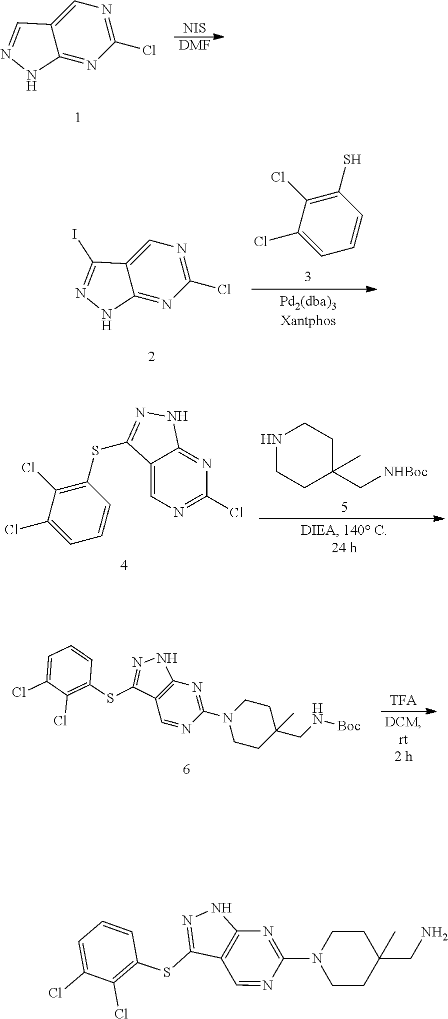

Step 1: preparation of 6-chloro-3-iodo-1H-pyrazolo[3,4-d]pyrimidine (2)

To a solution of compound 1 (780 mg, 1.0 eq) in DMF was added NIS (1300 mg, 1.2 eq) in portion at rt. The mixture was heated to 80° C. and stirred for 4 h. The reaction was quenched by water and worked up under standard operation to afford compound 2 as yellow solid (560 mg, 68%).

Step 2: preparation of 6-chloro-3-((2,3-dichlorophenyl)thio)-1H-pyrazolo[3,4-d]pyrimidine (4)

To a solution of compound 2 (280 mg, 1.0 eq) in dioxane were added Pd₂(dba)₃ (46 mg, 0.05 eq), XantPhos (58 mg, 0.1 eq) and DIEA (200 mg, 1.5 eq) at rt. The mixture was heated to 90° C. and stirred for 14 h. The reaction was quenched by water and worked up under standard operation to afford compound 4 as yellow solid (140 mg, 48%).

Step 3: preparation of tert-butyl ((1-(3-((2,3-dichlorophenyl)thio)-1H-pyrazolo[3,4-d]pyrimidin-6-yl)-4-methylpiperidin-4-yl)methyl)carbamate (6)

To a solution of compound 4 (145 mg, 1.0 eq) in DIEA was added compound 5 (300 mg, 3.0 eq) at rt. The mixture was heated at 140° C. for 24 h. The reaction was quenched by water and worked up under standard operation to give compound 6 as a yellow oil (110 mg, 48%).

Step 4: preparation of (1-(3-((2,3-dichlorophenyl)thio)-1H-pyrazolo[3,4-d]pyrimidin-6-yl)-4-methylpiperidin-4-yl)methanamine

To a solution of compound 6 (110 mg, 1.0 eq) in DCM was added TFA (1 mL). The mixture was stirred at rt for 2 h. The mixture was concentrated and neutralized with ammonium hydroxide (pH=7-8), purified by prep-HPLC to give the target compound as a white solid (HCOOH salt, 6 mg, 7%). LC-MS: [M+H]⁺=423.2. ¹H NMR (400 MHz, CD₃OD) δ 8.54 (s, 1H), 7.41 (dd, J=8.0, 1.2 Hz, 1H), 7.14 (t, J=8.0 Hz, 1H), 6.92 (dd, J=8.0, 1.2 Hz, 1H), 4.62 (br, 1H), 4.42-4.36 (m, 2H), 3.61-3.54 (m, 2H), 2.89 (s, 2H), 1.60-1.50 (m, 4H), 1.20 (s, 3H).

### Example 6

### Preparation of 6-Amino-5-((2,3-Dichlorophenyl)Thio)-2-(Hexahydropyrrolo[3,4-c]Pyrrol-2(1H)-Yl)-3-Methylpyrimidin-4(3H)-One

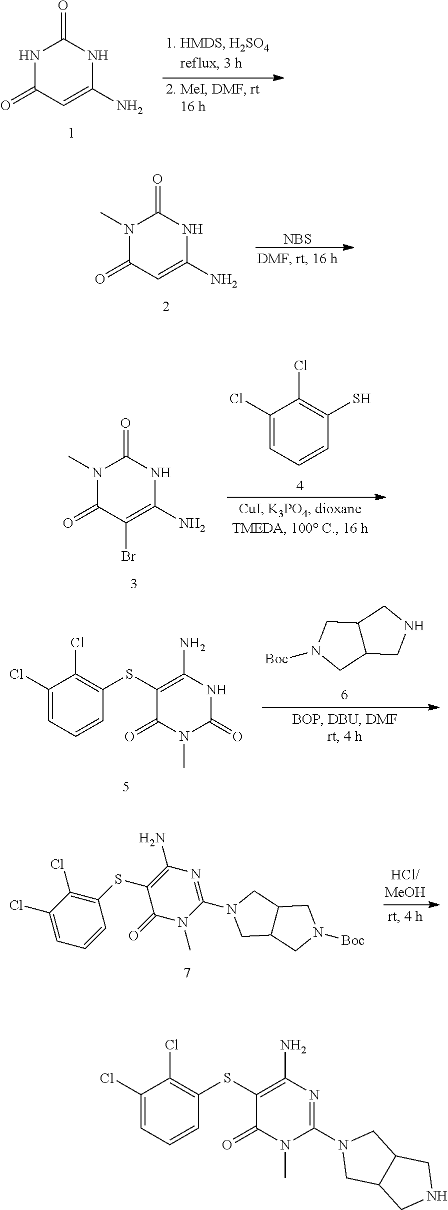

Step 1: Preparation of 6-amino-3-methylpyrimidine-2,4(1H,3H)-dione (2)

To a solution of compound 1 (31.08 g, 1.0 eq) in HMDS (150 mL) was added conc. H₂SO₄ (0.6 mL) at room temperature. The mixture was heated to reflux for 3 h. Then the mixture was concentrated in vacuo. The residue was dissolved in DMF (150 mL) and Mel (115.8 g, 3.4 eq) was added at rt. The mixture was stirred at rt for 16 h. The reaction was quenched by water and worked up under standard operation to give compound 2 as a white solid (16 g, 47%).

Step 2: preparation of 6-amino-5-bromo-3-methylpyrimidine-2,4(1H,3H)-dione (3)

To a solution of compound 2 (5.0 g, 1.0 eq) in DMF (50 mL) was added NBS (7.9 g, 1.25 eq) and the mixture was stirred at rt overnight. The reaction was quenched by water and worked up under standard operation to give compound 3 as a white solid (4.7 g, 60%).

Step 3: preparation of 6-amino-5-((2,3-dichlorophenyl)thio)-3-methylpyrimidine-2,4(1H,3H)-dione (5)

To a solution of compound 3 (500 mg, 1.0 eq) in dioxane (10 mL) was added compound 4 (813 mg, 2.0 eq), K₃PO₄ (1445 mg, 3.0 eq), TMEDA (105 mg, 0.4 eq) and Cul (86 mg, 0.2 eq). The resulting mixture was stirred at 100° C. under N₂ atmosphere for 2 h. The reaction was quenched by water and worked up under standard operation to give compound 5 as a brown solid (210 mg, 29%).

Step 4: preparation of tert-butyl 5-(4-amino-5-((2,3-dichlorophenyl)thio)-1-methyl-6-oxo-1,6-dihydropyrimidin-2-yl)hexahydropyrrolo[3,4-c]pyrrole-2(1H)-carboxylate (7)

To a solution of compound 5 (60 mg, 1.0 eq) in DMF (2 mL) was added compound 6 (60 mg, 1.5 eq), BOP (250 mg, 3.0 eq) and DBU (143 mg, 5.0 eq). The reaction was quenched by water and worked up under standard operation to give compound 7 as a white solid (55 mg, 57%).

Step 5: preparation of 6-amino-5-((2,3-dichlorophenyl)thio)-2-(hexahydropyrrolo[3,4-c]pyrrol-2(1H)-yl)-3-methylpyrimidin-4(3H)-one

A solution of compound 7 (50 mg, 1.0 eq) in HCl/MeOH (3 M, 3 mL) was stirred at rt for 4 h. The mixture was concentrated and purified by pre-HPLC to give the desired compound as HCOOH salt (white solid, 21 mg, 53%). LC-MS: [M+H]⁺=412. ¹H NMR (400 MHz, CD₃OD) δ 8.52 (br s, 1H), 7.24 (dd, J=8.0, 1.2 Hz, 1H), 7.09 (t, J=8.0 Hz, 1H), 6.75 (dd, J=8.0, 1.2 Hz, 1H), 3.74-3.61 (m, 4H), 3.60-3.50 (m, 2H), 3.45 (s, 3H), 3.30-3.13 (m, 4H).

The compounds in Table C below were synthesized in the similar manner using appropriate reagents and conditions. The compounds listed in Table C are merely non-limiting examples. Other subject compounds could also be made using similar methods.

TABLE C

| Compound | |
|---|---|
| ID | ¹H-NMR & MS [M + 1]⁺ |
| 52 | LC-MS: [M + H]⁺: 414.2 |
| | ¹H NMR (400 MHz, CD₃OD) δ 7.23 (dd, J = 8.0, 1.2 Hz, 1H), 7.09 (t, J = 8.0 Hz, 1H), 6.76 |
| | (dd, J = 8.0, 1.2 Hz, 1H), 3.77-3.70 (m, 2H), 3.56 (d, J = 10.8 Hz, 1H), 3.42 (s, 3H), 3.34-3.24 |
| | (m, 1H), 2.69 (s, 2H), 1.95-1.84 (m, 1H), 1.82-1.70 (m, 1H), 1.15 (s, 3H). |
| 60 | LC-MS: [M + H]⁺: 398.1 |
| | ¹H NMR (400 MHz, CD₃OD) δ 7.23 (dd, J = 8.0 Hz, 1.2 Hz, 1H), 7.10 (t, J = 8.0 Hz, 1H), 6.79 |
| | (d, J = 8.0 Hz, 1.2 Hz, 1H), 3.77-3.72 (m, 2H), 3.49-3.46 (m, 2H), 3.17-3.12 (m, 2H), 3.00 |
| | (brs, 2H), 2.82-2.80 (m, 2H). |
| 54 | LC-MS: [M + H]⁺: 400.1 |
| | ¹H NMR (400 MHz, CD₃OD) δ 7.22 (d, J = 8.0 Hz, 1H), 7.10 (t, J = 8.0 Hz, 1H), 6.81 (d, |
| | J = 8.0 Hz, 1H), 3.61 (br s, 2H), 3.42-3.39 (m, 1H), 3.25-3.23 (m, 1H), 2.67 (s, 2H), |
| | 1.96-1.93 (m, 1H), 1.79-1.76 (m, 1H), 1.14 (s, 3H). |
| 63 | LC-MS: [M + H]⁺: 426.1 |
| | ¹H NMR (400 MHz, CD₃OD) δ 7.22 (d, J = 7.6 Hz, 1H), 7.09 (t, J = 8.0 Hz, 1H), 6.76 (d, |
| | J = 8.0 Hz, 1H), 4.25-4.19 (m, 2H), 4.16-4.10 (m, 2H), 3.39-3.31 (m, 1H), 3.30 (s, 3H), |
| | 2.32-2.27 (m, 1H), 2.10-1.92 (m, 3H), 1.71-1.66 (m, 1H), 1.49-1.45 (m, 1H). |
| 64 | LC-MS: [M + H]⁺: 398.1 |
| | ¹H NMR (400 MHz, CD₃OD) δ 7.25 (d, J = 7.6 Hz, 1H), 7.10 (t, J = 7.6 Hz, 1H), 6.78 (d, |
| | J = 7.6 Hz, 1H), 3.94 (br s, 3H), 3.45-3.33 (m, 5H), 1.93 (br s, 2H). |
| 66 | LC-MS: [M + H]⁺: 384 |
| | ¹H NMR (400 MHz, CD₃OD) δ 7.24 (d, J = 8.0 Hz, 1H), 7.11 (t, J = 8.0 Hz, 1H), 6.84 (d, J = 8.0 |
| | Hz, 1H), 3.76-3.72 (m, 2H), 3.66-3.63 (m, 2H), 2.52 (t, J = 7.2 Hz, 1H), 1.83-1.82 (m, 2H). |
| 67 | LC-MS: [M + H]⁺: 412.1 |
| | ¹H NMR (400 MHz, CD₃OD) δ 7.23 (dd, J = 8.0 Hz, 2.0 Hz, 1H), 7.10 (t, J = 8.0 Hz, 1H), 6.79 |
| | (dd, J = 8.0 Hz, 2.0 Hz, 1H), 4.06-4.04 (m, 2H), 3.96-3.95 (m, 2H), 3.43-3.39 (m, 1H), 2.33-2.27 |
| | (m, 1H), 2.07-2.02 (m, 2H), 1.96-1.92 (m, 1H), 1.74-1.68 (m, 1H), 1.52-1.49 (m, 1H). |
| 58 | LC-MS: [M + H]⁺: 398.1 |
| | ¹H NMR (400 MHz, CD₃OD) δ 7.22 (d, J = 8.0, 1H), 7.08 (t, J = 8.0 Hz, 1H), 6.76 (d, J = 8.0, |
| | 1H), 5.17-5.15 (m, 1H), 4.42 (t, J = 8.4 Hz, 1H), 4.09-4.05 (m, 1H), 3.36 (s, 3H), 3.31-3.27 (m, |
| | 1H), 3.18 (d, J = 12.0 Hz, 1H), 3.08-3.05 (m, 1H), 2.77-2.72 (m, 1H), 2.62-2.61 (m, 1H), |
| | 2.59-2.58 (m, 1H). |
| 59 | LC-MS: [M + H]⁺: 384.1 |
| | ¹H NMR (400 MHz, CD₃OD) δ 7.22 (d, J = 8.0 Hz, 1H), 7.10 (t, J = 8.0 Hz, 1H), 6.79 (d, J = 8.0 |
| | Hz, 1H), 4.94-4.91 (m, 1H), 4.17 (t, J = 8.8 Hz, 1H), 3.71-3.68 (m, 1H), 3.45 (d, J = 12.8 Hz, |
| | 1H), 3.15-3.11 (m, 2H), 2.74-2.70 (m, 1H), 2.58-2.54 (m, 1H). |

### Example 7

### Preparation of N-(3-((5-(4-(Aminomethyl)-4-Methylpiperidin-1-Yl)-6-Oxo-1,6-Dihydropyrazin-2-Yl)Thio)-2-Chlorophenyl)-4-Hydroxy-1,5,5-Trimethyl-2-Oxo-2,5-Dihydro-1H-Pyrrole-3-Carboxamide

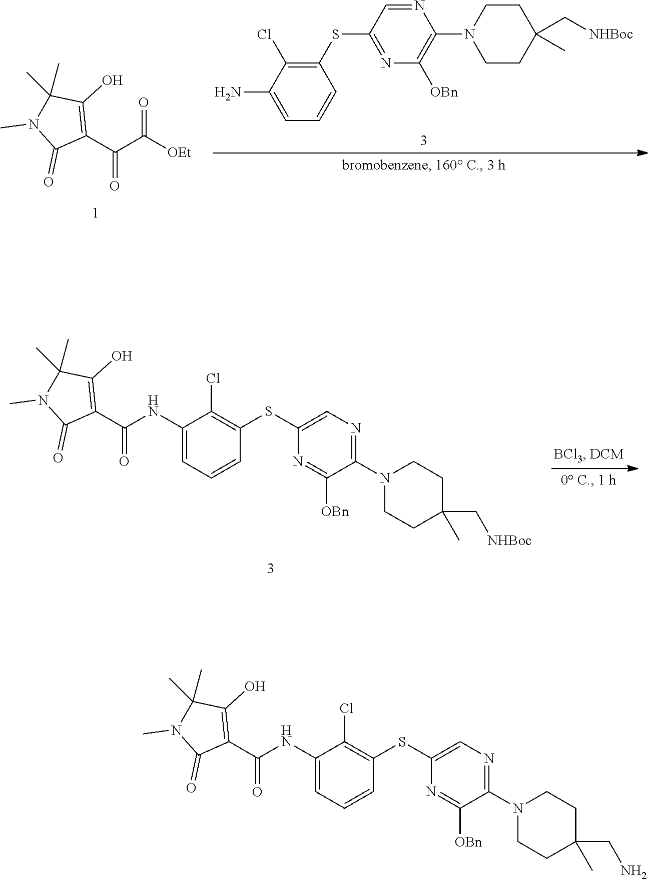 [SMILES: CCOC(=O)C(=O)C1=C(O)C(C)(C)N(C)C1=O ; CC1(CNC(=O)OC(C)(C)C)CCN(c2ncc(Sc3cccc(N)c3Cl)nc2OCc2ccccc2)CC1 ; CN1C(=O)C(C(=O)Nc2cccc(Sc3cnc(N4CCC(C)(CNC(=O)OC(C)(C)C)CC4)c(OCc4ccccc4)n3)c2Cl)=C(O)C1(C)C ; CN1C(=O)C(C(=O)Nc2cccc(Sc3cnc(N4CCC(C)(CN)CC4)c(OCc4ccccc4)n3)c2Cl)=C(O)C1(C)C]

Step 1: preparation of tert-butyl ((1-(3-(benzyloxy)-5-((2-chloro-3-(4-hydroxy-1,5,5-trimethyl-2-oxo-2,5-dihydro-1H-pyrrole-3-carboxamido)phenyl)thio)pyrazin-2-yl)-4-methylpiperidin-4-yl)methyl)carbamate (3)

To a mixture of compound 1 (80 mg, 1.0 eq) in bromobenzene (5 mL) was added compound 2 (107 mg, 0.5 eq) at r.t. and the mixture was degassed and protected with nitrogen. The reaction was stirred at 160° C. for 3 h. Solvent was removed to give crude compound 3 as an oil (140 mg, 100%).

Step 2: preparation of N-(3-((5-(4-(aminomethyl)-4-methylpiperidin-1-yl)-6-oxo-1,6-dihydropyrazin-2-yl)thio)-2-chlorophenyl)-4-hydroxy-1,5,5-trimethyl-2-oxo-2,5-dihydro-1H-pyrrole-3-carboxamide

To a solution of compound 3 (140 mg, 1.0 eq) in DCM (3 mL) was added BCl₃ (1.9 mL, 1.0 mol/L in DCM, 10.0 eq) at room temperature. The mixture was stirred at r.t. for 1 h. The mixture was quenched by MeOH (3 mL) followed by standard work up process. Desired compound was obtained after prep-HPLC as a slightly red solid (25 mg, 12%). LC-MS: [M+H]⁺=547. ¹H NMR (400 MHz, DMSO) δ 11.37 (br s, 1H), 8.46 (d, J=8.0 Hz, 1H), 8.30 (s, 1H), 7.18 (s, 1H), 7.09 (s, 1H), 6.49 (d, J=8.4 Hz, 1H), 4.24-4.21 (m, 2H), 3.55-3.48 (m, 2H), 2.74 (s, 3H), 2.65 (s, 2H), 1.55-1.50 (m, 2H), 1.38-1.33 (m, 2H), 1.12 (s, 6H), 1.02 (s, 3H).

The compounds in Table D below were synthesized by similar manners using appropriate reagents and conditions. The compounds listed in Table D are merely non-limiting examples. Other subject compounds could also be made using similar methods.

TABLE D

| Compound | |
|---|---|
| ID | ¹H-NMR & MS [M + 1]⁺ |
| 31 | LC-MS: [M + H]⁺: 574 |
| | ¹H NMR (400 MHz, DMSO) δ 13.28 (br s, 1 H), 8.42 (d, J = 7.6 Hz, 1H), 8.16 (s, 1H), 7.68 |
| | (d, J = 4.4 Hz, 1H), 7.21-7.17 (m, 2H), 7.01-6.98 (m, 1H), 6.54 (d, J = 8.0 Hz, 1H), 4.27-4.21 |
| | (m, 2H), 3.59-3.50 (m, 2H), 2.79 (s, 2H), 1.58-1.54 (m, 2H), 1.43-1.40 (m, 2H), 1.07 (s, 3H). |
| 29 | LC-MS: [M + H]⁺: 568.3 |
| | ¹H NMR (400 MHz, DMSO-d6) δ 12.15 (brs, 1H), 9.06 (d, J = 6.4 Hz, 1H), 8.30-8.17 (m, 1H), |
| | 7.72 (brs, 1H), 7.58 (brs, 2H), 7.46 (t, J = 6.4 Hz, 1H), 7.34(brs, 2H), 7.22(s, 1H), 6.76 (brs, 1H), |
| | 4.30-4.24 (m, 2H), 3.57-3.51 (m, 2H), 2.81-2.75 (m, 2H), 1.59-1.51 (m, 2H), 1.43-1.40 (m, 2H), |
| | 1.07 (s, 3H). |
| 32 | LC-MS: [M + H]⁺: 557 |
| | ¹H NMR (400 MHz, DMSO) δ 12.74 (br s, 1 H), 8.42-8.35 (m, 1H), 8.20 (s, 1H), 7.34 (s, 1H), |
| | 7.21 (s, 2H), 6.97-6.92 (m, 1H), 6.61-6.57 (m, 1H), 4.28-4.21 (m, 2H), 3.58-3.48 (m, 2H), 2.75 |
| | (s, 2H), 1.58-1.52 (m, 2H), 1.42-1.36 (m, 2H), 1.06 (s, 3H). |
| 30 | LC-MS: [M + H]⁺: 568.3 |
| | ¹H NMR (400 MHz, DMSO-d₆) δ 12.15 (brs, 1H), 9.06 (d, J = 6.4 Hz, 1H), 8.30-8.17 (m, 1H), |
| | 7.72 (brs, 1H), 7.58 (brs, 2H), 7.46 (t, J = 6.4 Hz, 1H), 7.34(brs, 2H), 7.22(s, 1H), 6.76 (brs, 1H), |
| | 4.30-4.24 (m, 2H), 3.57-3.51 (m, 2H), 2.81-2.75 (m, 2H), 1.59-1.51 (m, 2H), 1.43-1.40 (m, 2H), |
| | 1.07 (s, 3H). |
| 33 | LC-MS: [M + H]⁺: 572.2 |
| | ¹H NMR (400 MHz, MeOD) δ 8.31 (s, 1H), 7.25 (s, 2H), 6.81 (s, 1H), 4.39 |
| | (d, J = 13.3 Hz, 2H), 3.99 (s, 2H), 3.76-3.43 (m, 2H), 2.96-2.87 (m, 4H), |
| | 2.01-1.92 (m, 4H), 1.76-1.37 (m, 4H), 1.18 (s, 3H). |

### Example 8

### Preparation of Intermediate Tert-Butyl ((1-(3-(Benzyloxy)-5-Bromopyrazin-2-Yl)-4-Methylpiperidin-4-Yl)Methyl)Carbamate

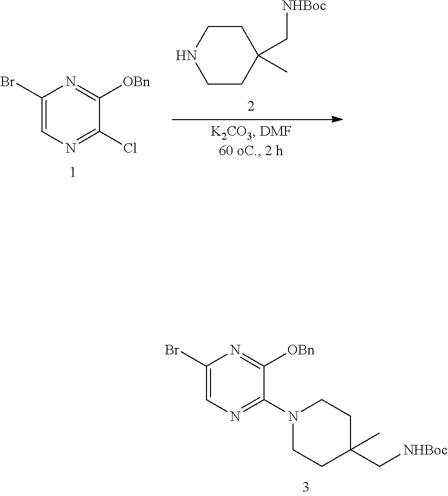 [SMILES: Clc1ncc(Br)nc1OCc1ccccc1 ; CC1(CNC(=O)OC(C)(C)C)CCNCC1 ; CC1(CNC(=O)OC(C)(C)C)CCN(c2ncc(Br)nc2OCc2ccccc2)CC1]

To a solution of compound 1 (320 mg, 1.0 eq) in DMF (5 mL) was added compound 2 (292 mg, 1.2 eq) and K₂CO₃ (441 mg, 3.0 eq) at rt. The reaction mixture was stirred at 60° C. under Ar atmosphere for 2 hours before it was quenched by water. The desired compound was obtained after standard work up process as a white solid (140 mg, 26%).

### Example 9

### Preparation of 1-(3-Aminocyclohexyl)-4-(2,3-Dichlorophenyl)Pyridin-2(1H)-One

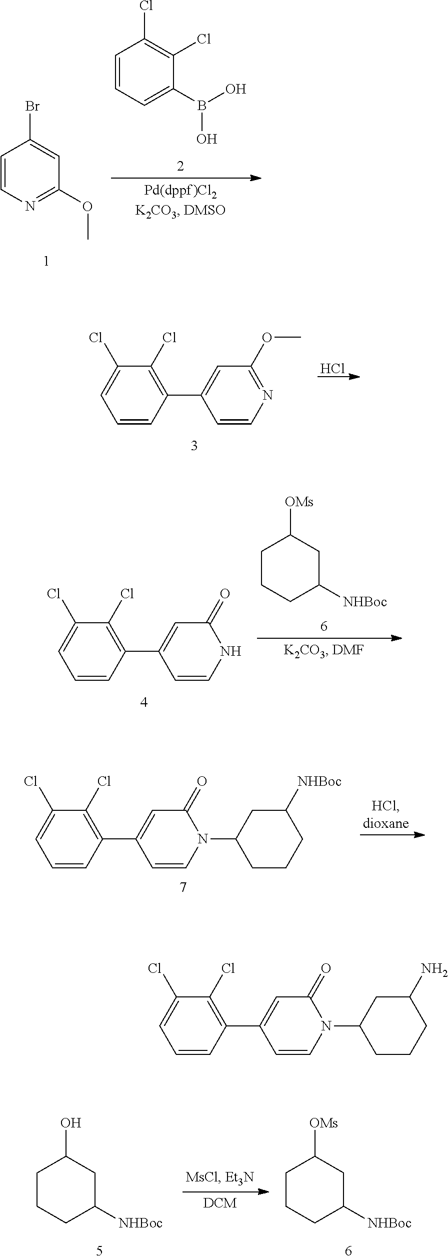

Step 1: Preparation of 4-(2,3-dichlorophenyl)-2-methoxypyridine (3)

To a solution of compound 1 (2.0 g, 1 eq) in DMSO were added compound 2 (1 g, 1 eq), PdCl₂(dppf) (0.77 g, 0.1 eq) and K₂CO₃(2.9 g, 2 eq). The mixture was stirred at 95° C. overnight. The reaction was quenched by water, and worked up under standard procedure. The residue was purified by flash column chromatography to give the title compound (2.1 g, yield: 78.9%).

Step 2: Preparation of 4-(2,3-dichlorophenyl)pyridin-2(1H)-one (4)

The mixture of compound 3 (1.0 g, 1 eq) in conc. HCl (20 mL) was stirred at 150° C. overnight. Ice-water was added into the mixture and pH was adjusted to 7. After standard work up procedure, title compound (0.94 g, yield: 100%) was obtained.

Step 3: Preparation of 3-((tert-butoxycarbonyl)amino)cyclohexyl methanesulfonate (6)

To a solution of compound 5 (1.0 g, 1.0 eq), Et₃N (1 ml, 1.5 eq) in DCM (20 mL) was added MsCl. The mixture was quenched with water (30 mL) 2 hours later. After standard work up procedure, the residue was purified by flash column chromatography to give the title compound (1.0 g, yield: 73.5%).

Step 4: Preparation of tert-butyl (3-(4-(2,3-dichlorophenyl)-2-oxopyridin-1(2H)-yl)cyclohexyl)carbamate (7)

To a solution of compound 4 (1.0 g, 1.0 eq) in DMF (10 mL) was added K₂CO₃ (1.7 g, 3.0 eq) and compound 6 (1.7 g, 1.2 eq), and the mixture was stirred at 110° C. overnight. The reaction was quenched with water (30 mL), followed by standard work up procedure. The residue was purified by flash column chromatography to give the title compound (170 mg, yield: 9.34%).

Step 5: Synthesis of 1-(3-aminocyclohexyl)-4-(2,3-dichlorophenyl)pyridin-2(1H)-one

The solution of compound 7 (170 mg, 1.0 eq) in 4M HCl/dioxane(5 mL) was stirred at room temperature for 30 min. NaOH solution was added to the mixture at 0° C., and the mixture was extracted with EA (3×30 mL). The organic layer was dried over Na₂SO₄, filtered and concentrated. The final compound was obtained after Pre-HPLC as HCOOH salt (100 mg, yield: 76.3%). LC-MS: [M+H]⁺=338.2. ¹H NMR (400 MHz, CD₃OD) S 8.22-8.21 (d, J=4.8 Hz 1H), 7.63-7.61 (d, J=7.6 Hz, 1H), 7.4-(m, 1H), 7.33-7.31 (m, 2H), 6.85 (s, 1H), 5.54 (s, 1H), 3.53 (m, 1H), 2.48-2.44 (d, J=13.2 Hz, 1H), 2.082-2.01 (m, 2H), 1.79 (m, 3H), 1.66 (m 3H).

### Example 10

### Preparation of (R)-2-Methyl-N-((3S,4S)-3-Methyl-2-Oxa-8-Azaspiro[4.5]Decan-4-Yl)Propane-2-Sulfinamide (INT-1)

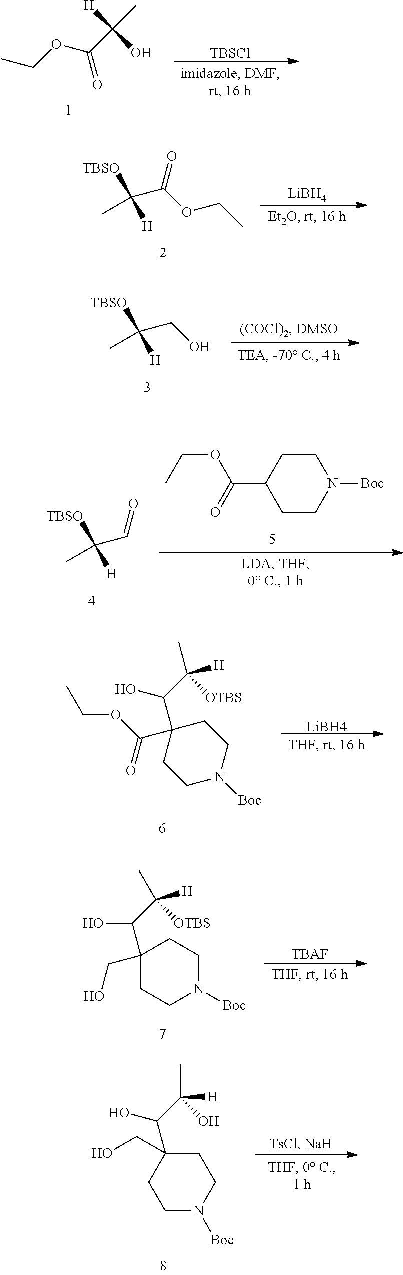

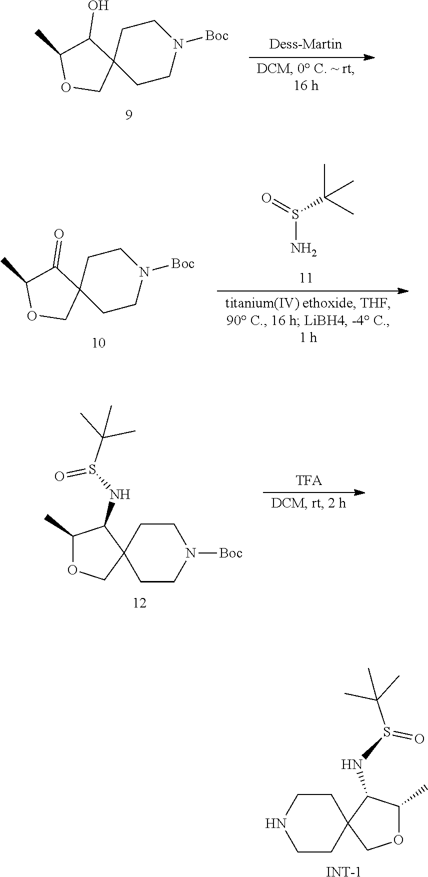

Step 1: preparation of (S)-ethyl 2-((tert-butyldimethylsilyl)oxy)propanoate (2)

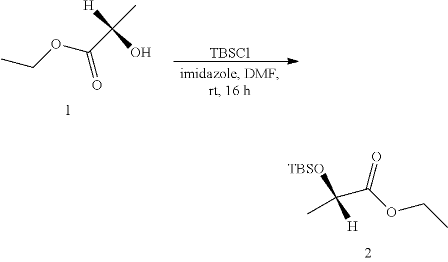

To a solution of compound 1 (30.0 g, 0.25 mol, 1.0 eq) in DMF (300 mL) was added TBSCl (45.9 g, 0.30 mol, 1.2 eq) and imidazole (34.5 g, 0.50 mol, 2.0 eq). The reaction mixture was stirred at rt under N₂ for 16 h. The mixture was extracted with EA, washed with Brine, dried with Na₂SO₄ concentrated and purified by column chromatography (PE/EtOAc=100:1) to give compound 2 as a colorless oil (55 g, 93% yield).

Step 2: preparation of (S)-2-((tert-butyldimethylsilyl)oxy)propan-1-ol (3)

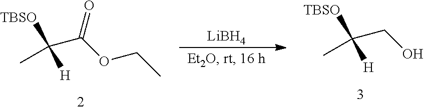

To a solution of compound 2 (43.0 g, 0.18 mol, 1.0 eq) in Et₂O (250 mL) was added LiBH₄ (8.06 g, 0.37 mol, 2.0 eq) portion wise. The reaction mixture was stirred at rt under N₂ for 16 h. The mixture was slowly added into NH₄Cl aq, then extracted with Et₂O, washed with Brine, dried with Na₂SO₄, concentrated (25° C.) to give compound 3 as a colorless oil (35 g, 99% yield).

Step 3: preparation of (S)-2-((tert-butyldimethylsilyl)oxy)propanal (4)

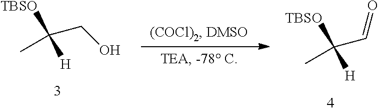

To a solution of (COCl)₂ (27.1 g, 0.21 mol, 1.1 eq) in DCM (400 mL) was added DMSO (36.5 g, 0.46 mol, 2.4 eq) dropwise at −78° C. under Ar. After stirring at −78° C. for 30 min, a solution of compound 3 (37.0 g, 0.19 mol, 1.0 eq) in DCM (50 mL) was slowly added into the mixture. After stirring at −78° C. for 30 min, TEA (98.0 g, 0.97 mol, 5.0 eq) in DCM (50 mL) was slowly added into the mixture. The reaction mixture was stirred at −78° C. for 30 min, then warmed to rt for 1 hour. The mixture was quenched with NH₄Cl aq at −78° C., extracted with DCM, washed with Brine, dried with Na₂SO₄, concentrated, and purified by column chromatography (PE/DCM=200:1) to give compound 4 as a colorless oil (26.0 g, 71% yield).

Step 4: preparation of 1-tert-butyl 4-ethyl 4-((2S)-2-((tert-butyldimethylsilyl)oxy)-1-hydroxypropyl)piperidine-1,4-dicarboxylate (6)

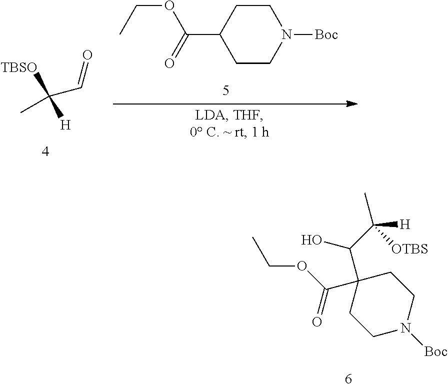

To a solution of compound 5 (32.3 g, 0.12 mol, 1.0 eq) in THF (150 mL) was added LDA (103 mL, 0.20 mol, 1.5 eq) dropwise at 0° C. under Ar. After stirring at 0° C. for 30 min, a solution of compound 4 (26.0 g, 0.14 mol, 1.1 eq) in THF (50 mL) was slowly added into the mixture. The reaction mixture was stirred at 0° C. for 1 hour, then warmed to rt for 1 hour. The mixture was quenched with NH4Cl aq, extracted with EA, washed with Brine, dried with Na₂SO₄ concentrated and purified by column chromatography (PE/EA=5:1) to give compound 4 as a colorless oil (16.5 g, 33% yield).

Step 5: preparation of tert-butyl 4-((2S)-2-((tert-butyldimethylsilyl)oxy)-1-hydroxypropyl)-4-(hydroxymethyl)piperidine-1-carboxylate (7)

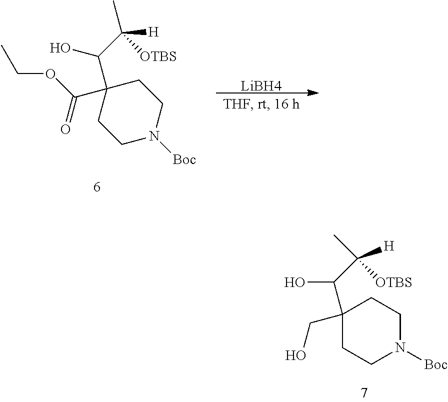 [SMILES: CCOC(=O)C1(C(O)[C@H](C)O[Si](C)(C)C(C)(C)C)CCN(C(=O)OC(C)(C)C)CC1 ; "LiBH4" ; "THF, rt, 16 h" ; C[C@H](O[Si](C)(C)C(C)(C)C)C(O)C1(CO)CCN(C(=O)OC(C)(C)C)CC1]

To a solution of compound 6 (16.5 g, 37.0 mmol, 1.0 eq) in THF (150 mL) was added LiBH₄ (1.61 g, 74.0 mmol, 2.0 eq) portionwise. The reaction mixture was stirred at rt under Ar for 16 h. The mixture was slowly added into NH₄Cl aq, then extracted with EA, washed with Brine, dried with Na₂SO₄, concentrated to give compound 7 as a colorless oil (13 g, 87% yield). Step 6: preparation of tert-butyl 4-((2S)-1,2-dihydroxypropyl)-4-(hydroxymethyl)piperidine-1-carboxylate (8)

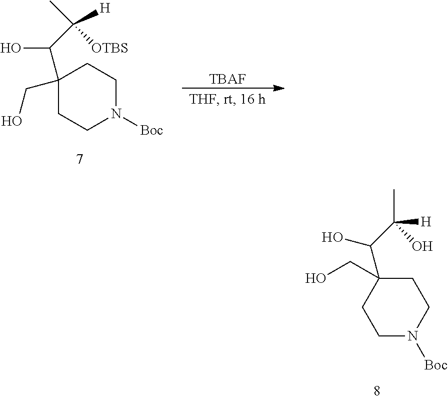 [SMILES: C[C@H](O[Si](C)(C)C(C)(C)C)C(O)C1(CO)CCN(C(=O)OC(C)(C)C)CC1 ; "TBAF" ; "THF, rt, 16 h" ; C[C@H](O)C(O)C1(CO)CCN(C(=O)OC(C)(C)C)CC1]

To a solution of compound 7 (13.0 g, 32.2 mmol, 1.0 eq) in THF (50 mL) was added TBAF (48 mL, 48.3 mmol, 1.5 eq). The reaction mixture was stirred at rt under Ar for 16 h. The mixture was added water, then extracted with EA, washed with Brine, dried with Na₂SO₄, concentrated and purified by column chromatography (EA) to give compound 8 as a colorless oil (7.5 g, 81% yield).

Step 7: preparation of (3S)-tert-butyl 4-hydroxy-3-methyl-2-oxa-8-azaspiro[4.5]decane-8-carboxylate (9)

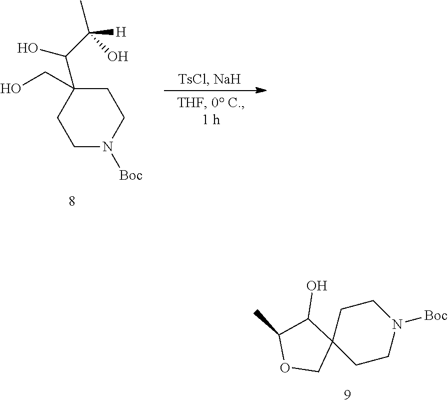

To a solution of compound 8 (3.65 g, 12.6 mmol, 1.0 eq) in THF (40 mL) was added TsCl (2.40 g, 12.6 mmol, 1.0 eq). After cooled to 0° C., NaH (2.0 g, 50.4 mmol, 4.0 eq) was slowly added into the mixture. The reaction mixture was stirred at 0° C. for 1 hour. The mixture was quenched with NH4Cl aq, extracted with EA, washed with Brine, dried with Na₂SO₄ concentrated and purified by column chromatography (EA) to give compound 9 as a colorless oil (1.5 g, 44% yield).

Step 8: preparation of ((S)-tert-butyl 3-methyl-4-oxo-2-oxa-8-azaspiro[4.5]decane-8-carboxylate (10)

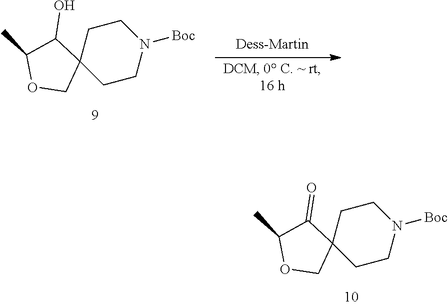 [SMILES: C[C@@H]1OCC2(CCN(C(=O)OC(C)(C)C)CC2)C1O ; C[C@@H]1OCC2(CCN(C(=O)OC(C)(C)C)CC2)C1=O]

To a solution of compound 9 (1.5 g, 5.52 mmol, 1.0 eq) in DCM (15 mL) was added Dess-Matin (3.0 g, 7.18 mmol, 1.3 eq) at 0° C. The reaction mixture was stirred at rt for 16 hour. The mixture was washed with Na₂CO₃ aq, extracted with DCM, washed with Brine, dried with Na₂SO₄, concentrated and purified by column chromatography (PE/EA=6:1) to give compound 10 as a white solid (1.1 g, 74% yield).

Step 9: preparation of (3S,4S)-tert-butyl 4-((R)-1,1-dimethylethylsulfinamido)-3-methyl-2-oxa-8-azaspiro[4.5]decane-8-carboxylate (12)

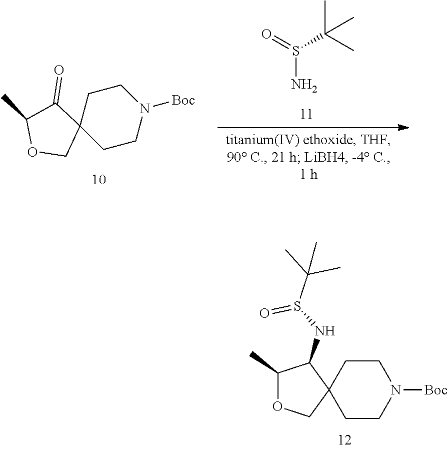

To a solution of compound 10 (1.3 g, 4.82 mmol, 1.0 eq) in THF (13 mL) was added compound 11 (1.17 g, 9.65 mmol, 2.0 eq) and Ti(OEt)₄ (1.21 g, 5.30 mmol, 1.1 eq). The reaction mixture was stirred at 90° C. for 16 hour. The mixture was cooled to −4° C., then LiBH₄ (210 mg, 9.65 mmol, 2.0 eq) was added portion wise. The reaction mixture was stirred at rt for 1 h. MeOH (5 mL) was slowly added into the mixture. The mixture was concentrated and purified by column chromatography (PE/EA=3:1) to give compound 12 as a colorless oil (850 mg, 47% yield).

Step 10: (R)-2-methyl-N-((3S,4S)-3-methyl-2-oxa-8-azaspiro[4.5]decan-4-yl)propane-2-sulfinamide (INT-1)

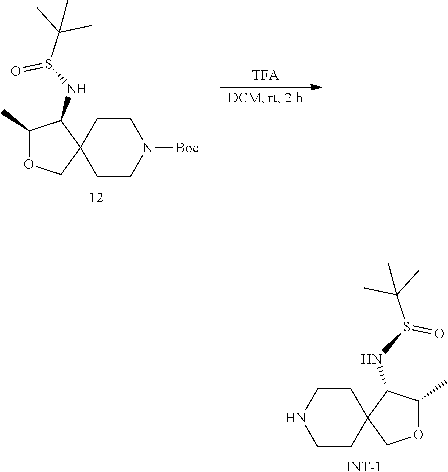

To a solution of compound 12 (372 mg, 0.99 mol, 1.0 eq) in DCM (2 mL) was added TFA (5 mL). The reaction mixture was stirred at rt under Ar for 2 h. The mixture was concentrated. The residue was treated NaHCO₃(aq.) and purified by column chromatography (DCM/MeOH=5:1) to give compound INT-1 as a colorless oil (148 mg, 54% yield). LC-MS: [M+H]⁺: 275.4

### Example 11

### Preparation of 3-(Benzyloxy)-5-Bromo-2-Chloropyrazine (INT-2)

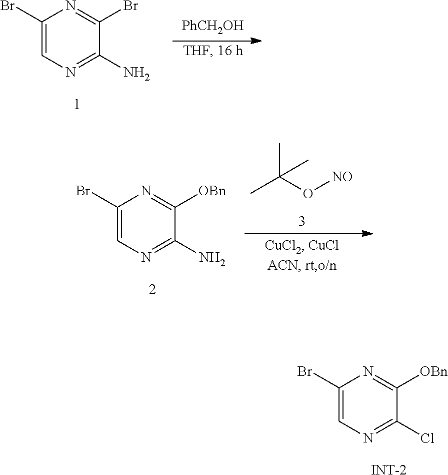

Step 1: preparation of 3-(benzyloxy)-5-bromopyrazin-2-amine (2)

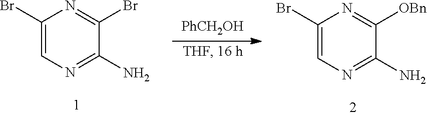 [SMILES: "PhCH2OH" ; Nc1ncc(Br)nc1Br ; "THF, 16 h" ; Nc1ncc(Br)nc1OCc1ccccc1]

A suspension of NaH (47.6 g, 1.19 mol, 2 eq) in dry THF (2 L) at 0° C. was stirred for 10 minutes before benzyl alcohol (123 mL, 1.19 mmol, 2 eq) was added and the mixture was stirred for 30 minutes. After this time, Compound 1 (150.0 g, 0.59 mol, 1 eq) was added, and the reaction was warmed to reflux and stirred for 10 hrs. The mixture was then cooled to 25° C., and the residue was poured into ice water. The aqueous phase was then extracted with ethyl acetate. The combined organic phase was washed with brine, dried with anhydrous Na₂SO₄, filtered, and concentrated under reduced pressure. The crude residue was purified by silica gel chromatography to give Compound 2 (161 g, 98% yield) as a yellow oil.

Step 2: preparation of 3-(benzyloxy)-5-bromo-2-chloropyrazine (INT-2)

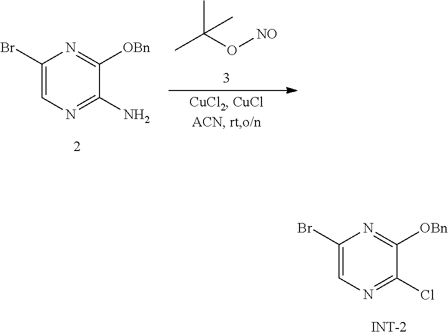

A suspension of compound 2 (161 g, 57.5 mmol), copper(II) chloride (233 g, 1.72 mol), copper(I) chloride (114 g, 115 mmol) in acetonitrile was stirred at rt for 10 min. compound 3 (200 mL, 1.72 mol) was added. The solution was stirred at rt overnight. Hydrochloric acid solution was added and the mixture was extracted with diethyl ether (1 L*3). The organic layer was dried, filtered and concentrated. The crude residue was purified by silica gel chromatography to give Compound INT-2 (100 g, 58% yield) as a white solid.

### Example 12

### Preparation of Methyl 3-((2-(Di-Boc-Amino)-3-Chloropyridin-4-Yl)Thio)Propanoate (INT4)

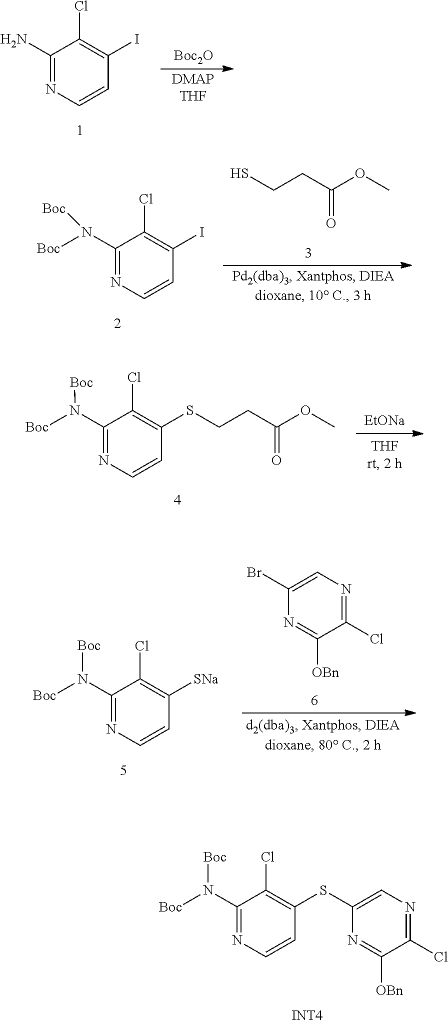

Step 1: 2-(di-Boc-amino)-3-chloro-4-iodopyridin (2)

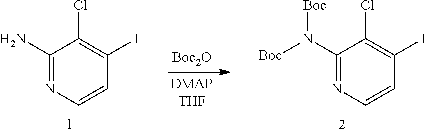 [SMILES: "Boc2O" ; Nc1nccc(I)c1Cl ; CC(C)(C)OC(=O)N(C(=O)OC(C)(C)C)c1nccc(I)c1Cl]

To a mixture of compound 1 (1.27 g, 5 mmol, 1 eq) and DMAP (1.22 g, 10 mmol, 2 eq) in THF was added (Boc)₂O (2.2 g, 10 mmol, 2 eq). The reaction mixture was stirred at 4 h for rt. Monitored by TLC, concentrated and purified by SiO₂ to afford compound 2 (1.57 g, yield: 88.7%)

Step 2: preparation of methyl 3-((2-(di-Boc-amino)-3-chloropyridin-4-yl)thio)propanoate (4)

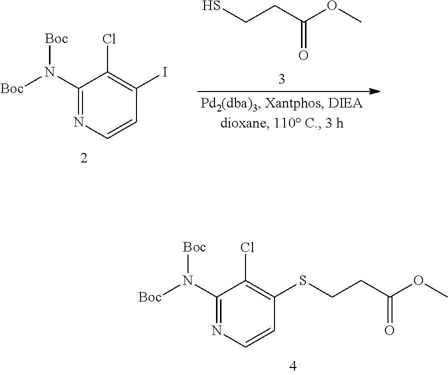

To a solution of compound 1 (1.57 g, 3.45 mmol, 1.0 eq) in 1,4-dioxane (30 ml) was added compound 2 (498 mg, 4.14 mmol, 1.2 eq), Pd2(dba)3 (300 mg, 0.34 mmol, 0.05 eq), Xant-phos (400 mg, 0.69 mmol, 0.1 eq) and DIEA (890 mg, 6.9 mmol, 2.0 eq). The reaction mixture was stirred at 110° C. under Ar for 3 h. The mixture was filtered and the filtered was concentrated. The residue was purified by column chromatography (PE/EtOAc=10:1) to give compound 4 as a yellow oil (1.23 g, 80.4% yield).

Step 3: preparation of sodium 2-(di-Boc-amino)-3-chloropyridine-4-thiolate (5)

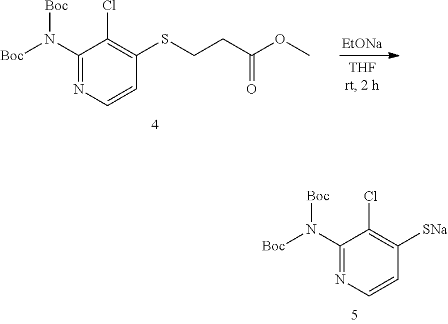

To a solution of compound 4 (1.23 g, 2.75 mmol, 1.0 eq) in THF (30 ml) was added EtONa (280 mg, 4.13 mmol, 1.5 eq). The reaction mixture was stirred at rt under N₂ for 1 hour. The mixture was concentrated. DCM (10 ml) was added into the residue, then filtered to give a white solid (1.05 g, crude). Used directly in the next step.

Step 4: preparation of methyl 3-((2-(di-Boc-amino)-3-chloropyridin-4-yl)thio)propanoate (INT4)

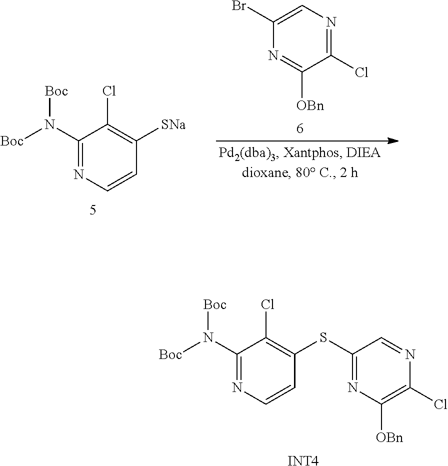

To a suspension of compound 5 (1.05 g, 2.7 mmol, 1.0 eq) in 1,4-dioxane (20 ml) was added 6 (819 mg, 2.7 mmol, 1.0 eq), Pd₂(dba)₃ (240 mg, 0.27 mmol, 0.1 eq), Xant-phos (200 mg, 0.27 mmol, 0.1 eq) and DIEA (697 mg, 5.4 mmol, 2.0 eq). The reaction mixture was stirred at 80° C. under N₂ for 2 h. The mixture was concentrated and purified by column chromatography (PE/EtOAc=10:1) to give INT4 as a yellow oil (780 mg, 50% yield). LC-MS: [M+H]⁺: 579

### Example 13

### Preparation of 3-(Benzyloxy)-2-Chloro-5-((2,3-Dichloropyridin-4-Yl)Thio)Pyrazine (INT5)

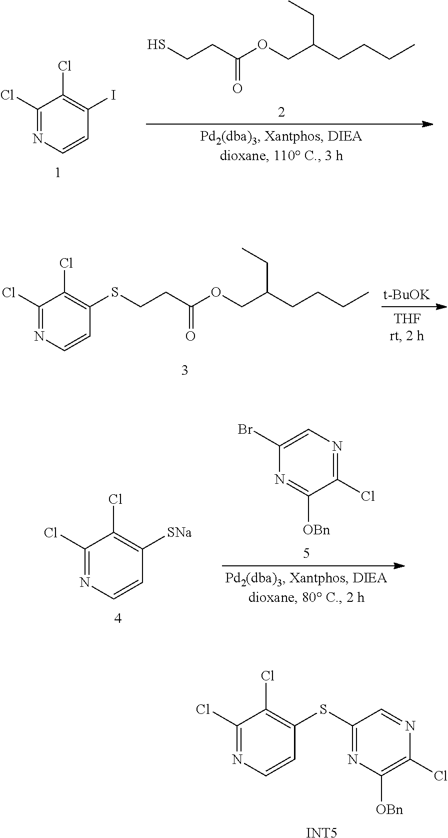

Step 1: preparation of 2-ethylhexyl 3-((2,3-dichloropyridin-4-yl)thio)propanoate (3)

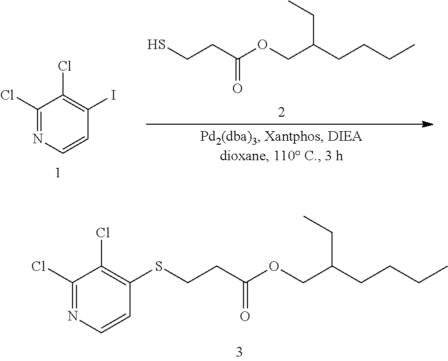 [SMILES: Clc1nccc(I)c1Cl ; CCCCC(CC)COC(=O)CCS ; CCCCC(CC)COC(=O)CCSc1ccnc(Cl)c1Cl]

To a solution of compound 1 (9.8 g, 35.9 mmol, 1.0 eq) in 1,4-dioxane (30 ml) was added compound 2 (10.2 g, 46.6 mmol, 1.3 eq), Pd₂(dba)₃ (2 g, 0.1 eq), Xant-phos (2 g, 3.59 mmol, 0.1 eq) and DIEA (9.3 g, 71.8 mmol, 2.0 eq). The reaction mixture was stirred at 110° C. under N₂ for 3 h. The mixture was filtered and the filtered was concentrated. The residue was purified by column chromatography (PE/EtOAc=10:1) to give compound 3 as a yellow oil (8.3 g, 62% yield).

Step 2: preparation of potassium 2,3-dichloropyridine-4-thiolate (4)

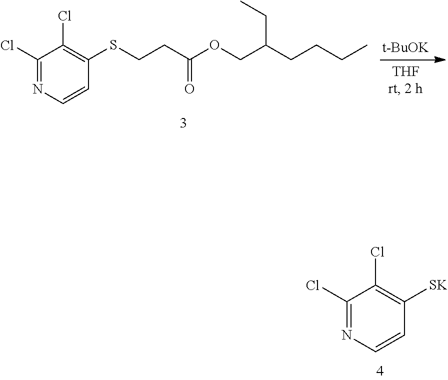

To a solution of compound 3 (8.3 g, 22.1 mmol, 1.0 eq) in THF (80 ml) was added t-BuOK (3.72 g, 433.2 mmol, 1.5 eq). The reaction mixture was stirred at rt under N₂ for 1 hour. The mixture was concentrated. DCM (10 ml) was added into the residue, then the mixture was filtered to give a white solid (4.5 g, crude), which as used directly in the next step.

Step 3: preparation of 3-(benzyloxy)-2-chloro-5-((2,3-dichloropyridin-4-yl)thio)pyrazine (INT5)

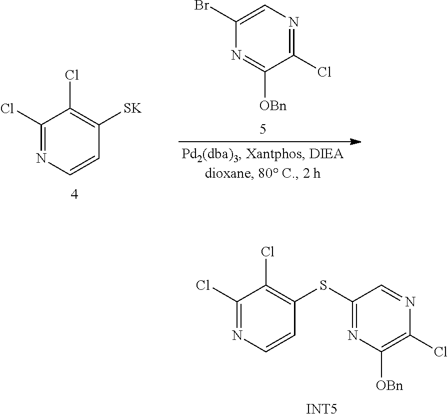

To a suspension of compound 4 (3 g, 13.7 mmol, 1.0 eq) in 1,4-dioxane (50 ml) was added 5 (4.1 g, 13.7 mmol, 1.0 eq), Pd₂(dba)₃ (400 mg, 0.1 eq), Xant-phos (800 mg, 1.37 mmol, 0.1 eq) and DIEA (3.54 g, 27.4 mmol, 2.0 eq). The reaction mixture was stirred at 80° C. under N₂ for 2 h. The mixture was concentrated and purified by column chromatography (PE/EtOAc=10:1) to give INT5 as a white solid (3.1 g, 57.4% yield). LC-MS: [M+H]⁺: 398

### Example 14

### Preparation of (S)-3-(4-Amino-2-Chloro-4, 6-Dihydrospiro [Cyclopenta[d]Thiazole-5, 4′-Piperidin]-1′-Yl)-6-((2, 3-Dichloropyridin-4-Yl) Thio) Pyrazin-2(1H)-One

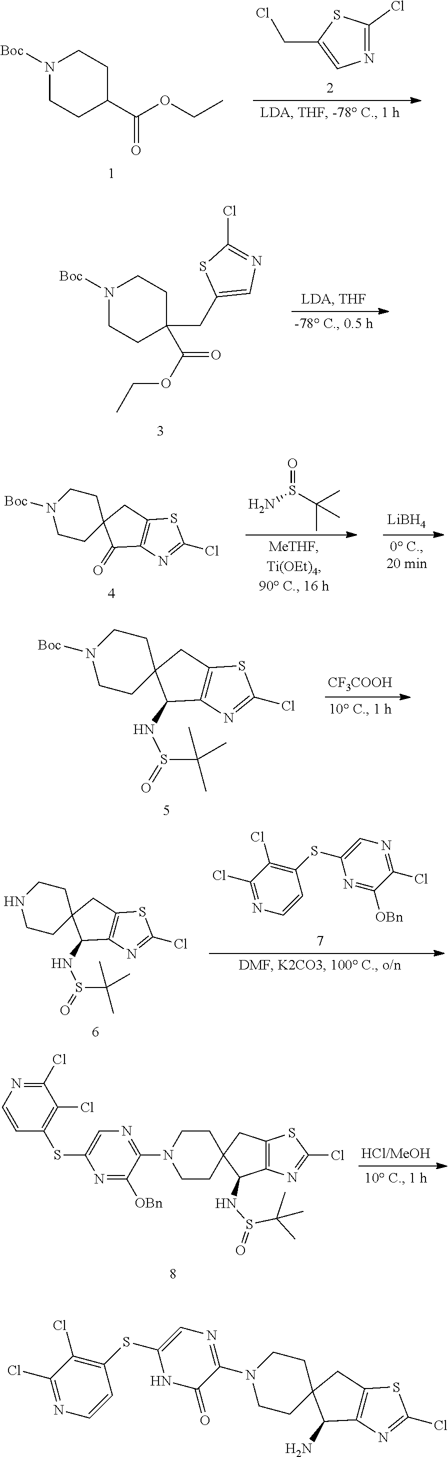

Step 1: Preparation of 1-(tert-butyl) 4-ethyl 4-((2-chlorothiazol-5-yl)methyl)piperidine-1,4-dicarboxylate (3)

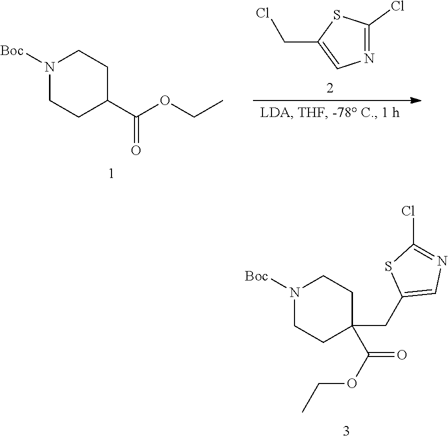 [SMILES: CCOC(=O)C1CCN(C(=O)OC(C)(C)C)CC1 ; ClCc1cnc(Cl)s1 ; CCOC(=O)C1(Cc2cnc(Cl)s2)CCN(C(=O)OC(C)(C)C)CC1]

To a solution of compound 1 (8.4 g, 33 mmol, 1.1 eq) in THF (150 mL) was added LDA (18 ml, 36 mmol, 1.2 eq) at −78° C. The mixture was stirred at −78° C. for 1 h. Then a solution of compound 2 (4.98 g, 3 mmol, 1 eq) was added and stirred another 2 h. The mixture was quenched with saturated solution of NH₄Cl (15 ml) and diluted with water (20 mL). Then it was extracted with DCM (100 mL), washed with brine (50 mL). The organic phase was dried over anhydrous Na₂SO₄, filtered and concentrated, purified by SGC eluting EA/PE to give compound 3 as a yellow solid. (6.2 g, 53.9% yield)

Step 2: Preparation of tert-butyl 2-chloro-4-oxo-4,6-dihydrospiro[cyclopenta[d]thiazole-5,4′-piperidine]-1′-carboxylate (4)

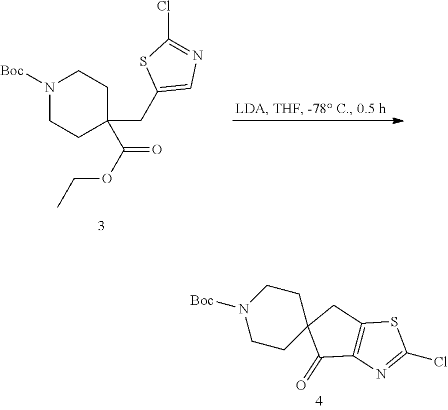 [SMILES: CCOC(=O)C1(Cc2cnc(Cl)s2)CCN(C(=O)OC(C)(C)C)CC1 ; CC(C)(C)OC(=O)N1CCC2(CC1)Cc1sc(Cl)nc1C2=O]

To a solution of compound 3 (6 g, 15 mmol, 1 eq) in the THF (200 mL) was added LDA (11.2 mL) at −78° C. The mixture was stirred at rt for 2 h. The solution was quenched with NH₄Cl and water, extracted with EA, dried over Na₂SO₄, concentrated and purified by SGC eluting EA/PEto give compound 4 as a yellow solid (1.9 g, 27.9% yield).

Step 3: preparation of tert-butyl (4S)-4-((tert-butylsulfinyl)amino)-2-chloro-4,6-dihydrospiro[cyclopenta[d]thiazole-5,4′-piperidine]-1′-carboxylate (5)

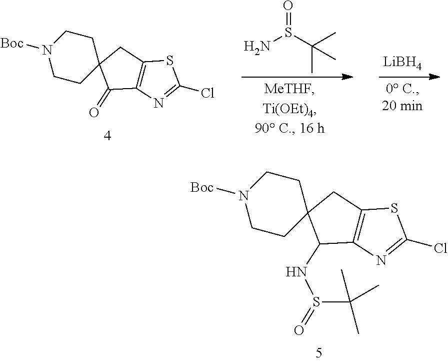

To a solution of compound 4 (400 mg, 1.17 mmol, 1 eq) in THF was added 2-methylpropane-2-sulfinamide (566 mg, 4.68 mmol, 4 eq), Ti(OEt)₄ (2.1 g, 9.36 mmol, 8 eq). The mixture was stirred at 90° C. for 16 h under N₂. The solution was cooled at 0° C., then a solution of LiBH₄ in THF (1 mL) was added and stirred for 0.5 h. The solution was quenched with MeOH at 0° C., then water (10 mL) was added to afford a suspension. The suspension was filtered, the filtrate was concentrated and extracted with EA. The organic phase was dried over anhydrous Na₂SO₄, then it was concentrated and purified by Pre-TLC to give compound 5 as a yellow solid. (180 mg, 34.4% yield)

Step 5: preparation of N—((S)-2-chloro-4,6-dihydrospiro[cyclopenta[d]thiazole-5,4′-piperidin]-4-yl)-2-methylpropane-2-sulfinamide (6)

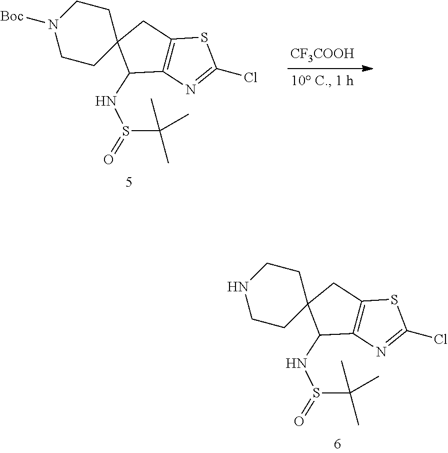 [SMILES: CC(C)(C)OC(=O)N1CCC2(CC1)Cc1sc(Cl)nc1C2NS(=O)C(C)(C)C ; "CF3COOH" ; CC(C)(C)S(=O)NC1c2nc(Cl)sc2CC12CCNCC2]

Compound 5 (180 mg, 0.40 mmol, 1 eq) was added CF₃COOH (2 mL) at 10° C. The mixture was stirred at 10° C. for 1 h. The solution was concentrated to give compound 6 as a yellow solid (139 mg, 100% yield) which was directly used in the next step.

Step 6: preparation of compound N—((S)-1′-(3-(benzyloxy)-5-((2,3-dichloropyridin-4-yl)thio)pyrazin-2-yl)-2-chloro-4,6-dihydrospiro[cyclopenta[d]thiazole-5,4′-piperidin]-4-yl)-2-methylpropane-2-sulfinamide (8)

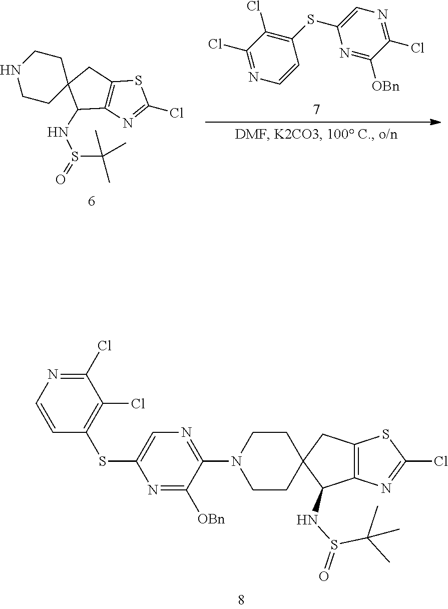 [SMILES: CC(C)(C)S(=O)NC1c2nc(Cl)sc2CC12CCNCC2 ; Clc1ncc(Sc2ccnc(Cl)c2Cl)nc1OCc1ccccc1 ; CC(C)(C)S(=O)N[C@@H]1c2nc(Cl)sc2CC12CCN(c1ncc(Sc3ccnc(Cl)c3Cl)nc1OCc1ccccc1)CC2]

To a solution of compound 6 (139 mg, 0.402 mmol, 1 eq) and compound 7 (123.2 mg, 0.402 mmol, 1 eq) in DMF (10 mL) was added K₂CO₃ (166.4 mg, 1.206 mmol, 3 eq) and stirred at 100° C. for overnight. The solvent was removed and purified by Pre-TLC (EA/PE=1.5/1) to give compound 8 as a yellow solid (60 mg, 21.2% yield).

Step 7: preparation of (S)-3-(4-amino-2-chloro-4,6-dihydrospiro[cyclopenta[d]thiazole-5,4′-piperidin]-1′-yl)-6-((2,3-dichloropyridin-4-yl)thio)pyrazin-2(1H)-one

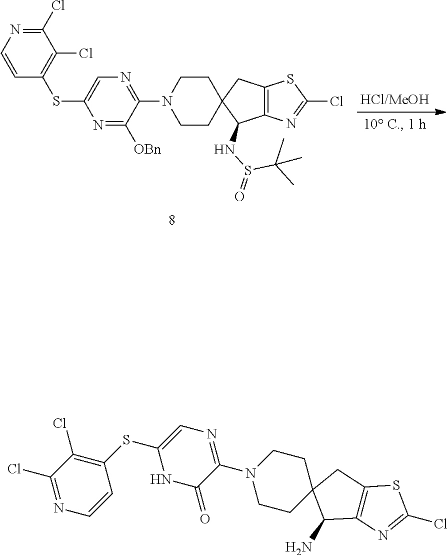 [SMILES: CC(C)(C)S(=O)N[C@@H]1c2nc(Cl)sc2CC12CCN(c1ncc(Sc3ccnc(Cl)c3Cl)nc1OCc1ccccc1)CC2 ; N[C@@H]1c2nc(Cl)sc2CC12CCN(c1ncc(Sc3ccnc(Cl)c3Cl)[nH]c1=O)CC2]

Compound 8 (60 mg, 0.085 mmol, 1 eq) was added in HCl/MeOH (2 mL). The mixture was stirred at 50° C. for 50 min. The solvent was removed and the residue was purified by prep-TLC (mobile phase: DCM/MeOH=8:1) to give (S)-3-(4-amino-2-chloro-4, 6-dihydrospiro [cyclopenta[d]thiazole-5, 4′-piperidin]-1′-yl)-6-((2, 3-dichloropyridin-4-yl) thio) pyrazin-2(1H)-one as a yellow solid (26 mg, 59.6%). LCMS [ESI] (M+H): 515. ¹H NMR (400 MHz, DMSO-d6) δ 8.20 (s, 1H), 7.25 (s, 1H), 7.05 (s, 1H), 4.42 (s, 2H), 3.80 (s, 1H), 2.83 (q, J=16.4, 15.8 Hz, 2H), 1.89 (s, 1H), 1.61 (d, J=37.1 Hz, 3H), 1.15 (d, J=58.2 Hz, 2H).

Below compounds were synthesized by a similar manner by using suitable amine derivatives and aryl derivatives;

TABLE E

| Compound | |
|---|---|
| ID | ¹H-NMR & MS [M + 1]⁺ |
| 117 | LCMS [ESI](M + H)⁺ 496 |
| | ¹H NMR (400 MHz, DMSO-d₆) δ 7.76 (d, J = 5.3 Hz, 1H), 7.20 (s, 1H), 6.37 (s, 2H), |
| | 6.09 (d, J = 5.4 Hz, 1H), 4.39 (d, J = 15.6 Hz, 2H), 3.80 (s, 1H), 3.63-3.42 (m, 3H), |
| | 2.96-2.71 (m, 2H), 1.97-1.83 (m, 1H), 1.74-1.50 (m, 3H), 1.25 (m, 2H). |
| 123 | LC-MS: [M + H]⁺ 476.1 |
| | ¹H NMR (400 MHz, DMSO-d6) δ 8.22 (d, J = 4.0 Hz, 1H), 7.34 (d, J = 8.0 Hz, 1H), |
| | 7.26 (s, 1H), 7.18-7.14 (m, 1H), 7.08 (d, J = 8.0 Hz, 1H), 6.90-6.86 (m, 1H), 6.79 |
| | (d, J = 8.0 Hz, 1H), 4.77-4.67 (m, 2H), 4.10 (s, 1H), 3.35-4.33 (m, 2H), 2.03- |
| | 1.71 (m, 4H). |
| 124 | LC-MS: [M + H]⁺ 457.1 |
| | ¹H NMR (400 MHz, DMSO-d₆) δ 7.76 (d, J = 5.3 Hz, 1H), 7.33 (d, J = 7.4 Hz, 1H), |
| | 7.22 (s, 1H), 7.15 (t, J = 7.7 Hz, 1H), 6.87 (t, J = 7.4 Hz, 1H), 6.79 (d, J = 7.9 Hz, |
| | 1H), 6.38 (s, 2H), 6.11 (d, J = 5.3 Hz, 1H), 4.71-4.67 (m, 2H), 4.10 (s, 1H), 3.55- |
| | 3.42 (m, 3H), 2.04-1.92 (m, 2H), 1.83-1.68 (m, 2H). |
| 126 | LC-MS: [M + H]⁺ 406.3 |
| | ¹H NMR (400 MHz, DMSO-d₆) δ 7.11 (s, 1H), 6.82 (td, J = 7.9, 0.9 Hz, 1H), 6.64 |
| | (td, J = 8.2, 1.6 Hz, 1H), 6.25 (ddd, J = 8.0, 6.5, 1.6 Hz, 1H), 5.28 (s, 2H), 4.20- |
| | 4.09 (m, 2H), 4.09-4.00 (m, 1H), 3.65 (d, J = 8.4 Hz, 1H), 3.58-3.36 (m, 3H), |
| | 2.89 (d, J = 5.1 Hz, 1H), 1.83-1.69 (m, 1H), 1.69-1.58 (m, 1H), 1.57-1.42 (m, |
| | 2H), 1.07 (d, J = 6.3 Hz, 3H). |
| 127 | LC-MS: [M + H]⁺ 407.2 |
| | ¹H NMR (400 MHz, DMS0-d6) δ 7.95 (t, J = 1.5 Hz, 1H), 7.58 (dd, J = 11.1, 1.9 Hz, |
| | 1H), 7.07 (s, 1H), 6.61 (s, 2H), 4.13-3.99 (m, 2H), 3.63 (d, J = 8.4 Hz, 1H), 3.51- |
| | 3.42 (m, 2H), 3.41-3.30 (m, 2H), 2.87 (d, J = 5.1 Hz, 1H), 1.78-1.66 (m, 1H), |
| | 1.66-1.56 (m, 1H), 1.53-1.38 (m, 2H), 1.06 (d, J = 6.3 Hz, 3H). |
| 128 | LC-MS: [M + H]⁺ 408.3 |
| | ¹H NMR (400 MHz, DMSO-d6) δ 8.53 (s, 1H), 8.35 (d, J = 5.3 Hz, 1H), 7.23 (s, 1H), |
| | 7.01 (d, J = 5.3 Hz, 1H), 4.31-4.18 (m, 3H), 4.11-4.02 (m, 1H), 3.68 (d, J = 8.4 |
| | Hz, 1H), 3.62-3.43 (m, 2H), 2.94 (d, J = 5.1 Hz, 1H), 1.83-1.73 (m, 1H), 1.72- |
| | 1.61 (m, 1H), 1.59-1.45 (m, 2H), 1.09 (d, J = 6.4 Hz, 3H). |
| 129 | LC-MS: [M + H]⁺ 451 |
| | ¹H NMR (400 MHz, DMSO-d₆) δ 7.99 (d, J = 5.3 Hz, 1H), 7.20 (s, 1H), 6.45 (d, J = |
| | 5.3 Hz, 1H), 4.29-4.15 (m, 2H), 4.13-4.01 (m, 1H), 3.67-3.45 (m, 4H), 2.90 (s, |
| | 6H), 1.84-1.43 (m, 4H), 1.09 (d, J = 6.4 Hz, 3H). |
| 130 | LCMS (M + H)⁺ 462 |
| 131 | LCMS [ESI](M + H)⁺ 503 |
| | ¹H NMR (400 MHz, DMSO) δ 8.24 (d, J = 2.6 Hz, 1H), 7.49 (dd, J = 8.0, 1.3 Hz, |
| | 1H), 7.33 (t, J = 8.0 Hz, 1H), 7.24-7.18 (m, 2H), 7.02-6.94 (m, 2H), 6.86 (d, J = |
| | 8.1 Hz, 1H), 4.73 (d, J = 11.0 Hz, 2H), 4.02 (s, 1H), 3.78 (s, 3H), 3.23 (s, 2H), 2.99 |
| | (d, J = 16.1 Hz, 1H), 2.71-2.62 (m, 1H), 1.75 (s, 2H), 1.48 (d, J = 13.1 Hz, 1H), |
| | 1.29 (d, J = 13.2 Hz, 1H). |
| 132 | LC-MS: [M + H]⁺ = 474.1 |
| | ¹H NMR (400 MHz, DMSO) δ 8.33 (s, 1H), 7.49 (dd, J = 8.0, 1.3 Hz, 1H), 7.33 (t, |
| | J = 8.0 Hz, 1H), 7.23 (s, 1H), 7.15 (d, J = 7.2 Hz, 1H), 7.00-6.92 (m, 2H), 6.56 (t, |
| | J = 7.3 Hz, 1H), 6.50 (d, J = 7.7 Hz, 1H), 6.16 (s, 1H), 4.61 (d, J = 12.2 Hz, 2H), 3.91 |
| | (s, 1H), 3.46 (s, 2H), 1.91-1.72 (m, 2H), 1.48 (dd, J = 38.6, 13.3 Hz, 2H). |
| 133 | LC-MS: [M + H]⁺ 407 |
| | ¹H NMR (400 MHz, DMSO) δ 7.74 (d, J = 5.3 Hz, 1H), 7.17 (s, 1H), 6.36 (s, 2H), |
| | 6.06 (d, J = 5.4 Hz, 1H), 4.64 (s, 2H), 3.11 (t, J = 12.5 Hz, 2H), 2.82 (d, J = 7.3 Hz, |
| | 1H), 1.94-1.82 (m, 1H), 1.75 (dd, J = 19.6, 14.2 Hz, 1H), 1.69-1.52 (m, 4H), |
| | 1.46 (dd, J = 24.1, 18.0 Hz, 2H), 1.26 (dd, J = 22.7, 13.8 Hz, 2H). |
| 134 | LCMS [ESI](M + H)⁺ 474 |
| | ¹H NMR (400 MHz, DMSO) δ 12.22 (s, 1H), 9.02 (s, 3H), 8.75 (d, J = 5.2 Hz, 1H), |
| | 8.54 (s, 1H), 7.76 (s, 1H), 7.50 (dd, J = 8.0, 1.3 Hz, 1H), 7.34 (t, J = 8.0 Hz, 1H), |
| | 7.25 (s, 1H), 7.01 (d, J = 8.0 Hz, 1H), 4.78 (s, 2H), 4.59 (s, 2H), 3.53 (d, J = 17.4 Hz, |
| | 1H), 3.37-3.23 (m, 3H), 1.90 (dt, J = 21.1, 12.1 Hz, 2H), 1.66 (dd, J = 33.5, 13.0 |
| | Hz, 2H). |
| 135 | LC-MS: [M + H]⁺ 426.0 |
| | ¹H NMR (400 MHz, DMSO) δ 8.33 (s, 1H), 8.20 (d, J = 5.3 Hz, 1H), 7.25 (s, 1H), |
| | 7.03 (d, J = 5.3 Hz, 1H), 4.75-4.59 (m, 2H), 3.15-3.09 (m, 2H), 3.00 (d, J = 5.6 |
| | Hz, 1H), 2.05-1.91 (m, 1H), 1.83-1.51 (m, 7H), 1.42-1.24 (m, 2H). |
| 135 | MS [M + 1]⁺ 442 |

The SHP2 inhibitors described herein provide advantageous features, either alone or in combination with other therapeutic agents, for the treatment of various disease, disorders, or condition associated with the aberrant activity of SHP2, including cancer and autoimmune disorders. Examples of the various testing results are shown below, which are in no mean to limit the therapeutic applications of the SHP2 inhibitors described herein. These non-limiting examples strongly demonstrated that the SHP2 inhibitors described herein could be used in the treatment of cancer and autoimmune disorders, etc.

### Pharmacological Testing

Some of the compounds disclosed herein were assessed for their ability to selectively inhibit SHP2 activity. The inhibitory properties of the compounds described herein can be evidenced by testing in the following assays.

### SHP2 Phosphatase Assays

IC₅₀ values were determined at room temperature in 384-well black polystyrene plate, using a final reaction volume of 15 μL and the following assay buffer conditions: 60 mM Hepes (pH=7.2), 75 mM NaCl, 75 mM KCl, and 1 mM EDTA, 0.05% P-20, 5 mM dithiothreitol (DTT). Full length SHP2 enzyme (diluted to 0.1 nM in reaction buffer) were co-incubated with 1 μM IRS-1 peptide and 0.01 nM to 10 μM compounds of the disclosure for 60 min. The surrogate substrate DiFMUP (5 μL, 100 μM) was added, and incubated at rt for 60 min. The reaction was then quenched by the addition of 5 μL of a 40 μM solution of bpV(Phen). The fluorescence signal was monitored using a microplate reader (Envision, Perkin-Elmer) using excitation and emission wavelengths of 360 nm and 450 nm, respectively. The inhibitor dose-response curves were analyzed using normalized IC₅₀ regression curve fitting with control-based normalization. The inhibitory activity results of the compounds of the disclosure are shown in Table 2.

TABLE 2

| Compound ID | IC₅₀ |
|---|---|
| 2 | +++ |
| 35 | +++ |
| 52 | +++ |
| 34 | +++ |
| 77 | +++ |
| 31 | +++ |
| 29 | +++ |
| 32 | +++ |
| 28 | +++ |
| 30 | +++ |
| 14 | +++ |
| 73 | +++ |
| 74 | +++ |
| 33 | +++ |
| 79 | +++ |
| 80 | +++ |
| 81 | +++ |
| 84 | +++ |
| 107 | +++ |
| 109 | +++ |
| 82 | +++ |
| 83 | +++ |
| 13 | +++ |
| 86 | +++ |
| 108 | +++ |
| 87 | +++ |
| 88 | +++ |
| 73 | +++ |
| 116 | +++ |
| 117 | +++ |
| 123 | +++ |
| 124 | +++ |
| 126 | +++ |
| 127 | + |
| 128 | +++ |
| 128 | +++ |
| 130 | +++ |
| 131 | +++ |
| 132 | +++ |
| 133 | +++ |
| 134 | +++ |
| 135 | +++ |
| 136 | +++ |

IC₅₀: +++: ≤50 nM; ++: ≤100 nM; +: ≤1 μM;

### P-ERK/Total ERK Cellular Assay

Cells were seeded in 384-well cell culture plate and incubated overnight. Test compounds were added to the cell plate and the plate was incubated for 2-6 hours. To detect p-ERK, the cell plate was used AlphaLISA SureFire Ultra p-ERK kit; to detect total ERK, the cell plate was used total ERK HTRF kit. The plate was read on the Envision. The inhibitory activity results of the compounds of the disclosure is shown in Table 3.

TABLE 3

| Compound ID | IC₅₀ |
|---|---|
| 2 | ++ |
| 35 | ++ |
| 34 | ++ |
| 10 | +++ |
| 31 | +++ |
| 29 | +++ |
| 30 | +++ |
| 14 | +++ |
| 73 | +++ |
| 33 | +++ |
| 79 | +++ |
| 80 | +++ |
| 81 | +++ |
| 84 | +++ |
| 109 | +++ |
| 82 | +++ |
| 83 | +++ |

IC₅₀: +++: ≤0.1 μM; ++: ≤0.5 μM; +: ≤1 μM;

### Cell Proliferation Assay

Firstly, the cells were seeded in 384-well cell culture plate and incubate overnight. The test compounds were added to the cell plate and incubated for 3-5 days. The cell plate was then detected using CellTiter Glo reagents. The inhibitory activity results of the compounds of the disclosure is shown in Table 4 and Table 5.

TABLE 4
KYSE-520 cell line

| Compound ID | IC₅₀ |
|---|---|
| 2 | ++ |
| 10 | ++ |
| 31 | ++ |
| 29 | ++ |
| 14 | +++ |
| 73 | ++ |
| 81 | +++ |
| 84 | +++ |
| 109 | ++ |
| 82 | ++ |
| 83 | +++ |

(IC₅₀: +++: ≤1 μM; ++: ≤5 μM; +: ≤10 μM)

TABLE 5

| | Compound |
|---|---|
| | ID: 14 |
| Cell Lines | IC50 (μM) |
| KYSE-520 (Esophageal) | +++ |
| NCl-H358 (Lung, KRAS^{G12C}) | ++ |
| NCl-H2122 (lung, KRAS^{G12C}) | + |
| Mia-pa-ca-2 (Pancreatic, KRAS^{G12C}) | ++ |
| NCl-H1838 (Lung, NF1^{l183fs}) | + |
| MeWo (Melanoma, NF1^{Q1336}) | + |
| NCl-H1666 (BRAF^{G466V/+}, Lung) | ++ |
| NCl-H508 (BRAF^{G596R/+}, Caecum) | +++ |

IC₅₀: +++: ≤1 μM; ++: ≤10 μM; +: ≤20 μM;

As shown in Tables 4 and 5, the compounds tested displayed superior anti-proliferation activities in various tumor types, for example lung cancer, esophageal cancer, pancreatic cancer, Caecum cancer.

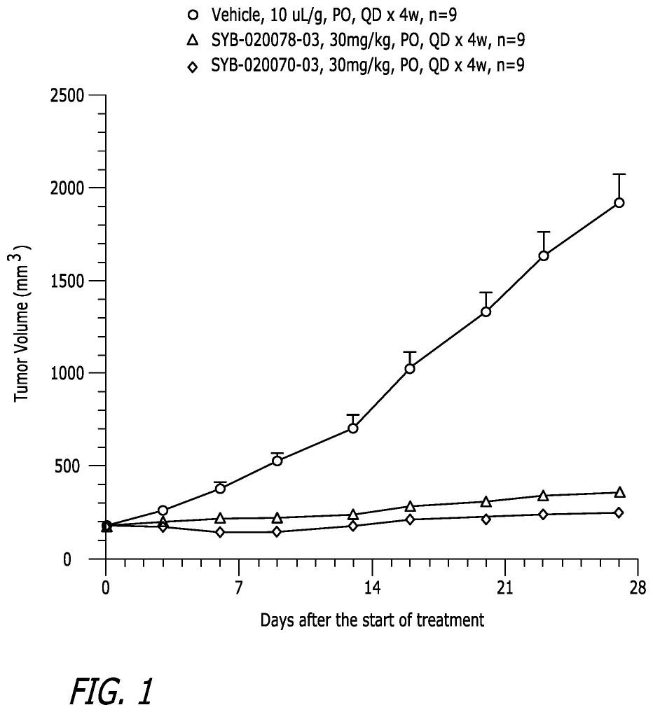

Many of the compounds described herein are very potent and selective, with enzymatic IC₅₀ less than 10 nM. The compounds tested also displayed superior anti-tumor activities in various in vivo animal models. In some embodiments, the dose amount per day falls within the range of 1 mg/kg to 100 mg/kg to achieve the tumor regression or >70% tumor growth inhibition. Examples of the anti-tumor activities in various in vivo animal models, such as KYSE-520 Xenograft Model, Lung cancer H-358 Xenograft Model, Pancreatic cancer Mia-Pa—Ca-2 Xenograft Model, and non-small cell lung cancer (NSCLC) with KRAS mutant Xenograft Model, are shown below (FIGS. 1-4), which are in no mean to limit the in vivo animal models in which the SHP2 inhibitors described herein could show anti-tumor activities. These non-limiting examples strongly demonstrated that the SHP2 inhibitors described herein could be used in the treatment of cancer and autoimmune disorders, etc. such as to effectively reduce various tumors or achieve tumor regression in mammals.

### Esophageal KYSE-520 Xenograft Model

KYSE-520 cells were expanded in culture, harvested and injected subcutaneously into 6-8 weeks old female BALB/c nude mice (5×10⁶ cell/each mouse, supplemented with Matrigel (1:1) for tumor development, n=9 per group). Subsequent administration of a compound by oral gavage started when the mean tumor size reached approximately 150-200 mm³. During the treatment (once a day for 4 weeks), the tumor volumes were measured using a caliper. Statistical analysis of difference in tumor volume among the groups were evaluated using a one-way ANOVA. Vehicle alone was the negative control. The testing results are shown in FIG. 1.

As shown in FIG. 1, the compounds tested displayed superior anti-tumor activities in the in vivo KYSE-520 Xenograft animal model. The oral administration of 30 mg/kg per day of compound SYB-020070 (compound ID: 14), once a day for 4 weeks in mice, achieved more than 90% tumor growth inhibition, and demonstrated superior anti-tumor activities than reference compound RMC-4550 (SYB-020078).

### Lung Cancer H-358 Xenograft Studies

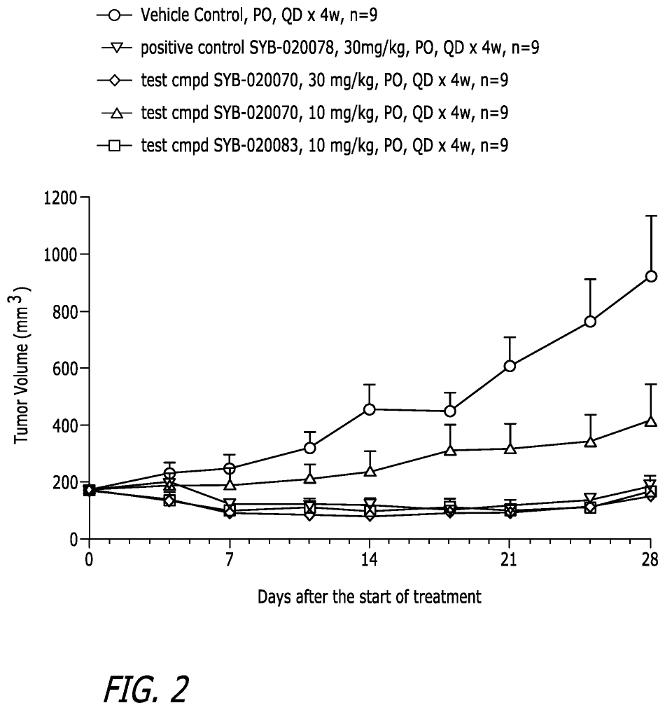

NCI-H358 cells were expanded in culture, harvested and injected subcutaneously into 6-8 weeks old female BALB/c nude mice (5×10⁶ cell/each mouse, supplemented with Matrigel (1:1) for tumor development, n=9 per group). Subsequent administration of a compound by oral gavage started when the mean tumor size reached approximately 150-200 mm³. During the treatment (once a day for 4 weeks), the tumor volumes were measured using a caliper. Statistical analysis of difference in tumor volume among the groups were evaluated using a one-way ANOVA. Vehicle alone was the negative control. The testing results are shown in FIG. 2.

As shown in FIG. 2, the compounds tested displayed superior anti-tumor activities in the in vivo Lung cancer H-358 Xenograft animal model. The oral administration of 10 mg/kg or 30 mg/kg, per day of SYB-020070 (compound ID: 14), or SYB-020083 (compound ID: 77), once a day for 4 weeks in mice, achieved more than 70% tumor growth inhibition, and in some cases, tumor regression.

### Pancreatic Cancer Mia-Pa—Ca-2 Xenograft Studies

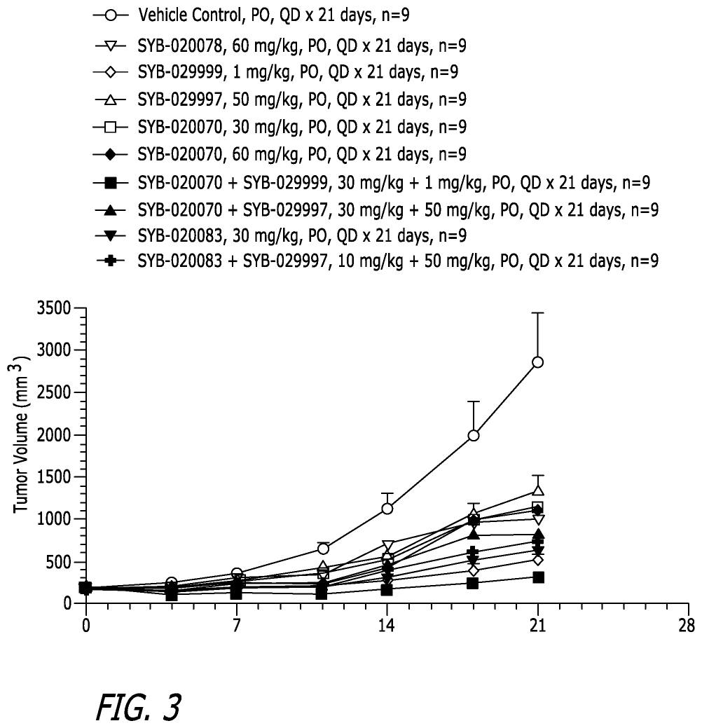

Mai-Pa-ca-2 cells were expanded in culture, harvested and injected subcutaneously into 6-8 weeks old female BALB/c nude mice (5×10⁶ cell/each mouse, supplemented with Matrigel (1:1) for tumor development, n=9 per group). Subsequent administration of a compound by oral gavage started when the mean tumor size reached approximately 150-200 mm³. During the treatment (once a day for 21 days), the tumor volumes were measured using a caliper. Statistical analysis of difference in tumor volume among the groups were evaluated using a one-way ANOVA. Vehicle alone was the negative control. The testing results are shown in FIG. 3.

As shown in FIG. 3, the compounds tested displayed superior anti-tumor activities in the in vivo Pancreatic cancer Mia-Pa—Ca-2 Xenograft animal model. The oral administration of 30 mg/kg or 60 mg/kg of compound SYB-020070 (compound ID: 14), or 30 mg/kg of compound SYB-020083 (compound ID: 77), once a day for 21 days in mice, achieved more than 70% tumor growth inhibition.

Additionally, the co-administration of SYB-020070 (compound ID: 14) (30 mg/kg per day) and MEK inhibitor SYB-020099 (1 mg/kg per day), or the co-administration of SYB-020070 (compound ID: 14) (30 mg/kg per day) and CDK4/6 inhibitor SYB-020097 (50 mg/kg per day), achieved significantly higher reduction of tumor growth than that for administration of each individual compound alone with the same dose amount demonstrating the synergistic effects.

Combination Treatment of a SHP2 Inhibitor with Other Therapeutic Agents

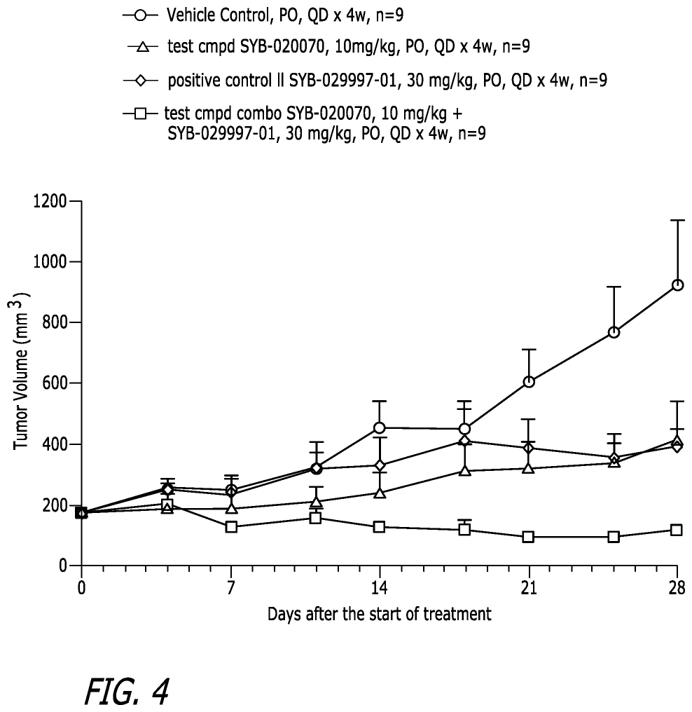

NCI-H358 cells were expanded in culture, harvested and injected subcutaneously into 6-8 weeks old female BALB/c nude mice (5×10⁶ cell/each mouse, supplemented with Matrigel (1:1) for tumor development, n=9 per group). Subsequent administration of a compound or combination of compounds by oral gavage started when the mean tumor size reached approximately 150-200 mm³. During the treatment (once a day for 4 weeks), the tumor volumes were measured using a caliper. Statistical analysis of difference in tumor volume among the groups were evaluated using a one-way ANOVA. Vehicle alone was the negative control. The testing results are shown in FIG. 4.

A pharmaceutical combination comprising a SHP2 inhibitor with other therapeutic agents is more potent in inhibiting tumor growth than a single agent alone in vivo xenograft study. For example, as shown in FIG. 4, the oral administration of the combination of SYB-020070 (compound ID: 14, 10 mg/kg per day) with CDK4/6 inhibitor SYB-020097 (30 mg/kg per day) in mice once daily for 28 days resulted in unexpected significant improvement (about more than 50% improvement) over the monotherapy with SHP2 inhibitor SYB-020070 or CDK4/6 inhibitor SYB-020097 alone respectively each with same dose amount as that in the combination, in the treatment of non-small cell lung cancer (NSCLC) with KRAS mutant demonstrating the synergistic effects.

Unless otherwise indicated, all numbers expressing quantities of ingredients, properties such as molecular weight, reaction conditions, and etc. used in herein are to be understood as being modified in all instances by the term “about.” Each numerical parameter should at least be construed in light of the number of reported significant digits and by applying ordinary rounding techniques. Accordingly, unless indicated to the contrary, the numerical parameters may be modified according to the desired properties sought to be achieved, and should, therefore, be considered as part of the disclosure. At the very least, the examples shown herein are for illustration only, not as an attempt to limit the scope of the disclosure.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illustrate embodiments of the present disclosure and does not pose a limitation on the scope of any claim. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the embodiments. Of course, variations on these described embodiments will become apparent to those of ordinary skill in the art upon reading the foregoing description. The inventor expects skilled artisans to employ such variations as appropriate, and the inventors intend for the embodiments of the present disclosure to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating a disease, a disorder, or a condition associated with the aberrant activity of SHP2, comprising administering a therapeutically effective amount of a compound represented by a formula, 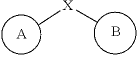
or a pharmaceutically acceptable salt thereof, to a patient in need thereof,
wherein X is S;
Ring A is an optionally substituted aryl having 6-10 ring carbon atoms; an optionally substituted 5-membered mono-cyclic heteroaryl comprising 0-4 ring nitrogen atoms, 0-1 ring oxygen atom, 0-1 ring sulfur atom, and at least one N, O, or S ring atom; an optionally substituted 6-membered mono-cyclic heteroaryl comprising 1-3 ring nitrogen atoms; or an optionally substituted bicyclic ring system having 5-10 ring carbon atoms, 0-4 ring nitrogen atoms, 0-1 ring oxygen atom, or 0-1 ring sulfur atom, wherein the bicyclic ring system is unsaturated or partially saturated;
Ring B is: 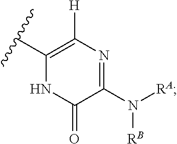
wherein R^{A} and R^{B} are independently H or C₁₋₁₂ hydrocarbyl, or —N(R^{A})(R^{B}) is an optionally substituted heterocyclic ring system, wherein the heterocyclic ring system is a mono-cyclic ring having 2-8 ring carbon atoms, 1-2 ring nitrogen atoms, 0-1 ring oxygen atom, and 0-1 ring sulfur atom; a bicyclic ring system having 5-12 ring carbon atoms, 1-2 ring nitrogen atoms, 0-1 ring oxygen atom, and 0-1 ring sulfur atom; or a tricyclic ring system having 8-16 ring carbon atoms, 1-2 ring nitrogen atoms, 0-1 ring oxygen atom, and 0-1 ring sulfur atom; wherein the bicyclic ring system or the tricyclic ring system is a spiro, fused, or bridged ring system, wherein the heterocyclic ring system is saturated or partially saturated;
wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof, and
wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.

2. (canceled)

3. The method of claim 1, wherein Ring A is optionally substituted phenyl.

4. The method of claim 1, wherein Ring A is optionally substituted pyridinyl.

5. The method of claim 1, wherein Ring A is optionally substituted pyridin-4-yl.

6. The method of claim 1, wherein Ring A is optionally substituted 2,3-dichlorophenyl.

7. The method of claim 1, wherein Ring A is optionally substituted 2,3-dichloro-pyridin-4-yl.

8. The method of claim 1, wherein Ring A is optionally substituted 2-amino-3-chloropyridin-4-yl.

9. The method of claim 1, wherein Ring A is:

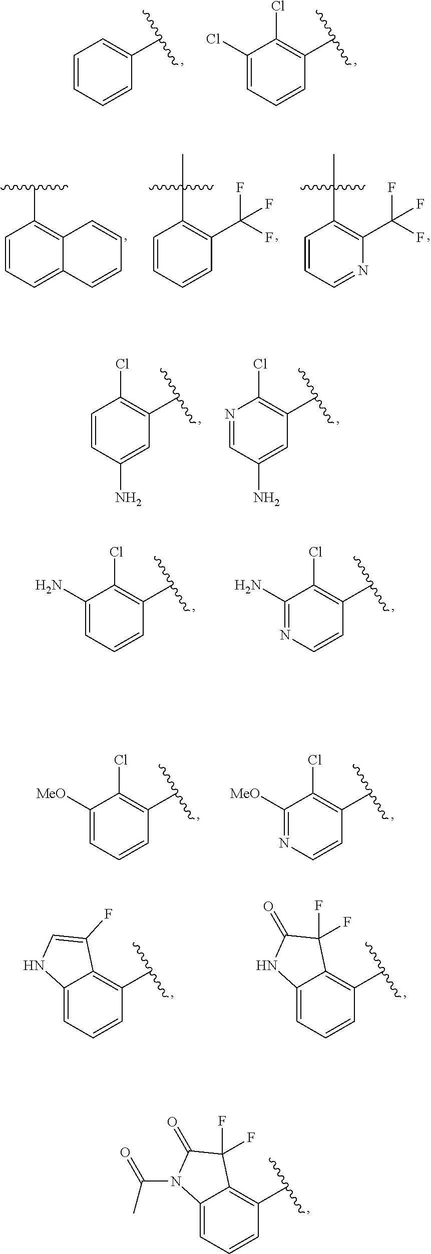

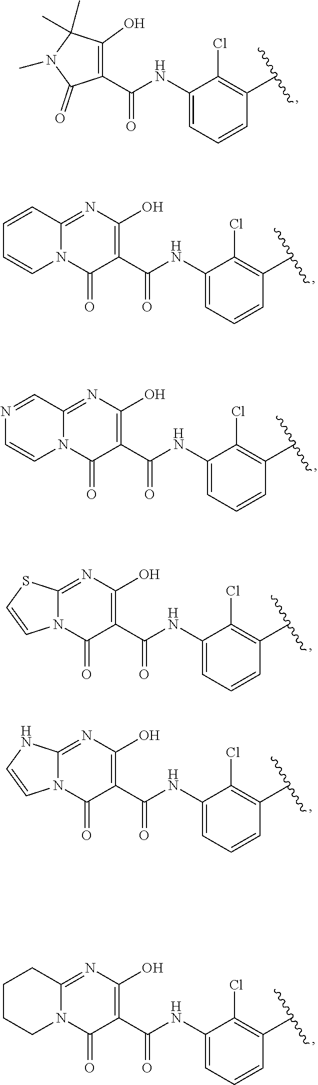 [SMILES: *c1cccc(NC(=O)C2=C(O)C(C)(C)N(C)C2=O)c1Cl ; *c1cccc(NC(=O)c2c(O)nc3ccccn3c2=O)c1Cl ; *c1cccc(NC(=O)c2c(O)nc3cnccn3c2=O)c1Cl ; *c1cccc(NC(=O)c2c(O)nc3sccn3c2=O)c1Cl ; *c1cccc(NC(=O)c2c(O)nc3[nH]ccn3c2=O)c1Cl ; *c1cccc(NC(=O)c2c(O)nc3n(c2=O)CCCC3)c1Cl]

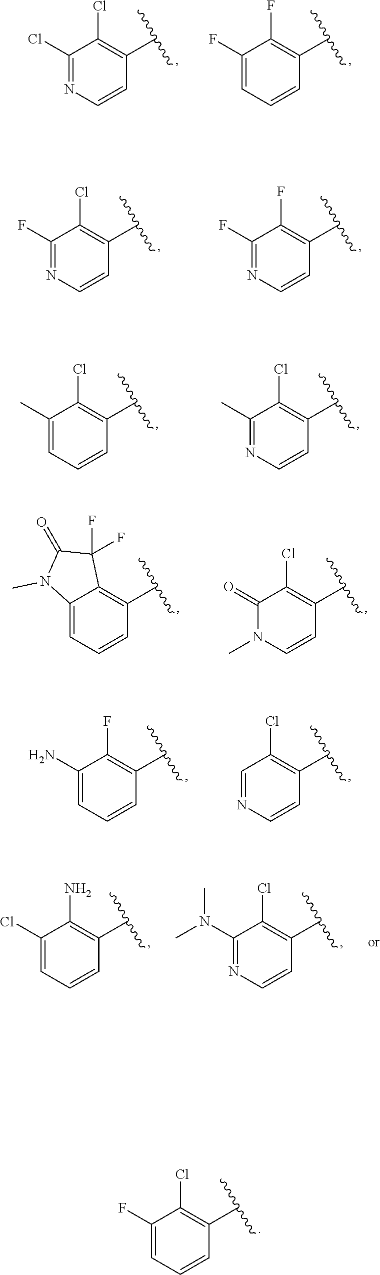

10. The method of claim 1, wherein Ring B is 5-((3S,4S)-4-amino-3-methyl-2-oxa-8-azaspiro[4.5]decan-8-yl)-6-oxo-1,6-dihydropyrazin-2-yl.

11. The method of claim 1, wherein Ring B is:

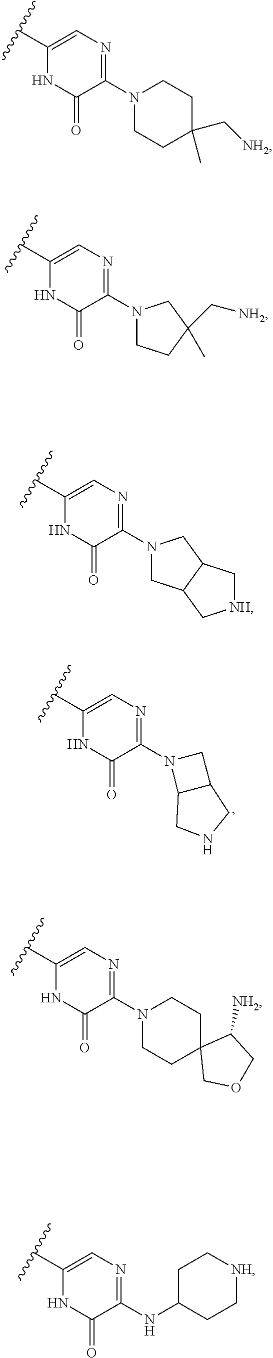 [SMILES: *c1cnc(N2CCC(C)(CN)CC2)c(=O)[nH]1 ; *c1cnc(N2CCC(C)(CN)C2)c(=O)[nH]1 ; *c1cnc(N2CC3CNCC3C2)c(=O)[nH]1 ; *c1cnc(N2CC3CNCC32)c(=O)[nH]1 ; *c1cnc(N2CCC3(CC2)COC[C@H]3N)c(=O)[nH]1 ; *c1cnc(NC2CCNCC2)c(=O)[nH]1]

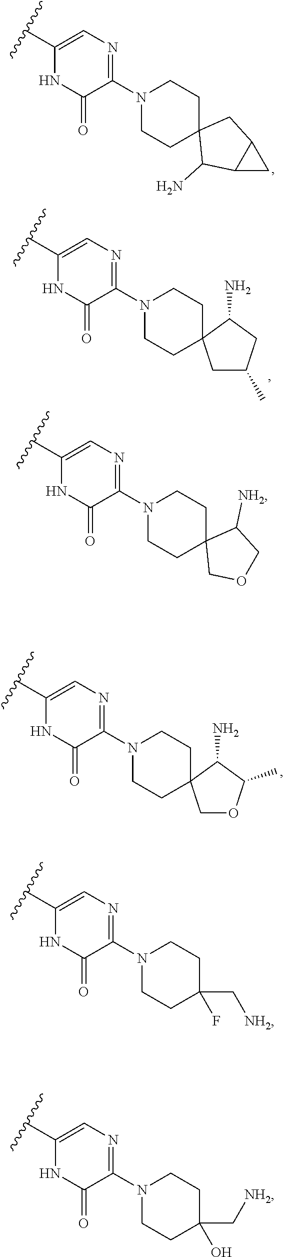 [SMILES: *c1cnc(N2CCC3(CC2)CC2CC2C3N)c(=O)[nH]1 ; *c1cnc(N2CCC3(CC2)C[C@@H](C)C[C@H]3N)c(=O)[nH]1 ; *c1cnc(N2CCC3(CC2)COCC3N)c(=O)[nH]1 ; *c1cnc(N2CCC3(CC2)CO[C@@H](C)[C@H]3N)c(=O)[nH]1 ; *c1cnc(N2CCC(F)(CN)CC2)c(=O)[nH]1 ; *c1cnc(N2CCC(O)(CN)CC2)c(=O)[nH]1]

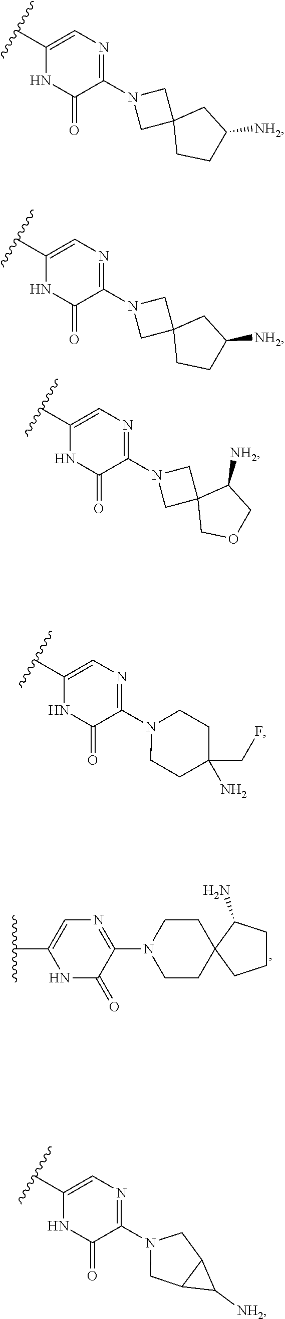 [SMILES: *c1cnc(N2CC3(CC[C@@H](N)C3)C2)c(=O)[nH]1 ; *c1cnc(N2CC3(CC[C@H](N)C3)C2)c(=O)[nH]1 ; *c1cnc(N2CC3(COC[C@@H]3N)C2)c(=O)[nH]1 ; *c1cnc(N2CCC(N)(CF)CC2)c(=O)[nH]1 ; *c1cnc(N2CCC3(CCC[C@H]3N)CC2)c(=O)[nH]1 ; *c1cnc(N2CC3C(N)C3C2)c(=O)[nH]1]

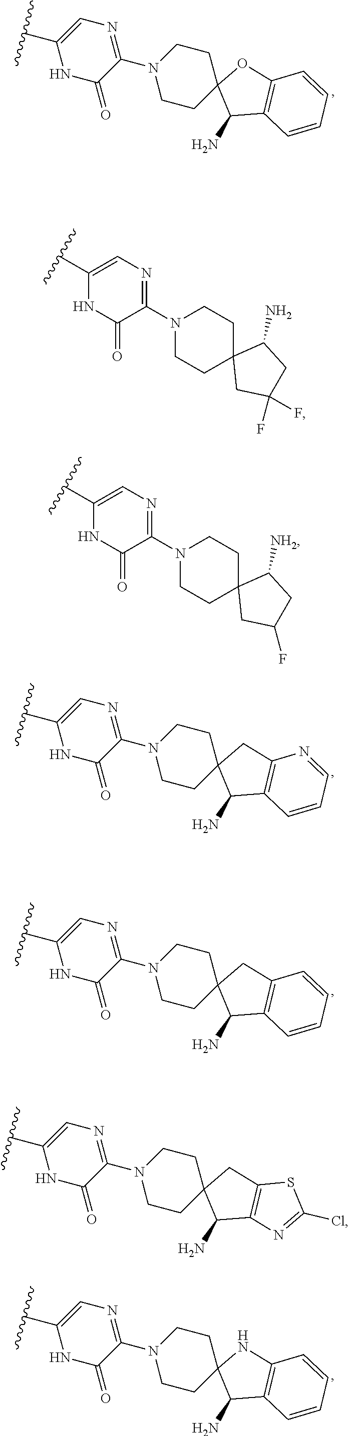

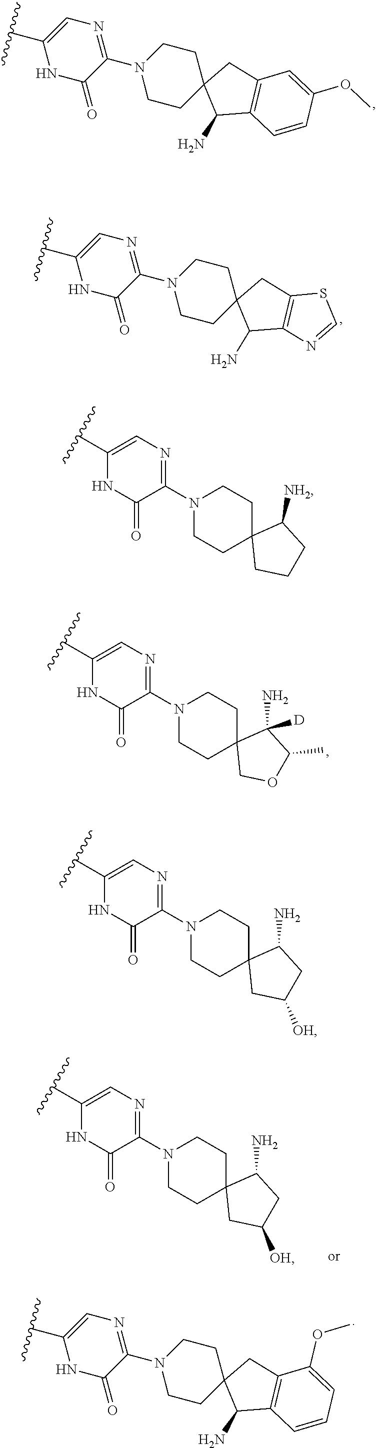

12. The method of claim 1, wherein the compound is an R-enantiomer.

13. The method of claim 1, wherein the compound is an S-enantiomer.

14. The method of claim 1, or a pharmaceutically acceptable salt thereof, wherein the compound is:

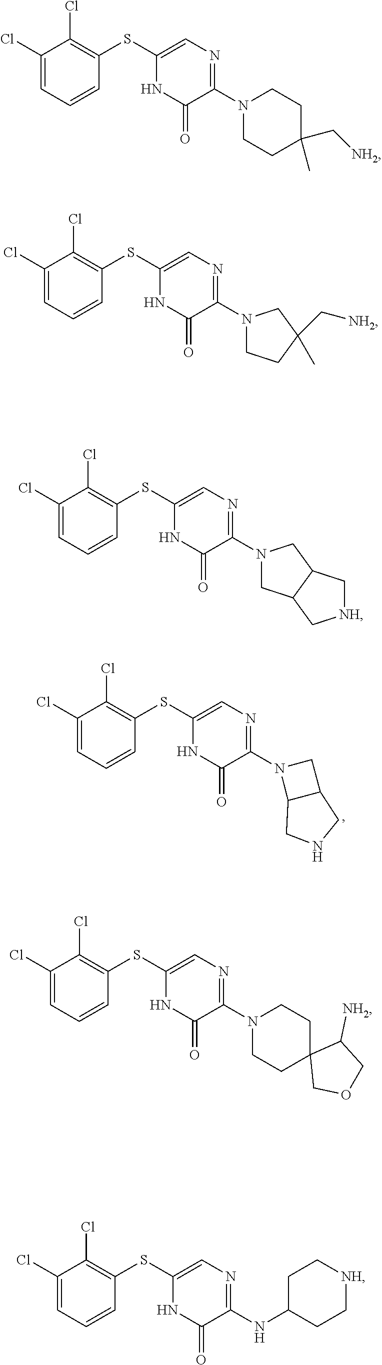 [SMILES: CC1(CN)CCN(c2ncc(Sc3cccc(Cl)c3Cl)[nH]c2=O)CC1 ; CC1(CN)CCN(c2ncc(Sc3cccc(Cl)c3Cl)[nH]c2=O)C1 ; O=c1[nH]c(Sc2cccc(Cl)c2Cl)cnc1N1CC2CNCC2C1 ; O=c1[nH]c(Sc2cccc(Cl)c2Cl)cnc1N1CC2CNCC21 ; NC1COCC12CCN(c1ncc(Sc3cccc(Cl)c3Cl)[nH]c1=O)CC2 ; O=c1[nH]c(Sc2cccc(Cl)c2Cl)cnc1NC1CCNCC1]

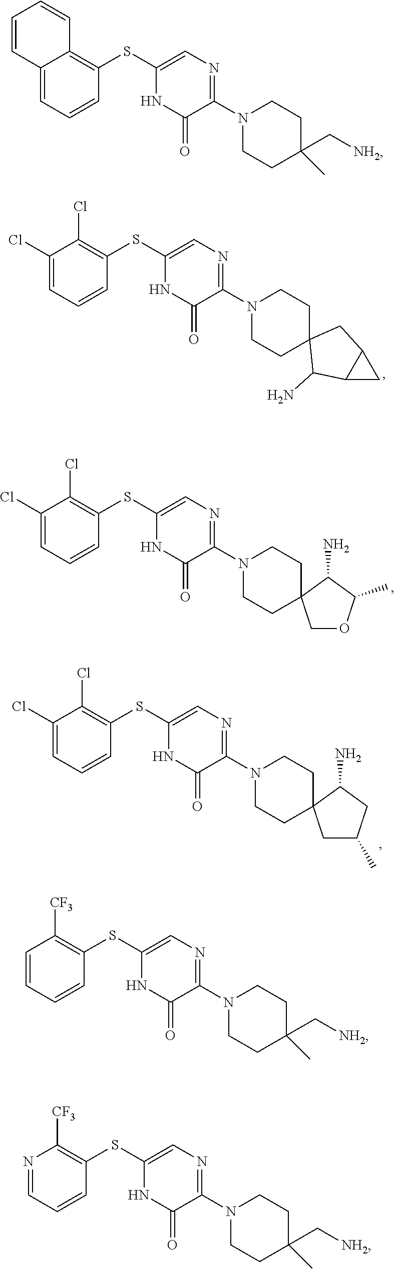 [SMILES: CC1(CN)CCN(c2ncc(Sc3cccc4ccccc34)[nH]c2=O)CC1 ; NC1C2CC2CC12CCN(c1ncc(Sc3cccc(Cl)c3Cl)[nH]c1=O)CC2 ; C[C@@H]1OCC2(CCN(c3ncc(Sc4cccc(Cl)c4Cl)[nH]c3=O)CC2)[C@@H]1N ; C[C@H]1C[C@@H](N)C2(CCN(c3ncc(Sc4cccc(Cl)c4Cl)[nH]c3=O)CC2)C1 ; CC1(CN)CCN(c2ncc(Sc3ccccc3C(F)(F)F)[nH]c2=O)CC1 ; CC1(CN)CCN(c2ncc(Sc3cccnc3C(F)(F)F)[nH]c2=O)CC1]

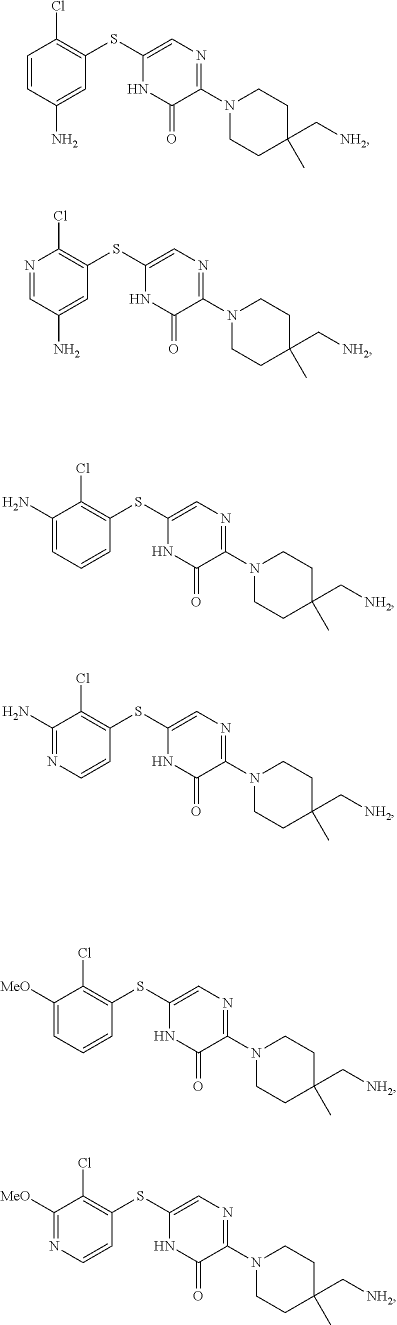 [SMILES: CC1(CN)CCN(c2ncc(Sc3cc(N)ccc3Cl)[nH]c2=O)CC1 ; CC1(CN)CCN(c2ncc(Sc3cc(N)cnc3Cl)[nH]c2=O)CC1 ; CC1(CN)CCN(c2ncc(Sc3cccc(N)c3Cl)[nH]c2=O)CC1 ; CC1(CN)CCN(c2ncc(Sc3ccnc(N)c3Cl)[nH]c2=O)CC1 ; COc1cccc(Sc2cnc(N3CCC(C)(CN)CC3)c(=O)[nH]2)c1Cl ; COc1nccc(Sc2cnc(N3CCC(C)(CN)CC3)c(=O)[nH]2)c1Cl]

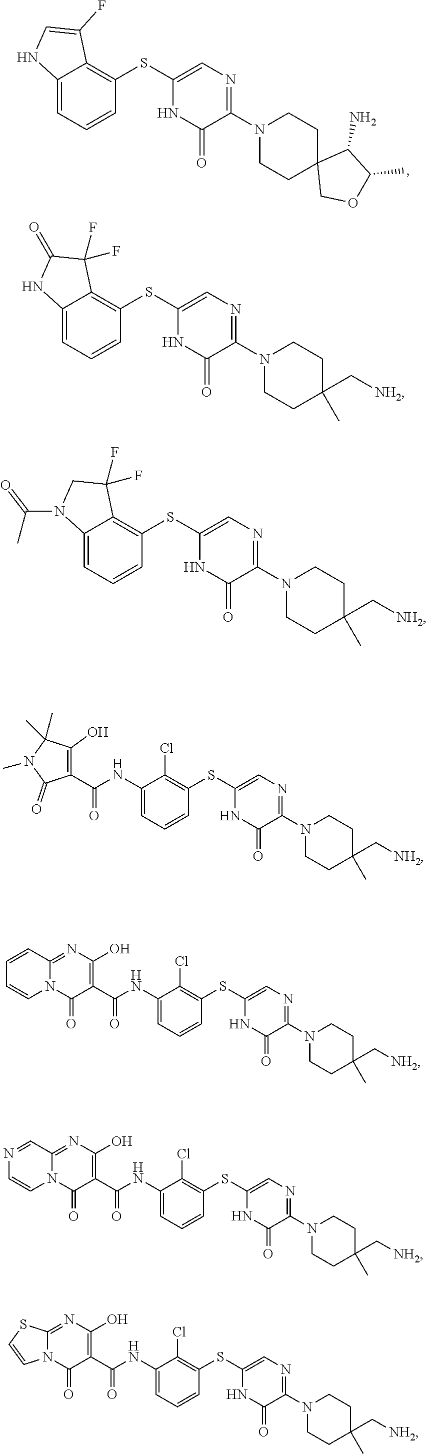

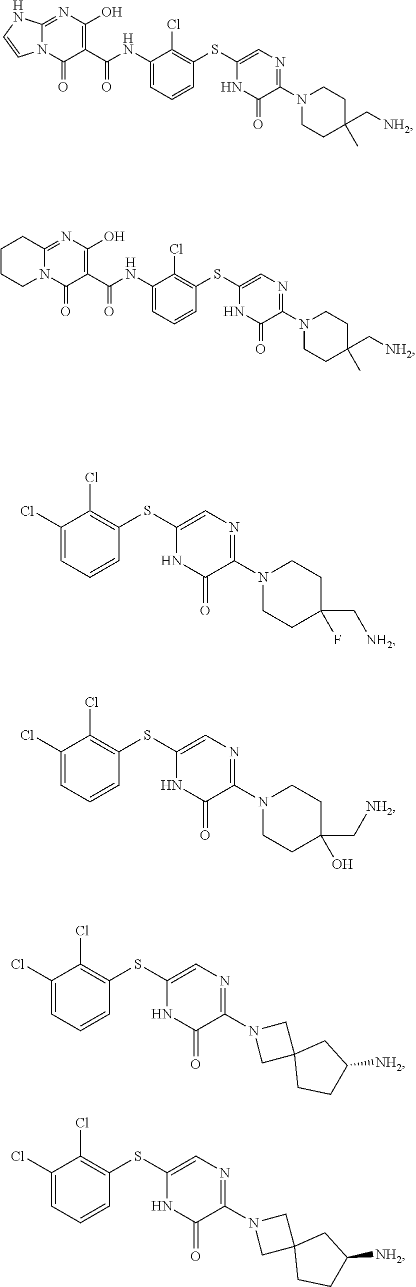 [SMILES: CC1(CN)CCN(c2ncc(Sc3cccc(NC(=O)c4c(O)nc5[nH]ccn5c4=O)c3Cl)[nH]c2=O)CC1 ; CC1(CN)CCN(c2ncc(Sc3cccc(NC(=O)c4c(O)nc5n(c4=O)CCCC5)c3Cl)[nH]c2=O)CC1 ; NCC1(F)CCN(c2ncc(Sc3cccc(Cl)c3Cl)[nH]c2=O)CC1 ; NCC1(O)CCN(c2ncc(Sc3cccc(Cl)c3Cl)[nH]c2=O)CC1 ; N[C@@H]1CCC2(C1)CN(c1ncc(Sc3cccc(Cl)c3Cl)[nH]c1=O)C2 ; N[C@H]1CCC2(C1)CN(c1ncc(Sc3cccc(Cl)c3Cl)[nH]c1=O)C2]

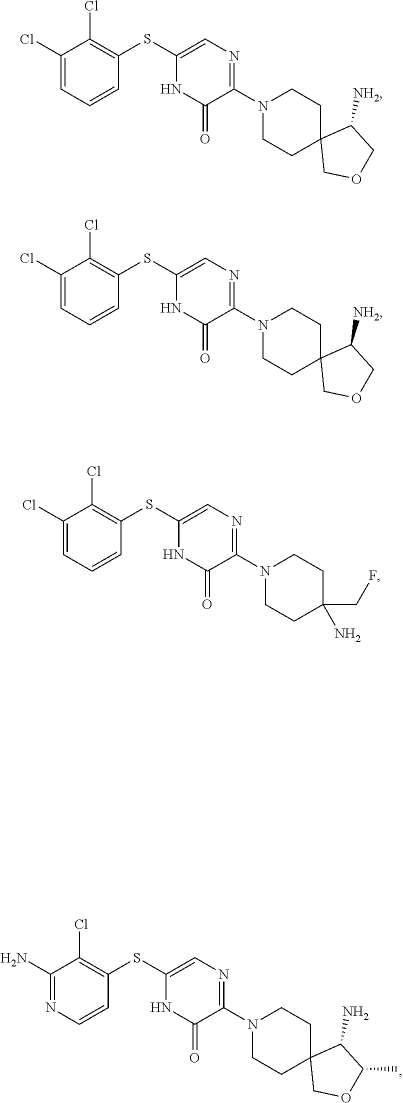 [SMILES: N[C@@H]1COCC12CCN(c1ncc(Sc3cccc(Cl)c3Cl)[nH]c1=O)CC2 ; N[C@H]1COCC12CCN(c1ncc(Sc3cccc(Cl)c3Cl)[nH]c1=O)CC2 ; NC1(CF)CCN(c2ncc(Sc3cccc(Cl)c3Cl)[nH]c2=O)CC1 ; C[C@@H]1OCC2(CCN(c3ncc(Sc4ccnc(N)c4Cl)[nH]c3=O)CC2)[C@@H]1N]

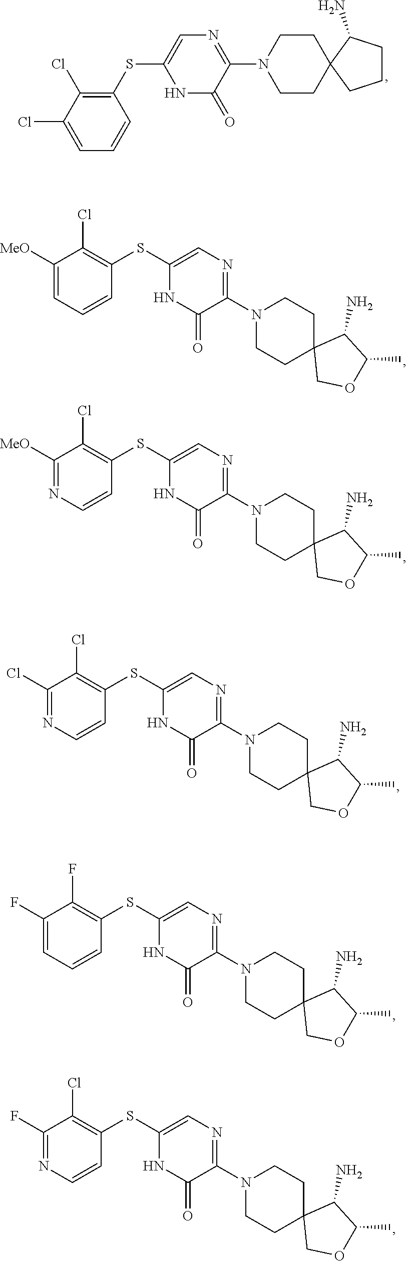 [SMILES: N[C@@H]1CCCC12CCN(c1ncc(Sc3cccc(Cl)c3Cl)[nH]c1=O)CC2 ; COc1cccc(Sc2cnc(N3CCC4(CC3)CO[C@@H](C)[C@H]4N)c(=O)[nH]2)c1Cl ; COc1nccc(Sc2cnc(N3CCC4(CC3)CO[C@@H](C)[C@H]4N)c(=O)[nH]2)c1Cl ; C[C@@H]1OCC2(CCN(c3ncc(Sc4ccnc(Cl)c4Cl)[nH]c3=O)CC2)[C@@H]1N ; C[C@@H]1OCC2(CCN(c3ncc(Sc4cccc(F)c4F)[nH]c3=O)CC2)[C@@H]1N ; C[C@@H]1OCC2(CCN(c3ncc(Sc4ccnc(F)c4Cl)[nH]c3=O)CC2)[C@@H]1N]

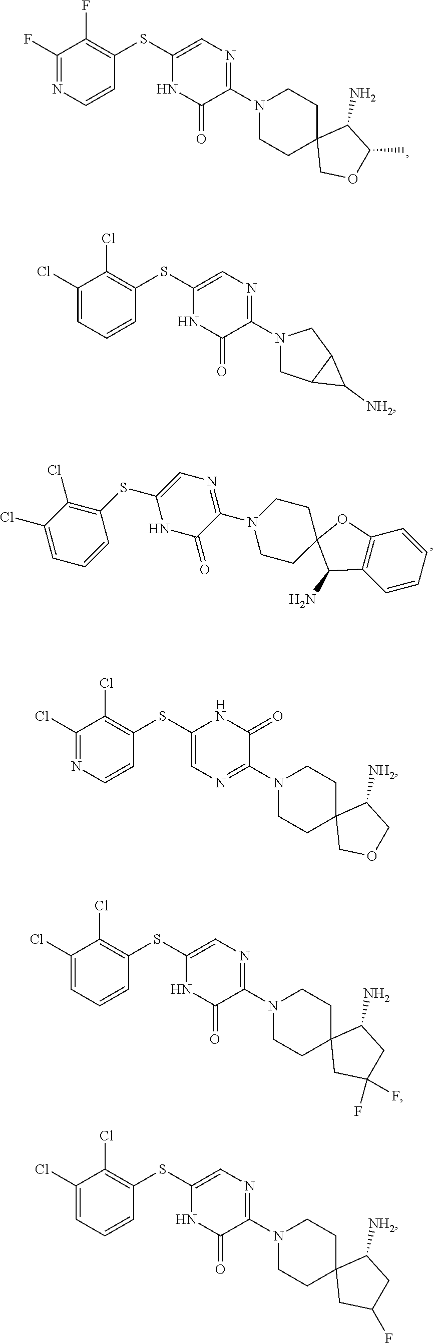 [SMILES: C[C@@H]1OCC2(CCN(c3ncc(Sc4ccnc(F)c4F)[nH]c3=O)CC2)[C@@H]1N ; NC1C2CN(c3ncc(Sc4cccc(Cl)c4Cl)[nH]c3=O)CC12 ; N[C@@H]1c2ccccc2OC12CCN(c1ncc(Sc3cccc(Cl)c3Cl)[nH]c1=O)CC2 ; N[C@@H]1COCC12CCN(c1ncc(Sc3ccnc(Cl)c3Cl)[nH]c1=O)CC2 ; N[C@@H]1CC(F)(F)CC12CCN(c1ncc(Sc3cccc(Cl)c3Cl)[nH]c1=O)CC2 ; N[C@@H]1CC(F)CC12CCN(c1ncc(Sc3cccc(Cl)c3Cl)[nH]c1=O)CC2]

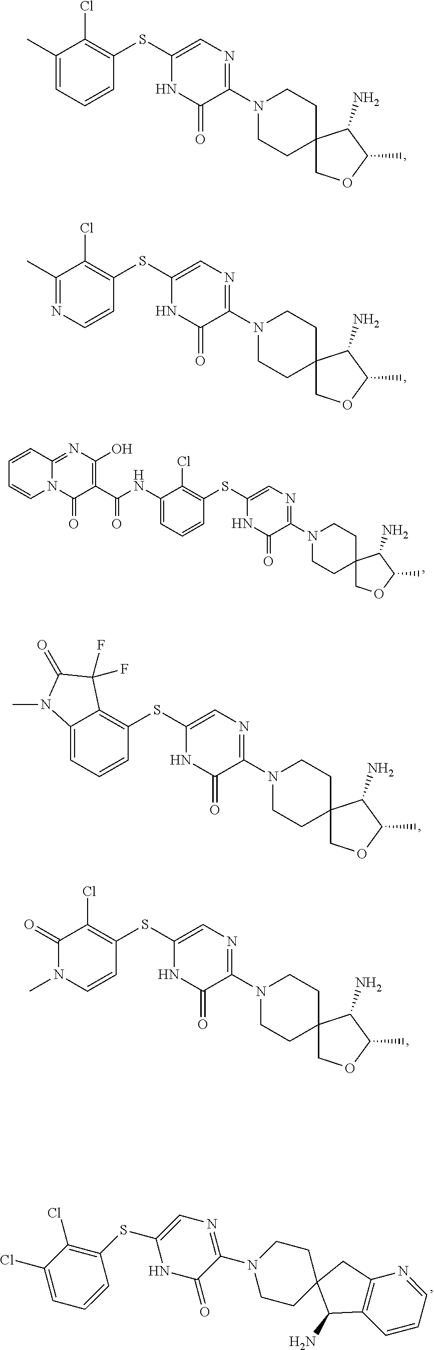

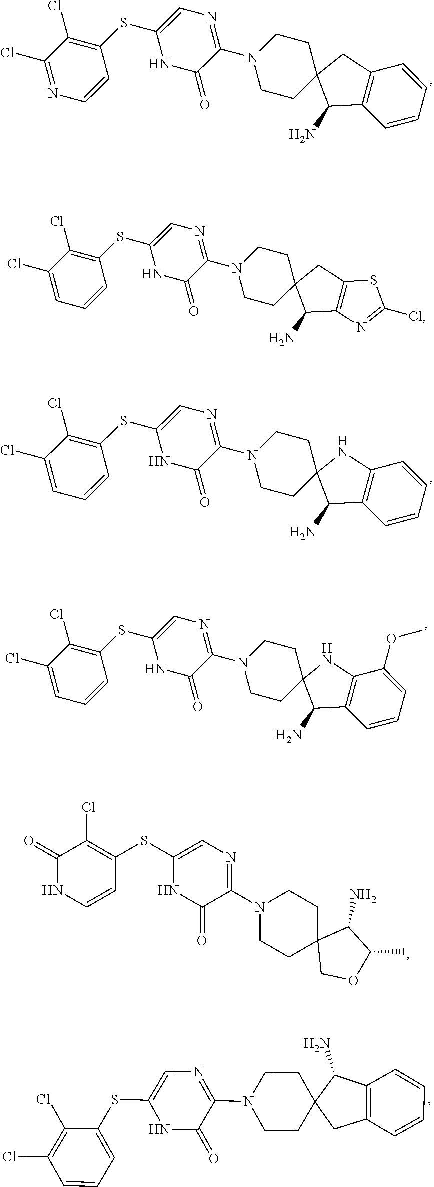 [SMILES: N[C@@H]1c2ccccc2CC12CCN(c1ncc(Sc3ccnc(Cl)c3Cl)[nH]c1=O)CC2 ; N[C@@H]1c2nc(Cl)sc2CC12CCN(c1ncc(Sc3cccc(Cl)c3Cl)[nH]c1=O)CC2 ; N[C@@H]1c2ccccc2NC12CCN(c1ncc(Sc3cccc(Cl)c3Cl)[nH]c1=O)CC2 ; COc1cccc2c1NC1(CCN(c3ncc(Sc4cccc(Cl)c4Cl)[nH]c3=O)CC1)[C@@H]2N ; C[C@@H]1OCC2(CCN(c3ncc(Sc4cc[nH]c(=O)c4Cl)[nH]c3=O)CC2)[C@@H]1N ; N[C@@H]1c2ccccc2CC12CCN(c1ncc(Sc3cccc(Cl)c3Cl)[nH]c1=O)CC2]

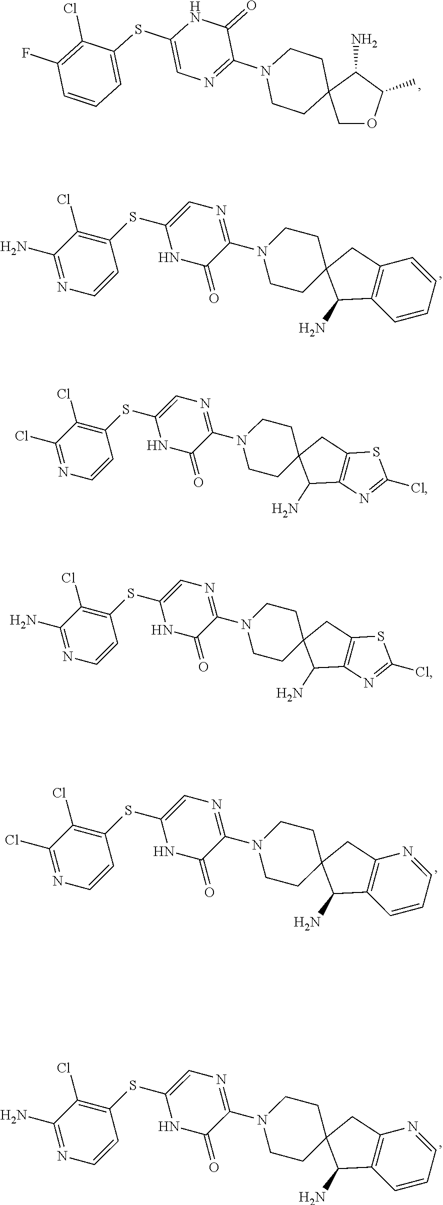 [SMILES: C[C@@H]1OCC2(CCN(c3ncc(Sc4cccc(F)c4Cl)[nH]c3=O)CC2)[C@@H]1N ; Nc1nccc(Sc2cnc(N3CCC4(CC3)Cc3ccccc3[C@H]4N)c(=O)[nH]2)c1Cl ; NC1c2nc(Cl)sc2CC12CCN(c1ncc(Sc3ccnc(Cl)c3Cl)[nH]c1=O)CC2 ; Nc1nccc(Sc2cnc(N3CCC4(CC3)Cc3sc(Cl)nc3C4N)c(=O)[nH]2)c1Cl ; N[C@@H]1c2cccnc2CC12CCN(c1ncc(Sc3ccnc(Cl)c3Cl)[nH]c1=O)CC2 ; Nc1nccc(Sc2cnc(N3CCC4(CC3)Cc3ncccc3[C@H]4N)c(=O)[nH]2)c1Cl]

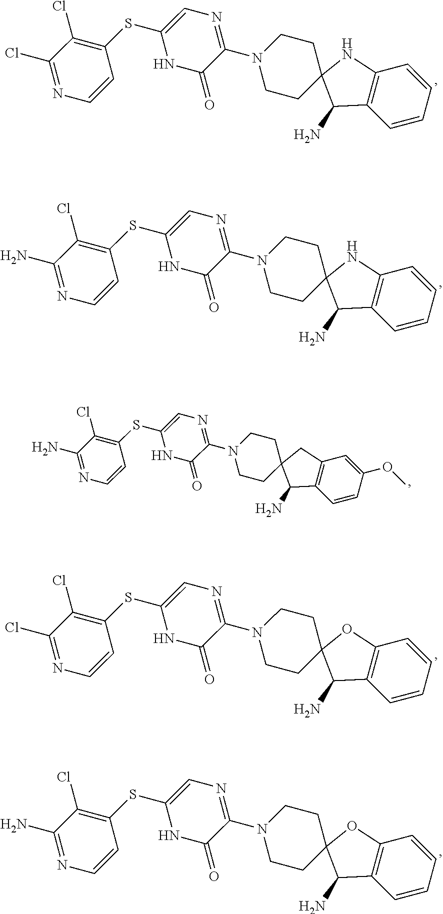 [SMILES: N[C@@H]1c2ccccc2NC12CCN(c1ncc(Sc3ccnc(Cl)c3Cl)[nH]c1=O)CC2 ; Nc1nccc(Sc2cnc(N3CCC4(CC3)Nc3ccccc3[C@H]4N)c(=O)[nH]2)c1Cl ; COc1ccc2c(c1)CC1(CCN(c3ncc(Sc4ccnc(N)c4Cl)[nH]c3=O)CC1)[C@@H]2N ; N[C@@H]1c2ccccc2OC12CCN(c1ncc(Sc3ccnc(Cl)c3Cl)[nH]c1=O)CC2 ; Nc1nccc(Sc2cnc(N3CCC4(CC3)Oc3ccccc3[C@H]4N)c(=O)[nH]2)c1Cl]

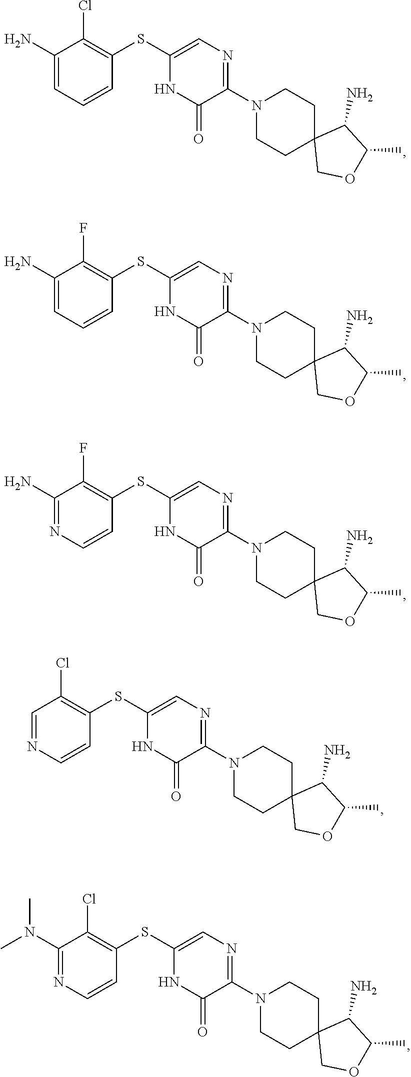

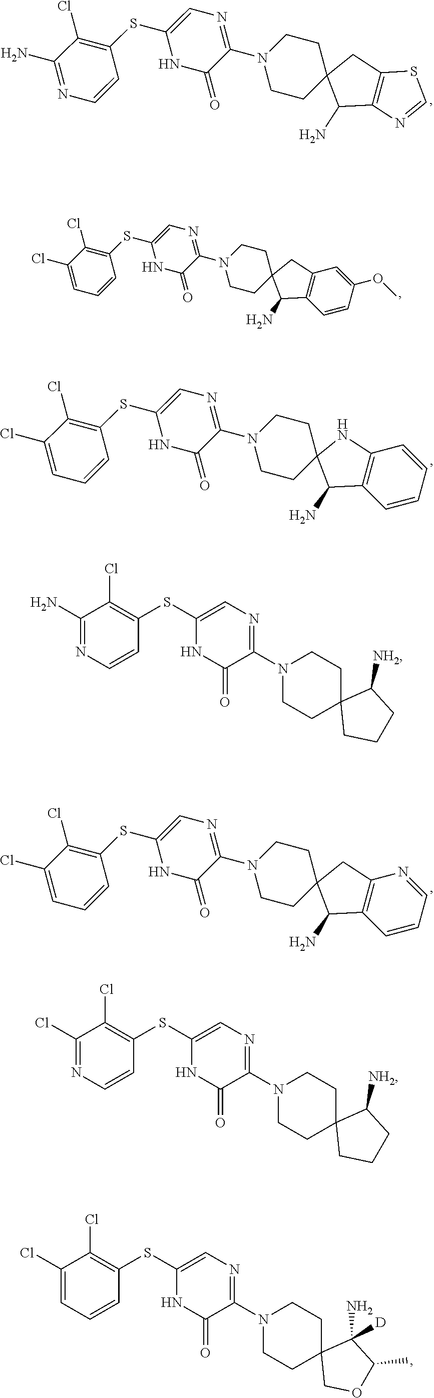

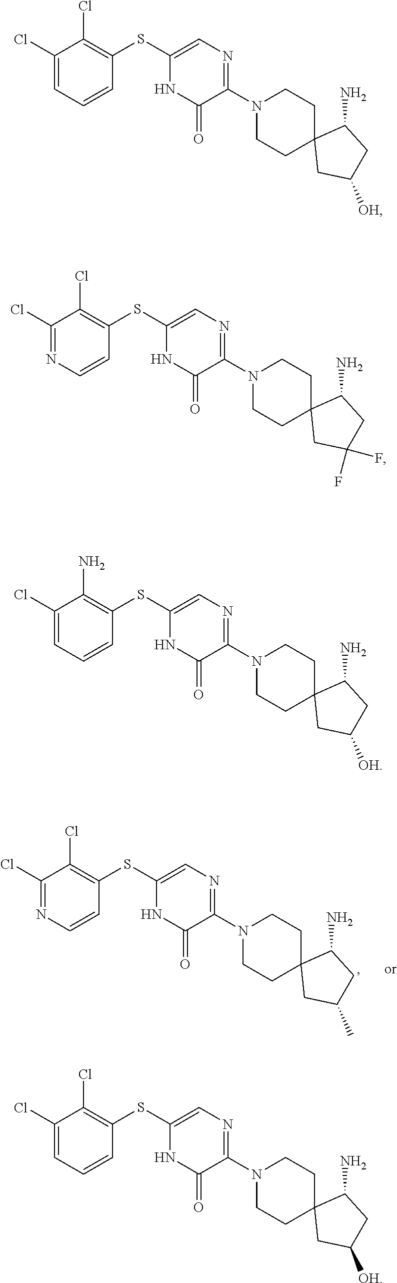 [SMILES: N[C@@H]1C[C@H](O)CC12CCN(c1ncc(Sc3cccc(Cl)c3Cl)[nH]c1=O)CC2 ; N[C@@H]1CC(F)(F)CC12CCN(c1ncc(Sc3ccnc(Cl)c3Cl)[nH]c1=O)CC2 ; Nc1c(Cl)cccc1Sc1cnc(N2CCC3(CC2)C[C@@H](O)C[C@H]3N)c(=O)[nH]1 ; C[C@H]1C[C@@H](N)C2(CCN(c3ncc(Sc4ccnc(Cl)c4Cl)[nH]c3=O)CC2)C1 ; N[C@@H]1C[C@@H](O)CC12CCN(c1ncc(Sc3cccc(Cl)c3Cl)[nH]c1=O)CC2]

15. The method of claim 1, wherein any substituent of the compound has a molecular weight of about 15 g/mol to about 500 g/mol.

16. The method of claim 1, wherein the compound is deuterated.

17. The method of claim 1, wherein the compound or a pharmaceutically acceptable salt thereof is in a dosage form comprising a pharmaceutically acceptable vehicle, diluent, or carrier.

18. A method of treating a disease, a disorder, or a condition associated with the aberrant activity of SHP2, comprising administering a therapeutically effective amount of a compound represented by a formula, 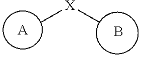
or a pharmaceutically acceptable salt thereof, to a patient in need thereof,
wherein X is S;
wherein Ring A is optionally substituted phenyl, optionally substituted naphthalen-1-yl, optionally substituted pyridin-3-yl, optionally substituted pyridin-4-yl, optionally substituted 2-oxo-1,2-dihydropyridin-4-yl, optionally substituted 1H-indol-4-yl, optionally substituted 2-oxoindolin-4-yl, optionally substituted indolin-4-yl, optionally substituted 3-(2-oxo-2,5-dihydro-1H-pyrrole-3-carboxamido)phenyl, optionally substituted 3-(4-oxo-4H-pyrido[1,2-a]pyrimidine-3-carboxamido)phenyl, optionally substituted 3-(4-oxo-4H-pyrazino[1,2-a]pyrimidine-3-carboxamido)phenyl, optionally substituted 3-(5-oxo-5H-thiazolo[3,2-a]pyrimidine-6-carboxamido)phenyl, optionally substituted 3-(5-oxo-1,5-dihydroimidazo[1,2-a]pyrimidine-6-carboxamido)phenyl, optionally substituted 2-amino-3-chlorophenyl, or optionally substituted 3-(4-oxo-6,7,8,9-tetrahydro-4H-pyrido[1,2-a]pyrimidine-3-carboxamido)phenyl;
wherein Ring B is optionally substituted 6-oxo-5-(piperidin-1-yl)-1,6-dihydropyrazin-2-yl, optionally substituted 6-oxo-5-(pyrrolidin-1-yl)-1,6-dihydropyrazin-2-yl, optionally substituted 5-(hexahydropyrrolo[3,4-c]pyrrol-2(1H)-yl)-6-oxo-1,6-dihydropyrazin-2-yl, optionally substituted 5-(3,6-diazabicyclo[3.2.0]heptan-6-yl)-6-oxo-1,6-dihydropyrazin-2-yl, optionally substituted 6-oxo-5-(2-oxa-8-azaspiro[4.5]decan-8-yl)-1,6-dihydropyrazin-2-yl, optionally substituted 6-oxo-5-(piperidin-4-ylamino)-1,6-dihydropyrazin-2-yl, optionally substituted 6-oxo-5-(spiro[bicyclo[3.1.0]hexane-3,4′-piperidin]-1′-yl)-1,6-dihydropyrazin-2-yl, optionally substituted 6-oxo-5-(8-azaspiro[4.5]decan-8-yl)-1,6-dihydropyrazin-2-yl, optionally substituted 6-oxo-5-(2-azaspiro[3.4]octan-2-yl)-1,6-dihydropyrazin-2-yl, optionally substituted 5-(3-azabicyclo[3.1.0]hexan-3-yl)-6-oxo-1,6-dihydropyrazin-2-yl, optionally substituted 6-oxo-5-(3H-spiro[benzofuran-2,4′-piperidin]-1′-yl)-1,6-dihydropyrazin-2-yl, optionally substituted 5-(5,7-dihydrospiro[cyclopenta[b]pyridine-6,4′-piperidin]-1′-yl)-6-oxo-1,6-dihydropyrazin-2-yl, optionally substituted 5-(1,3-dihydrospiro[indene-2,4′-piperidin]-1′-yl)-6-oxo-1,6-dihydropyrazin-2-yl, optionally substituted 5-(4,6-dihydrospiro[cyclopenta[d]thiazole-5,4′-piperidin]-1′-yl)-6-oxo-1,6-dihydropyrazin-2-yl, optionally substituted 6-oxo-5-(spiro[indoline-2,4′-piperidin]-1′-yl)-1,6-dihydropyrazin-2-yl, optionally substituted 3-(1-amino-5-methoxy-1,3-dihydrospiro[indene-2,4′-piperidin]-1′-yl)-2(1H)-one-6-yl, optionally substituted 3-(4-amino-4,6-dihydrospiro[cyclopenta[d]thiazole-5,4′-piperidin]-1′-yl)-2(1H)-one-6-yl, optionally substituted 3-(1-amino-8-azaspiro[4.5]decan-8-yl)pyrazin-2(1H)-one-6-yl, optionally substituted 3-(4-amino-3-methyl-2-oxa-8-azaspiro[4.5]decan-8-yl-4-d)pyrazin-2(1H)-one-6-yl, optionally substituted 3-(1-amino-3-hydroxy-8-azaspiro[4.5]decan-8-yl)pyrazin-2(1H)-one-6-yl, or optionally substituted 5-((3S,4S)-4-amino-3-methyl-2-oxa-8-azaspiro[4.5]decan-8-yl-4-d)-6-oxo-1,6-dihydropyrazin-2-yl; and
wherein substituted Ring A and substituted Ring B independently have one or more substituents; wherein each substituent of Ring A or Ring B is independently alkyl, alkenyl, alkynyl, —NR^{A}R^{B}, —OR^{A}, —S—R^{A}, aryl, heteroaryl, heterocyclyl, hydroxy, alkoxy, aryloxy, —C(O)—R^{A}, R^{A}—C(O)O— alkylcarboxylate, —SH, cyano, halogen, —C(═S)—R^{A}, —OC(O)—NR^{A}R^{B}, R^{A}—OC(O)—N(R^{A})—, —OC(═S)—NR^{A}R^{B}, R^{A}—OC(═S)—N(R^{A})—, —C(O)NR^{A}R^{B}, R^{A}—C(O)N(R^{A})—, (R^{A}R^{B})N—S(O)₂—, —N(R^{A})—S(O)₂—R^{A}, nitro, R^{A}—S(═O)—, —S(O)₂—R^{A}, haloalkyl, haloalkoxyl, —S(O)₂C(X′)₃ wherein X′ is halogen, —N(R^{A})S(O)₂C(X′)₃ wherein X′ is halogen, amino, —N(R^{A})C(O)-heteroaryl, —N(R^{A})C(O)-heterocyclyl, —C(O)N(R^{A})-heteroaryl-C(O)N(R^{A})-heterocyclyl, or a combination thereof; wherein each substituent has 0-20 carbon atoms and 0-10 heteroatoms, and wherein each heteroatom is independently N, O, S, F, Cl, or Br, provided that the substituent includes at least one C, N, O, S, F, Cl, or Br; and
wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

19. (canceled)

20. The method of claim 1, wherein the disease, the disorder, or the condition comprises lung cancer.

21. The method of claim 1, wherein the disease, the disorder, or the condition comprises non-small cell lung cancer.

22. The method of claim 1, wherein the disease, the disorder, or the condition comprises non-small cell lung cancer with KRAS mutant.

23. The method of claim 1, wherein the disease, the disorder, or the condition comprises esophageal cancer.

24. The method of claim 1, wherein the disease, the disorder, or the condition comprises pancreatic cancer.

25. The method of claim 1, wherein the disease, the disorder, or the condition comprises caecum cancer.

26. The method of claim 1, wherein the disease, the disorder, or the condition is head and neck cancer.

27. The method of claim 1, wherein the disease, the disorder, or the condition is colon cancer.

28. The method of claim 1, wherein the disease, the disorder, or the condition is melanoma.

29. The method of claim 1, wherein the disease, the disorder, or the condition is leukemia.

30. The method of claim 1, wherein the disease, the disorder, or the condition is a metastatic solid tumor.

31. The method of claim 1, wherein administration of a combination of two compounds of claim 1, provides higher efficacy in treating the disease, the disorder, or the condition than each single compound alone in the patient, and wherein the disease, the disorder, or the condition comprises lung cancer, non-small cell lung cancer, non-small cell lung cancer with KRAS mutant, esophageal cancer, pancreatic cancer, or caecum cancer, head and neck cancer, colon cancer, melanoma, leukemia, or other metastatic solid tumors.

32. The method of claim 1, wherein administration of a combination of the compound of claim 1, with other agent, provides higher efficacy in treating the disease, the disorder, or the condition than the compound or the agent alone in the patient, and wherein the disease, the disorder, or the condition comprises lung cancer, non-small cell lung cancer, non-small cell lung cancer with KRAS mutant, esophageal cancer, pancreatic cancer, caecum cancer, head and neck cancer, colon cancer, melanoma, leukemia, or other metastatic solid tumors.
